(19) United States
(12) Patent Application Publication
(10) Pub. No.: US 2024/0034757 A1
(43) Pub. Date: Feb. 1, 2024

(54) TICK CHEMOKINE BINDING PROTEINS FOR USE IN THERAPY AND DIAGNOSIS

(71) Applicant: Oxford University Innovation Limited, Oxford (GB)

(72) Inventor: Shoumo Bhattacharya, Oxford (GB)

(73) Assignee: Oxford University Innovation Limited, Oxford (GB)

(21) Appl. No.: 18/153,944

(22) Filed: Jan. 12, 2023

Related U.S. Application Data
(63) Continuation of application No. 16/784,920, filed on Feb. 7, 2020, now abandoned.
Application No. 16/784,920 is a continuation-in-part of application No. PCT/GB18/52331, filed on Aug. 16, 2018.

(30) Foreign Application Priority Data
Aug. 18, 2017 (GB) 1713284.6

(51) Int. Cl. C07K 14/435; C07K 16/18; G01N 33/68

(52) U.S. Cl. CPC C07K 14/43527; A61K 38/00; G01N 33/6863; C07K 16/18

(57) ABSTRACT

The described invention relates to tick chemokine binding polypeptides (tick CKBPs, typically tick Evasins) including hybrid CKBPs based on sequences from two or more tick CKBPs, and the uses of such polypeptides in inhibition of chemokines or detection of chemokine expression and inflammation.

### CROSS-REFERENCE TO RELATED APPLICATIONS

This application is a continuation of U.S. patent application Ser. No. 16/784,920 (filed Feb. 7, 2020, which is a continuation-in-part of PCT/GB2018/052331 (filed Aug. 16, 2108), which claims the benefit of priority to international application GB1713284.6 (filed Aug. 18, 2017). Each of these applications is incorporated herein by reference in its entirety.

### FIELD OF THE INVENTION

The invention relates to tick chemokine binding polypeptides (tick CKBPs, typically tick Evasins) including hybrid CKBPs based on sequences from two or more tick CKBPs, and the uses of such polypeptides in inhibition of chemokines or detection of chemokine expression and inflammation.

### BACKGROUND OF THE INVENTION

Chemokine-driven inflammation plays a major role in several disorders, including myocardial infarction[1], myocarditis[2], atherosclerotic plaque[3], and stroke[4], pulmonary inflammation and fibrosis, multiple sclerosis, rheumatoid arthritis, psoriasis, atopic dermatitis, inflammatory bowel disease, and cancer (reviewed in [5]).

Chemokines are a group of 45-50 secreted small extracellular proteins, classified as CC, CXC or CX3C based on the arrangement of cysteine residues at the N-terminus, that function via 19 G-protein coupled receptors, to recruit inflammatory and immune cells to injured or diseased tissues[6,7]. Properties of the chemokine network that make it robust to attack are the expression of multiple receptors on inflammatory cells[8], expression of several chemokines in diseased tissues[9], polyvalent chemokine-receptor interactions—with chemokines typically targeting more than one receptor, and receptors typically being activated by more than one chemokine[6], synergistic and cooperative interactions between chemokines[10] and chemokine receptors[11], and feed-forward loops that amplify the network response[12]. The robustness of the chemokine network is clearly demonstrated by the observation that targeting individual chemokines or receptors has failed as a strategy to develop effective therapeutics for inflammatory disorders[9,13].

Both CC and CXC chemokines are important mediators of inflammation in human disease. This is indicated in FIG. 1 with references provided in the description of this Figure below.

A number of pathogens, including viruses, helminths and ticks, produce structurally unrelated chemokine binding proteins (CKBPs) that polyvalently target multiple chemokines disrupting the chemokine network (reviewed in [13]). Viral and helminth CKBPs described to date do not appear to discriminate between CC and CXC chemokines[14,15]. Tick CKBPs identified to date fall into two structurally unrelated classes. These were originally identified from the brown dog tick Rhipicephalus sanguineus by Proudfoot and colleagues[16] as Evasin-1 and Evasin-4 which solely bind a subset of CC chemokines, and Evasin-3 which binds only a subset of CXC chemokines. Additional tick CKBP polypeptides have also been identified, PCT/GB2017/050563, [17,18].

Pre-clinical trials have indicated potential therapeutic efficacy of viral [19], helminth[15] and tick[13,16,20-34] CKBPs in inflammatory disease providing proof-of-concept of polyvalent targeting of the chemokine network as a therapeutic approach for inflammatory disease. Like other CKBPs, the ability of polyvalent tick CKBPs to disrupt the chemokine network provides an advantage over monoclonal antibodies that target single chemokines.

Properties of tick CKBPs which indicate that, like other naturally occurring tick peptides such as COVERSIN®[35], they could be clinically translated include a), systemic anti-chemokine effects following parenteral administration, b), ability to inhibit inflammation in a diverse range of pre-clinical animal models and c), lack of significant immunogenicity or toxicity in such studies[16].

The preferential binding of tick CKBPs to discrete subsets of chemokines (unlike viral CKBPs), could provide a method to precisely target the disease-relevant chemokine network without unnecessarily targeting all chemokines. The inadvertent targeting of chemokines that are not involved in the disease process however could increase the likelihood of off-target effects. Indeed several chemokines may play a beneficial role in the disease process, and targeting these may have undesirable effects. For instance, chemokines such as CCL19, CXCL5 and CXCL12 are known to be atheroprotective [3]. Loss of XCL1 leads to inflammation in the heart and other organs [36], and loss of CXCL10 leads to increased susceptibility to experimental autoimmune encephalitis [37].

There is a need to provide additional CKBPs for use in inhibition and detection of chemokines.

### SUMMARY OF THE INVENTION

The inventors provide CKBPs having previously undescribed chemokine binding properties. The CKBPs are based on sequences from tick salivary polypeptides. The CKBPs may be hybrid polypeptides representing sequences from two (or more) different CKBPs, or polypeptides comprising sequences derived from newly isolated CKBPs binding to CXC chemokines.

The inventors have unexpectedly demonstrated the ability to combine sequences from different tick CKBPs to form a hybrid polypeptide having unique chemokine binding properties. The hybrid polypeptide may combine different chemokine binding properties from two or more tick CKBPs together in a single polypeptide. The hybrid polypeptide may represent a specific chemokine binding activity derived from a first tick CKBP in the context of a second tick CKBP. The flexibility in combination of sequences from different CKBPs identified by the inventors provides the ability to specifically engineer desired chemokine binding properties for a CKBP. This advantageously allows for a CKBP to be matched as precisely as possible to the chemokine expression pattern of a given disease, and/or to avoid targeting of chemokines not involved in that disease. The hybrid polypeptides may also provide both CC and CXC binding functions in a single CKBP, which is not previously described for any tick CKBP polypeptide to the inventors' knowledge, and advantageously caters for the discussion of both CC and CXC chemokines in human disease.

Additionally, the inventors have isolated novel tick CKBP polypeptides with unique CXC binding functions, which are of further utility in provision of CKBPs with novel chemokine binding properties.

The invention therefore provides a hybrid polypeptide comprising an amino acid sequence of a first tick CKBP polypeptide or a variant thereof and an amino acid sequence of a second tick CKBP polypeptide or a variant thereof, wherein said hybrid polypeptide has an altered chemokine binding profile compared to the first or second tick CKBP polypeptide.

The invention further provides a polypeptide comprising (a) all or part of an amino acid sequence shown in any one of SEQ ID NOs 45-72 or (b) all or part of an amino acid sequence having at least 70% homology or identity to a sequence of (a) over its entire length, wherein said polypeptide binds at least one CXC chemokine.

The invention also provides a polypeptide comprising (a) all or part of an amino acid sequence shown in SEQ ID NO: 88, 89, 103 to 109 or (b) all or part of an amino acid sequence having at least 70% homology or identity to a sequence of (a) over its entire length, wherein said polypeptide binds at least one chemokine selected from CCL8, CCL7 and CCL18, preferably wherein said polypeptide binds all said chemokines.

The invention additionally provides a combination of two or more of the above polypeptides of the invention. References to polypeptides of the invention herein include both the hybrid polypeptide and polypeptide described above.

The invention also provides a polynucleotide which encodes a polypeptide of the invention.

The invention additionally provides a combination of two or more polynucleotides each of which encodes a polypeptide of the invention.

The invention further provides a vector which comprises a polynucleotide of the invention or a combination of two or more polynucleotides of the invention.

The invention also provides a host cell which comprises a polynucleotide of the invention, a combination of two or more polynucleotides of the invention or a vector of the invention.

The invention additionally provides a pharmaceutical composition comprising (a) a polypeptide of the invention, a combination of two or more polypeptides of the invention, a polynucleotide of the invention, a vector of the invention or a host cell of the invention and (b) a pharmaceutically acceptable carrier or diluent.

The invention further provides a method of producing a polypeptide of the invention or a combination of two or more polypeptides of the invention comprising, culturing a host cell of the invention under conditions which produce the polypeptide or the combination.

The invention also provides a method of inhibiting the signalling of one or more chemokines in an in vitro culture, the method comprising contacting the culture with a polypeptide of the invention, a combination of two or more polypeptides or polynucleotides of the invention, a polynucleotide of the invention, a vector of the invention or a host cell of the invention.

The invention additionally provides a method of inhibiting the signalling of one or more chemokines in a subject, the method comprising administering to the subject a polypeptide of the invention, a combination of two or more polypeptides or polynucleotides of the invention, a polynucleotide of the invention, a vector of the invention or a host cell of the invention.

The invention further provides a method of treating or preventing in a subject one or more diseases associated with one or more chemokines, the method comprising administering to the subject a polypeptide of the invention, a combination of two or more polypeptides or polynucleotides of the invention, a polynucleotide of the invention, a vector of the invention or a host cell of the invention.

The invention also provides a polypeptide of the invention, a combination of two or more polypeptides or polynucleotides of the invention, a polynucleotide of the invention, a vector of the invention or a host cell of the invention for use in a method of inhibiting the signalling of one or more chemokines in a subject

The invention further provides a polypeptide of the invention, a combination of two or more polypeptides or polynucleotides of the invention, a polynucleotide of the invention, a vector of the invention or a host cell of the invention for use in a method of treating in a subject one or more diseases associated with one or more chemokines.

The invention additionally provides an antibody or a fragment thereof which specifically binds a polypeptide of the invention.

The invention further provides a method of detecting one or more chemokines in a tissue, comprising contacting the tissue with a detectably-labelled polypeptide of the invention or a detectably-labelled combination of two or more polypeptides of the invention and detecting the binding of the polypeptide or the combination to one or more chemokines.

The invention also provides a detectably-labelled polypeptide of the invention or a detectably-labelled combination of two or more polypeptides of the invention for use in a method of detecting one or more chemokines in a tissue.

### DESCRIPTION OF THE FIGURES

FIG. 1. CC and CXC chemokine expression patterns in some human disease states. Filled boxes indicate chemokine expression reported in the literature.

Literature references are as follows: Myocarditis, including giant cell, viral, Chagas and lymphocytic myocarditis: [42-50]; myocardial infarction: [51,52], atherosclerosis: [53-67], vasculitis, including Takayasu disease, ANCA vasculitis, and giant cell arteritis: [68-73], stroke: [4], multiple sclerosis [74-76], Alzheimer's disease [77], primary biliary cirrhosis [78-84], primary sclerosing cholangitis [81,85], liver fibrosis [86,87], nonalcoholic steatohepatitis [88,89], paracetamol liver injury [90], alcoholic liver injury [91], idiopathic pulmonary fibrosis [92-102], acute lung injury [103,104], sarcoidosis [105-111], influenza [112-122], kidney fibrosis [86], inflammatory bowel disease [123-134], pancreatitis [135,136], rheumatoid arthritis [9], psoriasis [137-141], skin fibrosis [86], atopic dermatitis [137,142-147], breast cancer [148-154], colorectal cancer [155-159].

FIG. 2: Alignment of CC chemokine binding tick CKBPs previously disclosed (PCT/GB2017/050563 and [17]). Alignment of tick CKBP sequences that bind CC chemokines.

Alignment was performed using the MUSCLE algorithm in DNASTAR. The mature peptide sequences of Evasin-1 (EVA1_RHISA) and 4 (EVA4_RHISA) are published [20,21]. Other tick CKBP sequences were disclosed previously in PCT/GB2017/050563 and published in Singh et al[17]. Peptide sequence prefix indicates the identity, and suffix indicate the tick species as follows: RHISA and RHIPU—Rhipicephalus sanguineus and pulchellus respectively, and AMBPA, AMBCA, AMBMA, AMBTR—Amblyomma parvum, cajennense, maculatum, triste) respectively. Amino acid residues shaded as black are identical to EVA1_RHISA. Disulfide bonds (DSB) in Evasin-1 are indicated, and were taken from the analysis of the Evasin-1:CCL3 structure 3FPU provided in PDBSum[22]. The positions of the 8 conserved cysteines are indicated by arrows, and are conserved in all CC-chemokine binding tick CKBPs, which we term “8-Cys” tick chemokine binding proteins. The arrangement of Cys residues is C-x(14,17)-C-x(3)-C-x(11,16)-C-x(17,20)-C-x(4)-C-x(4)-C-x(8)-C, with numbers in parentheses indicating spacing between Cys residues. CKBPs in FIG. 2 are EVA1_RHISA (SEQ ID NO: 32), EVA4_RHISA (SEQ ID NO: 34), P467_RHIPU (SEQ ID NO: 1), P546_AMBCA (SEQ ID NO: 2), P672_RHIPU (SEQ ID NO: 3), P974_AMBCA (SEQ ID NO: 6), P983_AMBCA (SEQ ID NO: 7), P985_AMBPA (SEQ ID NO: 8), P991_AMBCA (SEQ ID NO: 9), P1180_AMBTR (SEQ ID NO: 20), P1181_AMBMA (SEQ ID NO: 21), P1182_AMBMA (SEQ ID NO: 22), P1183_AMBTR (SEQ ID NO: 23) and P1243_AMBAM (SEQ ID NO: 29).

FIG. 3: Alignment of CXC chemokine binding tick CKBPs. Alignment of tick CKBP sequences that bind CXC chemokines either using biolayer interferometry or yeast surface display. Alignment was performed using the MUSCLE algorithm in DNASTAR. The sequence of the Evasin-3 mature peptide (EVA3_RHISA) is published [21]. Scale bar at the top indicates amino acid residue positions in EVA3_RHISA. P943_IXORI, P1146_IXORI and P1156_IXORI were disclosed previously in PCT/GB2017/050563. Peptide sequence prefix indicates the identity, and suffix indicate the tick species as follows: IXORI—Ixodes ricinus, AMBCA, Amblyomma cajennense. Amino acid residues shaded as black are identical to EVA3_RHISA. The positions of the 6 conserved cysteines are indicated by arrows, and are conserved in all CXC-chemokine binding tick CKBPs, which we term “6-Cys” tick chemokine binding proteins. The arrangement of Cys residues is C-x(3)-C-x(6,10)-C-x(3,6)-C-x(1)-C-x(10,11)-C, with numbers in parentheses indicating spacing between Cys residues. CKBPs in FIG. 3 are EVA3_RHISA (SEQ ID NO: 3), P458_IXORI (SEQ ID NO: 45), P675_IXORI (SEQ ID NO: 46), P942_IXORI (SEQ ID NO: 47), P943_IXORI (SEQ ID NO: 5), P1074_IXORI (SEQ ID NO: 48), P1077_IXORI (SEQ ID NO: 49), P1078_IXORI (SEQ ID NO: 50), P1080_IXORI (SEQ ID NO: 51), P1086_IXORI (SEQ ID NO: 52), P1090_IXORI (SEQ ID NO: 4653 P1095_IXORI (SEQ ID NO: 54), P1096_IXORI (SEQ ID NO: 55), P1100_IXORI (SEQ ID NO: 56), P1101_IXORI (SEQ ID NO: 57), P1104_IXORI (SEQ ID NO: 58), P1124_IXORI (SEQ ID NO: 59), P1126_AMBCA (SEQ ID NO: 60), P1127_IXORI (SEQ ID NO: 61), P1128_IXORI (SEQ ID NO: 62), P1132_IXORI (SEQ ID NO: 63) P1134_IXORI (SEQ ID NO: 64) P1142_AMBCA (SEQ ID NO: 65), P1146_IXORI (SEQ ID NO: 18), P1156_IXORI (SEQ ID NO: 19), P1162_IXORI (SEQ ID NO: 66), P1166_IXORI (SEQ ID NO: 67), P1168_IXORI (SEQ ID NO: 68), P1170_IXORI (SEQ ID NO: 69), P1172_IXORI (SEQ ID NO: 70), P1174_IXORI (SEQ ID NO: 71) and P1229_IXORI (SEQ ID NO: 72).

FIG. 4: 3D structural models of CC chemokine binding tick CKBPs. 3D structural models were generated using the template Evasin-1: CCL3 structure (3FPU [22]), using MODELLER[160], within PYMOD2.0 [161], with default parameters, after alignment with the MUSCLE algorithm. In each case the tick CKBP is shown in black with the chemokine in grey. Residues that form the predicted interface were calculated using PISA [162], and residues predicted to form hydrogen or salt bridges modelled as sticks, and also shown to the right of each figure. Note that salt bridges were not predicted in models of P991_AMBCA and P984_AMBPA. Structure 1 in each case refers to the tick CKBP and structure 2 to the human chemokine. Note that both N-terminal and C-terminal residues of the tick CKBP may in some instances make contact with the chemokine. A. Model of P672_RHIPU with CCL8. B. Model of P991 AMBCA with CCL3. C. Model of P985 AMBPA with CCL2, D. Model of P1243_AMBAM with CCL13.

FIG. 5: Binding of tick CKBP substitution variant. Hybrid tick CKBP P672:EVA1 (P672_RHIPU (1-44): EVA1RHISA (29-94)) was created by linking P672_RHIPU residues 1 and 44 to EVA1_RHISA (29-94) as they contain most of the predicted interaction surface based on the model generated. A. Biolayer interferometry sensorgram showing P672:EVA1 binding to different doses (ranging from 300 nM to 0.4 nM) of CCL8. Plots display optical thickness (y-axis, nm) versus time (x-axis, seconds). Association (kₒₙ), dissociation (k_{off}), and affinity (K_{d}) constants (reported above) were determined by using the 1:1 binding-model, and global fitting. B. Neutralization of CCL8 induced THP-1 monocyte cell migration by P672:EVA1. Y-axis shows cell count migrating through to the bottom chamber in response to EC₈₀ dose of CCL8. Data (3 technical replicates, 3 biological replicates) are shown as mean±s.e.m. X-axis shows P672:EVA1 concentration (Log₁₀ Molar). IC₅₀ of P672:EVA1 against CCL8 was estimated at 4.8E-8M by fitting an agonist response curve with 4 parameters as described in[17].

FIG. 6: Binding of two-warhead CKBP. A. Arrangement of the two-warhead CKBP expression construct P1243:P1156 (not to scale). P1243_AMBAM was engineered in-frame with a GGGGS (G4S) flexible linker to P1156_IXORI. The construct was tagged at the C-terminus with a StrepII:8×His purification tag. B. Binding affinities (K_{d}, moles/litre) of P1243_AMBAM or P1243:P1156 two-warhead with human CC-chemokines, using biolayer interferometry. - indicates that binding was not detected at 300 nM chemokine concentration. ND indicates not done. C. Binding affinities (K_{d}, moles/litre) of P1243_AMBAM or P1243:P1156 two-warhead with human CXC-chemokines, using biolayer interferometry. - indicates that binding was not detected at 300 nM chemokine concentration. ND indicates not done. D. Neutralization of CCL5 (top panel) or CCL3 (bottom panel) induced THP-1 cell migration by P1243:P1156. Y-axis shows cell count of THP-1 cells migrating through to the bottom chamber in response to EC80 dose of CCL5 or CCL3. Data (3 technical replicates) are shown as mean±s.e.m. X-axis shows P1243:P1156 concentration (Log₁₀ Molar). IC₅₀ of P1243:P1156 was estimated at 6.8E-9M against CCL5 and 7E-9M against CCL3 by fitting an agonist response curve with 4 parameters 154. E. Neutralization of CXCL1 induced granulocyte cell migration by P1243:P1156. Y-axis shows cell count of granulocytes migrating through to the bottom chamber in response to EC₈₀ dose of CXCL1. Data (3 technical replicates) are shown as mean±s.e.m. X-axis shows P1243:P1156 concentration (Log₁₀ Molar). IC₅₀ of P1243:P1156 was estimated at 25.3E-9M against CXCL1 by fitting an agonist response curve with 4 parameters, as described in [17].

FIG. 7. Functional inhibition of CC chemokines by individual and “two-warhead” evasins. FIGS. 7(A), 7(B), and 7(C). Neutralization of CCL5 induced THP-1 cell migration by P1243 (SEQ ID NO: 29), P1243:G4S:P1156 (SEQ ID NO 74), and P1156:G4S:P1243 (SEQ ID NO: 81) respectively. Y-axis shows cell count of THP-1 cells migrating through to the bottom chamber in response to EC₈₀ dose of CCL5. In each case, data from a representative experiment are shown as mean±s.e.m of three technical replicates. X-axis shows CKBP concentration (Log₁₀ Molar). IC₅₀ values (M) indicated in each figure were estimated by fitting an agonist response curve with 4 parameters. FIGS. 7(D), 7(E), and 7(F). Summary IC₅₀ data (mean±s.e.m, and individual data points from three biological replicates) of the indicated CKBPs against CCL5, CCL3 and CCL3L1 respectively. Y-axis shows IC₅₀ (logarithmic scale, M), and x-axis shows each CKBP. CC chemokines were assayed using THIP-1 cell migration. Each chemokine was assayed at its EC₈₀ dose. There were no statistically significant differences between the mean IC₅₀ values in each figure. This data is reported in [163].

FIG. 8. Functional inhibition of CXC chemokines by individual and “two-warhead” evasins. FIGS. 8(A), 8(B), and 8(C). Neutralization of CXCL8 induced granulocyte cell migration by P1156 (SEQ ID NO: 19), P1243:G4S:P1156 (SEQ ID NO: 74) and P1156:G4S:P1243 (SEQ ID NO: 81) respectively. Y-axis shows cell count of granulocytes migrating through to the bottom chamber in response to EC₈₀ dose of CXCL8. Data from a representative experiment are shown as mean±s.e.m of three technical replicates. X-axis shows CKBP concentration (Log₁₀ Molar). IC₅₀ values (M) indicated in each figure were estimated by fitting an agonist response curve with 4 parameters [17]. FIG. (8D). Summary IC₅₀ data (mean±s.e.m, and individual data points from three biological replicates) of the indicated CKBPs against CXCL8. Y-axis shows IC₅₀ (logarithmic scale, M), and x-axis shows each CKBP. CXCL8 was assayed using granulocyte cell migration at its EC₈₀ dose. There were no statistically significant differences between the mean IC₅₀ values. This data is reported in[163].

FIG. 9. Summary data of the two-warhead evasins P991:G4S:P1156 (SEQ ID NO: 73), and P1156:G4S:P991 (SEQ ID NO: 80) binding to human chemokines, compared to their parental evasins, using biolayer interferometry. Binding affinities (K_{d} (M)) and target residence times (RT, minutes) of the ‘two-warheads’ are shown next to their parental evasin P991, which selectively binds only CC chemokines, for comparison. FIG. 9(A). CC-chemokine binding affinities and dissociation half-times of the indicated evasins. FIG. 9(B). CXC-chemokine binding affinities and dissociation half-times of the ‘two-warhead’ against their parental evasin P1156, which selectively binds CXC-chemokines. A dash (−) indicates binding affinity or residence time could not be detected. A (*) indicated the K_{d} could not be determined during this experiment because of difficulty in fitting the data in the software, so previously obtained data has been shown instead.

FIG. 10. Polyvalent binding of CC and CXC chemokines to two-warhead evasins. Biolayer interferometry binding assay showing, P1243:G4S:P1156 (SEQ ID NO: 74, left panel) and P1156:G4S:P1243 (SEQ ID NO: 81, right panel), binding to human CC- and CXC-chemokines. Y-axis shows wavelength shift, X-axis shows time (seconds). ‘Two-warhead’ evasins were first immobilized onto nickel-coated sensors and associated with human CCL5 (association 1). This was followed by association with CCL5+CXCL8 (light grey trace), or with CCL5+CXCL1 (dark grey trace) or CCL5+CCL3 (black trace) (association 2), and then dissociated in buffer (dissociation).

FIG. 11. Polyvalent binding of CC and CXC chemokines to two-warhead evasins. Biolayer interferometry binding assay showing, P991:G4S:P1156 (SEQ ID NO: 73, left panel) and P1156:G4S:P991 (SEQ ID NO: 80, right panel), binding to human CC- and CXC-chemokines. Y-axis shows wavelength shift, X-axis shows time (seconds). ‘Two-warhead’ evasins were first immobilized onto nickel-coated sensors and associated with human CCL5 (association 1). This was followed by association with CCL5+CXCL8 (light grey trace), or with CCL5+CXCL1 (dark grey trace) or CCL5+CCL2 (black trace) (association 2), and then dissociated in buffer (dissociation).

FIG. 12. Summary data of chemokine binding using biolayer interferometry by the indicated CXC chemokine binding evasins. Binding affinities (K_{d}, M) of immobilized purified evasins to human CXC-chemokines using biolayer interferometry. Chemokines and evasins are arranged by sequence-similarity based phylogeny. “ELR+” CXC chemokines are indicated. These contain a characteristic Glu-Leu-Arg motif in the N-terminal region that binds receptors CXCR1 and CXCR2 and activates neutrophil migration. A dash (−) indicates that binding was not detected at 300 nM chemokine concentration. An asterisk following a chemokine indicates that it was used for yeast surface display screening. Data for EVA3_RHISA (evasin 3) are shown for comparison. Evasin functional classes I and II are indicated.

FIG. 13. Functional inhibition of CXC chemokines by P1142_AMBCA (P1142) FIG. 13(A). Neutralization of mouse CXCL10 induced activated mouse T cell migration by P1142. Y-axis shows cell count of activated mouse T cells migrating through to the bottom chamber in response to an EC₈₀ dose of CXCL10. X-axis shows P1142 concentration (Log₁₀ Molar). Data from a representative experiment, with each data point being the mean of three technical replicates, is shown. FIG. 13(B) and 13(C). Neutralization of mouse CXCL1 or CXCL2 induced mouse bone marrow granulocyte cell migration by P1142. Y-axis shows cell count of mouse bone marrow granulocyte cells migrating through to the bottom chamber in response to an EC₈₀ dose of CXCL1 or CXCL2. X-axis shows P1142 concentration (Log₁₀ Molar). Data show representative experiments, with each data point being the mean of two technical replicates. FIG. 13(D). Summary IC₅₀ data (mean±s.e.m), and individual data points from four biological replicates (CXCL10) or three biological replicates (CXCL1 or CXCL2)) of P1142 against CXCL10, CXCL1 and CXCL2 respectively. Y-axis shows IC₅₀ (logarithmic scale, M), and x-axis shows each chemokine. CXCL10 was assayed using activated mouse T cell migration and CXCL1 and CXCL2 were assayed using mouse bone marrow (granulocyte) cell migration. Each chemokine was assayed at its EC₈₀ dose. IC₅₀ values were estimated by fitting an agonist response curve with 4 parameters and are 3.37±0.44 nM for CXCL10, 2.15±0.93 nM for CXCL1, and 0.66±0.11 nM for CXCL2.

FIG. 14. Characterisation of P672_PEP-FITC by fluorescence polarisation. P672_PEP consists of a mutant version of P672_RHIPU residues E17 to F32 (EDEDYEDFFKPVTAYF, SEQ ID NO: 88, peptide BK1.1). Residue C30 was mutated to A to avoid an unpaired cysteine residue. The residues corresponding to the above peptide are wholly contained within the CCL8-binding region of P672_RHIPU transferred in the hybrid evasin P672:EVA1 (SEQ ID 76, see FIG. 5). The peptide was used in experiments as a N-terminally FITC labelled and C-terminally amidated peptide P672_PEP-FITC, FITC-NH-EDEDYEDFFKPVTAYF (SEQ ID NO: 90).

FIG. 14(A). Fluorescence polarisation assay showing binding of CCL8 to P672_PEP-FITC. The x-axis shows CCL8 concentration, and Y-axis the resulting fluorescence anisotropy. The curve was fitted to the two site-total and non-specific binding model in GraphPad Prism 6. Data are shown as mean and SEM of three independent experiments, with each independent experiment performed as three technical replicates. The affinity constant K_{d} was calculated in GraphPad Prism from the curve fit with the standard error reported.

FIG. 14(B). Fluorescence polarisation assay showing binding of chemokines to P672_PEP-FITC. The measured fluorescence anisotropy (y-axis) of P672_PEP-FITC (50 nM) following binding to a fixed dose (1 μM)) of CCL7, CCL8, CCL18 and CXCL1 (negative control). Three independent data points are displayed for each chemokine, and mean and SEM are shown. Statistical significance was determined using one-way ANOVA with Sidak's correction for multiple comparisons using Graph Pad Prism 6. **=p<0.005, ****=P<0.00005, ns=not significant.

FIG. 15: Displacement of P672_PEP-FITC from chemokines by P672_PEP (BK1.1)

The following peptides were used in this experiment:

P672_PEP (C-terminally amidated) =
EDEDYEDFFKPVTAYF (SEQ ID NO 88, peptide BK1.1),
P672_PEP-FITC (C-terminally amidated) = FITC-NH-
EDEDYEDFFKPVTAYF (SEQ ID NO 90)
and
P672_PEP_SCRAM (C-terminally amidated) =
EFTEVYEFDFKYDAPD (SEQ ID NO 91).

FIG. 15(A). Displacement of P672_PEP-FITC from CCL7 by P672_PEP. FIG. 15(B). Displacement of P672_PEP-FITC from CCL8 by P672 PEP. FIG. 15(C). Displacement of P672_PEP-FITC from CCL18 by P672_PEP. In each case 100 μM (++) or 50 μM (+) of P672_PEP was incubated with P672_PEP-FITC (50 nM)/chemokine (1 μM CCL7 and CCL18; 500 nM CCL8) and the resulting anisotropy measured. A peptide with the P672_PEP sequence scrambled (100 μM) served as a negative control (P672_PEP_SCRAM; EFTEVYEFDFKYDAPD). Experiment carried out in triplicate where each data point is the average of one experiment carried out in technical triplicate. Mean with SEM shown and statistical significance was determined using one-way ANOVA with Sidak's correction for multiple comparisons using Graph Pad Prism 6. *=p<0.05, ***=p<0.0005, ****=P<0.00005, ns=not significant.

FIG. 16: Cell based characterisation of P672_PEP (BK1.1)

The following peptide was used in this experiment:

P672_PEP (C-terminally amidated) =
EDEDYEDFFKPVTAYF (SEQ ID NO 88, peptide BK1.1),
P672_PEP_SCRAM (C-terminally amidated) =
EFTEVYEFDFKYDAPD

FIG. 16(A). Effect of P672_PEP (peptide BK1.1) on CCL8-647 binding to THP-1 cells. Y-axis shows median fluorescence intensity (MFI). 60 μM (+) P672_PEP or P672_PEP_SCRAM were incubated with CCL8-647 (2.5 nM) for half an hour at 37° C. prior to adding to THP-1 cells and incubating everything together for a further half an hour at 37° C. Cells were then analysed using fluorescence assisted cell sorting (10,000 cells analysed) and MFI recorded. Experiment carried out in triplicate where each data point is the average of one experiment carried out in technical triplicate. Mean with SEM shown and statistical significance was determined using one-way ANOVA with Sidak's correction for multiple comparisons using Graph Pad Prism 6. ****=P<0.00005, ns=not significant.

FIG. 16(B). Representative IC₅₀ curve obtained when titrating in increasing amount of P672_PEP (peptide BK 1.1) with CCL8-647. Y-axis shows MFI, x-axis shows log[P672_PEP]M. IC₅₀ value is the mean of three independent experiments carried out in triplicate with SEM shown.

FIG. 16(C). Effect of P672_PEP (peptide BK1.1) on CCL7 migration in THP-1 cells. Y-axis shows cell count. CCL7 was incubated with 200 μM (+++), 100 μM (++) or 50 μM (+) P672_PEP or 200 μM (+++) P672_PEP_SCRAM and the resulting THP-1 migration after four hours was determined. Experiment carried out in triplicate where each data point is the average of one experiment carried out in technical triplicate. Mean with SEM shown and statistical significance was determined using one-way ANOVA with Sidak's correction for multiple comparisons using Graph Pad Prism 6. *=p<0.05, **=p<0.0005, ns=not significant. FIG. 16(D). Representative IC50 curve obtained for CCL8 migration in response to titrating in increasing concentrations of P672_PEP. Y-axis shows cell count, x axis shows log[P672_PEP)M. Experiment carried out in triplicate with mean and SEM shown. IC50 is the average of three independent experiments carried out in triplicate with error as SEM.

FIG. 17: Characterization of CCL8/P672 interface by HDX-MS

A. Surface representation (top) and ribbon diagram (bottom) of a homology model of P672 (darker grey) and CCL8 (lighter grey) complex. P672 and CCL8 in 1:1 ratio was pre-incubated for 1 h, then diluted in D₂O containing buffer and quenched at different time intervals (5 s, 30 s, 5 min, 60 min).

B. Surface representation in light grey (top) and ribbon diagram (bottom) of P672 and CCL8 complex at the time points indicated. Residues with statistically significant increased HDX rates, (exposed residues) are shown in darkest grey. Regions with statistically significant decreased HDX rates (protected residues) are shown in mid-greys for P672 and CCL8. All analyses were performed in triplicate.

C. Surface representations in light grey (top) and ribbon diagrams (bottom) of P672 (−90° rotated view along the y-axis of B). Residues protected at 5 s and 30 s time points (E22-F32) are indicated in mid-grey in the top panel. Exposed residues (G87-C94) are indicated in darkest grey in the bottom panel. The surface of the protected residues (E22-F32) is also shown in the bottom panel.

D. Surface representation in light grey (top) and ribbon diagram (bottom) of CCL8, with residues protected at all time points (R18-S27) indicated in mid-grey. Disulfide bonds are indicated in lightest grey in the bottom panel. The surface of the N-loop (residues C12-R24) is also shown in the bottom panel to show the overlap with protected residues.

E. Spectra of two representative peptides from the Y21-F32 region in P672 (mid-grey and black bars) that are protected from deuterium uptake upon complex formation. H/D exchange mass spectra was measured at t=5 s. These peptides display reduced relative deuterium uptake upon complex formation. Other peptides from this region are indicated as gray bars. Mass spectra is shown for control non-deuterated peptides (c-i, iv), unbound P672 deuterated peptides (c-ii, v), and P672 deuterated peptides when in complex with CCL8 (c-iii, vi).

FIG. 18: Design and biophysical analysis of a EVA1/P672 hybrid protein

A. Alignment of EVA1 (SEQ ID NO: 32), P672 (SEQ ID NO: 3) and EVA1/P672 (EVA1 containing P672E22-E32; SEQ ID NO: 95) hybrid protein using MUSCLE algorithm. Amino acids are color-coded according to physicochemical properties: aromatic (F, W, and Y); acidic (D and E); basic (R, H, and K); nonpolar aliphatic (A, G, I, L, M, P, and V); polar neutral (C, N, Q, and T). Amino acids that were protected from deuterium uptake in P672 are indicated with the right black box. The N-terminal acidic region is enclosed in the left black box.

B. Biolayer interferometry sensorgram obtained when either P672, EVA1/P672 or EVA1 is loaded onto the BLI sensor and exposed to 600 nM CCL8. Plots display wavelength shift (Y-axis, nm) versus time (X axis, seconds).

C. Biolayer interferometry sensorgram for EVA1(P672_{E22-E32}) hybrid binding to CCL8. Dotted lines indicate collected data, solid lines indicate modelled data. Plots display wavelength shift (Y-axis, nm) versus time (X axis, seconds).

FIG. 19: Development and biophysical analysis of P672-derived peptides

A. Design of a P672 peptide tiling array to identify CCL8-binding peptides. Positions of each residue within P672 are indicated, and the gray box indicates the CCL8 binding region identified by HDX-MS. P672 residues are shaded according to CCL8 binding affinity from the Ala-scanning mutagenesis (see text and Table 9). Y21A, E22A, F25A, P27A, V28A, and Y31A mutants lead to either complete or highly significant loss of activity (P<0.0001), D18A and F32A mutants lead to moderately significant loss of activity (P<0.05). Peptides synthesized (BK1.1-BK8) are indicated as gray bars.

B. Fluorescent peptides BK1.1-BK6 (50 nM) were incubated with CCL8 (1 μM) and the resulting anisotropy determined. A scrambled peptide (S, SCR_{FITC}) was used as a negative control. The anisotropy of each peptide after being incubated with CCL8 was compared to scrambled peptide using one-way ANOVA with Sidak's correction for multiple comparisons. **** indicates P≤0.0001.

C. Fluorescent polarization assay to determine binding of BK1.1_{FITC} to CCL8. The Y axis shows anisotropy, and X axis the dose of CCL8. Individual data points are indicated and the curve was generated as a non-linear fit with 3 parameters to estimate K_{D}.

D. Fluorescent polarization assay to assess effect of alanine-scanning mutagenesis of BK1.1_{FITC} on CCL8 binding. K_{D} values for each BK1.1_{FITC} Ala mutant are shown as mean±s.e.m of three biological replicates, which are individually indicated as points. Data for each mutant was compared to wild-type (WT) BK1.1, using a one-way ANOVA with Sidak's correction for multiple comparisons. ****=P≤0.0001, *=P≤0.05. The mutant P27A showed no detectable binding.

E. Mass spectrometry (MS) to assess effect of BK1.1 on CCL8 Top panel: Native MS of CCL8 homodimer. Mid panel: In-solution dissociation of CCL8 dimer and further binding of CCL8 to one and two BK-1. Confirmation of CCL8/BK-1 complex by HCD gas-phase dissociation of isolated precursor ions: Bottom panel, left: 2217 m/z corresponding to CCL8/BK-1 (1:1) and Bottom panel, left: 2555 m/z corresponding to CCL8/BK-1 (1:2). Buffers contained up to 0.5% DMSO. All analyses were performed in triplicate.

F. Fluorescent polarization assay to assess the binding of BK1.1_{FITC} against a CC-chemokine panel. Data are presented as mean±s.e.m of three biological replicates, which are individually indicated as points. Each biological replicate was performed as technical duplicate. CXCL1 was used as a negative control. CC-chemokine binding compared to the negative control using a one-way ANOVA with Sidak's correction for multiple comparisons. ****=P≤0.0001, * P<0.05.

G-I. Fluorescence polarization competition assay for BK1.1_{FITC} and CC-chemokine interactions. BK1.1_{FITC} (50 nM) was incubated with the indicated chemokine (1 μM) with or without unlabeled BK1.1 or SCR (BK1.1 scrambled) peptides (50 μM) for 30 min and the resulting anisotropy was measured. Data are presented as mean±s.e.m of three biological replicates, which are individually indicated as points. Each biological replicate was performed as technical duplicate. Statistical significance of differences (SCR versus BK1.1) were calculated using a one-way ANOVA. ****=P≤0.0001, ***=P≤0.001.

FIG. 20. Development and biophysical analysis of the BK1.1 peptide series

A. Sequences of peptides studied, with disulfide bond (BK1.3) or thioether cyclization (BK1.2, BK1.4) indicated by lines. SCR is a scrambled peptide based on the sequence of BK1.1 (SEQ ID NO: 88). Y21F32, C30A (SEQ ID NO: 103); Y21F32 (SEQ ID NO: 104); BK1.2 (SEQ ID NO: 105); BK1.3 (SEQ ID NO: 106); BK1.4 (SEQ ID NO: 107); SCR (SEQ ID NO: 91).

B-D. Effect of indicated peptides at a concentration of 100 μM on a His-tagged P672-biotinylated CCL8, CCL2 or CCL3 interaction respectively using an AlphaScreen assay. In each panel. Y axis shows intensity counts, and X axis the peptide. Data are presented as mean±s.e.m. of three independent experiments, shown as individual data points. Statistically significant differences (compared to chemokine+P672), using a one-way ANOVA with Sidak's multiple comparisons test are indicated by asterisks. ****=P≤0.0001, ***=P≤0.001, **=P≤0.01.

E-G. Representative dose-response AlphaScreen assay curves showing disruption of His-tagged P672 interactions with biotinylated human CCL8, CCL2 and CCL3 respectively by each member of the BK1.1 derived series. Y axis shows intensity counts, and X axis the peptide concentration (Log₁₀ Molar). Data are shown as mean of two technical replicates. Curves were fitted with 4 parameters to estimate IC₅₀.

H-J. Summary IC₅₀ values for inhibition of His-tagged P672 binding to human CCL8, CCL2 and CCL3 respectively by each member of the BK1.1 derived series, where these could be calculated. Y axis shows IC₅₀ (M). Data are presented as mean±s.e.m. of three independent experiments, each shown as individual data points. Each independent experiment was conducted as two technical replicates. Statistically significant differences (compared to BK1.1), using a one-way ANOVA with Sidak's multiple comparisons test, are indicated by black asterisks. Statistically significant differences (pairwise comparisons of BK1.2, BK1.3, BK1.4 and BK1.5) using one-way ANOVA with Tukey's multiple comparisons test are indicated with blue asterisks (comparisons to BK1.2), or green asterisks (comparisons to BK1.3). ***=P≤0.001, **=P≤0.01, *=P≤0.05.

FIG. 21. Cell-based assessment of P672-derived peptide activity.

A-D. Inhibition of human chemokine induced THP-1 cell migration by BK1.1, BK1.2, BK1.3, SCR (BK1.1 scrambled, negative control) peptides, each at 10 μM, and by P672 protein (positive control, 300 nM). Y axis in each panel shows % migration of THP-1 cells normalized to chemokine alone which was set at 100%. All experiments were performed at EC₈₀ doses of CCL8 (5.8 nM), CCL7 (7.2 nM), CCL3 (3.5 nM), and CCL2 (1.2 nM), respectively. Data are shown as mean±s.e.m. of three independent biological replicates, shown as individual data points. Each biological experiment was performed as three technical replicates. Statistically significant differences (compared to SCR), using a one-way ANOVA with Sidak's correction for multiple comparisons, are indicated by asterisks: ****=P≤0.0001, ***=P≤0.001, **=P≤0.01, *=P≤0.05.

E. Representative dose-response curves showing inhibition of human CCL8 induced THP-1 cell migration by BK1.1 (black), BK1.2 (darker grey), BK1.3 (lighter grey) peptides and by P672 protein (positive control, magenta). Y axis shows % migration of THP-1 cells normalized to CCL8 alone which was set at 100%. Data are shown as mean±s.e.m. of three technical replicates. X-axis shows inhibitor concentration (Log₁₀ Molar). Curves were fitted with 4 parameters to estimate IC₅₀.

F. Summary IC₅₀ values for inhibition of human CCL8 induced THP-1 cell migration by BK1.1, BK1.2, BK1.3, and P672 protein. Y axis shows IC₅₀ (M). Data are shown as mean s.e.m. of three biological replicates. Statistically significant differences (compared to BK1.1) using a one-way ANOVA with Sidak's correction for multiple comparisons, are indicated by asterisks: ****=P≤0.0001, ***=P≤0.001, **=P≤0.01, *=P≤0.05.

G. I. Representative dose-response curves showing inhibition of human CCL8-647 (g) and human CCL2-647 (i) induced THP-1 cell fluorescence by BK1.1 (black), BK1.2 (darker grey), BK1.3 (lighter grey), SCR (scrambled, negative control, light-grey without line) peptides and by P672 protein (positive control, mid grey). Y axis shows fluorescence (arbitrary units). Data are shown as mean of two technical replicates. X axis shows inhibitor concentration (Log₁₀ Molar). Curves were fitted with 4 parameters to estimate IC₅₀.

H. J. Summary IC₅₀ values for inhibition of human CCL8-647 (h) or CCL2-647 (i) induced THIP-1 cell fluorescence by BK1.1, BK1.2, BK1.3, and P672 protein. Y axis shows IC₅₀ (M). Data are shown as mean±s.e.m. of three biological replicates, shown as individual data points. Each biological experiment was conducted as two technical replicates. Statistically significant differences (compared to BK1.1) using a one-way ANOVA with Sidak's correction for multiple comparisons, are indicated by asterisks: ****=P≤0.0001, ***=P≤0.001, **=P≤0.01, *=P≤0.05.

FIG. 22: Chemokine expression in the zymosan air-pouch model

A. Experimental design to characterize the zymosan air-pouch model. A dorsal air-pouch (a.p.) was created by subcutaneous (s.c.) injection of air on day 0 and day 3. Zymosan or PBS (control) was injected into the air-pouch (a.p.) on day 6. Air-pouch exudate was collected and analysed at 4 and at 24 hours by a membrane assay.

B. Top: Images of membranes used to analyse air-pouch fluid chemokines at different time points and conditions. See example 19 for details. Bottom: arrangement of chemokines, positive (PC) and negative (NC) controls.

C. Chemokine expression relative to the positive control which was set at 100, at different time points and conditions (mean of two spots. Indicated in shades of grey) and fold change (FC) in chemokine expression at 4 and 24 hours compared to PBS control (indicated in shades of grey).

FIG. 23: Assessment of anti-inflammatory activity of locally or systemically administered peptide in a mouse dorsal air-pouch model.

A. Experimental design to assess efficacy of locally administered peptide. A dorsal air-pouch (a.p.) was created by subcutaneous (s.c.) injection of air on day 0 and day 3. Zymosan or PBS (control) was injected into the air-pouch (a.p.) on day 6. Peptide or protein (blue) was injected into the air-pouch on day 6 at the time of zymosan injection and repeated 9 hours later. Air-pouch exudate was collected and analysed on day 7 by flow cytometry (FC). Nine mice were studied in each of 5 study arms: PBS alone (PBS), zymosan (zymo), zymosan+scrambled peptide (SCR), zymosan+P672 (P672), and zymosan+BK1.3 (BK1.3).

B-F. Summary data for flow cytometry analysis for locally administered peptide. Y axis shows cell counts of total leucocytes (B), neutrophils (C), eosinophils (D), monocytes (E) and T-cells (F). Data are presented for each arm as mean±s.e.m. and with individual data points. Statistically significant differences (compared to zymosan) using a one-way ANOVA with Dunnett's correction for multiple comparisons, are indicated by asterisks: ****=P≤0.0001, ***=P≤0.001, **=P≤0.01, *=P≤0.05.

G. Experimental design to assess efficacy of intraperitoneally administered peptide. This is identical to that used for locally administered peptide (above) except that peptide or protein was administered intraperitoneally (i.p.). Nine mice were studied in each of 3 study arms: zymosan+SCR, zymosan+P672 (P672), and zymosan+BK1.3 (BK1.3).

H-L. Summary data for flow cytometry analysis for intraperitoneally administered peptide. Y axis shows cell counts of total leucocytes (H), neutrophils (I), eosinophils (J), monocytes (K) and T-cells (L). Data are presented for each arm as mean±s.e.m. and with individual data points. Statistically significant differences (compared to SCR) using a one-way ANOVA with Dunnett's correction for multiple comparisons, are indicated by asterisks: ****=P≤0.0001, ***=P≤0.001, **=P≤0.01, *=P≤0.05.

FIG. 24. Sequence coverage obtained for peptide-level HDX-MS experiments.

A. CCL8 protein (1-97)(SEQ ID NO: 110) aligned against the 44 peptides generated by peptic digestion of CCL8, yielding 96.9% coverage, and 8.0 redundancy (calculated as the average number of peptides in which each residue is found.

B. P672 protein (1-130)(SEQ ID NO: 111) aligned against the 38 peptides generated by peptic digestion of P672, yielding 100% sequence coverage, and 4.95 redundancy.

These peptides passed the identification criteria outlined example 19, and were used for further H/D exchange measurements. Adequate overlap is observed, except for CCL8 P2-N14 and A40-C52 regions for which only one peptide was identified.

FIG. 25. Relative fractional deuterium uptake at different time points for the P672:CCL8 complex.

Percentage of H/D exchange measured at four incubation time points (5 s, 30 s, 5 min, 60 min). Regions with increased HDX rates (exposed residues) are shown in green and regions with decreased HDX rates (protected residues) are shown as darker shades. Regions with no significant exchange are indicated as lighter grey. Protein tags are highlighted in grey box on sequence. All analyses were performed in triplicate. CCL8 (SEQ ID NO: 112); P672 (SEQ ID NO: 111).

FIG. 26. MALDI-MS analysis of BK1.3.

A. Mass spectrum of 20 μM BK1.3 after incubation for 4 h in RPMI media at 37° C. mimicking cell assay. Note that RPMI contains a mix of amino acids including Cys-SH (200 μM). The mass M-S—S-Cys is that of the peptide bonded to a cysteine.

B. Mass spectrum of 20 μM BK1.3 after incubation for 1 h in AlphaScreen buffer (50 mM HEPES, 150 mM NaCl, 0.1% BSA, 0.01% Tween20, pH7.5) at room temperature mimicking AlphaScreen assay.

FIG. 27. Gating strategy for analysing air pouch exudate

Representative flow cytometry data of zymosan air-pouch exudate from different arms of peptide in vivo efficacy experiments. Panel series from the top to bottom show scrambled (SCR) peptide, isotype control, SCR, P672 protein, BK1.3 peptide (see FIG. 23 for details). Panel series from left to right show all cells, single cells, CD45+ cells, Ly6C/Ly6G cells, CD3+ cells and SigF+ cells for each of the treatments. Boxed areas in each dataset represent the cell type. Arrows indicate the serial gating strategy used to identify different cell populations. For the identification of isolated immune cell populations, cells were stained with fluorophore-conjugated antibodies to specific extracellular marker proteins CD45, Ly6G, Ly6C, CD3, and Siglec F. Cell debris was excluded, and remaining cells included, using a forward scatter area (FSC-A) versus side scatter area (SSC-A) gate (All cells). Single cells were then selected on a FSC-A versus FSC-height plot (Single cells) to exclude signalling data from doublets. Leucocytes were gated from single cell populations of specific CD45 staining (CD45+ cells). Monocytes and neutrophils were gated from CD45+ cells population of Ly6C specific staining (Ly6+ cells) and Ly6G specific staining (Ly6G+ cells) respectively. T-cells were gated from CD45+ cells population of CD3 specific staining (CD3+ cells), and eosinophils were gated from Ly6G− cells population of Siglec F specific staining (SigF+ cells).

### DESCRIPTION OF THE SEQUENCE LISTING

SEQ ID NOs: 1 to 72 are shown in Tables 1, 4 and 5 below and in the electronic sequence listing.

SEQ ID NOs: 73 to 109 are shown above and in the Detailed Description and electronic sequence listing.

### DETAILED DESCRIPTION OF THE INVENTION

It is to be understood that different applications of the disclosed products and methods may be tailored to the specific needs in the art. It is also to be understood that the terminology used herein is for the purpose of describing particular embodiments of the invention only, and is not intended to be limiting.

In addition as used in this specification and the appended claims, the singular forms “a”, “an”, and “the” include plural referents unless the content clearly dictates otherwise. Thus, for example, reference to “a polypeptide” includes two or more such polypeptides, or reference to “a polynucleotide” includes two or more such polynucleotides and the like.

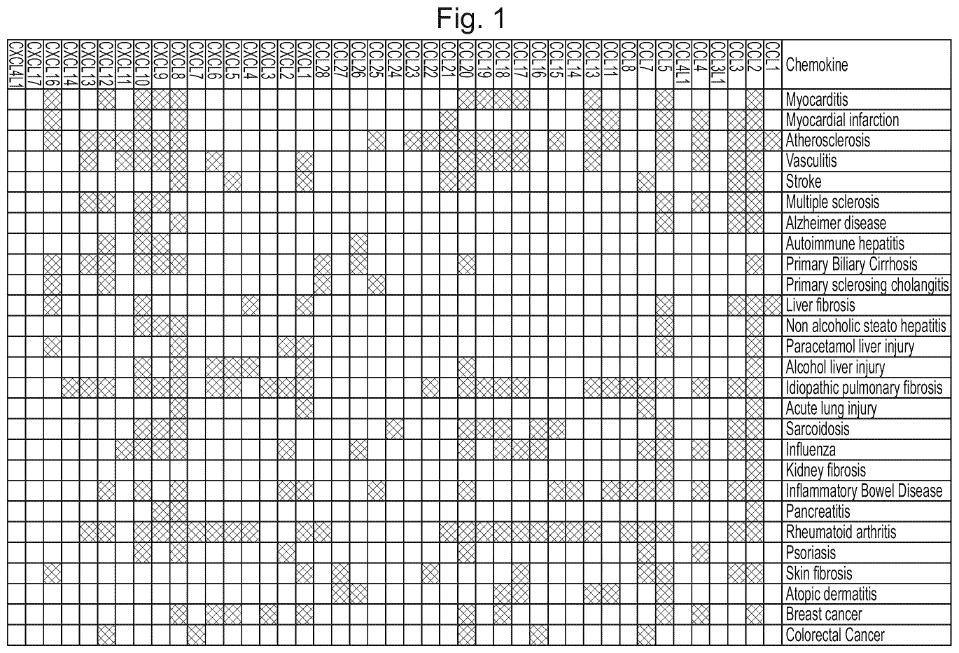

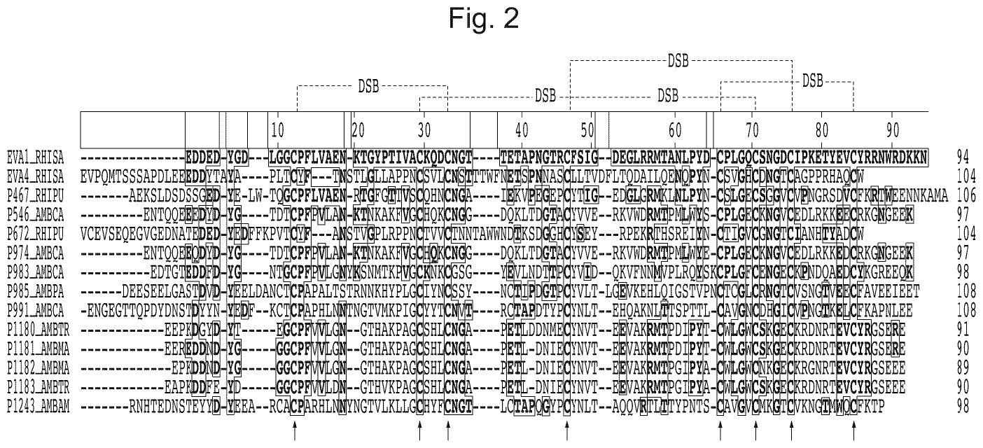

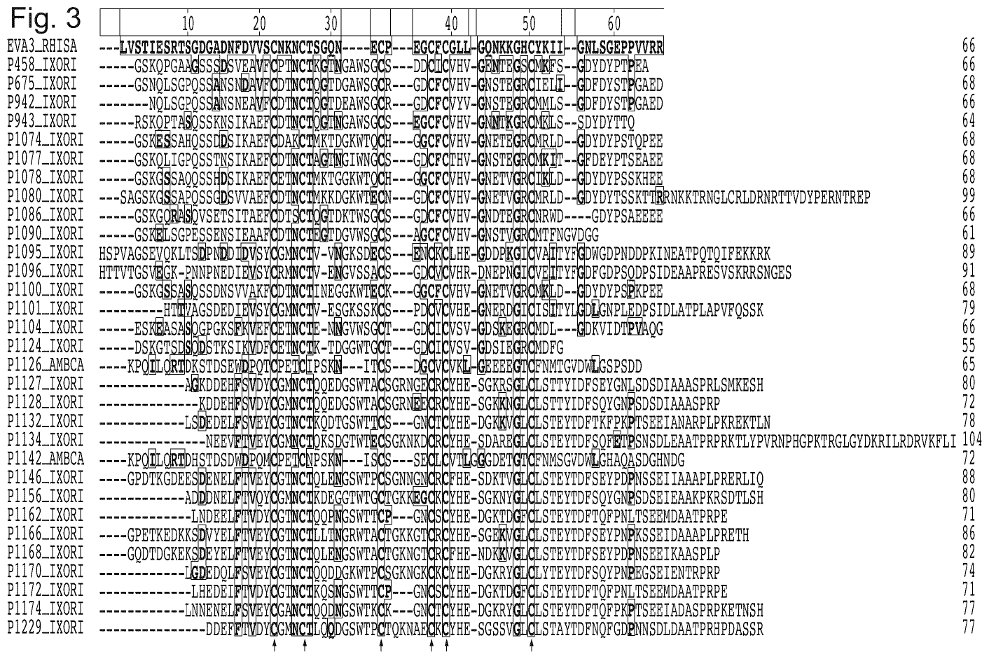

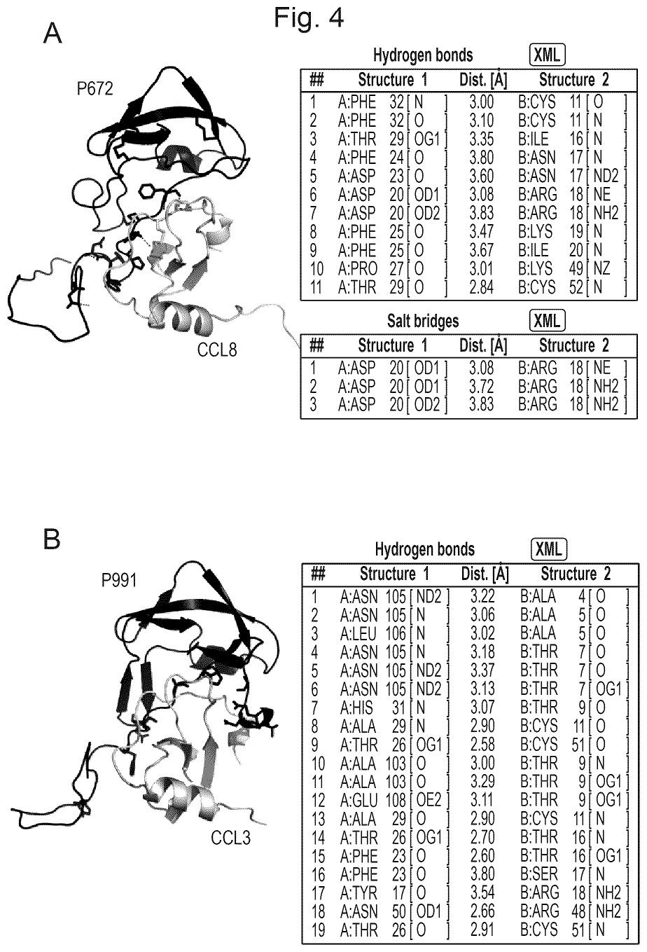

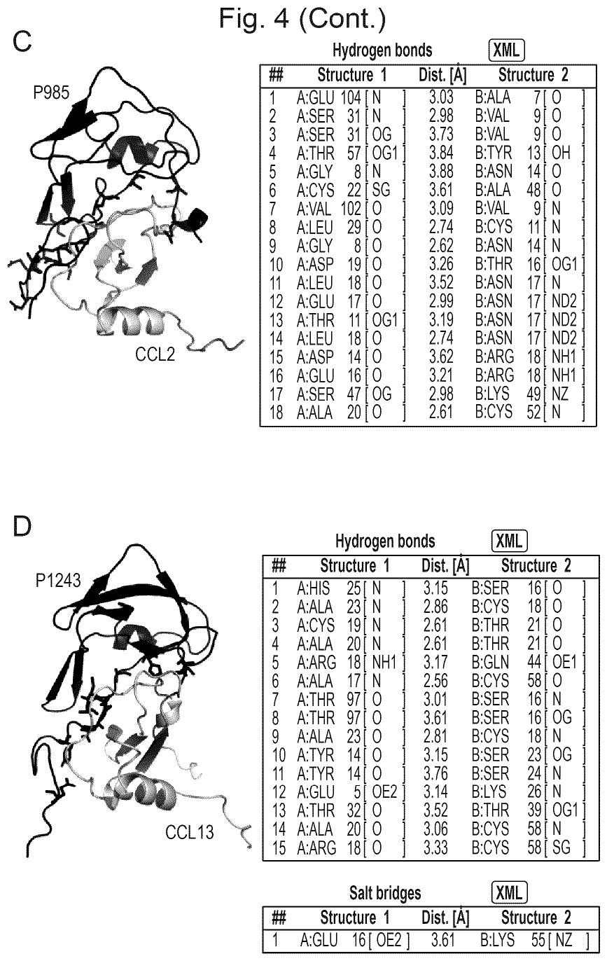

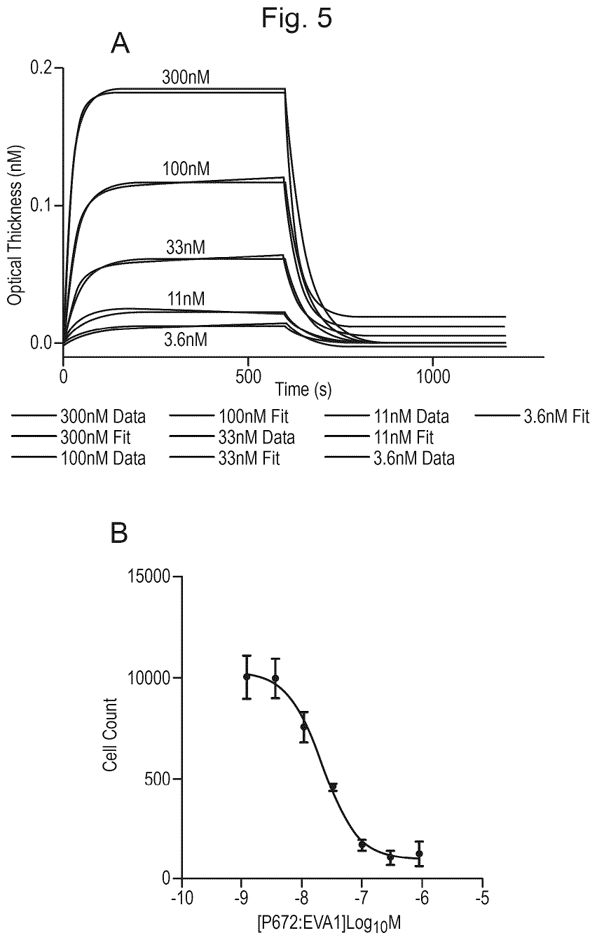

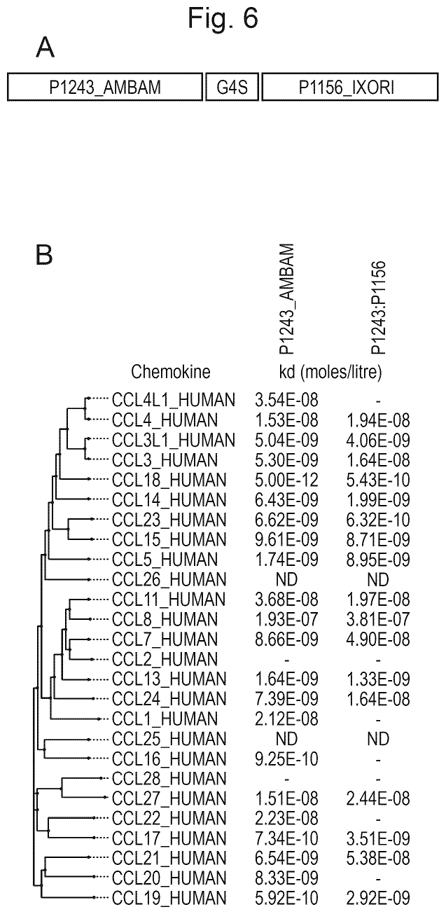

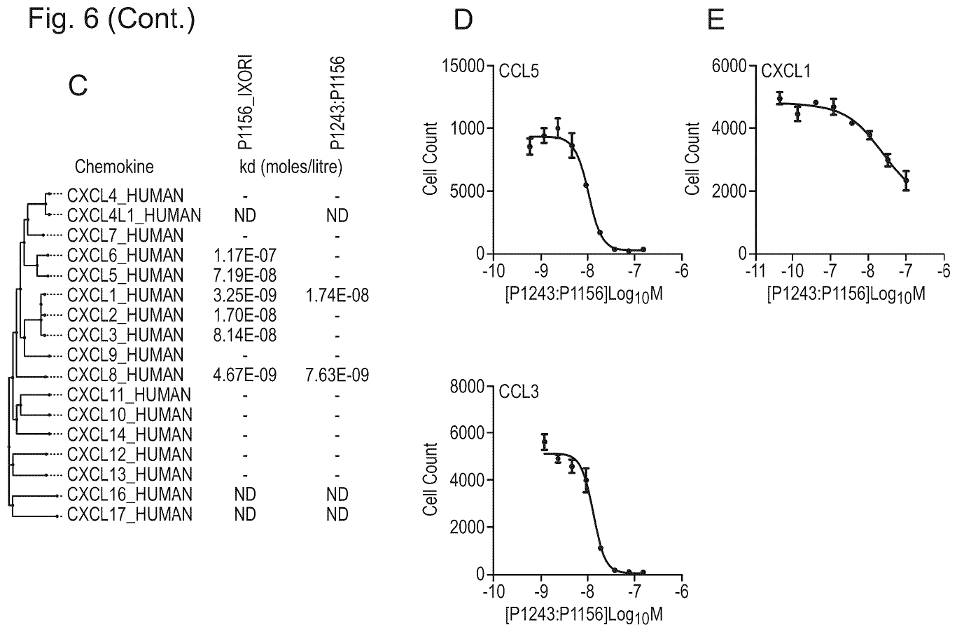

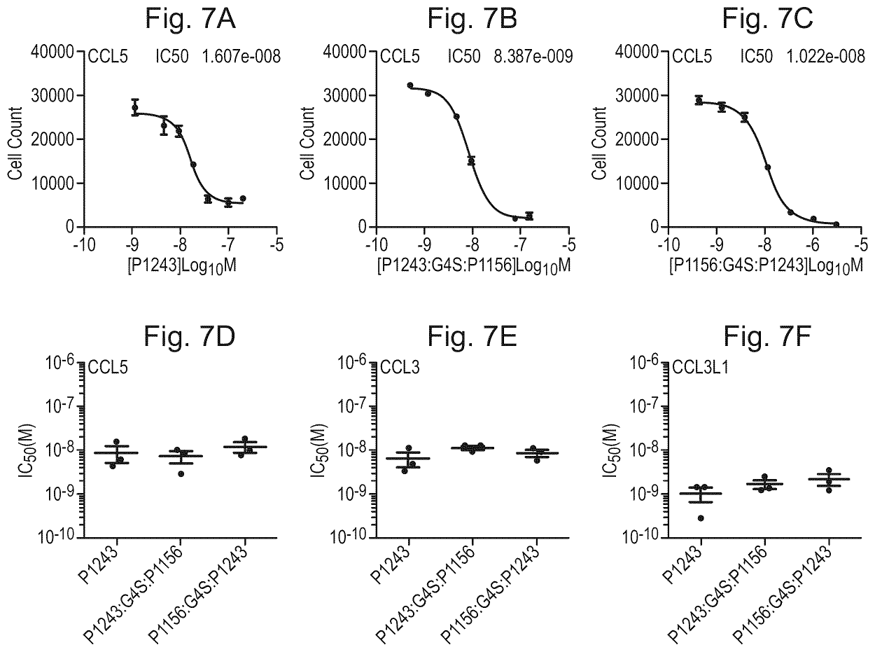

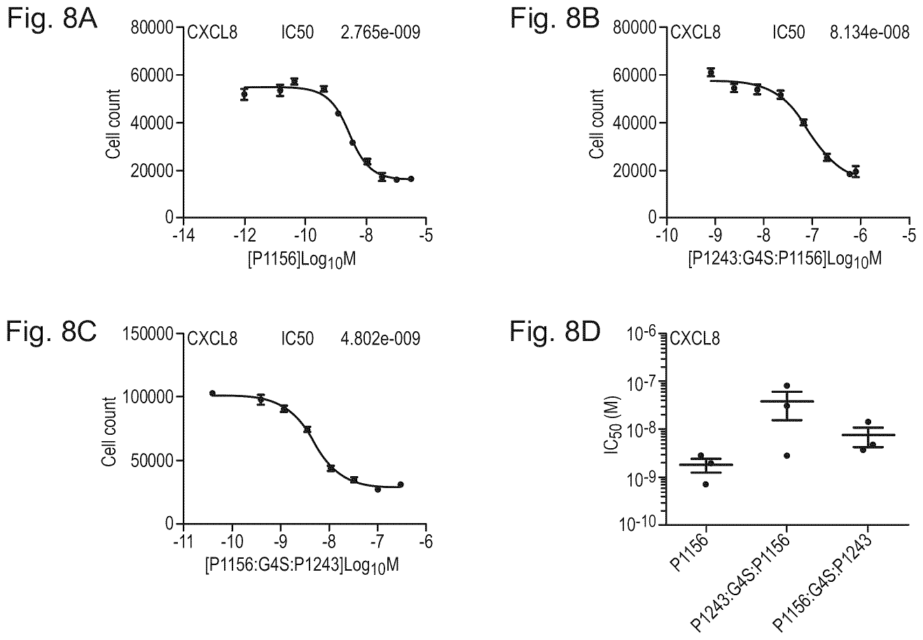

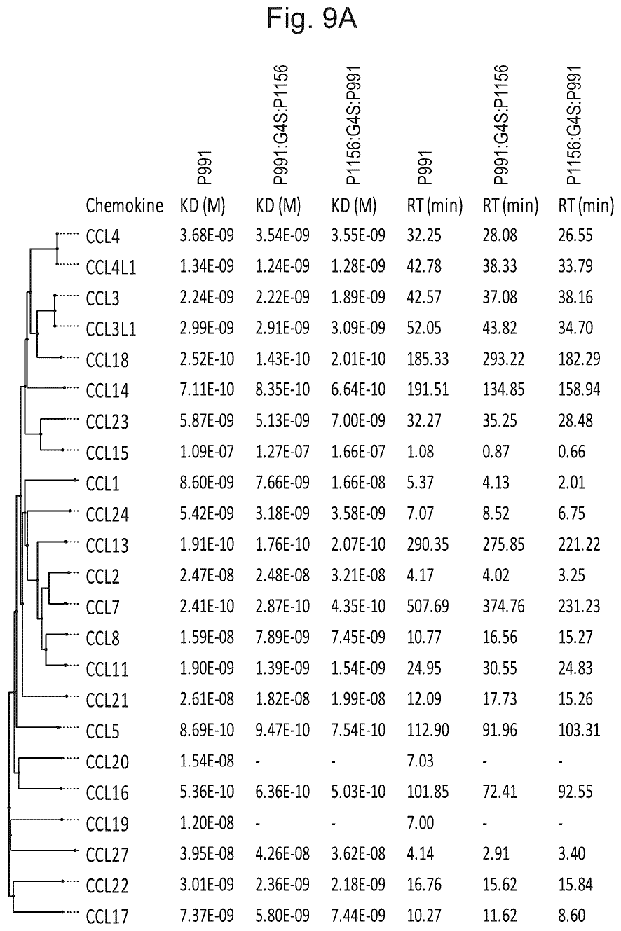

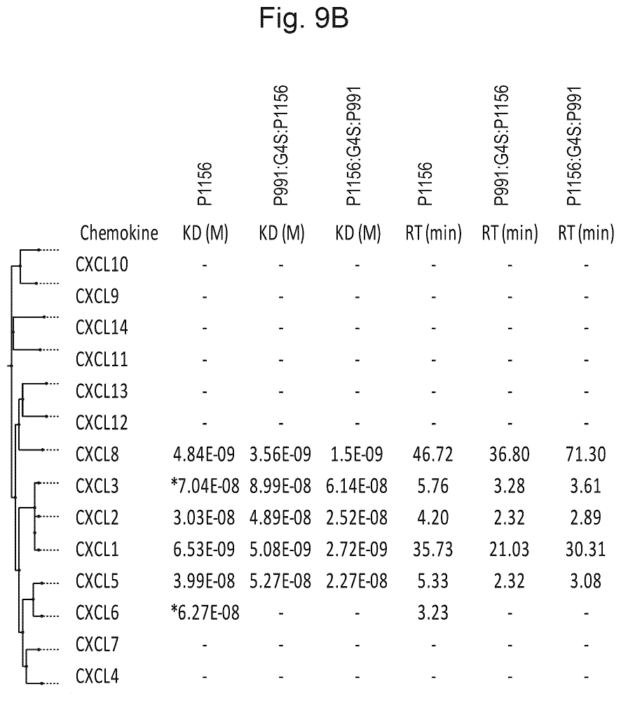

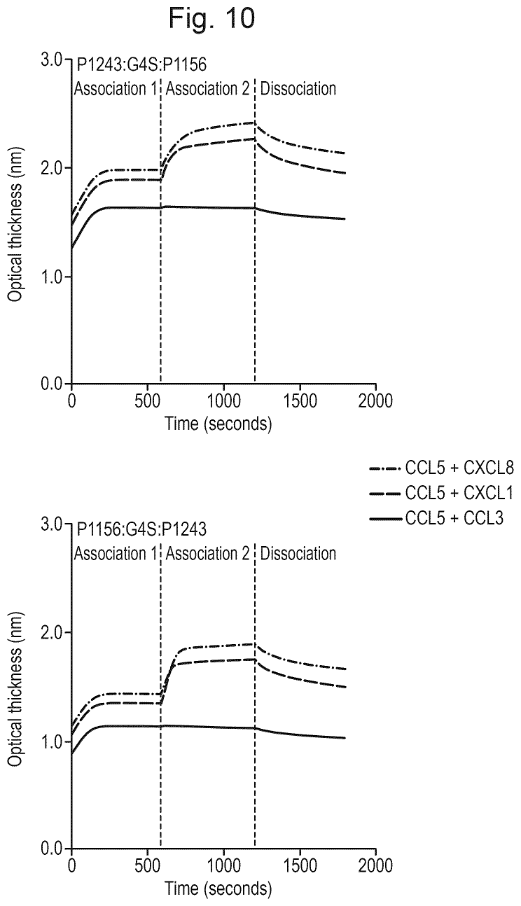

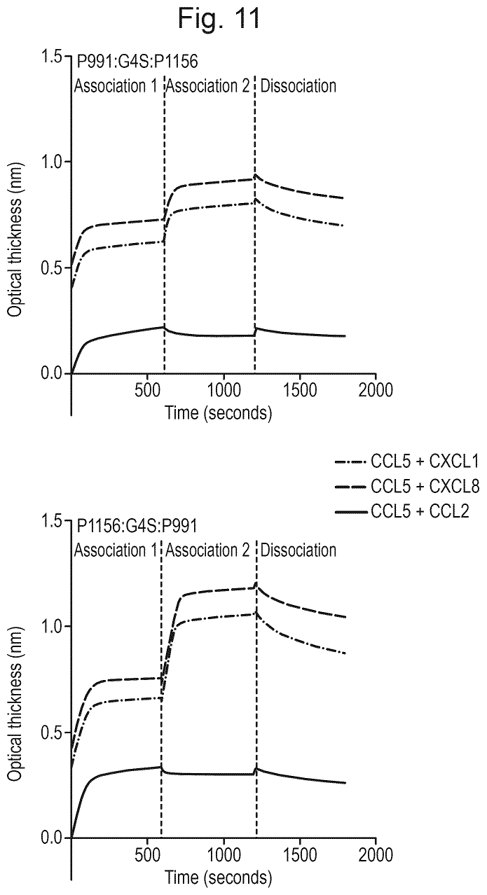

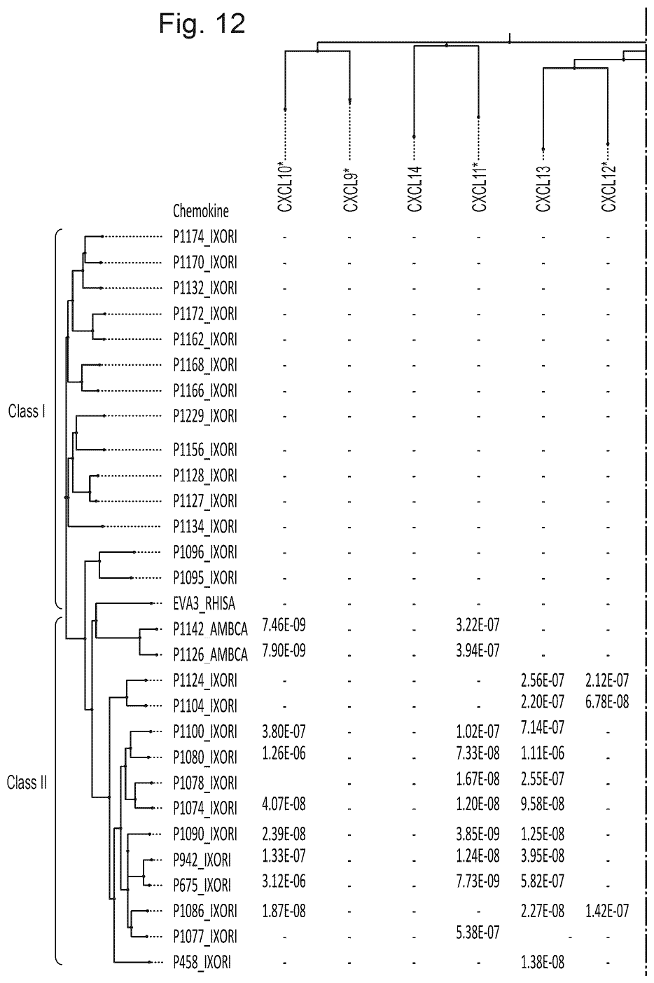

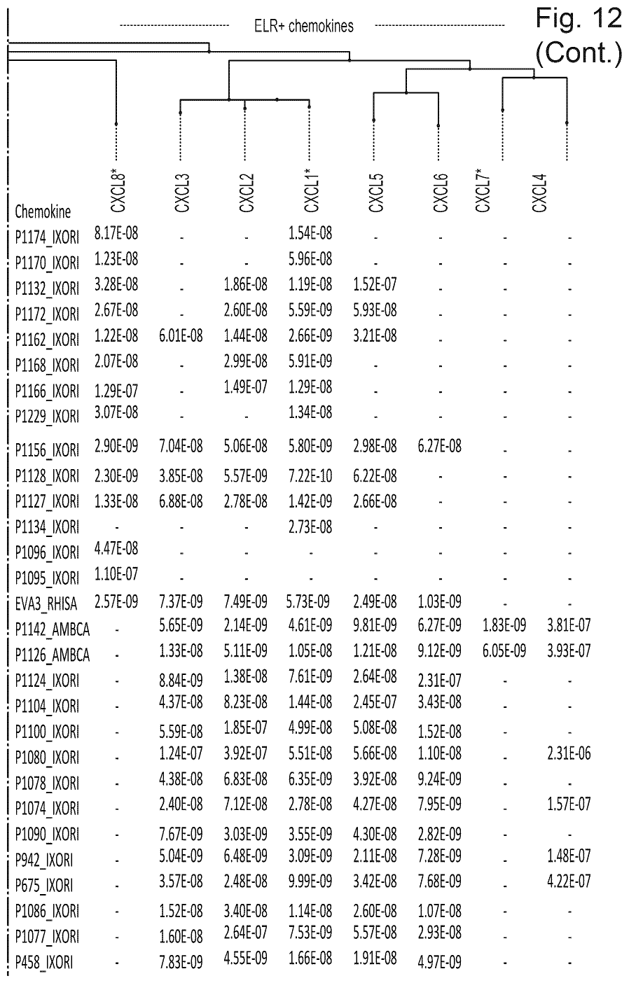

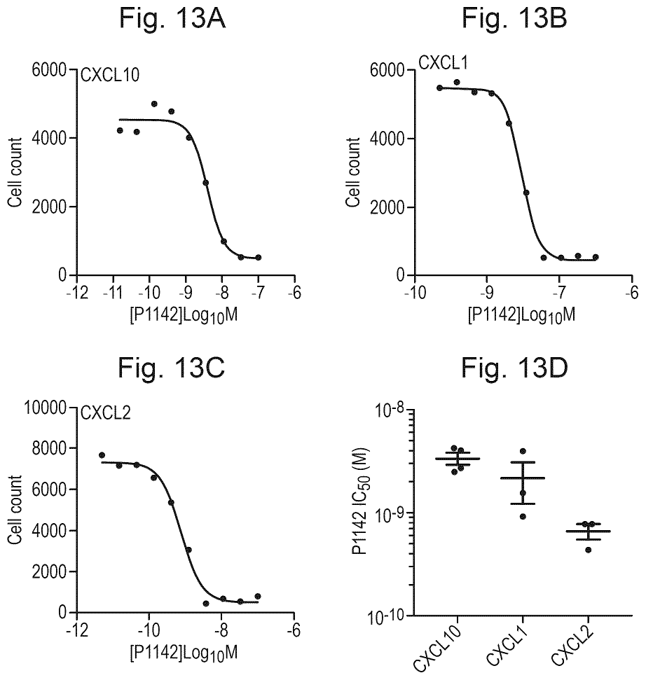

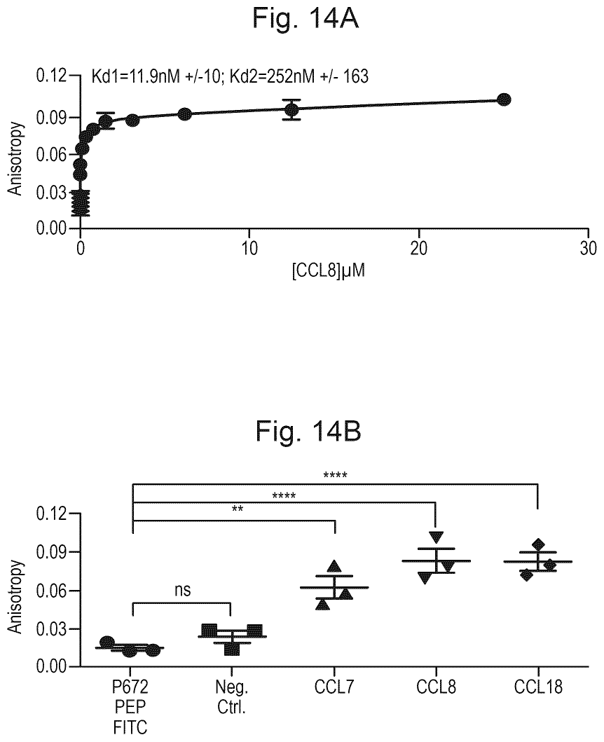

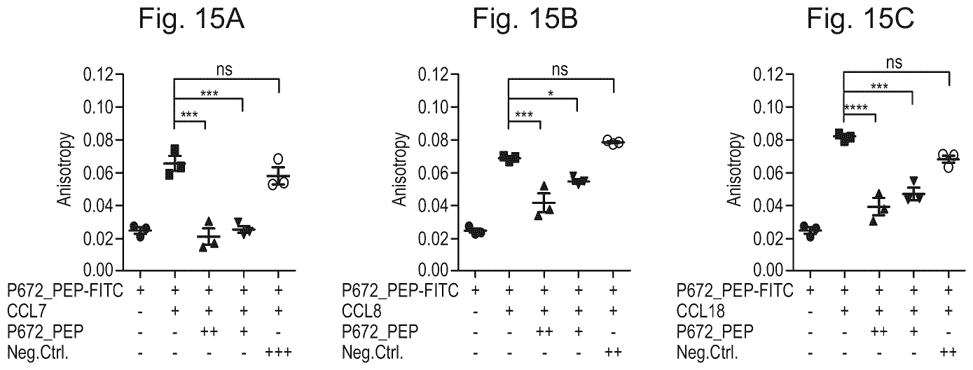

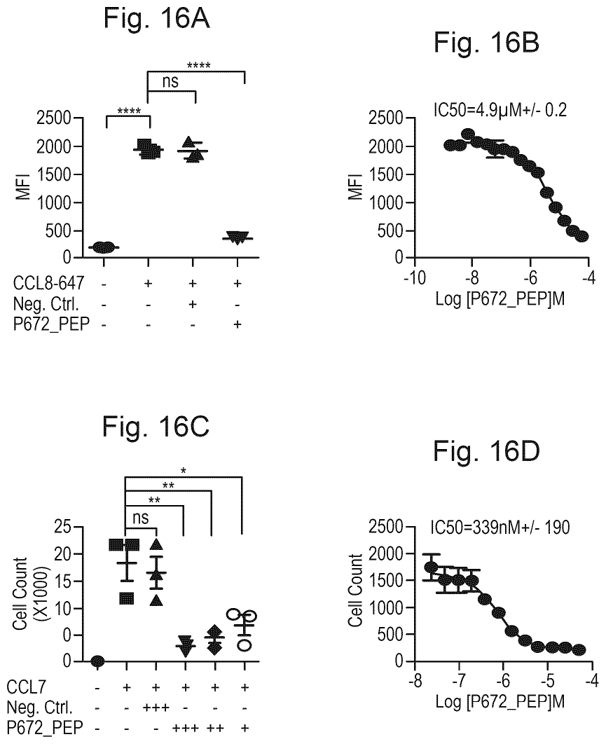

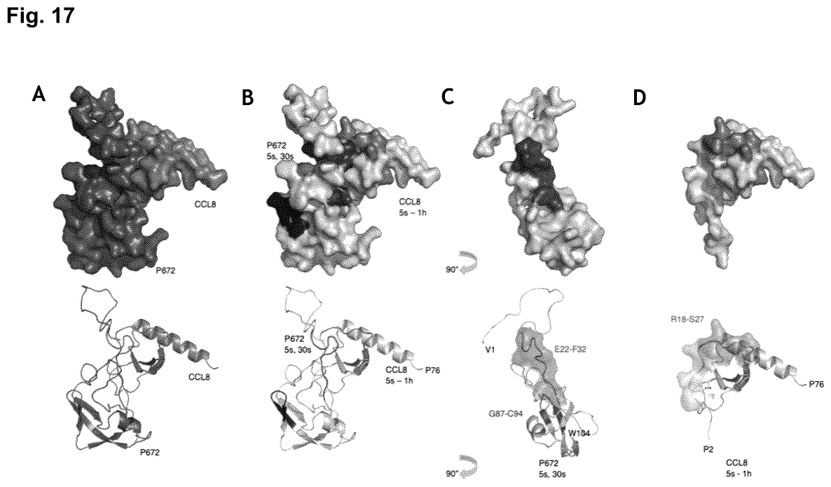

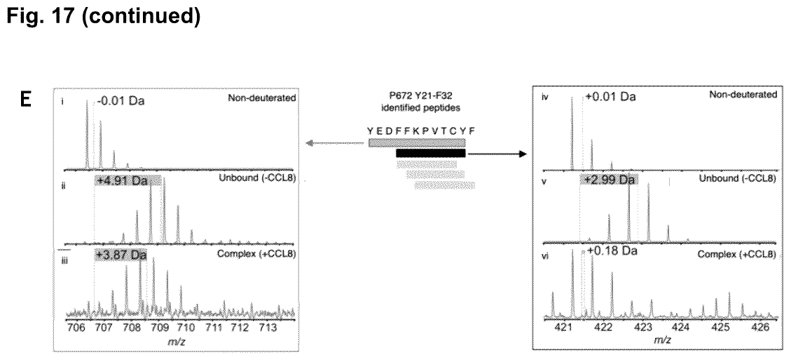

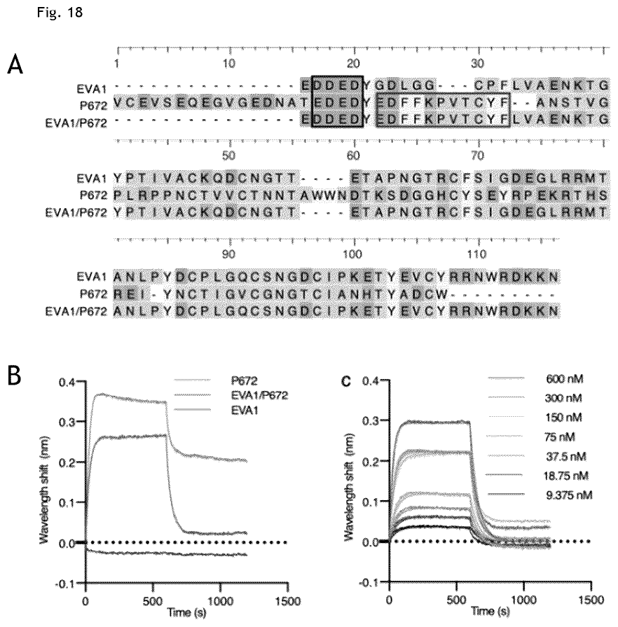

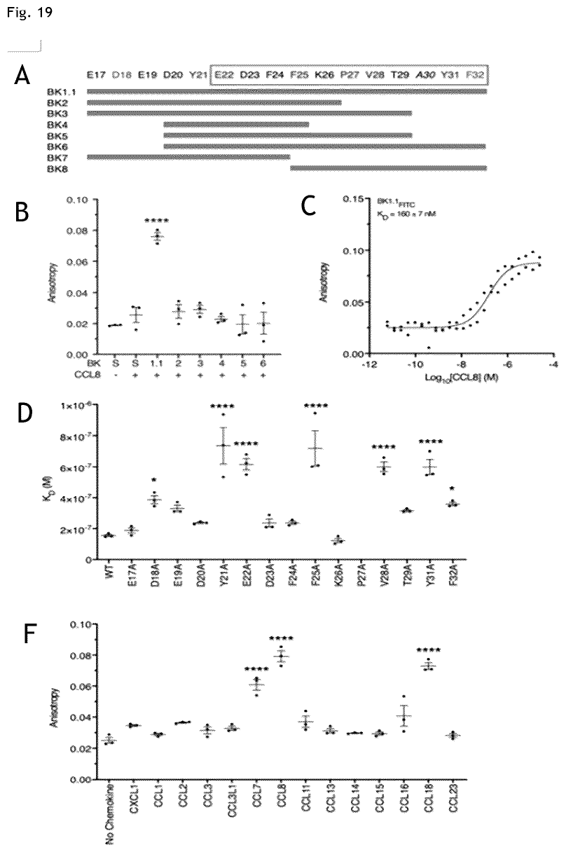

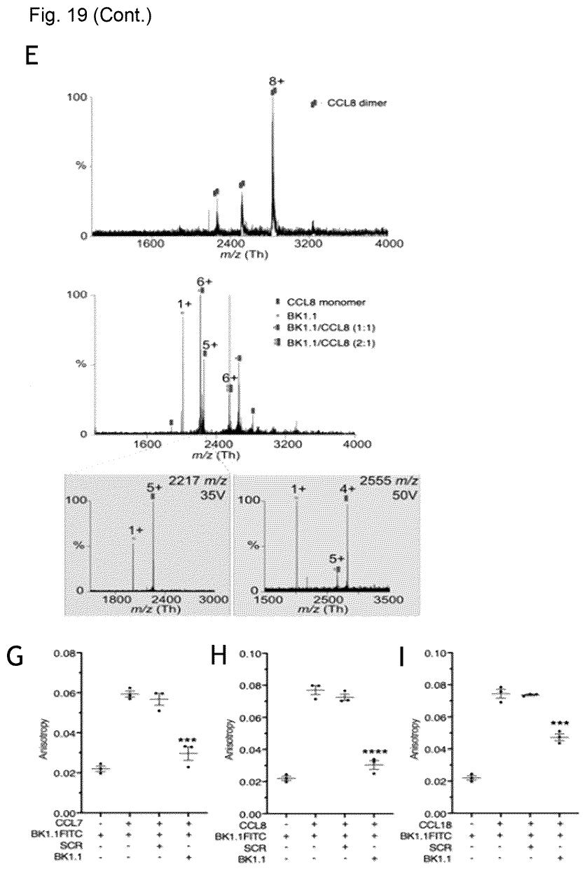

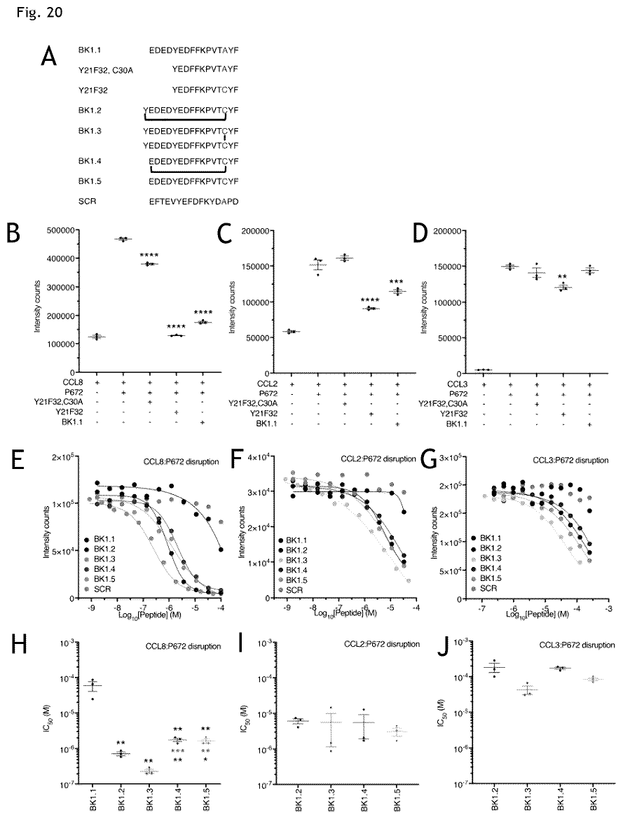

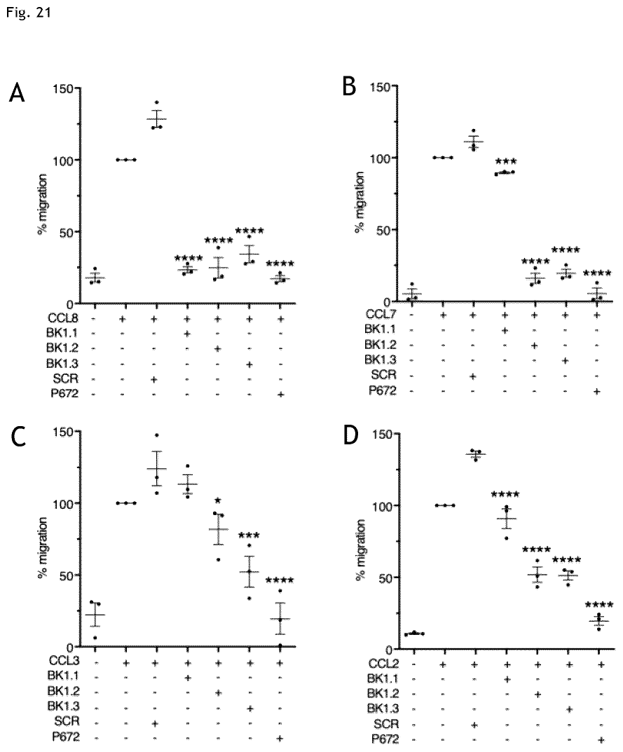

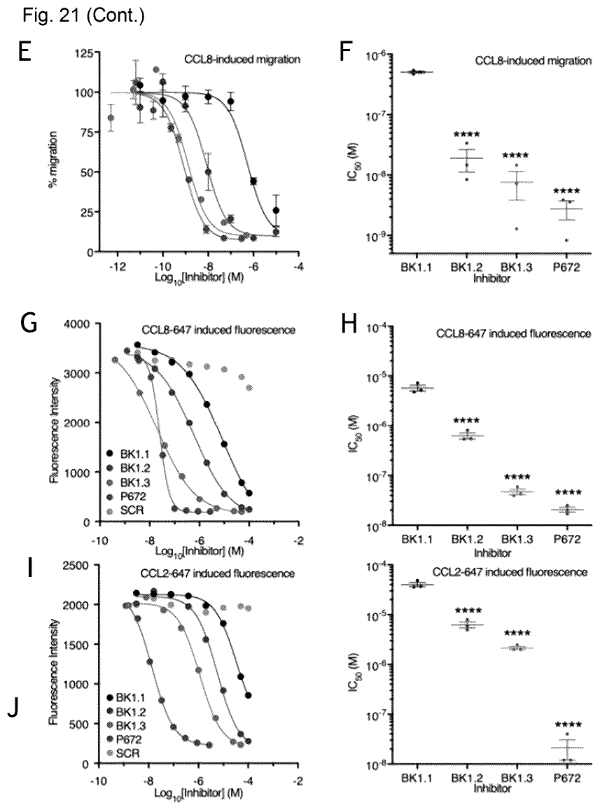

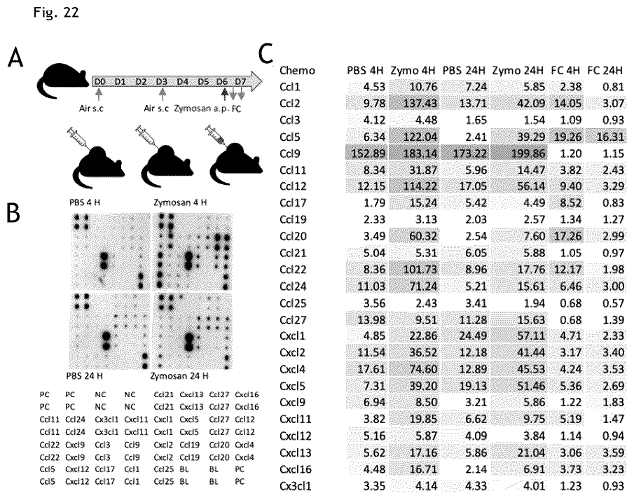

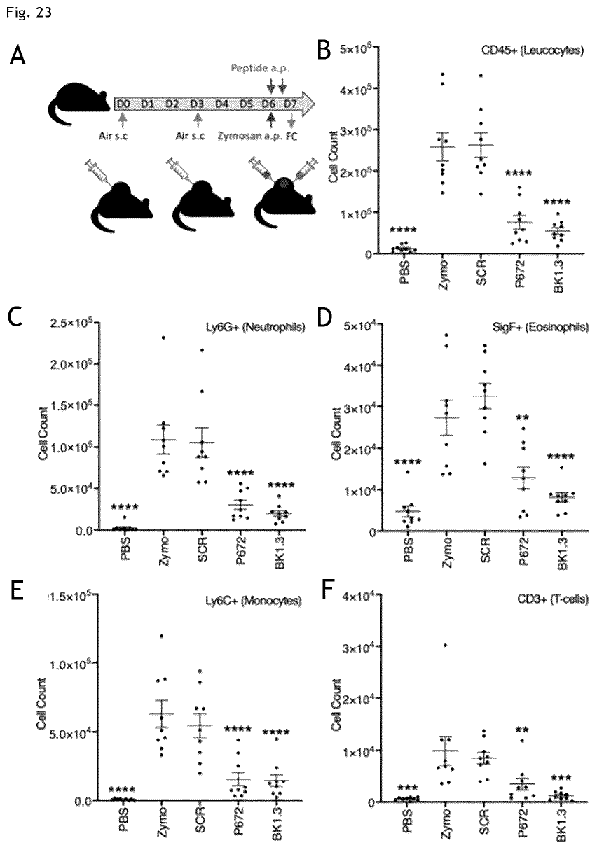

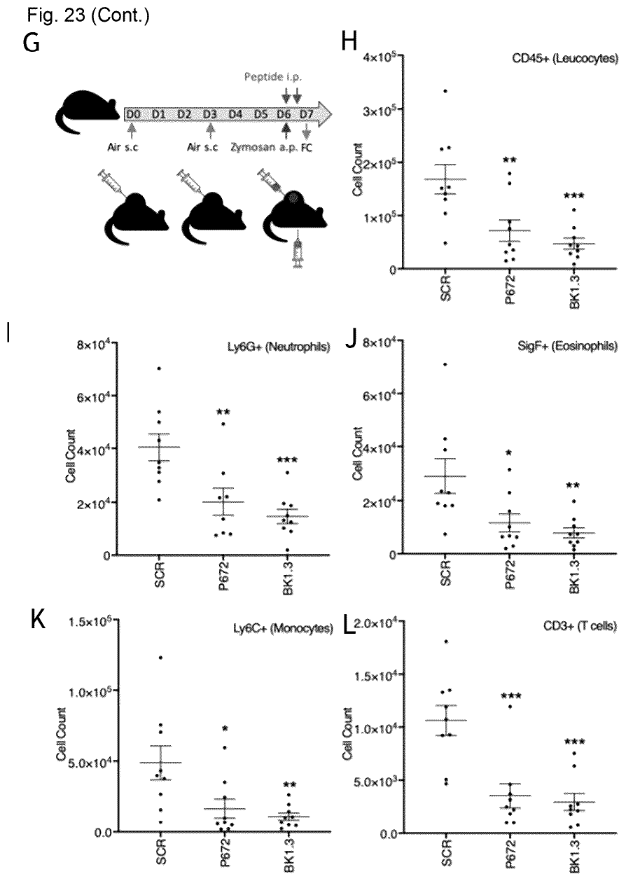

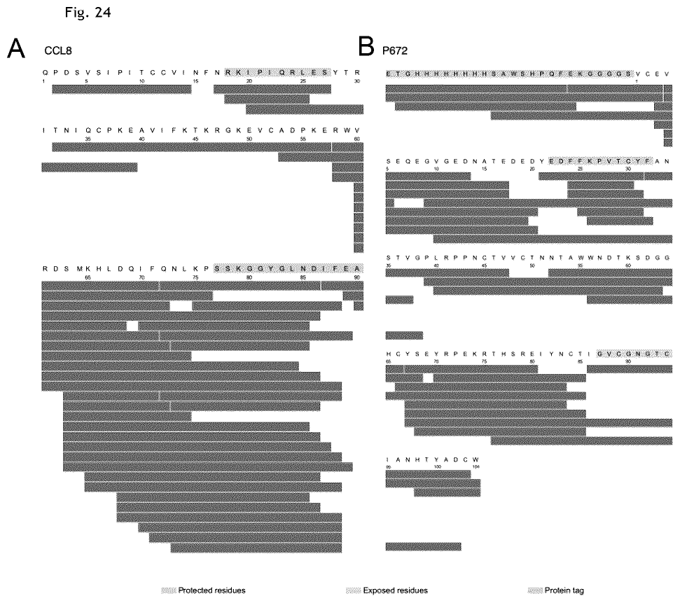

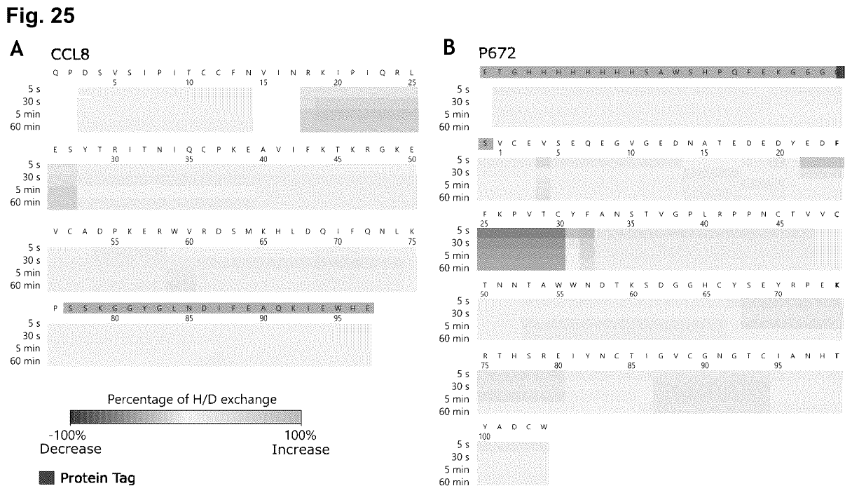

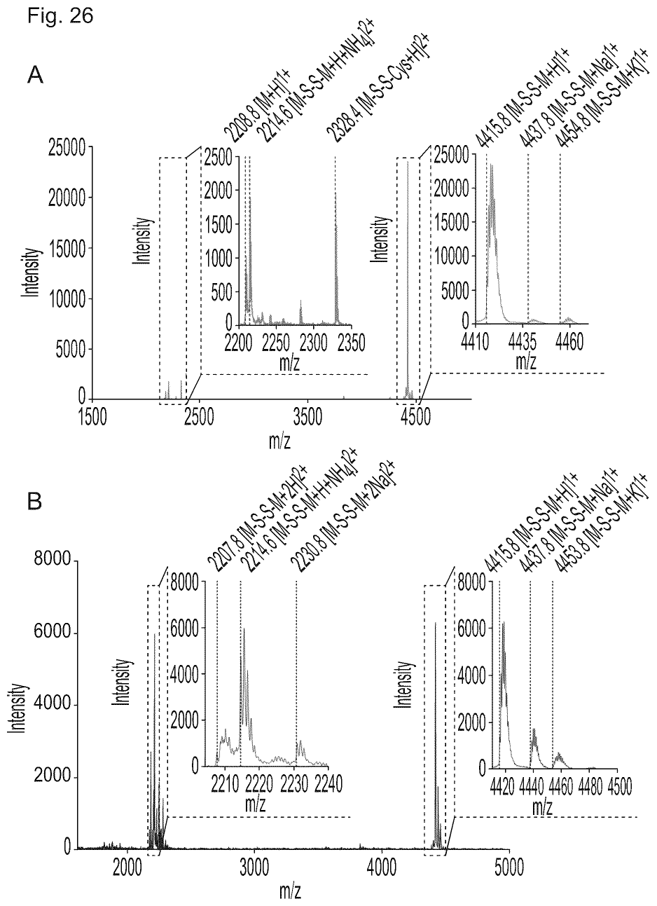

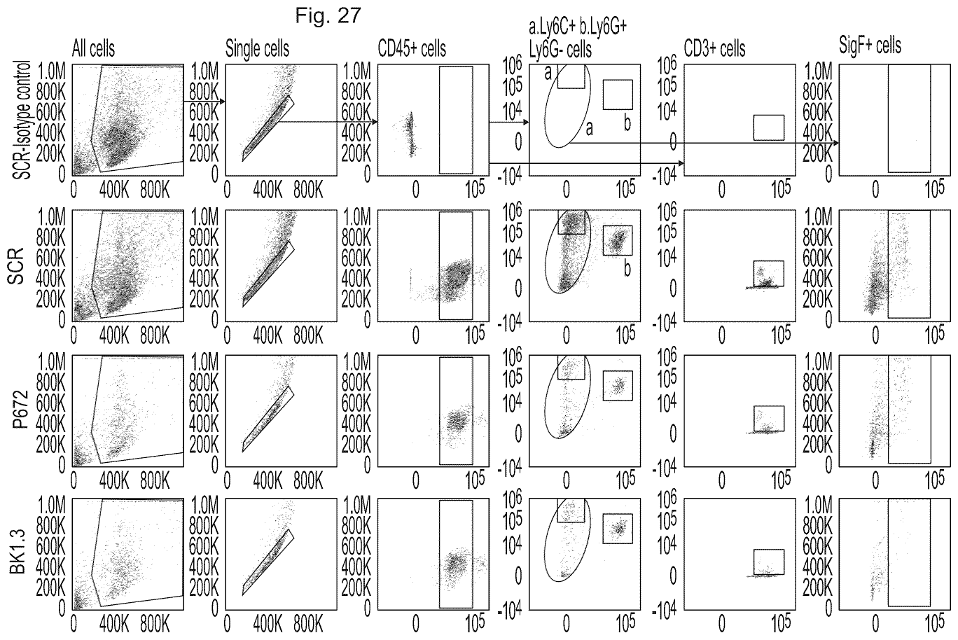

All publications, patents and patent applications cited herein, whether supra or infra, are hereby incorporated by reference in their entirety. The disclosure of PCT/GB2017/050563 in relation to SEQ ID NOs 1-31 is specifically incorporated by reference, including each of Tables 1-5 and FIGS. 1-5 thereof.

### Information on Tick CKBPs (Tables 1-8)

Table 1. Tick CKBPs described in PCT/GB2017/050563 and [17]. Tick peptide sequences isolated in yeast surface display fluorescent-activated cell sorting (FACS) screens using a labelled chemokine. Identity with Evasin-1, 4 or 3 was calculated using BLAST. Abbreviations: IXORI—Ixodes ricinus, RHISA—Rhipicephalus sanguineus, AMBMA—Amblyomma maculatum, AMBPA—Amblyomma parvum, AMBTR—Amblyomma triste, AMBAM—Amblyomma americanum, AMBCA—Amblyomma cajennense, RHIPU—Rhipicephalus pulchellus.

Table 2A-C. Binding characteristics of tick CKBPs previously disclosed (PCT/GB2017/050563 and [17]).

All members of the human chemokine family[164] are listed in column 1. Binding to human chemokines was determined for 14 tick CKBPs using biolayer interferometry (BLI) [17,165] with calculated K_{d} shown as Molar (Moles/Litre). Binding data for 17 other novel tick CKBPs was assayed using yeast surface display[17,166], with positive binding results shown as “YES”. For biolayer interferometry, His-tagged purified tick CKBP was bound to a Ni-NINTA sensor on an OctetRed® 384 system, and then binding to each chemokine listed (with the exception of CCL25, CCL26, CXCL16, CXCL17, CXCL4L1, XCL2) was assayed in a cross-binding screen at a chemokine concentration of 300 nM as described[17]. For those chemokines showing binding to a tick CKBP in the cross-binding screen, binding assays were repeated using different doses of chemokine. Association, equilibrium and dissociation data were analysed using Octet software to create corresponding fitted curves, and used to calculate K_{d}. For yeast surface display, (YSD) background fluorescence was controlled for by using either an empty vector or by omitting the chemokine (i.e. using streptavidin-Alexa647 alone). An arbitrary threshold of >3 fold over background mean fluorescence intensity was chosen to describe confirmed re-tests. Where binding was detected data are indicated as “YES”. For biolayer interferometry (BLI) data, empty cells in FIG. 2A-C represent chemokines where either binding assays were not done (CCL25, CCL26, CXCL16, CXCL17, CXCL4L1, XCL2); or where lack of binding was confirmed by biolayer interferometry. For yeast surface display data, empty cells represent chemokines that were not tested. Binding data published in relation to previously described tick CKBPs (Evasins-1, 4 and 3) is also shown for comparison in each of FIGS. 2A-C, with the relevant data obtained from publications: [20-22,32].

Table 3: Neutralisation of human chemokines by tick CKBPs previously disclosed (PCT/GB2017/050563 and [17]). Neutralising activity was determined using a quantitative THP1 cell migration assay in a 96-well Boyden chamber with chemokine in the bottom chamber as described[17]. Cells migrating through to the bottom chamber at 4 hours were counted using flow cytometry in a 96 well plate format. IC₅₀ for neutralisation was determined at the chemokine EC₈₀ dose as determined using a range of tick CKBP concentrations. Data was analysed using GraphPad Prism to determine IC₅₀, which is shown as Molar (Moles/Litre). Empty cells represent experiments not done.

Table 4—Other previously described tick CKBPs [18,20-22]. Tick CKBPs that were also described in PCT/GB2017/050563 and Singh et al. [17] are indicated in “Notes”.

Table 5.—Novel tick CKBPs of the invention.

Table 6. Binding characteristics of new CXC chemokine binding tick CKBPs shown in Table 3. Column 1 shows sequence ID, column 2 the name of the tick peptide. The peptide sequence prefix indicates the identity, and suffix indicate the tick species as follows: AMBCA, Amblyomma cajennense and IXORI, Ixodes ricinus. Column 3 shows the identity of the chemokine that was used in the yeast surface display screen to isolate the yeast clone displaying the peptide from a yeast library. Certain tick peptides e.g. P1074_IXORI were recovered from screens performed with more than one chemokine. Individual yeast clones recovered from the library were re-tested using a FACS (fluorescence activated cell sorting), experiment. The binding of the peptide to the chemokine was assayed by measuring the percent (%), of yeast cells expressing the given peptide that exceeded a background threshold set by measuring the fluorescence of the yeast library pool treated with streptavidin-Alexa467 alone.

When more than one independent yeast clone was isolated for a given peptide-chemokine combination, the mean percent shift was calculated and reported in column 3. Note that as binding of other chemokines to the indicated tick peptide have not yet been determined, the data in column 3 is necessarily incomplete. Column 4 shows the percent identity of each tick peptide to prior art tick CKBPs (EVA1, EVA4 or EVA3). This was calculated using the BLAST algorithm using default parameters, and is reported together with the alignment length in residues. A blank cell indicates that no homology was identified by BLAST.

Table 7. Potential disease applications of tick CKBPs in Table 1. Table based on binding and inhibition data shown above and on published chemokine expression in disease states as shown in FIG. 1. References for chemokine expression in disease are as discussed above in relation to FIG. 1.

Table 8. Potential disease applications of tick CKBPs in Table 5. Table based on binding and inhibition data shown above and on published chemokine expression in disease states as shown in FIG. 1. References for chemokine expression in disease are as discussed above in relation to FIG. 1.

### Hybrid Polypeptides of the Invention

The invention provides a hybrid polypeptide representing amino acid sequences derived from two or more different tick CKBPs. The hybrid polypeptide typically has different chemokine binding properties compared to any single tick CKBPs from which it is derived. The hybrid polypeptide may have different chemokine binding properties compared to any single tick CKBP.

The invention typically provides a hybrid polypeptide comprising an amino acid sequence of a first tick CKBP or a variant thereof and an amino acid sequence of a second tick CKBP or a variant thereof, wherein said hybrid polypeptide has an altered chemokine binding profile compared to the first or second tick CKBP. The first and second tick CKBP polypeptides are not identical.

The hybrid polypeptide comprises at least an amino acid sequence of a first tick CKBP polypeptide or variant thereof, and an amino acid sequence of a second tick CKBP polypeptide or variant thereof, but may also comprise amino acid sequences from one or more other tick CKBP polypeptides or variants thereof. Thus, the hybrid polypeptide may be derived from three, four, five or more different tick CKBP polypeptides. The discussion herein of selection of second tick CKBP polypeptides by comparison with first tick CKBP polypeptides for provision of sequences for a hybrid polypeptide is also applicable to selection of any additional tick CKBP polypeptide to be represented in the hybrid polypeptide. Thus, an additional sequence to be provided from a further (for example, third) tick CKBP polypeptide may be selected to provide an additional chemokine-binding activity for the hybrid polypeptide compared to those provided by sequences derived from the other (for example, first and second) tick CKBP polypeptides.

Chemokine Binding

The altered chemokine binding profile for the hybrid polypeptide comprises the ability to bind a different selection of chemokines as compared to those bound by the first or second tick CKBP polypeptide individually. The hybrid polypeptide may thus be able to bind one or more chemokines not bound by the first or second tick CKBP polypeptide individually. The hybrid polypeptide may not exhibit binding to one or more chemokines that are bound by the first or second tick CKBP polypeptide. It should be understood that the altered chemokine binding profile for the hybrid polypeptide is by comparison to that of any single tick CKBP polypeptide from which it is derived, taken individually. Thus, the hybrid polypeptide displays an altered chemokine binding profile compared to any single tick CKBP polypeptide whose sequence it represents. In some aspects, the chemokine binding profile of the hybrid polypeptide may in contrast be substantially identical or identical to the cumulative (combined) chemokine binding profile of each of the individual tick CKBP polypeptides whose sequences it represents.

The hybrid polypeptide may bind at least one additional chemokine compared to a first tick CKBP polypeptide from which it is derived. The additional chemokine binding for the hybrid polypeptide is provided by the presence of at least one chemokine binding sequence derived from a different (second) tick CKBP polypeptide to the first tick CKBP polypeptide. The second tick CKBP polypeptide thus binds one or more different chemokines compared to the first tick CKBP polypeptide. The hybrid polypeptide may bind at least two, at least three, at least four, at least five, at least six, or at least eight additional chemokines as compared to the first tick CKBP polypeptide.

The hybrid polypeptide may bind in total at least two, at least three, at least four, at least five, at least six, at least eight, at least ten, at least twelve, at least fourteen or more different chemokines, The hybrid polypeptide may bind up to five, up to ten, up to twelve, up to fifteen or up to twenty different chemokines. The hybrid polypeptide may bind two to five, two to eight, two to ten, two to twelve, two to fifteen, or two to twenty different chemokines. The hybrid polypeptide may bind five to ten, five to fifteen, or five to twenty different chemokines.

The hybrid polypeptide may bind all chemokines bound by the two or more different tick CKBP polypeptides from which it is derived.

The hybrid polypeptide may alternatively bind a reduced number of chemokines compared to the total number of chemokines that are bound by the two or more different tick CKBP polypeptides from which it is derived. The reduced chemokine binding for the hybrid polypeptide may be provided by the loss of one or more chemokine binding sequences present in the two or more different tick CKBP polypeptides from which it is derived. In this aspect, the hybrid polypeptide may not bind at least one, at least two, at least three, at least four, at least five, at least six or at least eight of the chemokines that are bound (jn combination) by the two or more different tick CKBP polypeptides from which it is derived. In some aspects, the hybrid polypeptide may have reduced chemokine binding (bind to a reduced number of different chemokines) compared to any individual tick CKBP from which it is derived. Thus, it may only bind one chemokine, two chemokines, three chemokines, four chemokines, or five different chemokines. It may bind up to two, up to three, up to four or up to five different chemokines.

The chemokines may be selected from any known chemokines or chemokines newly identified in the future which are bound by tick CKBP polypeptides. The chemokines are preferably human chemokines. However chemokines may also be selected from other animals of veterinary importance (e.g. dog, cat, pig, sheep, cow, horse) and scientific importance (e.g. mouse, rat, monkey).

It is preferred that a hybrid polypeptide bind at least one CC chemokine and at least one CXC chemokine, i.e. at least one chemokine of the CC class and at least one chemokine of the CXC class. The known human CC and CXC chemokines are indicated in Table 2 and the hybrid polypeptide may bind any of the CC and/or CXC chemokines shown in Table 2. Certain CC and CXC chemokines are not known to be bound by tick CKBPs described to date (including ones detailed here). These include: CCL28, CXCL13, CXCL14, CXCL16, CXCL17, CXCL4, CXCL4L1.

Binding of at least one CC and at least one CXC chemokine is of particular utility in matching to chemokine expression in disease where both CC and CXC chemokines are expressed. A CC chemokine may be selected from any of the disease expressed CC chemokines shown in FIG. 1. A CXC chemokine may be selected from any of the disease expressed CXC chemokines shown in FIG. 1. The hybrid polypeptide may bind at least two CC chemokines and at least one CXC chemokine, at least three CC chemokines and at least one CXC chemokine, at least five CC chemokines and at least one CC chemokine, at least six CC chemokines and at least one CXC chemokine, at least eight CC chemokines and at least one CXC chemokine, at least ten CC chemokines and at least one CXC chemokine, at least twelve CC chemokines and at least one CXC chemokine, at least fourteen CC chemokines and at least one CXC chemokine, or at least sixteen CC chemokines and at least one CXC chemokine. The hybrid polypeptide may bind any of the above minimum numbers of different CC chemokines and at least two different CXC chemokines, at least three CXC chemokines, at least four CXC chemokines, at least five CXC chemokines, or at least six CXC chemokines. The hybrid polypeptide may bind one CC class chemokine and at least two, at least three, at least four, at least five CXC or at least six CXC chemokines.

Ideally a hybrid polypeptide should bind CC and CXC chemokines expressed and relevant to a particular disease. The hybrid polypeptide may be designed to bind CX3C and CC chemokines or CX3C and CXC chemokines, or CX3C, CC and CXC chemokines if the CX3C chemokine is expressed in the disease, and thought to be relevant to the disease.

Tick CKBPs

The tick CKBP polypeptides from which the hybrid polypeptide is derived may be selected from any tick CKBP polypeptides, including currently described tick CKBPs and tick CKBPs identified in the future. A tick CKBP polypeptide may be derived from any tick species, preferably a tick species that infects humans. The tick species may be selected from any of Amblyomma, Anomalohimalaya, Bothriocroton, Cosmiomma, Cornupalpatum, Compluriscutula, Dermacentor, Haemaphysalis, Hyalomma, Ixodes, Margaropus, Nosomma, Rhipicentor, Rhipicephalus, Nuttalliella, Antricola, Argas, Nothoaspis, Ornithodoros, and Otobius genera. A tick CKBP polypeptide binds one or more chemokines, preferably one or more human chemokines. A tick CKBP polypeptide typically binds multiple chemokines, such as at least two different chemokines.

The tick CKBP family is characterised by low sequence identity between members (although more closely related tick CKBPs may display greater sequence identity). Conserved structural features though exist allowing for ready classification of chemokine-binding proteins as tick CKBPs. A tick CKBP is typically a tick Evasin polypeptide. A tick CKBP may be a previously described tick Evasin or tick Evasin variant or a tick Evasin or tick Evasin variant identified in the future. An example of a previously described tick Evasin variant is provided by the sequence having the accession number EZ406190.1, which may be used in place of native Evasin-1 (SEQ ID NO: 32) in any sequence combination based on SEQ ID NO: 32 described herein.

A tick CKBP polypeptide may thus display a conserved set of eight cysteine residues (typically forming four disulphide bonds), which can be aligned with corresponding cysteine residues in known tick CKBP having a set of eight cysteines. Tick CKBPs of this type are typically CC binding tick CKBPs. Examples include Evasin-1 and Evasin-4 (SEQ ID NOs 32 and 34) and SEQ ID NOs 1-3, 6-9, 20-23, 29, and 35-44.

An illustration of a sequence alignment of tick CKBPs of this type against Evasin-1, showing the conserved eight cysteine positions, is provided in FIG. 2. An illustration of identification of novel tick CKBP polypeptides having this conserved cysteine pattern is also provided in [17]. Any known CC-binding tick CKBP sequence as described above (or multiple such sequences) may be aligned with the sequence of a candidate tick CKBP polypeptide to assist its identification.

Alternatively, a tick CKBP polypeptide may display a conserved set of six cysteine residues which can be aligned against sequences of known tick CKBPs also having a corresponding set of six cysteines. Tick CKBPs of this type are typically CXC binding CKBPs. Examples of CXC-binding tick CKBPs include Evasin-3 (SEQ ID NO: 33) and SEQ ID NOs 5, 18, 19, and 45-72. An illustration of a sequence alignment of tick CKBPs of this type against Evasin-3, showing the conserved six cysteine positions, is provided in FIG. 3. Any known CXC-binding tick CKBP sequence as described above (or multiple such sequences) may be aligned with the sequence of a candidate tick CKBP polypeptide to assist its identification. A CXC binding tick CKBP may bind ELR+ CXC-chemokines including CXCL1 and/or CXCL8, and may be a Class I CXC-binding Evasin as shown in FIG. 12. A CXC binding tick CKBP may bind ELR− and ELR+ CXC-chemokines and not bind CXCL8, and may be a Class II CXC-binding Evasin as shown in FIG. 12. Such evasins may not bind CC chemokines.

Tick CKBP polypeptides may also be selected from any of polypeptides comprising the amino acid sequence of any one of SEQ ID NOs 1-72 or naturally occurring homologues thereof, including homologues present in any tick species discussed above. Such naturally occurring homologues may comprise an amino acid sequence having at least 20%, at least 30%, at least 40%, at least 50%, at least 60%, at least 70%, at least 80%, at least 85%, at least 90%, at least 95% or at least 99% homology or identity to the amino acid sequence of any one of SEQ ID NOs 1-72. Preferably, the above homology or identity is measured over the full length of the homologue.

As discussed above, a first tick CKBP polypeptide represented in the hybrid polypeptide is selected to differ from the second tick CKBP polypeptide represented in the hybrid polypeptide (and any additional tick CKBP polypeptides represented in the hybrid polypeptide are also selected to differ from other tick CKBPs represented). However, the first and second (and additional) tick CKBP polypeptides may otherwise be selected from any tick CKBP polypeptides discussed above. Each tick CKBP polypeptide selected as a basis for provision of the hybrid polypeptide typically has at least one differing chemokine-binding activity. Thus, for example a first tick CKBP polypeptide may be a CC-binding tick CKBP and the second tick CKBP polypeptide a CXC-binding tick CKBP. Alternatively, the first and second tick CKBP polypeptides may both be CC-binding tick CKBPs, but with at least one differing CC-binding activity between them. Thus, for example the first tick CKBP may bind CCL8 and the second tick CKBP CCL5, or the first tick CKBP may bind CCL8 and the second tick CKBP CCL5 and CCL8. In another aspect, the first and second tick CKBP polypeptides may both be CXC-binding tick CKBPs, but with at least one differing CXC-binding activity between them. Thus, for example the first tick CKBP may bind CXCL8 and the second tick CKBP CXCL12, or the first tick CKBP may bind CXCL1 and CXCL8 and the second tick CKBP CXCL1, CXCL3 and CXCL8.

Examples of differing chemokine-binding activities for individual tick CKBP polypeptides are provided in Tables 2-4 and 6. Combinations of two or more tick CKBPs may accordingly be selected together to provide a desired combination of chemokine-binding activities in a hybrid polypeptide, starting from consideration of the individual binding activities displayed by each tick CKBP. The combination of chemokine-binding activities may be selected to reflect chemokine expression in particular disease, such as any chemokine disease expression pattern shown in FIG. 1, as discussed further below.

Particular CC-binding tick CKBPs are provided by SEQ ID NOs 1-3, 6-9, 20-23, 29. 32, and 34-44. Particular CXC-binding tick CKBPs are provided by SEQ ID NOs 5, 18, 19, 33 and 45-72. Thus a hybrid polypeptide may comprise (i) an amino acid sequence of a first tick CKBP polypeptide selected from any one of SEQ ID NOs 1-3, 6-9, 20-23, 32, and 34-44, or a variant of any thereof, and (ii) an amino acid sequence of a second tick CKBP polypeptide selected from any one of SEQ ID NOs 5, 18, 19, 33 and 45-72 or a variant of any thereof. A preferred variant of SEQ ID NO: 3 is the peptide of SEQ ID NO: 89 (EDEDYEDFFKPVTCYF) or a variant thereof, such as SEQ ID NO: 88 (EDEDYEDFFKPVTAYF). A variant of SEQ ID NO: 89 typically binds CCL8, CCL7 and CCL18. SEQ ID NO: 89 or a variant thereof as above may be used in place of SEQ ID NO: 3 in any hybrid polypeptide described herein including an amino acid sequence of a tick CKBP polypeptide selected from SEQ ID NO: 3 or a variant thereof.

A hybrid polypeptide may alternatively comprise first and second tick CKBP amino acid sequences or variants thereof each selected from group (i) above, or first and second tick CKBP amino acid sequences each selected from group (ii) above.

Specific examples of hybrid polypeptides based on the above tick CKBP sequences are provided by the hybrid polypeptides comprising amino acid sequences as shown in SEQ ID NOs: 7-74 and 80-81 shown below.

SEQ ID NO: 73 (linker region bold and underlined):
ENGEGTTQPDYDNSTDYYNYEDFKCTCPAPHLNNTNGTVMKPIGCYYTCN
VTRCTAPDTYPCYNLTEHQAKNLTTSPTTLCAVGNCDHGICVPNGTKELC
FKAPNLEEGGGGSADDDNELFTVQYCGMNCTKDEGGTWTGCTGKKEGCKC
YHESGKNYGLCLSTEYTDFSQYGNPSDSEIEAAKPKRSDTLSH
SEQ ID NO: 74 (linker region bold and underlined):
RNHTEDNSTEYYDYEEARCACPARHLNNTNGTVLKLLGCHYFCNGTLCTA
PDGYPCYNLTAQQVRTLTTYPNTSCAVGVCMKGTCVKNGTMEQCFKTPGG
GGSADDDNELFTVQYCGMNCTKDEGGTWTGCTGKKEGCKCYHESGKNYGL
CLSTEYTDFSQYGNPSDSEIEAAKPKRSDTLSH
SEQ ID NO: 80 (linker region bold and underlined):
ADDDNELFTVQYCGMNCTKDEGGTWTGCTGKKEGCKCYHESGKNYGLCLS
TEYTDFSQYGNPSDSEIEAAKPKRSDTLSHGGGGSENGEGTTQPDYDNST
DYYNYEDFKCTCPAPHLNNTNGTVMKPIGCYYTCNVTRCTAPDTYPCYNL
TEHQAKNLTTSPTTLCAVGNCDHGICVPNGTKELCFKAPNLEE
SEQ ID NO: 81 (linker region bold and underlined):
ADDDNELFTVQYCGMNCTKDEGGTWTGCTGKKEGCKCYHESGKNYGLCLS
TEYTDFSQYGNPSDSEIEAAKPKRSDTLSHGGGGSRNHTEDNSTEYYDYE
EARCACPARHLNNTNGTVLKLLGCHYFCNGTLCTAPDGYPCYNLTAQQVR
TLTTYPNTSCAVGVCMKGTCVKNGTMEQCFKTP

SEQ ID NO: 73 comprises a first (CC-binding) tick CKBP amino acid sequence shown in SEQ ID NO: 9 and a second (CXC-binding) tick CKBP amino acid sequence shown in SEQ ID NO: 19. SEQ ID NO: 80 comprises these two tick CKBP amino acid sequences in the alternative order. More generally, a hybrid polypeptide may comprise the amino acid sequence of SEQ ID NO: 9 or a variant thereof and the amino acid sequence of SEQ ID NO: 19 or a variant thereof.

SEQ ID NO: 74 comprises a first (CC-binding amino acid sequence shown in SEQ ID NO: 29 and a second (CXC-binding) tick CKBP amino acid sequence shown in SEQ ID NO: 19. SEQ ID NO: 81 comprises these two tick CKBP amino acid sequences in the alternative order. A hybrid polypeptide may more generally comprise the amino acid sequence of SEQ ID NO: 29 or a variant thereof and the amino acid sequence of SEQ ID NO: 19 or a variant thereof.

Hybrid polypeptides comprising first, second and third (or more) chemokine binding sequences are also provided herein, including the 3-warhead evasins described below. SEQ ID NO: 92 comprises a first chemokine-binding sequence shown in SEQ ID 65, a second chemokine-binding sequence shown in SEQ ID 19, and a third chemokine-binding sequence shown in SEQ ID NO: 1, with intervening GGGGS linkers. A related hybrid polypeptide may more generally comprise the amino acid sequence of SEQ ID NO: 65 or a variant thereof, the amino acid sequence of SEQ ID NO: 19 or a variant thereof, and the amino acid sequence of SEQ ID NO: 1 or a variant thereof. The three amino acid sequences may be present in any order and may be fused contiguously or separated by any suitable linkers.

SEQ ID NO: 93 comprises a first chemokine-binding sequence shown in SEQ ID 65, a second chemokine-binding sequence shown in SEQ ID 19, and a third chemokine-binding sequence shown in SEQ ID NO: 9, with intervening GGGGS linkers. A related hybrid polypeptide may more generally comprise the amino acid sequence of SEQ ID NO: 65 or a variant thereof, the amino acid sequence of SEQ ID NO: 19 or a variant thereof, and the amino acid sequence of SEQ ID NO: 9 or a variant thereof. The three amino acid sequences may be present in any order and may be fused contiguously or separated by any suitable linkers.

Specific diseases that could be suitable for targeting with a hybrid polypeptide, and additional tick CKBP sequence combinations are described in the section “therapeutic methods of the invention” below.

Engineering of Hybrid Polypeptides

A hybrid polypeptide may be engineered from first and second tick CKBP polypeptides in any manner. A hybrid polypeptide may comprise a fusion of an amino acid sequence of a first tick CKBP polypeptide or a variant thereof and an amino acid sequence of a second tick CKBP polypeptide or a variant thereof. The amino acid sequences or variants thereof may be fused directly or separated by a suitable linker. Suitable linkers include, but are not limited to, chemical crosslinkers and peptide linkers. Peptide linkers are preferred if the polypeptide of the invention and second peptide or polypeptide are genetically fused. Preferred linkers are amino acid sequences (i.e. peptide linkers). A peptide linker may be of any amino acid sequence composition or length. A linker may be at least three, at least four, or at least five amino acids in length. The length, flexibility and hydrophilicity of the peptide linker are typically designed such that it does not to disturb the functions of the polypeptide of the invention. A linker is preferably selected to be conformationally flexible and may comprise one or more glycine residues, and optionally one or more serine residues. A linker may comprise in sequence at least two, at least three or least four glycine residues. A linker may consist essentially of or consist of glycine residues. Preferred flexible peptide linkers are stretches of 2 to 20, such as 4, 6, 8, 10 or 16, serine and/or glycine amino acids. Other preferred flexible linkers include (SG)1, (SG)2, (SG)3, (SG)4, (SG)5 and (SG)8 wherein S is serine and G is glycine. A particularly preferred linker sequence is GGGGS (SEQ ID NO: 75).

A hybrid polypeptide may comprise a fusion of a first tick CKBP amino acid sequence of SEQ ID NOs 1-72 or variant thereof and a second, different amino acid sequence selected from any one of SEQ ID NOs 1-72 or a variant thereof. A hybrid polypeptide may comprise a fusion of (i) an amino acid sequence selected from any one of SEQ ID NOs 1-3, 6-9, 20-23, 29, 32, and 34-44 or a variant thereof and (ii) an amino acid sequence selected from any one of SEQ ID NOs 5, 18, 19, 33 and 45-72. The amino acid sequence or variant of (i) and the amino acid sequence or variant of (ii) may be in either orientation; thus the amino acid sequence or variant of (i) may be N-terminal or C-terminal to the amino acid sequence or variant of (ii). Examples of hybrid polypeptides which are fusions of first and second tick CKBP amino acid sequences are provided by SEQ ID NOs 73, 74 and 80-81 described above.

Alternatively, a hybrid polypeptide may comprise the amino acid sequence of a second tick CKBP polypeptide or a variant thereof substituted into the amino acid sequence of a first tick CKBP polypeptide or variant thereof. Such a hybrid polypeptide comprises a substituted derivative of the amino acid sequence of the first tick CKBP polypeptide or variant thereof.

The substitution may introduce a chemokine-binding sequence provided by the amino acid sequence of the second tick CKBP polypeptide or variant thereof into the amino acid sequence of the first tick CKBP polypeptide or variant thereof. Alternatively or additionally, the substitution may remove a chemokine-binding sequence present in the amino acid sequence of the first tick CKBP polypeptide or variant thereof. The introduced chemokine-binding sequence may bind one or more chemokines. The chemokine binding sequence may bind at least one CC chemokine and/or at least one CXC chemokine.

The substitution may introduce a CXC chemokine-binding sequence from a first tick CKBP polypeptide or variant thereof into an amino acid sequence of a second tick CKBP polypeptide or variant thereof. The second tick CKBP polypeptide may not previously have any CXC-chemokine binding activity. Alternatively, the substitution may introduce an additional CXC-chemokine binding activity. The CXC chemokine-binding sequence may bind one or more of CXCL1-14, 16.

Alternatively, the substitution may introduce a CC chemokine-binding sequence from a first tick CKBP polypeptide or variant thereof into an amino acid sequence of a second tick CKBP polypeptide or variant thereof. The second tick CKBP polypeptide may not previously have any CC-chemokine binding activity. Alternatively, the substitution may introduce an additional CC-chemokine binding activity. The CC chemokine-binding sequence may bind one or more of CCL1-24, 28.

The substitution may result in a hybrid polypeptide only having chemokine-binding activity from the introduced chemokine-binding sequence. The substitution may introduce a single chemokine-binding activity. The substitution may introduce a single chemokine-binding activity and reduce or remove the original chemokine-binding activity of the tick CKBP amino acid sequence or variant thereof into which the substitution is made. The substitution may result in a hybrid polypeptide binding a reduced number of chemokines compared to the second tick CKBP polypeptide or variant thereof.

The substitution may comprise exchange of any sequence region in the amino acid sequence of the second tick CKBP polypeptide or variant thereof for any sequence region in the amino acid sequence of the first tick CKBP polypeptide or variant thereof. The substitution may be of a chemokine-binding sequence in the amino acid sequence of the first tick CKBP polypeptide or variant thereof for a chemokine-binding sequence in the amino acid sequence of the second tick CKBP polypeptide or variant thereof. Alternatively, the substitution may introduce an additional chemokine-binding sequence provided by the first tick CKBP polypeptide or variant thereof into a region of the amino acid sequence of the second tick CKBP polypeptide or variant thereof not comprising a chemokine-binding sequence.

A hybrid polypeptide may comprise a substitution of a first tick CKBP amino acid sequence of SEQ ID NOs 1-72 or variant thereof into a second, different amino acid sequence selected from any one of SEQ ID NOs 1-72 or a variant thereof. A hybrid polypeptide may comprise a substitution of (i) an amino acid sequence selected from any one of SEQ ID NOs 1-3, 6-9, 20-23, 29, 32, and 34-44 or a variant thereof into (ii) an amino acid sequence selected from any one of SEQ ID NOs 5, 18, 19, 33 and 45-72, or vice-versa. Such a hybrid polypeptide may comprise a substitution of a chemokine-binding sequence from an amino acid sequence of (i) into an amino acid sequence of (ii) or a variant thereof. Alternatively, a chemokine-binding sequence from an amino acid sequence of (ii) may be substituted into an amino acid sequence of (i) or a variant thereof.

A specific example of a hybrid polypeptide comprising a substitution of an amino acid sequence of a second tick CKBP polypeptide into the amino acid sequence of a first tick CKBP polypeptide is provided by a polypeptide comprising the amino acid sequence of SEQ ID NO: 76, shown below.

SEQ ID NO: 76 (first (introduced) tick CKBP
sequence bold and underlined; residual recipient
second tick CKBP sequence in italics):
VCEVSEQEGVGEDNATEDEDYEDFFKPVTCYFANSTVGPLRPPNCKQD
CNGTTETAPNGTRCFSIGDEGLRRMTANLPYDCPLGQCSNGDCIPKET
YEVCYRRNWRDEKN.

The introduced chemokine binding sequence comprised in SEQ ID NO: 76 is derived from SEQ ID NO: 3 and shown below as SEQ ID NO: 77. SEQ ID NO:77 provides a chemokine-binding sequence binding CCL8. The full-length recipient tick CKBP sequence substituted to create SEQ ID NO: 76 is a variant sequence of Evasin-1 of SEQ ID NO: 32 shown in EZ406190.1 (as discussed above), with the residual recipient sequence remaining after the substitution shown below as SEQ ID NO: 78. The sequence removed from SEQ ID NO: 32 by the substitution is shown below as SEQ ID NO: 79. A chemokine-binding sequence comprising SEQ ID NO: 79 may be used to provide one or more chemokine-binding functions of Evasin-1.

SEQ ID NO: 77:
VCEVSEQEGVGEDNATEDEDYEDFFKPVTCYFANSTVGPLRPPN
SEQ ID NO: 78:
CKQDCNGTTETAPNGTRCFSIGDEGLRRMTANLPYDCPLGQCSNGDCI
PKETYEVCYRRNWRDEKN
SEQ ID NO: 79:
EDDEDYGDLGGCPFLVAENKTGYPTIVA

The hybrid polypeptide of SEQ ID NO: 76 binds CCL8 by virtue of the introduced chemokine-binding sequence from the tick CKBP of SEQ ID NO: 3, whereas Evasin-1 natively does not have CCL8-binding activity. Accordingly, the inventors have shown the ability to isolate an independent binding function from a first tick CKBP and transport this into a second tick CKBP, resulting in a hybrid tick CKBP polypeptide with an altered chemokine binding profile.

Also provided is a hybrid polypeptide comprising the amino acid sequence of SEQ ID NO: 76 or a variant thereof substituted into the amino acid sequence of any tick CKBP polypeptide. A variant of SEQ ID NO: 76 is selected to have CCL8-binding activity. The tick CKBP polypeptide may be selected from any tick CKBP described above. The tick CKBP amino acid sequence into which the amino acid sequence of SEQ ID NO: 76 or a variant thereof is substituted may be selected from any of SEQ ID Nos 1-72 or variants thereof. The tick CKBP amino acid sequence is typically one which does not have CCL8-binding activity, such as SEQ ID NO: 32, 39, 41. The tick CKBP amino acid sequence may be selected from one having a conserved set of eight cysteines as described above, for example any one of SEQ ID NOs 1-3, 6-9, 20-23, 29, 32 and 34-44.

Also provided is a hybrid polypeptide comprising the amino acid sequence of P672_RHIPU E22-F32 (SEQ ID NO: 108) or of any one of SEQ ID NOs 88, 89 and 103-107, or a variant of any thereof substituted into the amino acid sequence of any tick CKBP polypeptide described herein. The tick CKBP polypeptide may be SEQ ID NO: 32. In one aspect, the hybrid polypeptide comprises the amino acid sequence of SEQ ID NO: 95 or a variant thereof.

Further provided is a hybrid polypeptide comprising a chemokine binding sequence of a first tick CKBP amino acid sequence or a variant thereof fused (directly or by a linker as described above) to SEQ ID NO: 78 or a variant thereof. A variant of SEQ ID NO: 78 (or of any other recipient sequence fragment derived from Evasin-1 described herein) includes the corresponding sequence fragment from Evasin-1 of SEQ ID NO: 32 (without the K92E substitution discussed herein). SEQ ID NO: 78 represents an amino acid sequence derived from Evasin-1 able to functionally accommodate a chemokine-binding sequence from another tick CKBP. The chemokine-binding sequence to be provided upstream of SEQ ID NO: 78 or a variant thereof typically binds one or more chemokines that are not bound by SEQ ID NO: 32. The chemokine-binding sequence may be derived from a first tick CKBP amino acid sequence selected from any one of SEQ ID NOs 1-3, 6-9, 20-23, 29, 32 and 34-44.

The inventors have also shown that a greater extent of sequence may be introduced from the tick CKBP of SEQ ID NO: 3 into Evasin-1, and a lesser extent of recipient sequence retained, while providing a hybrid polypeptide binding CCL8. This demonstrates flexibility in substitution of chemokine-binding sequences from one tick CKBP into another tick CKBP. Thus, the additional substituted hybrid polypeptides of SEQ ID NOs 82 and 83 are provided, as shown below. The first (introduced) tick CKBP sequence is bold and underlined; and the residual recipient second tick CKBP sequence in italics): The introduced and recipient tick CKBP sequences are shown below as SEQ ID NOs 84-85 (derived from SEQ ID NO: 82) and SEQ ID NOs: 86-87 (derived from SEQ ID NO: 83).

SEQ ID NO: 82:
VCEVSEQEGVGEDNATEDEDYEDFFKPVTCYFANSTVGPLRPPNCTVV
CTNNTAWWNDTKSDGGHCYSEYRPEKRTHSREIYNCTIGVCGNGDCIP
KETYEVCYRRNWRDEKN
SEQ ID NO: 83:
VCEVSEQEGVGEDNATEDEDYEDFFKPVTCYFANSTVGPLRPPNCTVV
CTNNTAWWNDTKSDGGHCFSIGDEGLRRMTANLPYDCPLGQCSNGDCI
PKETYEVCYRRNWRDEKN
SEQ ID NO: 84:
VCEVSEQEGVGEDNATEDEDYEDFFKPVTCYFANSTVGPLRPPNCTVV
CTNNTAWWNDTKSDGGHCYSEYRPEKRTHSREIYNCTIGVCGNG
SEQ ID NO: 85:
DCIPKETYEVCYRRNWRDEKN
SEQ ID NO: 86:
VCEVSEQEGVGEDNATEDEDYEDFFKPVTCYFANSTVGPLRPPNCTVV
CTNNTAWWNDTKSDGGHC
SEQ ID NO: 87:
FSIGDEGLRRMTANLPYDCPLGQCSNGDCIPKETYEVCYRRNWRDEKN

Also provided herein is a hybrid polypeptide comprising the amino acid sequence of SEQ ID NO: 84 or 86 or a variant of either thereof substituted into the amino acid sequence of any tick CKBP polypeptide. A variant of SEQ ID NO: 84 or 86 is selected to have CCL8-binding activity. Additionally described herein is a hybrid polypeptide comprising a chemokine binding sequence of a first tick CKBP amino acid sequence or a variant thereof fused (directly or by a linker as described above) to SEQ ID NO: 85 or 87 or a variant of either thereof.

Thus, a range of hybrid polypeptides may be provided based on substitution of a chemokine-binding sequence of a first tick CKBP polypeptide into the amino acid sequence of a second tick CKBP polypeptide. Hybrid polypeptides may also be provided which comprise one or more substituted tick CKBP amino acid sequences as described above (comprising a chemokine-binding sequence derived from a first tick CKBP amino acid sequence) fused directly or via a linker region with one or more additional tick CKBP amino acid sequences or variant thereof. Thus, a hybrid polypeptide may comprise a chemokine-binding sequence derived from a first tick CKBP polypeptide and additional tick CKBP amino acid sequences, for example one or two additional tick CKBP amino acid sequences or variants thereof. Such additional tick CKBP amino acid sequences may be selected from any one of SEQ ID NOs 1-72. The combination of one or more chemokine-binding sequences (which may be specific for a single chemokine) and one or more additional tick CKBP amino acid sequences may assist provision of a specific chemokine-binding profile of interest.

Chemokine-Binding Sequences

Identification of a suitable chemokine-binding sequence and selection of a region for substitution may be performed by various means. The inventors have identified that discrete contiguous sequence regions of tick CKBP polypeptides encode chemokine-binding activity. Thus, a tick CKBP polypeptide may be truncated N- or C-terminally and a series of truncated polypeptides then screened for binding activity for one or more chemokines bound by the full-length tick CKBP. Where the tick CKBP polypeptide has a conserved set of eight cysteine residues as discussed above, the inventors' analysis in relation to polypeptides of this group (SEQ ID Nos 3 and 32) indicates that one or more chemokine binding activities are typically present in an N-terminal region. Thus, C-terminal truncations of SEQ ID NOs 1-3, 6-9, 20-23, 29, 32 and 34-44 may be preferable when providing a chemokine-binding sequence based on any of the above tick CKBP polypeptides.

The chemokine-binding sequence may be identified by hydrogen-deuterium exchange mass spectrometry (HDX-MS). The amino acid residues involved in the interface between a chemokine and a tick CKBP polypeptides can be identified and a chemokine-binding sequence inferred. Thus, a tick CKBP polypeptide may result from N- and/or C-terminal truncations of a tick CKBP polypeptide. A tick chemokine binding polypeptide may be truncated N- and/or C-terminally and/or be chemically modified. A series of truncated and/or modified polypeptides may be screened for binding activity for one or more chemokines bound by the full-length tick CKBP. A tick chemokine binding polypeptide may be extended N- and/or C-terminally and/or chemically modified.

The chemokine binding sequence may be at least 10, at least 11, at least 12, at least 15, at least 16, at least 17, at least 20, at least 30, at least 40, at least 50, at least 80 or at least 90 amino acids in length, depending on the particular tick CKBP. The chemokine binding sequence may be of 20-100, 20-90, 20-70, 20-60, 20-50, 10-100, 11-100, 12-100, 16-100, 11-70, 11-60, 11-50, 11-17 amino acids in length. Corresponding N- or C-terminal truncations may be made to any tick CKBP polypeptide described herein to provide a chemokine-binding sequence, and also a recipient sequence able to accommodate a chemokine-binding sequence derived from another tick CKBP.

A chemokine-binding sequence may thus be identified by performing a chemokine binding assay on truncation variants of a tick CKBP polypeptide, such as the biointerferometry assay described in Table 2A, and also in [17]. Other suitable binding assays include HDX-MS, mass spectrometry (MS) dimerization, Alphascreen, surface plasmon resonance, microscale thermophoresis, fluorescent polarization, and FRET based assays. The tick CKBP polypeptide may be truncated to provide a minimal chemokine-binding sequence (for one or more chemokines of interest), and not include other sequence of the tick CKBP polypeptide not essential for the relevant chemokine-binding activity. Truncation variants of a first tick CKBP amino acid sequence that comprise chemokine-binding sequences may also be screened for their ability to inhibit or neutralize chemokine activity, for example by performing a chemokine-induced cell migration assay, for example the assay as described in Table 3, or as described in FIGS. 5 and 6. An example of a functional truncation variant is provided by SEQ ID NO: 89, a truncation variant of parental evasin SEQ ID NO: 3. As shown in FIGS. 14-16, the truncated peptide (used in experiments as an alanine substitution mutant, SEQ ID NO: 88) retained parental binding activity for CCL8, CCL7 and CCL18. Accordingly, SEQ ID NO: 89 or a variant thereof (such as SEQ ID NO:88) may be used alone as a chemokine-binding agent or as a chemokine-binding sequence in any hybrid polypeptide described herein.

A variant of SEQ ID NOs: 88, 89 or 105 to 107 preferably retains one or more, two or more, three or more, preferably four N-terminal acidic amino acid residues. A variant of SEQ ID NOs: 88, 89 or 103 to 107 preferably does not substitute Pro27 for another amino acid (numbering according to the P672_RHIPU parental evasin, SEQ ID NO: 3). A variant of SEQ ID NOs: 89 or 104 to 107 preferably does not substitute Cys30 for alanine (numbering according to the P672_RHIPU parental evasin, SEQ ID NO: 3). A variant of SEQ ID NOs: 88, 89 or 103 to 107 may comprise a substitution of one or more other amino acid residues to alanine or a similar amino acid residue. Such changes do not significantly affect the ability of the peptide to specifically bind CCL8 (see FIG. 19D, Table 9). Similarly, up to all four N-terminal acidic residues may be removed from SEQ ID NO: 89 without abolishing chemokine binding activity (see FIG. 20A-D) and Cys30 may be substituted for alanine without abolishing chemokine activity (see FIG. 20A-J).

A truncated tick CKBP polypeptide such as SEQ ID NO: 89 may also be extended N- and/or C-terminally and variants screened for their ability to inhibit or neutralize chemokine activity, for example by performing suitable binding assays such as those described above. For example, the fluorescent polarization, MS dimerization, Alphascreen and cell migration assays described in FIGS. 19 to 21. The truncated tick CKBP polypeptide may be extended by one or more amino acids, such as two or more, three or more, four or more, five or more, 10 or more, 11 or more, 12 or more, or 16 or more amino acids. Examples of functional extended variants are provided by peptides BK1.2 and BK1.3 (SEQ ID NO:105 and 106) which retained or improved upon parental (BK1.5, SEQ ID NO: 89) specific binding activity for CCL2, CCL3, CCL7 and CCL8.

Examples of functional substituted variants are provided by peptide BK1.1 (SEQ ID NO: 88). Peptide BK1.1 retained the ability of the parental (BK1.5, SEQ ID NO: 89) peptide to bind chemokines CCL7, CCL8 and CCL18, which is retained with or without an N-terminal FITC molecule (FIG. 19F-I). Examples of functional truncated variants are provided by peptides Y21F32C30A and Y21F32 (SEQ ID NO: 103 and 104) which retained or improved upon parental (BK1.1, SEQ ID NO: 88) specific binding activity for CCL2, CCL3 and CCL8. Accordingly, provided herein is a method of inhibiting the signaling of one or more chemokines in a subject, the method comprising administering to the subject a polypeptide selected from any of SEQ ID NOs: 88, 89, and 103 to 109, or a variant thereof. Also provided is a method of inhibiting the signaling of one or more chemokines in an in vitro culture, the method comprising contacting the culture with a peptide selected from any of SEQ ID NOs: 88, 89, and 103 to 109, or a variant thereof. The one or more chemokines are preferably selected from CCL2, CCL3, CCL7, CCL8, and/or CCL18, preferably comprising CCL8. The polypeptide is preferably SEQ ID NO: 106.

Also provided is a method of treating or preventing in a subject one or more diseases associated with CCL2, CCL3, CCL7, CCL8 and/or CCL18, the method comprising administering to the subject a polypeptide selected from any of SEQ ID NOs: 88, 89, 103 to 109, or a variant thereof. The disease is preferably a disease associated with CCL8. The polypeptide is preferably SEQ ID NO: 106.

A tick CKBP polypeptide may also be modified. Examples of modification include addition of an N-terminal tyrosine residue or presence of an intra- or inter-polypeptide disulfide bond (see for example BK1.3 peptide, FIG. 20) or thioether cyclisation (see for example BK1.4 peptide, FIG. 20). Examples of functional modified variants are provided by peptides BK1.2 and BK1.4 (SEQ ID NO: 105 and 107) which retained parental (BK1.3, SEQ ID NO: 106; BK1.5, SEQ ID NO: 89) specific binding activity for CCL2, CCL3 and CCL8. Surprisingly, the inventors found that the addition of an N-terminal tyrosine amino acid residue to a chemokine binding peptide of the invention may improve specific binding to chemokine. Similarly, the inventors surprisingly discovered that peptide BK1.3 (SEQ ID NO: 106) forms an inter-peptide disulphide bond, via Cys30 (numbered according to the P672_RHIPU parental evasin, SEQ ID NO: 3). The N-terminal tyrosine and/or the peptide-dimer may contribute to the improved chemokine inhibition observed.

A consensus sequence for a functional variant peptide based on SEQ ID NO: 88, 89 or 103 to 107 is also provided herein. A variant of SEQ ID NO: 88, 89 or 105 to 107 may retain one or more N-terminal acidic amino acid residues, such as EDED. A variant of SEQ ID NO: 88, 89 or 103 to 107 may retain Pro27 and/or Cys30 (numbering according to the parental P672_RHIPU evasin, SEQ ID NO: 3). Other amino acid residues may be removed, added and/or substituted, such as for alanine or a similar amino acid residue. Said variant preferably specifically binds CCL8.

SEQ ID NOs: 103-107, and more preferably SEQ ID NO: 106 may be used alone as a chemokine-binding agent or as a chemokine-binding sequence in any hybrid polypeptide described herein.

The hybrid polypeptide having a chemokine-binding sequence introduced from the first tick CKBP polypeptide can be confirmed as having the transferred chemokine-binding activity by performing similar binding, inhibition and/or neutralization assays for the relevant chemokine(s).

Additionally, chemokine-binding sequences may be identified based on sequence alignment and structural modelling of tick CKBPs. FIGS. 2 and 4 illustrate how the conserved cysteine sets present in tick CKBP polypeptides allow for alignment of their sequences. Thus, the position of a chemokine-binding sequence identified in one tick CKBP polypeptide (such as SEQ ID NO: 77 derived from SEQ ID NO: 3) can be aligned against other tick CKBP polypeptides of the same sub-family to identify a region putatively comprising a chemokine-binding sequence. Regions of predicted secondary structure or comprising key conserved residues (such as the conserved cysteine residues) are typically avoided for disruption by a truncation. Truncation analysis as described above may then be used to confirm whether the relevant region comprises a chemokine-binding sequence.

Structural modelling may also be used to assist determination of chemokine-binding sequences. A published structure is available for Evasin-1 (3FPU, SEQ ID NO: 32)[22]), in complex with CCL3. As described in FIG. 4, structural models for other tick CKBP polypeptides can be generated using this template, thereby predicting residues in the modelled tick CKBP that form an interface with a chemokine, and a location for a chemokine-binding sequence in the primary sequence. Exemplary models for the tick CKBP polypeptides having the amino acid sequences of SEQ ID NOs against Evasin-1 are shown in FIG. 4. The interacting residues predicted by PISA are indicated in FIG. 4 as sticks, and are listed in each figure subpart. Such interacting residues on a tick CKBP may be mutated to affect binding characteristics, and also provide a guide to identify the transportable domain of the tick CKBP.

Structural analysis may also identify residues that make inter-chain hydrogen or salt bridges and suitable points of transfer that do not disrupt structural folds or motifs, assisting selection of a discrete chemokine-binding sequence and a position for introduction of a chemokine-binding sequence in a recipient tick CKBP amino acid sequence. The models shown in FIG. 4 were obtained using MODELLER [160,167] followed by application of the PISA web server [162] to identify residues predicted to form hydrogen or salt bridges. Similar models can also be obtained also using other modelling software such as I-TASSER [168] or Phyre2 [169]. Where a structural model does not exist for a tick CKBP, this may also be obtained, for example by crystallization or using NMR.

### Novel Tick CKBPs of the Invention

The invention further provides a polypeptide comprising (a) all or part of an amino acid sequence shown in any one of SEQ ID NOs 45-72 or (b) all or part of an amino acid sequence having at least 70% homology or identity to a sequence of (a) over its entire length, wherein said polypeptide binds at least one CXC-class chemokine. SEQ ID NOs 45-72 represent tick CKBP amino acid sequences newly identified and functionally characterised as binding CXC chemokines by the inventors.

The sequence of (a) may be an amino acid sequence shown in any one of SEQ ID NOs 45-60 and 64-65. In such an embodiment, the polypeptide binds one or more human chemokines selected from CXCL7, CXCL9, CXCL10, CXCL11 and CXCL12.

The sequence of (a) may be an amino acid sequence shown in any one of SEQ ID NOs 45-48, 51-53, 56, 59, 60, and 65. In this embodiment, the polypeptide binds one or more human chemokines selected from CXCL7, CXCL9, and CXCL11.

The polypeptide can be any length. The polypeptide is preferably at least 40 amino acids in length, such as at least 50, at least 60, at least 70 or at least 80 amino acids in length. The polypeptide is preferably 250 amino acids or fewer in length, such as 200 amino acids or fewer, 150 amino acids or fewer or 100 amino acids or fewer in length. The length of the polypeptide typically depends on the length of any one of SEQ ID NOs 45-72. Deletions and/or extension are allowable in accordance with the invention as discussed in detail below. The polypeptide is typically from 40 to 250 amino acids in length, such as from 45 to 200 amino acids in length or from 50 to 160 amino acids in length.

The polypeptide is typically formed from naturally-occurring amino acids. The polypeptide may contain non-naturally-occurring amino acids. The polypeptide typically comprises L-amino acids. The polypeptide may comprise D-amino acids.

The selection of variants of SEQ ID NOs: 45 to 72 as discussed below is also applicable to selection of variants of any of SEQ ID NOs 1-44, 73-74 and 76-94. A variant of any one of SEQ ID NOs: 45 to 72 is a polypeptide that has an amino acid sequence which varies from that of any one of SEQ ID NOs: 45 to 72 and has the ability to bind to one or more chemokines. A variant of any one of SEQ ID NOs: 45 to 72 may be a polypeptide that has an amino acid sequence which varies from that of any one of SEQ ID NOs: 45 to 72 and has the ability to bind to and inhibit one or more chemokines.

The variant preferably binds and preferably inhibits one or more or all of the same chemokines as the sequence on which the variant is based. For instance, a variant of SEQ ID NO: 45 is a polypeptide that has an amino acid sequence which varies from that of SEQ ID NO: 45 and has the ability to bind to the chemokine shown in SEQ ID NO: 45's row in Table 6 (CXCL9). The same is true for any of SEQ ID NOs: 46 to 72. Thus, variants of the tick CKBPs as described above preferably bind to and preferably inhibit the same chemokines as the sequence on which the variant is based.

The ability of a variant to bind to and preferably inhibit a chemokine can be assayed using any method known in the art. Suitable methods are described in the Examples and Figures, and include yeast surface display and biolayer interferometry (for binding) and chemotaxis assays (for inhibition).

The variant may be a naturally occurring variant which is expressed naturally, for instance in ticks. Alternatively, the variant may be expressed in vitro or recombinantly as discussed below. Variants also include non-naturally occurring variants produced by recombinant technology.

Over the entire length of the amino acid sequence of any one of SEQ ID NOs: 45 to 72 (or SEQ ID NOs 1-44, 73-74 and 76-94), a variant will preferably be at least 70% homologous or identical to that sequence. More preferably, the variant may have at least 75%, at least 80%, at least 85%, at least 90% and more preferably at least 95%, 97% or 99% homology or amino acid identity to the amino acid sequence of any one of SEQ ID NOs: 35 to 62 over the entire sequence. There may be at least 80%, for example at least 85%, 90% or 95%, homology or amino acid identity over a stretch of 20 or more, for example 30, 40, 50, 60, 70, or more, contiguous amino acids (“hard homology” or “hard identity”).

Standard methods in the art may be used to determine homology. For example the UWGCG Package provides the BESTFIT program, which can be used to calculate homology, for example used on its default settings (Devereux et al (1984) Nucleic Acids Research 12, p387-395). The PILEUP and BLAST algorithms can be used to calculate homology or line up sequences (such as identifying equivalent residues or corresponding sequences (typically on their default settings)), for example as described in Altschul S. F. (1993) J Mol Evol 36:290-300; Altschul, S. F et al (1990) J Mol Biol 215:403-10. Software for performing BLAST analyses is publicly available through the National Center for Biotechnology Information (ncbi.nlm.nih.gov).

Amino acid substitutions may be made to the amino acid sequences of SEQ ID NOs: 45 to 72 (or SEQ ID NOs 1-44, 73-74 and 76-107), for example up to 1, 2, 3, 4, 5, 10, 20, 30 or 50 substitutions. Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 9 below. Where amino acids have similar polarity, this can also be determined by reference to the hydropathy scale for amino acid side chains in Table 10.

TABLE 9
Chemical properties of amino acids

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

TABLE 10
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

One or more amino acids of the amino acid sequence of any one of SEQ ID NOs: 45 to 72 may additionally be deleted from the polypeptides described above. Up to 1, 2, 3, 4, 5, 10, 20 or 30 amino acids may be deleted, or more.

Variants may include fragments of any one of SEQ ID NOs: 45 to 72. Such fragments typically retain a chemokine-binding sequence (for one or more chemokines) of any one of SEQ ID NOs: 45 to 72. Fragments may be at least 40, at least 50, at least 60, at least 70, at least 80, at least 90 or at least 100 amino acids in length.

One or more amino acids may be alternatively or additionally added to the polypeptides described above. Put another way, the polypeptide may comprise a sequence consisting of any one of SEQ ID NOs: 45 to 72 or a variant thereof having an N-terminal and/or C-terminal extension of a number of amino acids. The N-terminal and/or C-terminal extension may comprise 1, 2, 3, 4, 5, 6, 7, 8, 9 or 10 amino acids or more, such as 15, 20, 30, 40, 50 or 100 amino acids.

### Variants of Other Tick CKBP Amino Acid Sequences

Variants of other tick CKBP amino acid sequences described herein (such as SEQ ID NOs 1-44) are typically selected according to the same principles described above for variants of SEQ ID NOs 45-72. Thus, a variant of a first tick CKBP amino acid sequence selected for inclusion in a hybrid polypeptide, and a variant of a second tick CKBP polypeptide also selected for inclusion in the hybrid polypeptide (as described above) may comprise (a) part of the relevant tick CKBP amino acid sequence or (b) all or part of an amino acid sequence having at least 70% homology or identity to the relevant tick CKBP amino acid sequence over its entire length. The variant may comprise any extent of length of the tick CKBP amino acid sequence as described above. The variant may comprise any degree of homology or identity to the relevant tick CKBP amino acid sequence as described above, such as at least 75%, at least 80%, at least 85%, at least 90% and more preferably at least 95%, 97% or 99% homology or amino acid identity to the amino acid sequence of the relevant tick CKBP amino acid sequence over the entire sequence. The variant may comprise substitutions or represent a fragment or extension of the tick CKBP amino acid sequence as described above. Typically the variant binds to and preferably inhibits one or more of the same chemokines as the tick CKBP amino acid sequence on which it is based. Chemokine binding for SEQ ID NOs 1-72 is shown in Tables 2, 4 and 6 above. Thus, a variant of a given tick CKBP amino acid sequence selected from SEQ ID NOs 1-72 may bind to (and preferably inhibit) one or more of, or all of the chemokines shown to be bound by the relevant tick CKBP polypeptide in Tables 2, 4 and 6.

The invention additionally provides variants of the hybrid polypeptides of SEQ ID NOs 73, 74, 76, 80-82 and 92-95, or of the chemokine-binding and recipient sequences of SEQ ID NOs 76-78, 84-89 and 103-107 selected accorded to similar principles to those described above. Such variants may be selected to have the same chemokine binding as the above hybrid polypeptides or chemokine-binding sequences as described herein, or the same ability to act as recipient for a chemokine binding sequence, and for example to comprise a degree of identity or homology to SEQ ID NOs 73, 74, 76-78, 80-89, 92-94 and 103-107 as described above.

### Polypeptides

Any references to polypeptides herein encompass the hybrid polypeptides discussed above, and the novel tick CKBP polypeptides described above, unless indicated otherwise.

The invention encompasses any pharmaceutically acceptable salt of a polypeptide described herein. Said pharmaceutically acceptable salts include, for example, mineral acid salts such as chlorides, hydrochlorides, hydrobromides, phosphates, sulfates, and the like; and the salts of organic acids such as acetates, propionates, malonates, benzoates, and the like; and salts of monocationic metal ions such as sodium and potassium and the like; and salts of bases such as ammonia. A hydrochloride salt or an acetate salt is preferred.

The polypeptide may be labelled with a detectable label. The detectable label may be any suitable label which allows the polypeptide to be detected. Suitable labels include, but are not limited to, fluorescent molecules, radioisotopes, e.g. ¹²⁵I, ³⁵S, enzymes, antibodies, antigens, polynucleotides and ligands such as biotin. The label is preferably a tracer that is suitable for positron emission tomography (PET), such as fluorine (¹⁸F). The label is preferably a tracer suitable for magnetic resonance imaging (MRI), such as fluorine (¹⁹F). The label may be a Fluorescein isothiocyanate (FITC) moiety.

The polypeptides of the invention may be made in any way. They may be made in accordance with the invention as discussed in more detail below.

The polypeptides described herein can be prepared by any suitable technique.

Alternatively, the polypeptide may be made by solid-phase peptide synthesis (SPPS) is a preferred technique. This involves formation of the peptide on small solid beads. Using SPPS, the polypeptide remains covalently attached to a bead during synthesis. The polypeptide is synthesised using repeated cycles of coupling-washing-deprotection-washing. In particular, the free N-terminal amine of a solid-phase attached polypeptide is coupled to a single N-protected amino acid unit. This unit is then deprotected, revealing a new N-terminal amine to which a further protected amino acid is attached. These steps are repeated until the polypeptide is complete. The polypeptide is then cleaved from the beads using a suitable reagent.

Suitable protecting groups, reagents, solvents and reaction conditions for SPPS are well known to those skilled in the art and as such conditions can be determined by one skilled in the art by routine optimization procedures.

Pharmaceutically acceptable salts of polypeptides can be prepared by any suitable technique. Typically, salification involves reaction of the polypeptide or a salt thereof with a suitable reagent, typically acid, to obtain the pharmaceutically acceptable salt selected.

For example, a hydrochloride salt of a polypeptide can be prepared by initially cleaving the polypeptide from the solid phase using trifluoroacetic acid. The polypeptide will thus initially be a trifluoroacetate salt. The trifluoroacetate salt can then be converted into a hydrochloride salt by any known technique, such as ion exchange on a suitable column using hydrochloric acid as an eluent.

The polypeptide or polypeptide salt products can be purified, where required, by any suitable technique. High pressure liquid chromatography (HPLC) can be used, for example.

The term “polypeptide” includes not only molecules in which amino acid residues are joined by peptide (—CO—NH—) linkages but also molecules in which the peptide bond is reversed. Such retro-inverso peptidomimetics may be made using methods known in the art, for example such as those described in Meziere et al (1997) J. Immunol. 159, 3230-3237. This approach involves making pseudopolypeptides containing changes involving the backbone, and not the orientation of side chains.

Similarly, the peptide bond may be dispensed with altogether provided that an appropriate linker moiety which retains the spacing between the carbon atoms of the amino acid residues is used; it is particularly preferred if the linker moiety has substantially the same charge distribution and substantially the same planarity as a peptide bond. It will also be appreciated that the peptide may conveniently be blocked at its N-or C-terminus so as to help reduce susceptibility to exoproteolytic digestion. For example, the N-terminal amino group of the polypeptides may be protected by reacting with a carboxylic acid and the C-terminal carboxyl group of the peptide may be protected by reacting with an amine. Other examples of modifications include glycosylation and phosphorylation. Another potential modification is that hydrogens on the side chain amines of R or K may be replaced with methylene groups (—NH₂→

—NH(Me) or —N(Me)₂). Other potential modifications include thioether cyclization and intra- and/or inter-peptide disulphide bonds.

Polypeptides according to the invention may also include peptide variants that increase or decrease the polypeptide's half-life in vivo. Examples of analogues capable of increasing the half-life of polypeptides used according to the invention include peptoid analogues of the peptides, D-amino acid derivatives of the peptides, and peptide-peptoid hybrids. A further embodiment of the variant polypeptides used according to the invention comprises D-amino acid forms of the polypeptide. The preparation of polypeptides using D-amino acids rather than L-amino acids greatly decreases any unwanted breakdown of such an agent by normal metabolic processes, decreasing the amounts of agent which needs to be administered, along with the frequency of its administration.

The polypeptides may also be derived from amino acid mutants, glycosylation variants and other covalent derivatives of the parent polypeptides. Exemplary derivatives include molecules wherein the polypeptides of the invention are covalently modified by substitution, chemical, enzymatic, or other appropriate means with a moiety other than a naturally occurring amino acid. Further included are naturally occurring variant amino acid sequences of the parent polypeptides. Such a variant amino acid sequence may be encoded by an allelic variant or represent an alternative splicing variant.

Modifications as described above may be prepared during synthesis of the peptide or by post-production modification, or when the polypeptide is in recombinant form using the known techniques of site-directed mutagenesis, random mutagenesis, or enzymatic cleavage and/or ligation of nucleic acids.

The polypeptides described herein may also be modified to improve physicochemical characteristics. Thus, for example, original amino acid sequences may be altered to improve their solubility, and accordingly a polypeptide of the invention having a variant sequence will preferably be more soluble than a polypeptide having the corresponding original amino acid sequence under equivalent conditions. Methods for evaluating the solubility of polypeptides are well known in the art.

The present invention also provides a fusion polypeptide comprising fusion polypeptide comprising a polypeptide of the invention linked to a second peptide or polypeptide. The polypeptide of the invention may be any of those discussed above. The polypeptide of the invention is typically covalently linked to the second peptide or polypeptide. The polypeptide of the invention is typically genetically fused to the second peptide or polypeptide. The polypeptide of the invention is genetically fused to the second peptide or polypeptide if the whole construct is expressed from a single polynucleotide sequence. The coding sequences of the polypeptide of the invention and the second peptide or polypeptide may be combined in any way to form a single polynucleotide sequence encoding the construct. They may be genetically fused in any configuration. They are typically fused via their terminal amino acids. For instance, the amino terminus of the polypeptide of the invention may be fused to the carboxy terminus of the second peptide or polypeptide and vice versa.

The polypeptide of the invention may be attached directly to the second peptide or polypeptide. The polypeptide of the invention is preferably attached to the second peptide or polypeptide using one or more linkers. The one or more linkers may be designed to constrain the mobility of the polypeptides. Suitable linkers include, but are not limited to, chemical crosslinkers and peptide linkers. Peptide linker are preferred if the polypeptide of the invention and second peptide or polypeptide are genetically fused. Preferred linkers are amino acid sequences (i.e. peptide linkers). The length, flexibility and hydrophilicity of the peptide linker are typically designed such that it does not to disturb the functions of the polypeptide of the invention. Preferred flexible peptide linkers are stretches of 2 to 20, such as 4, 6, 8, 10 or 16, serine and/or glycine amino acids. More preferred flexible linkers include (SG)1, (SG)2, (SG)3, (SG)4, (SG)5 and (SG)8 wherein S is serine and G is glycine. Preferred rigid linkers are stretches of 2 to 30, such as 4, 6, 8, 16 or 24, proline amino acids. More preferred rigid linkers include (P)12 wherein P is proline. The polypeptide of the invention may be attached to the second peptide or polypeptide via the side chains of the amino acid residues. Such attachments include thioether and disulphide bonds.

The polypeptide of the invention may be transiently attached to the second peptide or polypeptide by a hex-his tag or Ni-NTA. They may also be modified such that they transiently attach to each other. The polypeptide of the invention may also be attached to the second peptide or polypeptide via cysteine linkage. This can be mediated by a bi-functional chemical linker or by a polypeptide linker with a terminal presented cysteine residue.

The second peptide or polypeptide may be any peptide or protein. The second protein is preferably a fragment crystallizable region (Fc region). The Fc region may be from any of the types of subject discussed below. Fc region is preferably human. The Fc region may derived from any isotype of antibody, such as IgA, IgD, IgG, IgE or IgM.

The second peptide or polypeptide may be an epitope tag or purification tag or cell-surface display tag or a tag that enables or facilitates systemic peptide delivery or delivery and targeting to a specific organ or to a tumour, or facilitates transfer across a barrier such as skin or gut or blood brain barrier. Suitable tags are known in the art. Suitable tags include, but are not limited to, AviTag, calmodulin-tag, polyglutamate tag, E-tag, FLAG-tag, HA-tag, His-tag, Myc-tag, S-tag, SBP-tag, Softag 1, Softag 3, Strep-tag, TC tag, V5 tag, VSV-tag, Xpress tag, Isopeptag, SpyTag, SnoopTag, BCCP (Biotin Carboxyl Carrier Protein), Glutathione-S-transferase-tag, Green fluorescent protein-tag, Halo-tag, Maltose binding protein-tag, Nus-tag, Thioredoxin-tag, Strep-tag, Skin permeating and cell entering (SPACE)-tag, TD1-tag, magainin tag, TAT-tag, penetratin-tag, cell penetrating peptide (CPP)-tag, Fc tag. The second peptide or polypeptide may be a signal peptide, such as an IgK peptide.

The fusion polypeptide may be labelled with a detectable label. The detectable label may be any of those discussed above.

### Polypeptide Combinations of the Invention

The invention also provides a combination of two or more polypeptides of the invention, i.e. two or more different polypeptides of the invention. The combination may comprise two or more polypeptides of the invention, two or more fusion polypeptides of the invention or a two or more of both types of polypeptide.

The combination may comprise any number of different polypeptides of the invention. For instance, the combination may comprise 3, 4, 5, 6, 7, 8, 9, 10, 11, 12, 13, 14, 15, 16, 17, 18, 19, 20, 21, 22, 23, 24, 25, 26, 27, 28, 29, 30 or 31 different polypeptides of the invention. The combination may comprise 10 or more, 20 or more, 30 or more, 40 or more or 50 or more polypeptides of the invention.

One or more of, such as all of, the polypeptides in the combination may be labelled with a detectable label. The label may be any of those discussed above. Different polypeptides in the combination may be labelled with the same detectable label or different detectable labels.

### Polynucleotides of the Invention

The invention also provides a polynucleotide which encodes a polypeptide of the invention. The polypeptide may be any of those discussed above.

The invention also provides a polynucleotide which encodes a fusion polypeptide of the invention. The fusion polypeptide is preferably genetically fused as discussed above.

The invention also provides a polynucleotide which encodes a combination of the invention. The coding sequences for the two or more polypeptides in the combination may be present in a single polynucleotide of the invention. This is typically the case when the combination is encoded by a single vector of the invention.

A polynucleotide, such as a nucleic acid, is a polymer comprising two or more nucleotides. The nucleotides can be naturally occurring or artificial. A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The nucleotide is typically a ribonucleotide or deoxyribonucleotide. The nucleotide typically contains a monophosphate, diphosphate or triphosphate. Phosphates may be attached on the 5′ or 3′ side of a nucleotide.

Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (cAMP), cyclic guanosine monophosphate (cGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides are preferably selected from AMP, TMP, GMP, UMP, dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hyrdroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light.

The nucleotides in the polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups as in nucleic acids. The nucleotides may be connected via their nucleobases as in pyrimidine dimers.

The polynucleotide can be a nucleic acid, such as deoxyribonucleic acid (DNA) or a ribonucleic acid (RNA). The polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains. The polynucleotide may be single stranded or double stranded.

The polynucleotide sequence encodes the relevant polypeptide(s) on the basis of the genetic code, including its degeneracy.

The polynucleotide may be a ribonucleic acid modified to reduce immunogenicity and increase stability for instance by substitution of uridine and cytidine with 1-methylpseudouridine and 5-methylcytidine, and/or placing an Anti-Reverse Cap Analog (ARCA) cap at the 5′ end. Such modified ribonucleic acids can be delivered using nanoparticles and other transfection reagents ([38-41]).

Polynucleotide sequences may be derived and replicated using standard methods in the art, for example using PCR involving specific primers. It is straightforward to generate polynucleotide sequences using such standard techniques. These are discussed in more detail below.

### Polynucleotide Combinations of the Invention

The invention also provides a combination of two or more polynucleotides each of which encodes a polypeptide of the invention, i.e. each of which encodes a different polypeptide of the invention. The combination may encode two or more polypeptides of the invention, two or more fusion polypeptides of the invention or a two or more of both types of polypeptide.

The combination may comprise any number of different polynucleotides. For instance, the combination may comprise 3, 4, 5, 6, 7, 8, 9, 10, 11, 12, 13, 14, 15, 16, 17, 18, 19, 20, 21, 22, 23, 24, 25, 26, 27, 28, 29, 30 or 31 different polynucleotide of the invention. The combination may comprise 10 or more, 20 or more, 30 or more, 40 or more or 50 or more polynucleotides of the invention.

### Vectors of the Invention

The invention also provides a vector comprising a polynucleotide of the invention or a combination of two or more polynucleotides of the invention.

The vector may be a cloning vector. The amplified sequences may be incorporated into a recombinant replicable vector such as a cloning vector. The vector may be used to replicate the polynucleotide in a compatible host cell. Thus polynucleotide sequences may be made by introducing the polynucleotide into a replicable vector, introducing the vector into a compatible host cell, and growing the host cell under conditions which bring about replication of the vector.

The vector may be recovered from the host cell. Suitable host cells for cloning of polynucleotides are known in the art and described in more detail below. The vector may be an expression vector. The polynucleotide sequence may be cloned into any suitable expression vector. In an expression vector, the polynucleotide of the invention or the combination of the invention is typically operably linked to a control sequence which is capable of providing for the expression of the polynucleotide or the combination by the host cell. Such expression vectors can be used to express one or more polypeptides of the invention.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. Multiple copies of the same or different polynucleotide may be introduced into the vector.

The term “control sequence” is intended to include promoters, enhancers, internal ribosomal entry sites (IRES), and other expression control elements (e.g. transcription termination signals, such as polyadenylation signals and poly-U sequences). Such control sequences are described, for example, in Goeddel, GENE EXPRESSION TECHNOLOGY: METHODS IN ENZYMOLOGY 185, Academic Press, San Diego, Calif (1990). Control sequences include those that direct constitutive expression of a nucleotide sequence in many types of brain cell and those that direct expression of the nucleotide sequence only in certain brain cells. A non-limiting example of a suitable neuron-specific promoters include the neurofilament promoter; Byrne and Ruddle, 1989. Proc. Natl. Acad. Sci. USA 86: 5473-5477.

Control sequences may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EFla promoter. Also encompassed by the term “control sequence” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. With regards to control sequences, mention is made of U.S. patent application Ser. No. 10/491,026. With regards to promoters, mention is made of PCT publication WO 2011/028929 and U.S. application Ser. No. 12/511,940.

The expression vector may then be introduced into a suitable host cell. Thus, polypeptide of the invention can be produced by inserting a polynucleotide or a combination into an expression vector, introducing the vector into a compatible bacterial host cell, and growing the host cell under conditions which bring about expression of the polynucleotide or combination. The vectors may be for example, plasmid, virus or phage vectors provided with an origin of replication, optionally a promoter for the expression of the said polynucleotide or combination and optionally a regulator of the promoter. The vectors may contain one or more selectable marker genes, for example an ampicillin resistance gene. Promoters and other expression regulation signals may be selected to be compatible with the host cell for which the expression vector is designed. A T7, trc, lac, ara or λ_{L} promoter is typically used.

The vector may be used to administer a polynucleotide of the invention or a combination of two or more polynuclelotides to a subject as discussed in more detail below. Conventional viral and non-viral based gene transfer methods can be used to introduce the polynucleotide or combination into cells. Non-viral vector delivery systems include DNA plasmids, RNA, naked nucleic acid, and nucleic acid complexed with a delivery vehicle, such as a liposome. Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Conventional viral based expression systems could include retroviral, lentivirus, adenoviral, adeno-associated (AAV) and herpes simplex virus (HSV) vectors for gene transfer. Methods for producing and purifying such vectors are know in the art.

Exemplary vector systems for using the invention are a virus, such as rAAV, that comprises or consists essentially of an exogenous polynucleotide encoding the polypeptide, fusion polypeptide or polypeptide combination of the invention, e.g., a cassette comprising or consisting essentially of a promoter, a polynucleotide encoding the polypeptide, fusion polypeptide or polypeptide combination of the invention and a terminator.

Since AAV is a DNA virus, the polynucleotides used in AAV or rAAV are advantageously DNA.

The vector may be delivered using nanoparticle delivery systems. Such delivery systems include, but are not limited to, lipid-based systems, liposomes, micelles, microvesicles, exosomes, and gene gun. With regard to nanoparticles that can deliver RNA, see, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93. Lipid Nanoparticles, Spherical Nucleic Acid (SNA™) constructs, nanoplexes and other nanoparticles (particularly gold nanoparticles) are also contemplated as a means for delivery of a polynucleotide or a polynucleotide of the invention. The invention provides any of these deliver systems comprising a vector of the invention, a polynucleotide of the invention or a polynucleotide combination of the invention.

In some embodiments, the vector may form a component of an inducible system. The inducible nature of the system would allow for spatiotemporal control of expression of a polypeptide of the invention or a combination of such polypeptides using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc), or light inducible systems (Phytochrome, LOV domains, or cryptochrome).

As will be clear from below, the polynucleoide of the invention or a polynucleotide combination of the invention or any expression vector containing these components may be present in a population of cells. The cells may be administered to the subject. Suitable ways of modifying and administering cells are known in the art.

### Host Cells of the Invention

The invention also provides a host cell which comprises a polynucleotide of the invention, a polynucleotide combination of the invention or a vector of the invention. The host cell may be used to replicate the polynucleotide, combination or vector. The host cell may be used to express a polypeptide of the invention or a combination of polypeptides of the invention in vitro. The host cell may be used to deliver the polynucleotide, combination or vector to a subject in need thereof as discussed below.

Host cells will be chosen to be compatible with the cloning or expression vector used to transform the cell. Suitable conditions are known in the art (see, for instance, Sambrook, J. and Russell, D. supra).

Suitable cells for use in the invention include prokaryotic cells and eukaryotic cells. The prokaryotic cell is preferably a bacterial cell. Suitable bacterial cells include, but are not limited to, Escherichia coli, Corynebacterium and Pseudomonas fluorescens. Any E. coli cell with a DE3 lysogen, for example C41 (DE3), BL21 (DE3), JM109 (DE3), B834 (DE3), TUNER, Origami and Origami B, can express a vector comprising the T7 promoter.

Suitable eukaryotic cells include, but are not limited to, Saccharomyces cerevisiae, Pichia pastoris, filamentous fungi, such as Aspergillus, Trichoderma and Myceliophthora thermophila C1, baculovirus-infected insect cells, such as Sf9, Sf21 and High Five strains, non-lytic insect cells, Leishmania cells, plant cells, such as tobacco plant cells, and mammalian cells, such as Bos primigenius cells (Bovine), Mus musculus cells (Mouse), Chinese Hamster Ovary (CHO) cells, Human Embryonic Kidney (HEK) cells, Baby Hamster Kidney (BHK) cells and HeLa cells. Other preferred mammalian cells include, but are not limited to, PC12, HEK293, HEK293A, HEK293T, CHO, BHK-21, HeLa, ARPE-19, RAW264.7 and COS cells.

The host cell is preferably HEK293T.

If the cell is being administered to a subject, the cell is preferably derived from the subject or a subject of the same species. For instance, a human cell is typically administered to a human subject. The host cell is preferably autologous. In other words, the cell is preferably derived from the subject into which the cell will be administered. Alternatively, the host cell is preferably allogeneic. In other words, the cell is preferably derived from a patient that is immunologically compatible with the patient into which the cell will be administered.

The cell may be isolated, substantially isolated, purified or substantially purified. The cell is isolated or purified if it is completely free of any other components, such as culture medium or other cell types. The cell is substantially isolated if it is mixed with carriers or diluents, such as culture medium and others discussed above and below, which will not interfere with its intended use. Alternatively, the host cell of the invention may be present in a growth matrix or immobilized on a surface as discussed below.

### Pharmaceutical Compositions of the Invention

The invention also provides a pharmaceutical composition comprising (a) a polypeptide of the invention, a polypeptide combination of the invention, a polynucleotide of the invention, a vector of the invention or a host cell of the invention and (b) a pharmaceutically acceptable carrier or diluent. The carrier or diluent may be any of those discussed above with reference to the vectors of the invention.

The carrier(s) or diluent(s) present in the pharmaceutical composition must be “acceptable” in the sense of being compatible with the other ingredients of the composition and not deleterious to the recipient thereof. Typically, carriers for injection, and the final formulation, are sterile and pyrogen free. Preferably, the carrier or diluent is water. A pharmaceutically acceptable carrier or diluent may comprise as one of its components thioglycerol or thioanisole.

Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol, thioglycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in Remington's Pharmaceutical Sciences (Mack Pub. Co., N.J. 1991).

The active agents are typically present at 0.1% to 50% by weight in the pharmaceutical composition, more preferably at 0.1% to 5% by weight. They may be present at less than 0.1% by weight in the pharmaceutical composition.

The pharmaceutically acceptable carrier or diluent is typically present at 50% to 99.9% by weight in the pharmaceutical composition, more preferably at 95% to 99.9% by weight. The pharmaceutically acceptable carrier or diluents may be present at more than 99.9% by weight in the pharmaceutical composition.

Pharmaceutical compositions include, but are not limited to pharmaceutically acceptable solutions, lyophilisates, suspensions, emulsions in oily or aqueous vehicles, pastes, and implantable sustained-release or biodegradable compositions. Such pharmaceutical compositions may further comprise one or more additional ingredients including, but not limited to, suspending, stabilizing, or dispersing agents. A lyophilisate may comprise one or more of trehalose, thioglycerol and thioanisole. In one embodiment of a pharmaceutical composition for parenteral administration, the active ingredient is provided in dry form (e.g., a lyophilisate, powder or granules) for reconstitution with a suitable vehicle (e.g., sterile pyrogen-free water) prior to parenteral administration of the reconstituted pharmaceutical composition.

The pharmaceutical composition may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable compositions may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono-or di-glycerides.

Other parenterally-administrable pharmaceutical compositions which are useful include those which comprise the active ingredient in microcrystalline form, in a liposomal preparation, or as a component of a biodegradable polymer systems. Pharmaceutical compositions for sustained release or implantation may comprise pharmaceutically acceptable polymeric or hydrophobic materials such as an emulsion, an ion exchange resin, a sparingly soluble polymer, or a sparingly soluble salt.

For example, solid oral forms may contain, together with the active substance, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, gum arabic, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical compositions. Such pharmaceutical preparations may be manufactured in known manner, for example, by means of mixing, granulating, tabletting, sugar-coating, or film-coating processes.

Liquid dispersions for oral administration may be syrups, emulsions or suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol. The suspensions or solutions for intramuscular injections may contain, together with the active substance, a pharmaceutically acceptable carrier, e.g. sterile water, olive oil, ethyl oleate, glycols, e.g. propylene glycol, and if desired, a suitable amount of lidocaine hydrochloride.

Solutions for intravenous administration or infusion may contain as carrier, for example, sterile water or preferably they may be in the form of sterile, aqueous, isotonic saline solutions.

For suppositories, traditional binders and carriers may include, for example, polyalkylene glycols or triglycerides; such suppositories may be formed from mixtures containing the active ingredient in the range of 0.5% to 10%, preferably 1% to 2%.

Oral compositions include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release compositions or powders and contain 10% to 95% of active ingredient, preferably 25% to 70%. Where the pharmaceutical composition is lyophilised, the lyophilised material may be reconstituted prior to administration, e.g. a suspension. Reconstitution is preferably effected in buffer.

Capsules, tablets and pills for oral administration to an individual may be provided

with an enteric coating comprising, for example, Eudragit “S”, Eudragit “L”, cellulose acetate, cellulose acetate phthalate or hydroxypropylmethyl cellulose.

Polynucleotides may be present in combination with cationic lipids, polymers or targeting systems.

Uptake of polynucleotide or oligonucleotide constructs may be enhanced by several known transfection techniques, for example those including the use of transfection agents. Examples of these agents include cationic agents, for example, calcium phosphate and DEAE-Dextran and lipofectants, for example, lipofectamine and transfectam. The dosage of the polynucleotide or oligonucleotide to be administered can be altered.

Alternatively, the active agent may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The composition will depend upon factors such as the nature of the active agent and the method of delivery. The pharmaceutical composition may be administered in a variety of dosage forms. It may be administered orally (e.g. as tablets, troches, lozenges, aqueous or oily suspensions, dispersible powders or granules), topically, parenterally, subcutaneously, by inhalation, intravenously, intramuscularly, intralymphatically (such as to lymph nodes in the groin), intrasternally, transdermally, intradermally, epidermally, sublingually, intranasally, buccally or by infusion techniques. The administration may be intratonsillar. The administration may be as suppositories. The administration may be made by iontophoresis. Preferably, the administration is intradermal, epidermal or transdermal. The administration may be made by a patch, such as a microtine patch. Administration is discussed in more detail below.

A physician will be able to determine the required route and means of administration for each particular individual.

The pharmaceutical compositions of the invention are preferably provided sealed in a container. The pharmaceutical compositions are typically provided in unit dose form, for example single dose form. They may alternatively be provided in multi-dose form. Where the pharmaceutical composition is a pharmaceutically acceptable solution, the solution may be provided in an ampoule, sealed vial, syringe, cartridge, flexible bag or glass bottle. Where the pharmaceutical composition is a lyophilisate, it is preferably provided in a sealed vial.

The pharmaceutical compositions of the invention will comprise a suitable concentration of each agent to be effective without causing adverse reaction. Where the pharmaceutical composition is for example a lyophilisate, the relevant concentration will be that of each polypeptide following reconstitution. Typically, the concentration of each agent in the pharmaceutical composition when in solution will be in the range of 0.03 to 200 nmol/ml. The concentration of each agent may be more preferably in the range of 0.3 to 200 nmol/ml, 3 to 180 nmol/ml, 5 to 160 nmol/ml, 10 to 150 nmol/ml, 50 to 200 nmol/ml or 30 to 120 nmol/ml, for example about 100 nmol/ml. The pharmaceutical composition should have a purity of greater than 95% or 98% or a purity of at least 99%.

In an embodiment where the invention involves combines therapy, the other therapeutic agents or adjuvants may be administered separately, simultaneously or sequentially. They may be administered in the same or different pharmaceutical compositions. A pharmaceutical composition may therefore be prepared which comprises an agent of the invention and also one or more other therapeutic agents or adjuvants. A pharmaceutical composition of the invention may alternatively be used simultaneously, sequentially or separately with one or more other therapeutic compositions as part of a combined treatment.

### In Vitro Methods of the Invention

The invention also provides a method of inhibiting the signalling of one or more chemokines in an in vitro culture, the method comprising contacting the culture with a polypeptide of the invention, a combination of the invention, a polynucleotide of the invention, a vector of the invention or a host cell of the invention.

The method may comprise inhibiting any number of chemokines, such as 1, 2, 3, 4, 5, 6, 7, 8, 9, 10, 11, 12, 13 or 14 chemokines. The chemokines may be selected from any of those in Tables 2, 4, and 6. When inhibiting the one or more chemokines in a particular row in the above Tables, a polypeptide, combination, polynucleotide, vector or host cell of the invention based on the tick CKBP in the same row is preferably used in the method of the invention. For instance, when inhibiting CCL8 employing a chemokine-binding sequence from a CCL8-binding tick CKBP, a polypeptide, combination, polynucleotide, vector or host cell of the invention based on the sequence shown in SEQ ID NO: 8 may be used. A hybrid polypeptide comprising the amino acid sequence of SEQ ID NO: 76, 82, 83 or 95 may also be used.

Similarly, when inhibiting CCL2 or CCL1/CCL2/CCL3/CCL5 employing applicable chemokine-binding sequences from tick CKBPs described herein, a polypeptide, combination, polynucleotide, vector or host cell of the invention based on the sequence shown in SEQ ID NO: 1 is preferably used. When inhibiting one or more of CCL2, CC13 and/or CCL20 employing applicable chemokine binding sequences from tick CKBPs described herein, a polypeptide, combination, polynucleotide, vector or host cell of the invention based on the sequence shown in any one of SEQ ID NOs 1-3, 6-9, 20-23 and 29 is preferably used. When inhibiting one or more of CXCL3, CXCL10 and/or CXCL12 employing applicable chemokine binding sequences from tick CKBPs described herein, a polypeptide, combination, polynucleotide, vector or host cell of the invention based on the sequence shown in SEQ ID NO 5 or 19 is preferably used.

When inhibiting one or more of CXCL1, CXCL7, CXCL8, CXCL9, CXCL10, CXCL11 or CXCL12, a polypeptide, combination, polynucleotide, vector or host cell of the invention based on the sequence shown in any one of SEQ ID NOs: 45-72 and indicated in Table 6 as binding the relevant chemokine(s) may be used. For example, products of the invention as above based on SEQ ID NO: 45 may be used to inhibit CXCL9. Products of the invention as above based on one of SEQ ID NOs 45-48, 51-53, 56, 59-60 and 65 may be used to inhibit one or more of CXCL7, CXCL9 and CXCL11, as indicated in Table 6.

The in vitro culture is preferable a culture of cells capable of undergoing chemotaxis. The in vitro culture is preferably a chemotactic assay. The culture may be present in a culture flask or the wells of a flat plate, such as a standard 96 or 384 well plate. Such plates are commercially available Fisher scientific, VWR suppliers, Nunc, Starstedt or Falcon. Conditions for culturing cells are known in the art.

The polypeptide, combination, polynucleotide, vector or host cell of the invention may be administered in any of the forms discussed above.

### Therapeutic Methods of the Invention

The invention also provides a method of inhibiting the signalling of one or more chemokines in a subject, the method comprising administering to the subject a polypeptide of the invention, a combination of the invention, a polynucleotide of the invention, a vector of the invention or a host cell of the invention. The invention also provides a polypeptide of the invention, a combination of the invention, a polynucleotide of the invention, a vector of the invention or a host cell of the invention for use in a method of inhibiting the signalling of one or more chemokines in a subject. The invention also provides use of a polypeptide of the invention, a combination of the invention, a polynucleotide of the invention, a vector of the invention or a host cell of the invention in the manufacture of a medicament for use in inhibiting the signalling of one or more chemokines in a subject.

The method may comprise inhibiting any number of chemokines, such as 1, 2, 3, 4, 5, 6, 7, 8, 9, 10, 11, 12, 13 or 14 chemokines. The chemokines may be selected from any of those in Tables 2, 4 and 6. When inhibiting the one or more chemokines in a particular row in the above Tables, a polypeptide, combination, polynucleotide, vector or host cell of the invention based on the tick CKBP in the same row is preferably used in the method of the invention. The examples of selection of particular tick CKBP amino acid sequences for in vitro inhibition of particular chemokines provided above are also applicable to selection of tick CKBP amino acid sequences for in vivo inhibition of the same chemokines.

The skilled person can design combinations of tick CKBPs to inhibit specific combinations of chemokines.

The invention also provides a method of treating or preventing in a subject one or more diseases associated with one or more chemokines, the method comprising administering to the subject a polypeptide of the invention, a combination of the invention, a polynucleotide of the invention, a vector of the invention or a host cell of the invention. The invention also provides a polypeptide of the invention, a combination of the invention, a polynucleotide of the invention, a vector of the invention or a host cell of the invention for use in a method of treating or preventing in a subject one or more diseases associated with one or more chemokines. The invention also provides use of a polypeptide of the invention, a combination of the invention, a polynucleotide of the invention, a vector of the invention or a host cell of the invention in the manufacture of a medicament for treating or preventing in a subject one or more diseases associated with one or more chemokines.

A disease is associated with one or more chemokines if the disease has a chemokine component. In other words, one or more symptoms of the disease may be treated or prevented by inhibiting one or more chemokines. Any number of chemokines may be involved as discussed above. The chemokines are preferably selected from those shown in any of Tables 2, 4 and 6 and also from those shown in FIG. 1.

The method may comprise treating or preventing any number of diseases associated with one or more chemokines, such as 1, 2, 3, 4, 5, 6, 7, 8, 9, 10, 11, 12 or 13 diseases. The chemokines may be selected from any of those in Tables 2, 4 and 6. The one or more diseases may be as identified in Table 7 or 8 or FIG. 1. When treating or preventing the one or more diseases in a particular row of Table 7 or 8, a polypeptide, combination, polynucleotide, vector or host cell of the invention representing an amino acid sequence from the tick CKBP in the same row (or an amino acid sequence of another tick CKBP shown in Table 6 as binding one or more of the chemokines indicated in the above row of Table 7) is preferably used in the method of the invention. When treating or preventing any specific disease shown in FIG. 1, a polypeptide, combination, polynucleotide, vector or host cell of the invention representing amino acid sequence(s) from one or more tick CKBPs shown to bind chemokines associated with that disease (as shown in FIG. 1) is preferably used. Chemokine-binding properties of each of SEQ ID NOs 1-72 are shown in Tables 2, 4 and 6 and FIG. 12.

As seen from FIG. 1 and Tables 7 and 8, diseases that may be treated or prevented by polypeptides representing amino acid sequences derived from SEQ ID NOs 1-3, 6-9, 20-23 and 29 as described above (and related polynucleotides/combinations/host cells) include diseases where CCL2 is known to be expressed including myocarditis, myocardial infarction, skin fibrosis, vasculitis, atherosclerosis, stroke, multiple sclerosis, Alzheimer disease, primary biliary cirrhosis, liver fibrosis, non alcoholic steato hepatitis, paracetamol liver injury, alcohol liver injury, idiopathic pulmonary fibrosis, kidney fibrosis, inflammatory bowel disease, rheumatoid arthritis, and breast cancer; where CCL13 is known to be expressed, including giant cell myocarditis, myocardial infarction, vasculitis, atherosclerosis, idiopathic pulmonary fibrosis, and rheumatoid arthritis, and where CCL20 is known to be expressed including myocarditis, vasculitis, atherosclerosis, stroke, primary biliary cirrhosis, alcohol liver injury, idiopathic pulmonary fibrosis, inflammatory bowel disease, rheumatoid arthritis, psoriasis, breast cancer and colorectal cancer.

Diseases that may be treated or prevented by polypeptides representing amino acid sequences derived from SEQ ID NOs 5 or 19 (and related polynucleotides/combinations/host cells) include diseases where CXCL3 is known to be expressed, including, idiopathic pulmonary fibrosis and breast cancer, where CXCL10 is known to be expressed, including atherosclerosis, inflammatory bowel disease, rheumatoid arthritis, liver fibrosis, idiopathic pulmonary fibrosis, multiple sclerosis, psoriasis, Alzheimer disease, myocarditis, primary biliary cirrhosis, autoimmune hepatitis, vasculitis, non-alcoholic steatohepatitis, myocardial infarction, and alcohol liver injury, or where CXCL12 is expressed, as in atherosclerosis, inflammatory bowel disease, rheumatoid arthritis, idiopathic pulmonary fibrosis, multiple sclerosis, colorectal cancer, myocarditis, primary biliary cirrhosis, primary sclerosing cholangitis, and autoimmune hepatitis.

A hybrid polypeptide of the invention may be used to treat or prevent a disease comprising expression of multiple chemokines, such as five or more, eight or more or ten or more chemokines. The multiple chemokines may preferably comprise both CC and CXC chemokines. A hybrid polypeptide binding both a CC chemokine and a CXC chemokine may be used to inhibit chemokine signalling in, and to treat or prevent, any of myocarditis, myocardial infarction, atherosclerosis, vasculitis, stroke, multiple sclerosis, Alzheimer's disease, autoimmune hepatitis, primary biliary cirrhosis, primary sclerosing cholangitis, liver fibrosis, non-alcoholic steatohepatitis, paracetamol liver injury, alcohol liver injury, idiopathic pulmonary fibrosis, acute lung injury, cardiac allograft vasculopathy, sarcoidosis, influenza, inflammatory bowel disease, pancreatitis, rheumatoid arthritis, psoriasis, skin fibrosis, breast cancer and colorectal cancer, which all comprise expression of both CC and CXC chemokines, as shown in FIG. 1. A hybrid polypeptide of the invention may bind all or substantially all chemokines associated with any particular disease as shown in FIG. 5. More generally, a hybrid polypeptide binding both a CC chemokine and a CXC chemokine may be used to inhibit chemokine signalling in, and to treat or prevent, any disease associated with both CC and CXC chemokines, such as any inflammatory disease.

Exemplary therapeutic indications suitable for the hybrid polypeptide of SEQ ID 74, as seen in FIG. 1 and FIG. 6, include myocarditis (CCL5, CCL13, CCL17, CCL18, CCL19, and CXCL8); myocardial infarction (CCL3, CCL4, CL5, CCL11, CCL13, CCL21 and CXCL8); atherosclerosis (CCL3, CCL4, CCL5, CCL11, CCL13, CCL15, CCL17, CCL18, CCL19, CCL21, CCL23, CXCL8); idiopathic pulmonary fibrosis (CCL3, CCL4, CCL5, CCL7, CCL8, CCL11, CCL13, CCL17, CCL18, CCL19, CXCL1, CXCL8); acute lung injury (CCL7, CXCL1, CXCL8); inflammatory bowel disease (CCL3, CCL4, CCL5, CCL7, CCL8, CCL11, CCL14, CCL15, CXCL1, CXCL8); rheumatoid arthritis (CCL3, CCL5, CCL7, CCL8, CCL13, CCL14, CCL15, CCL17, CCL18, CCL19, CCL21, CXCL1, CXC L8).

First and second tick CKBP amino acid sequences selected in combination for provision of a hybrid polypeptide for binding chemokines expressed in a particular disease may be selected to individually bind multiple chemokines expressed in that disease. For instance, when treating rheumatoid arthritis, a hybrid polypeptide, combination, polynucleotide, vector or host cell of the invention representing a tick CKBP amino acid sequence shown in SEQ ID NO: 3 may be used. Similarly, when treating or preventing one or more of atherosclerosis, rheumatoid arthritis, inflammatory bowel disease, liver fibrosis, lung fibrosis, kidney fibrosis, skin fibrosis, multiple sclerosis, breast cancer, or Alzheimer's disease, a hybrid polypeptide, combination, polynucleotide, vector or host cell of the invention representing a tick CKBP amino acid sequence shown in SEQ ID NO: 1 may be used.

Where the disease to be treated or prevented is myocarditis, giant cell myocarditis, myocardial infarction, stroke or idiopathic pulmonary fibrosis, a hybrid polypeptide, combination, polynucleotide, vector or host cell of the invention representing a tick CKBP amino acid sequence shown in SEQ ID NO: 29 and/or a tick CKBP amino acid sequence shown in SEQ ID NO: 9 may be used. A hybrid polypeptide, combination, polynucleotide, vector or host cell of the invention representing a tick CKBP amino acid sequence shown in SEQ ID NO: 1 may also be used for treatment or prevention of the above diseases.

Where the disease comprises expression of one or more of CXCL1, CXCL7, CXCL8, CXCL9, CXCL10, CXCL11 and CXCL2, a polypeptide, combination, polynucleotide, vector or host cell of the invention representing a tick CKBP amino acid sequence shown in any one of SEQ ID NOs 45-72 or a variant thereof may be used for treatment or prevention of the disease. The polypeptide may be a hybrid polypeptide comprising a binding sequence for one or more of the above CXC chemokines derived from any one of SEQ ID NOs 45-72 or a variant thereof. Alternatively, the polypeptide may comprise the full-length sequence of any one of SEQ ID NOs 45-72 or a variant thereof. Where the disease comprises expression of one or more of CXCL7, CXCL9, CXCL10, CXCL11 and CXCL12, a chemokine-binding sequence comprised in the hybrid polypeptide may be derived from, or the polypeptide may comprise, the amino acid sequence of any one of SEQ ID NOs 45-60 and 64-65 or a variant thereof. Where the disease comprises expression of one or more of CXCL7, CXCL9 and CXCL11 a chemokine-binding sequence comprised in the hybrid polypeptide may be derived from, or the polypeptide may comprise, the amino acid sequence of any one of SEQ ID NOs 45-48, 51-53, 56, 59, 60 and 65 or a variant thereof. The disease to be treated or prevented by one or more of the above CXC-binding chemokines may be one in which multiple CXC chemokines are expressed, such as rheumatoid arthritis, atherosclerosis or pancreatitis.

The skilled person can provide hybrid tick CKBPs having appropriate combinations of chemokine-binding activities from first and second tick CKBP amino acid sequences or variants thereof, or select novel tick CKBP polypeptides described herein to treat or prevent specific diseases or combinations of diseases. The skilled person can further provide hybrid tick CKBPs having chemokine-binding activities from three different tick CKBP amino acid sequences or variants thereof, as illustrated by the 3-warhead tick CKBPs described herein.

The 3-warhead tick CKBPs of SEQ ID NOs 92 and 93 or variants thereof as described herein are particularly suitable for treatment of diseases in which one or more, preferably three or more of CCL2, CCL5, CCL8, CXCL8, CXCL10 and CXCL1 are expressed, including any such disease described above. For example, acute lung injury (also referred to as acute respiratory distress syndrome) occurs in the context of smoke inhalation, toxins, aspiration, severe burns, pneumonia, sepsis, pancreatitis, trauma, transplant donor ischemia, and cardiopulmonary bypass. CC and CXC chemokines (e.g. CCL2, CCL5, CCL7, CXCL1, CXCL3, CXCL5, CXCL7, CXCL8, CXCL9, CXCL10, CXCL11) are expressed in the lung following acute injury.[103,104,170-177]. The three-warhead evasins described above would be predicted to be of therapeutic benefit in acute lung injury.

A major complication of heart transplantation is cardiac allograft vasculopathy (CAV) which reduces graft and recipient survival. Chemokines that drive CAV include CXC chemokines (CXCL1, CXCL2, CXCL5, CXCL8, CXCL9) and CC-chemokines (CCL1, CCL2, CCL3, CCL4, CCL5), which drive the influx of neutrophils, NK cells and monocyte/macrophages [178,179]. The three-warhead evasins described above would also be predicted to be of therapeutic benefit in CAV.

The skilled person can also provide truncated forms of evasins retaining chemokine-binding activity for use in treatment of diseases, such as the truncated peptide of SEQ ID NO: 89 or a variant thereof. The truncated peptide or variant may be modified for example by cyclisation, or an inter-peptide disulphide bond or be in a stapled form, and/or may be bound or fused to a carrier, such as albumin. Such a truncated peptide or variant thereof may also be used to provide CCL8, CCL7 and CCL18 binding activity in a hybrid evasin polypeptide of the invention. SEQ ID NO: 89 and variants thereof are useful for treatment of diseases comprising expression of one or more of CCL8, CCL7 and CCL18, including any such disease described above. A peptidomimetic of SEQ ID NO: 89 or a variant thereof may also be provided and used in the above treatments. Suitable variants include SEQ ID NOs: 88, 103-109, comprising truncations and/or other modifications as described above. In preferred embodiments, the peptide variant is BK1.2 or BK1.3 (SEQ ID NOs: 105 or 106), preferably BK1.3. The truncated variant peptide may be modified for example by cyclisation, inter-peptide disulphide bond or be in a stapled form, and/or may be bound or fused to a carrier, such as albumin. The variant peptide may comprise SEQ ID NO: 106 fused to a second variant peptide via a disulphide bond at Cys 30 of each peptide. Particular diseases in which the above chemokines are expressed are also as follows: CCL18—atherosclerosis, rheumatoid arthritis, myocarditis, sarcoidosis, idiopathic pulmonary fibrosis, vasculitis, atopic dermatitis, breast cancer, influenza; CCL7—acute lung injury, stroke, idiopathic pulmonary fibrosis, Psoriasis, colorectal cancer, skin fibrosis, rheumatoid arthritis, inflammatory bowel disease; CCL8—rheumatoid arthritis, inflammatory bowel disease, idiopathic pulmonary fibrosis.

Any subject may be treated in accordance with the invention. The subject is typically human. However, the subject can be another animal or mammal, such as a research animal, such as a rat, a mouse, a rabbit or a guinea pig, a commercially farmed animal, such as a horse, a cow, a sheep or a pig, or a pet, such as a cat, a dog or a hamster.

The subject may be asymptomatic. A prophylactically effective amount of the polypeptide, combination, polynucleotide, vector or host cell is administered to such a subject. A prophylactically effective amount is an amount which prevents the onset of one or more, preferably all of, symptoms of the one or more diseases.

Alternatively, the subject may be in need thereof. That is, the subject may exhibit one or more symptoms of the one or more diseases. A therapeutically effective amount of the polypeptide, combination, polynucleotide, vector or host cell is administered to such a subject. A therapeutically effective amount is an amount which is effective to ameliorate one or more of, preferably all of, the symptoms of the one or more diseases.

The polypeptide, combination, polynucleotide, vector or host cell may be administered to the subject in any appropriate way. In the invention, the polypeptide, combination, polynucleotide, vector or host cell may be administered in a variety of dosage forms. Thus, it can be administered orally, for example as tablets, troches, lozenges, aqueous or oily suspensions, dispersible powders or granules. It may also be administered by enteral or parenteral routes such as via buccal, anal, pulmonary, intravenous, intra-arterial, intramuscular, intraperitoneal, intraarticular, topical or other appropriate administration routes. A physician will be able to determine the required route of administration for each particular subject.

The polypeptide, combination, polynucleotide, vector or host cell may be in any of the forms discussed above with reference to the pharmaceutical composition of the invention.

Methods for gene delivery are known in the art. See, e.g., U.S. Pat. Nos. 5,399,346, 5,580,859 and 5,589,466. The nucleic acid molecule or a modified nucleic acid molecule can be introduced directly into the recipient subject, such as by standard intramuscular or intradermal or intravenous or intra coronary artery or intramyocardial injection; transdermal particle delivery; inhalation; topically, or by oral, intranasal or mucosal modes of administration. The molecule alternatively can be introduced ex vivo into cells that have been removed from a subject. For example, a polynucleotide, expression cassette or vector of the invention may be introduced into APCs of an individual ex vivo. Cells containing the nucleic acid molecule of interest are re-introduced into the subject such that an immune response can be mounted against the peptide encoded by the nucleic acid molecule. The nucleic acid molecules used in such immunization are generally referred to herein as “nucleic acid vaccines.”

The dose may be determined according to various parameters, especially according to the substance used; the age, weight and condition of the subject to be treated; the route of administration; and the required regimen. Again, a physician will be able to determine the required route of administration and dosage for any particular subject. A typical daily dose is from about 0.1 to 50 mg per kg of body weight, such as 5 mg per kg of body weight, according to the activity of the specific inhibitor, the age, weight and conditions of the subject to be treated and the frequency and route of administration. The dose may be provided as a single dose or may be provided as multiple doses, for example taken at regular intervals, for example 2, 3 or 4 doses administered hourly. Preferably, dosage levels of inhibitors are from 5 mg to 2 g.

Typically polynucleotide or oligonucleotide inhibitors are administered in the range of 1 pg to 1 mg, preferably to 1 pg to 10 μg nucleic acid for particle mediated delivery and 10 μg to 1 mg for other routes.

The polypeptide, the combination, the polynucleotide, the vector or the host cell is preferably administered in combination with another therapy

The inhibitor may be used in combination with one or more other therapies intended to treat the same subject. By a combination is meant that the therapies may be administered simultaneously, in a combined or separate form, to the subject. The therapies may be administered separately or sequentially to a subject as part of the same therapeutic regimen. For example, the polypeptide, the combination, the polynucleotide, the vector or the host cell be used in combination with another therapy intended to treat the one or more disease. The other therapy may be a general therapy aimed at treating or improving the condition of the subject. For example, treatment with methotrexate, glucocorticoids, salicylates, nonsteroidal anti-inflammatory drugs (NSAIDs), analgesics, other DMARDs, aminosalicylates, corticosteroids, and/or immunomodulatory agents (e.g., 6-mercaptopurine and azathioprine) may be combined with the inhibitor. The other therapy may be a specific treatment directed at the one or more diseases. Such treatments are known in the art. For instance in the treatment of rheumatoid arthritis this may include anti-TNFα [180] or other biologics targeting other cytokines (e.g. IL7, IL17, IL17) or their receptors (e.g. IL1-R, IL-6R), that are in clinical use or development [181]. In the treatment of inflammatory bowel disease we may use biologics such as vedolizumab [182]. For atherosclerosis simvastatin or other statins may be used.

### Antibodies of the Invention

The invention also provides an antibody or a fragment thereof which specifically binds a polypeptide comprising (a) an amino acid sequence shown in any one of SEQ ID NOs: 45 to 72 or (b) an amino acid sequence having at least 70% homology or amino identity to a sequence of (a) over its entire length. The antibody or fragment thereof preferably specifically binds a polypeptide comprising an amino acid sequence shown in any one of SEQ ID NOs: 45 to 72.

An antibody “specifically binds” to a polypeptide when it binds with preferential or high affinity to that polypeptide but does not substantially bind, does not bind or binds with only low affinity to other polypeptides. For instance, an antibody “specifically binds” to SEQ ID NO: 45 or a variant thereof when it binds with preferential or high affinity to SEQ ID NO: 45 or a variant thereof but does not substantially bind, does not bind or binds with only low affinity to other polypeptides. The same applies to any one of SEQ ID NOs: 46 to 72.

An antibody binds with preferential or high affinity if it binds with a Kd of 1×10-7 M or less, more preferably 5×10-8 M or less, more preferably 1×10-8 M or less or more preferably 5×10-9 M or less. An antibody binds with low affinity if it binds with a Kd of 1×10-6 M or more, more preferably 1×10-5 M or more, more preferably 1×10-4 M or more, more preferably 1×10-3 M or more, even more preferably 1×10-2 M or more. A variety of protocols for competitive binding or immunoradiometric assays to determine the specific binding capability of compounds, such as antibodies or antibody constructs and oligonucleotides are well known in the art (see for example Maddox et al, J. Exp. Med. 158, 1211-1226, 1993).

The antibody may be, for example, a monoclonal antibody, a polyclonal antibody, a single chain antibody, a chimeric antibody, a CDR-grafted antibody or a humanized antibody. The antibody may be an intact immunoglobulin molecule or a fragment thereof such as a Fab, F(ab′)₂ or Fv fragment. Furthermore, the antibodies and fragment thereof may be chimeric antibodies, CDR-grafted antibodies or humanised antibodies.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, NY. For example, an antibody may be produced by raising an antibody in a host animal against the whole polypeptide or a fragment thereof, for example an antigenic epitope thereof, hereinafter the “immunogen”. The fragment may be any of the fragments mentioned herein (typically at least 10 or at least 15 amino acids long).

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified. A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256, 495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat, mouse, guinea pig, chicken, sheep or horse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

### Diagnostic Methods of the Invention

The invention also provides a method of detecting one or more chemokines in a tissue, comprising contacting the tissue with a detectably-labelled polypeptide of the invention or a detectably-labelled polypeptide combination of the invention and detecting the binding of the polypeptide or the combination to one or more chemokines in the tissue. The polypeptide may be a fusion polypeptide of the invention. The tissue may be in vitro or in vivo. The invention also provides a detectably-labelled polypeptide of the invention or a detectably-labelled combination of the invention for use in a method of detecting one or more chemokines in a tissue. The invention also provides use of a detectably-labelled polypeptide of the invention or a detectably-labelled combination in the manufacture of medicament for detecting one or more chemokines in a tissue.

Any method of detecting binding may be used. The method may be positron emission tomography (PET) or magnetic resonance imaging (MRI).

The tissue may be any tissue. The tissue is preferably in a subject. The subject may be any those discussed above. The polypeptide or combination may be administered to the subject in any of the forms discussed above.

Any of the polypeptides of the invention or combinations of the invention discussed above may be used. Suitable detectable labels are also discussed above. The label is preferably a tracer that is suitable for positron emission tomography (PET), such as fluorodeoxyglucose (¹⁸F). The label is preferably a tracer suitable for magnetic resonance imaging (MRI), such as fluorine (¹⁹F).

The method may comprise detecting any number of chemokines, such as 1, 2, 3, 4, 5, 6, 7, 8, 9, 10, 11, 12, 13 or 14 chemokines. The chemokines may be selected from any of those in Table 2, 4 and 6. When detecting one or more of CXCL1, CXCL7, CXCL8, CXCL9, CXCL10, CXCL11 and CXCL2, a hybrid polypeptide comprising a binding sequence for one or more of the above CXC chemokines derived from any one of SEQ ID NOs 45-72 or a variant thereof may be used. Alternatively, a polypeptide comprising the full length sequence of any one of SEQ ID NOs 45-72 or a variant thereof may be used. When detecting both one or more CC and one or more CXC chemokines, a hybrid polypeptide of the invention binding a CC and a CXC chemokine may be used.

The method is preferably for diagnosing or prognosing one or more diseases associated with one or more chemokines. The method may comprise diagnosing or prognosing any number of diseases associated with one or more chemokines, such as 1, 2, 3, 4, 5, 6, 7, 8, 9, 10, 11, 12 or 13 diseases. The one or more diseases may as be identified in Table 7 and 8 or FIG. 1. When diagnosing or prognosing the one or more diseases in a particular row of Table 7 and 8, a polypeptide, combination, polynucleotide, vector or host cell of the invention representing an amino acid sequence from the tick CKBP in the same row (or representing an amino acid sequence from a tick CKBPs shown in Table 2 or 6 as binding one or more of the chemokines indicated in the above row of Table 7 or 8) is preferably used in the method of the invention. When diagnosing or prognosing any specific disease shown in FIG. 1, a polypeptide, combination, polynucleotide, vector or host cell of the invention representing amino acid sequence(s) from one or more tick CKBPs shown to bind chemokines associated with that disease (as shown in FIG. 1) is preferably used.

Particular selections of tick CKBP amino acid sequences for diagnosis or prognosis of particular diseases may be made according to the same criteria discussed above in relation to medical uses. Thus, a hybrid polypeptide binding both a CC chemokine and a CXC chemokine may be used to diagnose or prognose any of myocarditis, myocardial infarction, atherosclerosis, vasculitis, stroke, multiple sclerosis, Alzheimer's disease, autoimmune hepatitis, primary biliary cirrhosis, primary schlerosing cholangitis, liver fibrosis, non-alcoholic steatohepatitis, paracetamol liver injury, alcohol liver injury, idiopathic pulmonary fibrosis, acute lung injury, sarcoidosis, influenza, inflammatory bowel disease, pancreatitis, rheumatoid arthritis, psoriasis, skin fibrosis, breast cancer and colorectal cancer, which all comprise expression of both CC and CXC chemokines, as shown in FIG. 1. A polypeptide representing a tick CKBP amino acid sequence shown in any one of SEQ ID NOs 45-72 or a variant thereof may be used to diagnose or prognose a disease comprising expression of one or more of CXCL1, CXCL7, CXCL8, CXCL9, CXCL10, CXCL11 and CXCL2, such as a disease comprising expression of one or more of CXCL7, CXCL9 and CXCL11. The disease may be one in which multiple CXC chemokines are expressed, such as rheumatoid arthritis, atherosclerosis or pancreatitis

The skilled person can provide hybrid tick CKBP polypeptides having appropriate combinations of chemokine-binding activities from first and second tick CKBP amino acid sequences or variants thereof, or select novel tick CKBP polypeptides described herein or combinations of the above to diagnose or prognose specific diseases or combinations of diseases.

### Examples

1. Identification of Chemokine-Binding Tick CKBPs.

To identify protein-protein interactions between extracellular proteins we adapted yeast surface display technology, originally developed for the identification of single chain antibodies[17,183]. Here candidate proteins are expressed in yeast and displayed on the cell wall. Fluorescent-activated cell sorting (FACS) is used to select a desired yeast cell that bind a fluorescent-labelled target. To identify tick CKBPs we created yeast surface display libraries that express mature peptides identified in tick salivary transcriptomes, we systematically screened the above libraries using the chemokines CCL1, CCL2, CCL3, CCL4, CCL5, CCL8, CCL11, CCL15, CCL17, CCL18, CCL19, CCL20, CCL22, CCL25, CX3CL1, CXCL8, CXCL10, CXCL11, CXCL12, CXCL13. We obtained interacting clones that were retested and bound to one or more chemokines confirmed using FACS (SEQ ID NOs: 1 to 31) Table 1). This method can be used also to alter affinities and binding characteristics of a tick CKBP e.g. through mutagenesis and FACS selection.

2. Characterisation of Tick CKBP Binding to Chemokines.

Characterisation of binding of certain tick CKBPs identified in Example 1 against all known human chemokines (with exception of CCL25, CCL26, CXCL16, CXCL17, CXCL4L1, XCL2) was carried out using biolayer interferometry. The data for their binding properties are shown in Table 2, alongside published K_{d} data in relation to binding of human chemokines for previously described tick CKBPs (Evasins 1, 3 and 4). Other binding data for the tick CKBPs obtained using yeast surface display is also summarised. From this data three classes of novel tick CKBPs were identified, as shown in Table 2. Class I tick CKBPs bind CC-class chemokines CCL2, CCL13 or CCL20 in addition to other CC chemokines as indicated. Class III tick CKBPs bind CXC-class chemokines CXC-chemokines CXCL3, CXCL10 or CXCL12 in addition to other CXC chemokines as indicated. Class III tick CKBPs have other chemokine-binding characteristics.

3. Characterisation of Inhibition of Chemokine Function by a Tick CKBP.

Evaluation of the neutralisation activity of certain tick CKBPs identified in Example 1 against particular human chemokines was carried out using a THP1 transwell cell migration assay, with results (IC₅₀ data) shown in Table 3. The results illustrate neutralisation of function of multiple chemokines by certain tick CKBPs.

4. Isolation and Characterisation of Novel Tick CKBP Polypeptides

28 novel CXC-binding tick CKBP polypeptides (SEQ IDS 45-72) were isolated in an additional yeast surface display screening carried out in accordance with Example 1, with results shown in Tables 5 and 6.

5. Hybrid Tick CKBP Comprising a Substituted Chemokine-Binding Sequence.

Alignment of P672_RHIPU to EVA1_RHISA (FIG. 2) and modelling (FIG. 4A) of P672_RHIPU and CCL8 to the Evasin 1:CCL3 structure 3FPU using MODELLER [160] and PISA web server [162] suggested that the N-terminal 44 residues of P672_RHIPU may possess a similar structure to the first 29 residues of Evasin-1, and may carry an independent transportable function of binding CCL8.

We exchanged the N-terminal 29 residues of Evasin-1, with the N-terminal 44 residues of P672_RHIPU to generate a hybrid tick CKBP having the amino acid sequence shown in SEQ ID NO: 76. The nucleotide sequence used here is EZ406190.1, which encodes the Evasin-1 peptide variant K92E. Evasin-1 (EVA1_RHISA) binds CCL3, CCL3L1, CCL4, CCL4L1, CCL14 and CCL18 but not CCL8[20,21]. We have confirmed using biolayer interferometry that the Evasin-1 peptide encoded by EZ406190.1 also does not bind CCL8 at a concentration of 300 nM CCL8. P672_RHIPU binds CCL8 with K_{d}=3.7 nM. The hybrid tick CKBP (P672:EVA1) binds CCL8 with K_{d}=223 nM (FIG. 5A), and neutralizes CCL8 in a migration assay (FIG. 5B). These results show that the CCL8 binding properties of P672_RHIPU lie, at least in part, in its 44 N-terminal residues, and that this region and its CCL8-binding properties can be transferred to evasin-1 which does not bind CCL8. Additional hybrid tick CKBP representing greater extents of P672_RHIPU and lesser extents of P672_RHIPU (EZ406190.1), as shown in SEQ ID NOs 82 and 83 have also been generated and characterised as having a CCL8-binding function.

6. Two-Warhead Tick CKBPs

We genetically engineered P991_AMBCA and P1243_AMBAM (CC binding tick CKBPs) to link each via a flexible GGGGS linker to P1156_IXORI (CXC binding tick CKBP) to create “2-warhead” tick CKBPs, shown respectively in SEQ ID NO: 73, and 74, We show that the “2-warhead” tick CKBPs retain some of the properties of each of the parental tick CKBPs by binding and neutralizing both CC and CXC chemokines as shown in the results in FIG. 6. Additional related 2-warhead polypeptides shown in SEQ ID NOs 80 and 81 have also been generated. An additional 2-warhead polypeptide related to SEQ ID NO: 80 is provided by SEQ ID NO: 94 shown in the sequence listing. References to SEQ ID NO: 80 and variants thereof made herein may be substituted for SEQ ID NO: 94 and variants thereof.

The results obtained for a 2-warhead tick CKBP indicate, that two (or more) tick CKBPs can be physically linked e.g. via a flexible linker or linkers, of variable length or design, to create a non-natural peptide that that retains the properties of the two parent tick CKBPs. Novel artificial chemokine binding peptides with desired properties that match CC and CXC chemokine expression patterns in disease, can thus be created by mixing and matching a number of CC or CXC binding natural tick CKBPs.

2-warhead evasins (SEQ ID NOs 74 and 81) were further investigated for their ability to functionally inhibit CC and CXC chemokines as compared to individual (parental) evasins represented in the 2-warhead molecules. Results are shown in FIGS. 7 and 8, illustrating that 2-warhead evasins can functionally inhibit both CC and CXC chemokines in either orientation.

2-warhead evasins (SEQ ID NOs 73 and 80) were also further analysed for their binding to human chemokines compared to parental evasins, with summary data shown in FIG. 9. The data indicated that the two-warhead evasins bound both CC and CXC chemokines.

The ability of 2-warhead evasins to engage in polyvalent binding of CC and CXC chemokines was further determined. Results are shown in FIGS. 10 and 11. FIG. 10 shows results for SEQ ID NOs 74 and 81. The increase in wavelength shift (nm) on the y-axis observed during association 2 with CXCL8 and CXCL1 indicate that the two warheads can associate simultaneously with CCL5 and either CXCL8 or CXCL1. However the lack of change when incubated with CCL3, indicates that the evasin can only bind one CC chemokine at a time. FIG. 11 shows results for SEQ ID NOs 73 and 80. The increase in wavelength shift (nm) on the y-axis observed during association 2 with CXCL8 and CXCL1 indicate that the two warheads can associate simultaneously with CCL5 and either CXCL8 or CXCL1. However the lack of change when incubated with CCL2, indicates that the evasin can only bind one CC chemokine at a time.

7. CXC Chemokine Binding Evasins

The various CXC chemokine binding evasins described in the application were analysed in more detail for their chemokine binding activity using biolayer interferometry. This permitted identification of two functional classes of CXC chemokine binding evasins. Results are shown in FIG. 12. The data indicate that the CXC-binding evasins can be grouped by function into two classes. Class I, which includes the class founder EVA3_RHISA, binds ELR+ CXC-chemokines including CXCL1 and/or CXCL8, while class II binds a broader range of ELR+ and ELR− chemokines, but does not bind CXCL8. These evasins do not bind CC chemokines using biolayer interferometry.

The functional inhibition of CXC chemokines by P1142_AMBCA was also further investigated. Results from chemokine-induced cell migration assays are shown in FIG. 13. The experiments showed that P1142_AMBCA was able to functionally inhibit chemokines CXCL10, CXCL1 and CXCL2.

8. Analysis of a Truncated Evasin Peptide with Chemokine-Binding Activity

A truncated peptide P672 PEP (SEQ ID NO: 88, peptide BK1.1) was generated consisting of residues E17 to E32 of P672_RHIPU, within the CCL8-binding region of this evasin. Residue C30 was mutated to A to avoid an unpaired cysteine residue. The peptide was N-terminally labelled with FITC (P672_PEP-FITC) to allow for characterisation of chemokine binding by fluorescence polarisation. Results are shown in FIG. 14. The data indicated that P672_PEP-FITC was able to bind CCL8, CCL7 and CCL18 but not CXCL1. Thus the binding of the significantly truncated evasin peptide was specific, and yet displayed a one-to-many binding mechanism characteristic of the parental evasin P672_RHIPU.

The binding specificity of the truncated peptide was further investigated in displacement assays, with results shown in FIG. 15. The experiment showed that unlabeled peptide P672_PEP (peptide BK1.1) was able to displace labelled peptide bound to chemokine and therefore indicates that the unlabeled peptide can also bind CCL7, CCL8 and CCL18 whereas a peptide with the sequence scrambled (P672_PEP_SCRAM) cannot, confirming binding specificity.

Additionally, functional inhibition by the truncated evasin peptide in cell migration assays was investigated, with results shown in FIG. 16. The experiments with results shown in panels A and B showed that P672_PEP (peptide BK1.1) could inhibit CCL8-647 binding to THP-1 cells. The experiments with results shown in panels C and D showed that P672_PEP could inhibit THP-1 cell migration in response to CCL7 and CCL8.

9. 3-Warhead Evasins

The ability to design 3-warhead evasins representing sequences from three different individual parental evasins was investigated.

Three warhead evasin P1820 was created by genetically fusing P1142_AMBCA (SEQ ID 65), P1156_IXORI (SEQ ID 19), and P467_RHIPU (SEQ ID 1) with intervening GGGGS linkers (bold and underlined in sequence below) to create P1142:G4S:P1156:G4S:P467 (SEQ ID NO: 92, P1820).

Three warhead evasin P1821 was created by genetically fusing P1142_AMBCA (SEQ ID 65), P1156_IXORI (SEQ ID 19), and P991_AMBCA (SEQ ID 9) with intervening GGGGS linkers (bold and underlined in sequence below) to create P1142:G4S:P1156:G4S:P991 (SEQ ID NO: 93, P1821).

The sequences of the three warhead evasins are shown below:

P1820:
KPQILQRTDHSTDSDWDPQMCPETCNPSKNISCSSECLCVTLGGGDET
GTCFNMSGVDWLGHAQASDGHNDGGGGGSADDDNELFTVQYCGMNCTK
DEGGTWTGCTGKKEGCKCYHESGKNYGLCLSTEYTDFSQYGNPSDSEI
EAAKPKRSDTLSHGGGGSAEKSLDSDSSGEDYELWTQGCPFLVAENRT
GFGTTVSCQHNCNGAIEKVPEGEPCYTIGEDGLGRMKLNLPYNCSLGE
CSGGVCVPNGRSDVCFKRTWEENNKAMA
P1821:
KPQILQRTDHSTDSDWDPQMCPETCNPSKNISCSSECLCVTLGGGDET
GTCFNMSGVDWLGHAQASDGHNDGGGGGSADDDNELFTVQYCGMNCTK
DEGGTWTGCTGKKEGCKCYHESGKNYGLCLSTEYTDFSQYGNPSDSEI
EAAKPKRSDTLSHGGGGSENGEGTTQPDYDNSTDYYNYEDFKCTCPAP
HLNNTNGTVMKPIGCYYTCNVTRCTAPDTYPCYNLTEHQAKNLTTSPT
TLCAVGNCDHGICVPNGTKELCFKAPNLEE

The binding affinities (K_{d}, M) of immobilized purified 3 warhead evasins P1820 (P1142:G4S:P1156:G4S:P467) and P1821 (P1142:G4S:P1156:G4S:P991) to exemplar human CC and CXC-chemokines were then determined using biolayer interferometry, with results shown below.

| | CCL2 | CCL5 | CCL8 | CXCL1 | CXCL8 | CXCL10 |
|---|---|---|---|---|---|---|
| P1820 | 2.86E−09 | 4.10E−10 | 1.82E−08 | 1.12E−09 | 1.11E−09 | 1.01E−09 |
| P1821 | 1.18E−08 | 1.00E−12 | 1.41E−08 | 1.86E−09 | 1.55E−09 | 1.44E−09 |

The 3-warhead evasins thus were found to bind CCL2, CCL5 and CCL8 (bound by each of the parental evasins P467_RHIPU and P991_AMBCA), CXCL8 (bound by the parental evasin P1156_IXORI), CXCL10 (bound by the parental evasin P1142_AMBCA) and CXCL1, bound by both P1156_IXORI and P1142_AMBCA. Based on the ability to combine each of the individual binding activities of the parental evasins in a single 3-warhead evasin, the potential therapeutic indications for each 3-warhead evasin represent a combination of the individual indications for the parental evasins. The use of a 3-warhead evasin thus extends the therapeutic application of the parental evasins. Additionally, increasing the molecular weight (e.g. in 2, 3 or multi-warheads) in comparison to each parental evasin and may be expected to have advantageous pharmacokinetic effects such as reduced renal clearance resulting in prolonged half-life [184,185]. This would be expected to result in reduction of dose required to be therapeutically effective and resulting also in a prolongation of intervals between doses which would enhance patient acceptability.

10. Hydrogen/Deuterium Exchange Mass Spectrometry (HDX-MS) of the P672:CCL8 Complex Interface

Peptide-resolution HDX-MS was performed to characterise the interaction between P672_RHIPU (referred to hereinafter as “P672”) and CCL8. Deuterium uptake of free P672, free CCL8 and of each protein upon complex formation was measured. Sequence mapping and coverage of each protein (100% for P672, 96.9% for CCL8, FIG. 24) was obtained and deuterium uptake of the free species with that of the P672:CCL8 complex species (5 s, 30 s, 5 min and 60 min incubation time points, FIG. 25) was measured. The results were mapped on to a homology model of the P672:CCL8 complex (see FIG. 17A-B). A significant decrease in H/D exchange was observed in R18-S27 of CCL8 (% relative deuterium uptake (% D) ranging from −6 to −18), which lies in the N-terminal extended loop/β1-region (Blaszczyk, J et al., Biochemistry [2000]), and in the N-terminal unstructured (predicted) region of P672 (E22-F32, D % up to −58%), indicating protection of these regions from solvent exposure when in complex (FIG. 17B-D, FIG. 25). All residues in CCL8 and most in P672 (F25-C30, F32) from these regions were protected at all time points (FIG. 25).

Spectra of two representative P672 peptides showing a reduction in deuterium incorporation for this protected region upon complex formation are shown in FIG. 17E. An increase in relative deuterium uptake was observed for the C-terminal region of P672 (G87-C94, % D ranging from 15 to 18%), indicating higher exposure to solvent water after complex formation (FIG. 17C and FIG. 25). These results indicate that the P672 (E22-F32) and CCL8 (R18-S27) regions may be involved in P672:CCL8 complex formation. The protected regions of P672 and CCL8 overlap the binding interface predicted by the homology model of P672:CCL8 (Eaton, J. R. O. et al., JBC [2018]) suggesting that these residues are involved in protein-protein interactions. Changes in the deuterium uptake in these regions showed little time-dependent change (5 s-60 min, FIG. 25), in agreement with the tight-binding kinetics of P672:CCL8 interaction (K_{d}=8.5 nM, residency time (RT)=27 min) (Eaton, J. R. O. et al., JBC [2018]).

In this study we used HDX-MS and identified a 11-residue region (E22-F32) of P672 that was protected from deuterium uptake upon complexing with CCL8. The HDX-MS result also indicated that CCL8 residues R18-S27, which overlap the N-terminal loop (C12-R24), interact with P672. A key function of the N-terminal loop of CC chemokines is receptor binding, and it is targeted by several pathogenic chemokine-binding proteins. For example, the viral chemokine binding protein VV-35 kDa targets K19 and R24 of CCL2 and the viral chemokine binding protein vCCI targets R18 and R24 of CCL2. This common mechanism suggests the convergent evolution of these proteins to target the residues found in this region. The binding of P672 to this region would competitively prevent CCL8 binding to its receptor, explaining how CCL8 function is neutralised. The N-terminal loop of CCL8 and other CC chemokines is also part of the homodimerization interface, and binding to this loop explains the prevention of CCL8 dimerization by P672 reported previously.

11. Residues E22-F32 in P672 Contain a Transferable CCL8 Binding Activity

The P672 (E22-F32) region was swapped with the corresponding segment of EVA1_RHISA, which is a related CC-chemokine binding evasin that does not bind CCL8 (Eaton, J. R. O. et al., JBC [2018]) (FIG. 18A). The CCL8 binding activity of the hybrid protein EVA1(P672₂₂₋₃₂) (FIG. 18B) was analysed using biolayer interferometry, finding that EVA1(P672₂₂₋₃₂) bound CCL8, whereas, consistent with previously reported results (Eaton, J. R. O. et al., JBC [2018]), the parental evasin EVA1 did not. Dose-titration experiments indicated that EVA1(P672₂₂₋₃₂) bound CCL8 with modest affinity, K_{d}=490 nM.

Taken together with the HDX-MS analysis, these experiments indicate that P672(E22-F32) is involved in forming protein-protein interactions with CCL8, and that this function can be transferred to another evasin.

Swapping the P672₂₂₋₃₂ region into EVA1, an evasin that does not bind CCL8, transferred CCL8 binding activity to the hybrid protein. These results indicate that this 11-residue region binds CCL8.

12. Development of BK1.1, a CCL8 Binding Peptide

A number of tiled peptide fragments spanning the E17-F32 region in P672 were tested for CCL8 binding (FIG. 19A). Y21 and the four acidic residues N-terminal to Y21 were also included in this array, as both P672 and EVA1 share this region. All peptides were synthesised with Cys30 replaced by Ala and an N-terminal fluorescein isothiocyanate (FITC) (BK1-6, FIG. 19B). The longest test peptide P672(E17-F32) was termed BK1.1_{FITC}, and a corresponding scrambled sequence was generated as a negative control (SCR_{FITC}). Only the full contiguous peptide (BK1.1_{FITC}), displayed an increase in anisotropy upon incubation with CCL8 (at a concentration of 1 μM) compared to control, indicating a binding interaction. No changes in anisotropy were observed for Y21-F32 (BK6_{FITC}) under the conditions tested. Fluorescent polarization and dose-titration of CCL8 were used to estimate the affinity (K_{d}) for BK1.1_{FITC} to be approximately 160 nM (FIG. 19C). Alanine-scanning mutagenesis, where each residue of BK1.1_{FITC} was replaced with Ala, was performed to investigate the mechanism of BK1.1 binding. Each mutant was tested for binding to CCL8 using the fluorescent anisotropy assay to measure binding affinity. Key residues that contribute to CCL8 binding were identified (FIG. 19D, Table 9). Significant differences were observed when aromatic residues Tyr and Phe (Tyr21, Tyr31, Phe25 and Phe32) were mutated to Ala. The D18A mutation also showed reduced affinity, supporting the peptide tiling data and indicating the importance of interactions outside of Y21-F32 region. Notably, P27A completely abolished binding to CCL8 indicating that it has a key function.

13. BK1.1 Disrupts CCL8 Homodimerization

As shown by native mass spectrometry CCL8 exists as a homodimer (FIG. 19E). After incubation with BK1.1, both 1:1 and 2:1 species of the BK1.1:CCL8 complex were observed, together with CCL8 monomer and BK1.1. The presence of CCL8 monomer and BK1.1 species may be due to partial dissociation of the complex. The stoichiometry observed was supported by dissociation of these complexes using higher-energy collisional dissociation (HCD). BK1.1 can thus form a stable 1:1 complex with CCL8 and disrupt CCL8 homodimerization in line with our P672:CCL8 native mass spectrometry analysis (Eaton, J. R. O. et al., JBC [2018]). The presence of low levels of 2:1 BK1.1:CCL8 complex indicate a possible second site of BK1.1 binding.

14. BK1.1 Promiscuously Binds Three CC Class Chemokines

BK1.1_{FITC} was screened for binding against the 13 CC-chemokines known to bind to P672 (Eaton, J. R. O. et al., JBC [2018])(FIG. 19F). CCL7, CCL8 and CCL18 caused significant increase in anisotropy of the emitted light compared to the negative control CXCL1, a chemokine that does not bind P672 (Eaton, J. R. O. et al., JBC [2018]), suggesting a binding interaction between BK1.1_{FITC} and these chemokines. Fluorescent polarisation displacement assays with unlabelled BK1.1 indicate its binding to CCL7, CCL8 and CCL18 (FIG. 19G-I).

15. Engineering of Peptides with Improved Potency and Promiscuous CC-Chemokine Binding

The role of the four acidic residues N-terminal to Y21, and the impact of C30A mutation introduced into BK1.1, were explored. Two shorter peptides, Y21-F32 with or without the C30A mutation (FIG. 20A) were compared to BK1.1 in their ability to disrupt the interaction between P672 and CCL8 using an AlphaScreen assay. All three peptides were found to significantly disrupt the interaction, with the effect of Y21F32, and BK1.1 far exceeding that of peptide Y21F32, C30A (FIG. 20B). Only Y21F32 and BK1.1 disrupted the P672-CCL2 interaction, and only Y21F32 disrupted the P672-CCL3 interaction (FIG. 20C-D). These results implied that the four acidic residues N-terminal to Y21 and the Cys residue were important for chemokine binding. A series of peptides (BK1.2-BK1.6) were designed based on BK1.1 (FIG. 20A) that maintained the four acidic residues N-terminal to Y21 and also Cys at position 30. A cyclic version (BK1.2) was designed, with Cys30 cyclised to a N-terminal Tyr residue that was introduced (McAllister, T. E. et al., Chem Sci [2018])(Kawamura, A. et al., Nat Comm. [2017]). As a control, a non-cyclised version of this peptide, BK1.3, was designed. The binding of these peptides to CCL8 was assayed by examining their ability to disrupt the P672:CCL8 interaction using an AlphaScreen assay (FIG. 20E, H). Both BK1.2 (IC₅₀=729 nM) and BK1.3 (IC₅₀=238 nM) had significantly improved ability to disrupt the P672:CCL8 interaction in comparison to BK1.1 (IC₅₀=59.1 μM). Further peptides, BK1.4 (cyclised) and BK1.5 (linear), were created that lacked the N-terminal Tyr. BK1.4 was cyclised to Cys30 through the N-terminal Glu17 residue. These modifications resulted in a significant reduction of binding activity in comparison to BK1.3 (FIG. 20E,H). These results suggest that cyclisation itself is not critical but instead that the N-terminal Tyr is important. Examination of the peptides by MS revealed that BK1.3 readily oxidised to form a disulphide-bonded dimer, whereas BK1.5 was monomeric (FIG. 26). BK1 derivatives were tested for their ability to inhibit P672 interactions with CCL2 and CCL3 using AlphaScreen (FIG. 20 F, G, I, J). BK1.1 did not inhibit, in line with the lack of binding observed against CCL2 and CCL3 in FP assays (FIG. 19F), all other BK derivatives showed good inhibition against CCL2, and weaker inhibition against CCL3. The IC₅₀ of BK1.3 against CCL2 was 5.7 μM and against CCL3 was 43 μM.

The addition of four acidic N-terminal residues and Tyr21 was necessary to be able to detect binding under these conditions, suggesting these acidic residues may be needed for increased affinity, or that the shorter peptides were sterically hindered from binding by the FITC moiety. Alanine scanning mutagenesis of the 16-residue peptide BK1.1 indicated that binding to CCL8 was mediated by Tyr and Phe residues, and also by the acidic residues at the N-terminus. Notably, Tyr and Phe are both found in protein interaction “hot-spots”, and complementarity in surface charge mediated by acidic residues can modulate protein interactions. A notable finding was that the Pro residue is critical for binding. Pro residues are found in turns, and can undergo cis-trans isomerisation, making it likely that the Pro residue is of structural importance for BK1.1. BK1.1 was observed to prevent CCL8 homodimerization, suggesting that it likely employs a similar mechanism as P672 in binding CCL8, i.e. to the N-loop region. The fluorescent polarisation studies reported indicate that BK1.1 also binds the chemokines CCL7, and CCL18, but not several others.

Given the role of Pro in protein conformation, cyclisation was employed as a strategy for restricting conformational flexibility. A surprising finding was that the addition of an N-terminal Tyr residue, introduced for the purpose of thioether cyclisation, enhanced affinity. The role of the added N-terminal Tyr is supported by the increased affinity of the peptides (BK1.2, BK1.3) that carry it compared to the ones that do not (BK1.1, BK1.4, BK1.5). The role of the re-introduced Cys30 is supported by BK1.5, which differs by a single residue in comparison to BK1.1, and has marked improvement in affinity. The substantial enhancement of activity of BK1.3 thus likely arises from addition of Tyr and re-introduction of Cys30. In addition, it is likely that the unexpected formation of a Cys linked dimer in BK1.3 enhances the functional affinity or avidity of the molecule. Cyclisation in these experiments did not appear to enhance affinity, as evidenced by the lack of improvement of BK1.4 in comparison to BK1.5 or BK1.2 in comparison to BK1.3. This may be, in part, due to non-optimised cyclisation points and/or forced constraint.

16. Engineered Peptides Promiscuously Neutralize Chemokine Function

The effect of BK1.1, 1.2 and 1.3 on CCL8, CCL7, CCL3 and CCL2 induced cell migration was explored. P672 has previously been shown to neutralise these chemokines in analogous experiments (Eaton, J. R. O. et al., JBC [2018]). THP-1 cells express CCR1, CCR2 and CCR5 (Parker, L. C. et al., Journal of immunology [2004])(Achour, L. et al., Blood [2009]), which are activated by CCL8, CCL7 and CCL2, while CCL3 activates CCR1 and CCR5 (Harding, S. D. et al., Nucleic Acids Res [2018]). The experiments were performed with a single concentration of peptide (10 μM, FIG. 21 A-D). P672 (300 nM) was included as a positive control, and a scrambled version of BK1.1 (SCR) as a negative control. BK1.1 reduced CCL8-induced migration to background levels, and had a modest but significant effect on CCL7-induced migration, consistent with its ability to bind these chemokines in the fluorescent polarisation assay (FIG. 21 A, B). There was no significant effect on CCL3 induced migration (FIG. 21C). BK1.1 had a modest but significant effect in inhibiting CCL2 induced cell migration (FIG. 21D). Like BK1.1, BK1.2 and BK1.3 also reduced CCL8 induced cell migration to baseline levels, and had a stronger effect on CCL7 and CCL2-induced migration (FIG. 21, A, B, D). Unlike BK1.1, BK1.2 and BK1.3 also significantly reduced CCL3 induced cell migration (FIG. 21C). Dose titration experiments were performed to establish the relative potencies (IC₅₀) of the engineered peptides against CCL8 (FIG. 21 E, F). BK1.1, BK1.2 and BK1.3 had IC₅₀ values for CCL8 inhibition of 510 nM, 19 nM and 8 nM respectively, correlating well with the increased binding affinity. The positive control, P672 had an IC₅₀ of 3 nM. These results indicated that the engineered peptides promiscuously neutralise different CC class chemokines, with BK1.3 possessing the most potent activity.

17. Engineered Peptides Prevent Cellular Chemokine Binding

To explore the effect of BK1.1 and derivatives on chemokine ligand-cell interactions, a fluorescent-chemokine cell binding assay was developed. Fluorescent chemokine (conjugated to AlexaFluor-647) binding to THP-1 cells results in an increase in the cellular fluorescence intensity, which is quantitatively measured using flow cytometry. In dose-response assays, increasing doses of peptide suppressed CCL8-647 and CCL2-647 induced cellular fluorescence (FIG. 21 G-J). IC₅₀ values for BK1.1, BK1.2 and BK1.3 against CCL8-647 were found to be 5.8 μM, 630 nM and 47 nM respectively, P672 had an IC₅₀ of 21 nM (FIG. 21 G, H). In similar assays IC₅₀ values for BK1.1, BK1.2 and BK1.3 against CCL2-647 were found to be 45 μM, 6.3 μM and 2.2 μM respectively, while P672 had an IC₅₀ of 21 nM (FIG. 211, J). These results indicate that the engineered peptides not only bind chemokines promiscuously, but neutralize their chemotactic function by preventing them from binding to cells.

The improvement in binding to CCL8 observed in the BK1.1-BK1.3 peptide series, as well as their ability to inhibit P672:CC-chemokine interactions (CCL8, CCL2, CCL3), correlated with increased chemokine neutralization potency and promiscuity. In cell-based chemotaxis assays, it was found that the improvement in binding affinity for CCL8 translated into increased potency for inhibiting CCL8-induced cell migration, as evidenced by the reduced IC₅₀. In addition to neutralizing CCL8 and CCL7, which was predicted by the BK1.1 fluorescent polarisation binding study, the peptides BK1.2 and BK1.3 were also able to neutralize CCL2 and CCL3 induced chemotaxis. The inhibition of chemokine binding to cells indicate that the mechanism of neutralization is the prevention of chemokine binding to the cells, likely by preventing chemokine-receptor interactions.

18. Engineered Peptide BK1.3 has In Vivo Anti-Inflammatory Activity

The results suggested that the chemokine-neutralizing properties of the engineered peptides may translate into anti-inflammatory activity in vivo. To study this, the lead peptide BK1.3 was tested in a mouse short-term inflammation model. In this model, zymosan, a yeast cell wall derived PAMP, activates cytokine and chemokine production, and leucocyte infiltration, when injected into an artificially created subcutaneous air-pouch (Coates, N. J. et al., Journal of immunology [2001])(El-Achkar, G. A. et al., PLoS One [2019]). Characterization of this model showed that Ccl9 is expressed at a high basal level but is not induced by zymosan. Ccl2, 5, 11, 12, 20, 22, 24, and Cxcl1, 2, 4, 5, 11, 13, 16 are expressed (>3 fold) at 4 hours following zymosan, and Ccl2, 5, 12, and Cxcl2, 4, 13, 16 are expressed (>3 fold) at 24 hours (FIG. 22). BK1.3, control SCR peptides, and the positive control P672 were injected directly into the air-pouch at 0 and 9 h following zymosan injection. The air-pouch exudate was characterized using flow cytometry at 24 hours after zymosan injection to assess the severity and nature of inflammation. Both BK1.3 and P672 showed a strong and significant reduction in the number of neutrophils, eosinophils, monocytes and T-cells recruited to the air-pouch (FIG. 23 A-F).

Systemic administration of BK1.3 peptide would have anti-inflammatory activity. BK1.3, control SCR peptides, and the positive control P672 were injected intraperitoneally at 0 and 9 h following zymosan injection, and the air-pouch exudate characterized at 24 hours after zymosan injection. Both BK1.3 and P672 showed a substantial and significant reduction in the number of neutrophils, eosinophils, monocytes and T-cells recruited to the air-pouch (FIG. 23 G-L). These results show that the engineered peptide BK 1.3 has in vivo local and systemic anti-inflammatory activity.

A critical step in the clinical translation of novel anti-inflammatory therapeutics is the demonstration of efficacy in vivo, using a model where many components of the immune-inflammatory network are activated. A short-term inflammation model using the well characterized PAMP, zymosan, which activates TLR2 signalling, was used, and resulted in the production of cytokines, chemokines, and complement. The results indicate that zymosan-induced inflammation is significantly inhibited by both local as well as systemic administration of BK1.3. It is likely that the in vivo mechanism of action of BK1.3 includes the inhibition of CC-class chemokines which not only are chemoattractants for leucocyte recruitment, but also heterodimerize and synergise with certain CXC-class chemokines. Our work indicates that peptides with promiscuous chemokine-binding and anti-inflammatory activity can be developed from tick evasins. Such peptides could provide a route to the development of new anti-inflammatory therapeutics.

19. Materials and Methods for Examples 10 to 18

Reagents

All chemokines, unless otherwise stated, were purchased from Peprotech (UK). Fluorescent chemokines were purchased from Almac (UK). THP-1 cells (ECACC 88081201) were maintained in RPMI-1640 media supplemented with 10% fetal calf serum and 4 mM L-glutamine. Cultures were maintained between 3×10⁵ and 1×10⁶ cells/ml in a 37° C. incubator with 5% CO₂. HEK 293F cells (Thermo Fisher) were maintained between 3×10⁵ and 1×10⁶ cells/ml in a 37° C. incubator with 8% CO₂ and 130 RPM agitation in FreeStyle™ 293 Expression Medium.

Plasmids

Evasins were cloned in the expression vector pHLSec. P672 (N-terminal 8×His-StrepII tag) expression vector and EVA1 (C-terminal Strep-8×HisII tag) have been described previously (Eaton, J. R. O. et al., JBC [2018]). The expression vector EVA1(P672₂₁₋₃₂) was constructed using PCR and infusion cloning as described¹⁵, and has a N-terminal 8×His-StrepII tag. Plasmid sequences were confirmed by Sanger sequencing (Source Bioscience, UK). The CCL8 expression plasmid in vector pNIC-BIO3 has been described previously (Eaton, J. R. O. et al., JBC [2018]). Plasmids and sequences are available on request.

Protein Expression

Evasin proteins were expressed as described previously using a mammalian expression system (Eaton, J. R. O. et al., JBC [2018]). Briefly, HEK293F cells were transiently transfected with expression vectors using polyethylenimine and incubated for five days. The supernatant was collected and the recombinant proteins isolated using nickel affinity chromatography followed by size exclusion chromatography. Fractions showing absorbance at 280 nm were analyzed on SDS-PAGE and protein containing fractions were combined for future use. Recombinant CCL8 was expressed as described previously as a SUMO fusion protein from Escherichia coli RosettaGami™ 2 (DE3) cells (Novagen)(Eaton, J. R. O. et al., JBC [2018]). To produce biotinylated CCL8 the same protocol was followed except the cells were also transformed with a plasmid encoding for BirA and 500 μM biotin was also added at the same time as IPTG⁵¹. Following IPTG induction, the over-expressed protein was isolated from the soluble fraction using nickel-charged IMAC Sepharose 6 Fast Flow resin (GE Healthcare). SUMO protease was added to partially purified protein and left overnight at 30° C. The SUMO tag was separated from CCL8 using cation exchange chromatography and the chemokine purified further using size exclusion chromatography. Fractions showing absorbance at 280 nm were analyzed on SDS-PAGE and protein containing fractions were combined for future use.

Hydrogen Deuterium Exchange Analysis

Working solutions of CCL8 and P672 were prepared at a concentration of 35 μM in 50 mM ammonium bicarbonate buffer pH=6.5. For estimation of HDX in the heterodimer state, solutions of CCL8 and P672 were mixed in a (1:1) ratio to reach a final concentration of 17.5 μM and incubated at 4° C. for 1 h (Eaton, J. R. O. et al., JBC [2018]). For estimation of HDX in the unbound state, working solution were diluted to 17.5 μM with 50 mM ammonium bicarbonate pH=6.5. Aliquots of 4.3 μL of heterodimer or unbound proteins were mixed with 48.2 μL of D₂O containing 50 mM ammonium bicarbonate buffer adjusted to pH=6.5 with DCl (final content of D₂O of 91.8%) and incubated for 5 s, 30 s, 5 min and 60 min at RT. HDX was quenched by adding 22.5 μL of 10% formic acid to reach a final volume of 75 μL and pH=2.5, corresponding to a final concentration of 1 μM. Samples were then rapidly flash frozen in liquid nitrogen and stored at −80° C. for up to 5 days before analysis.

An Acquity M class ultra-high performance liquid chromatographer with a nanoAcquity HDX manager coupled to a Synapt G2-Si time-of-flight mass spectrometer (Waters) was used and controlled using the MassLynx 4.1 software. Samples were loaded at 200 μL/min into an Enzymate pepsin column (2.1 mm×30 mm, 5 μm particle size) where the proteins were quickly digested at 20° C. Peptides were then captured for 2 min into a BEH C18 trap column (300 μm×30 mm, 1.7 μm particle size) at 0° C. and then separated in a BEH C18 analytical column (2.1 mm×50 mm, 1.7 μm particle size) at 40 μL/min and 0° C. under a 12 minutes linear gradient from 4 to 85% of acetonitrile with 0.1% formic acid. The MS^{E} approach was used for peptide mapping of non-deuterated proteins with trap collision energies of 15 to 35 V. Deuterated samples were analysed in scan mode only. Source parameters included: cone voltage 30V, capillary voltage 2.8 KV, source temperature 80° C., desolvation temperature 150° C., gas cone flow rate 80 L/h and desolvation gas 250 L/h.

The ProteinLynx Global Server 3.0.2 software was used for peptide mapping. Spectra were searched against a custom database containing the protein sequence of interest, requiring a non-specific digestion enzyme and allowing for variable modifications (i.e. N-terminus pyroglutamic acid from glutamine and deamidation or HexNAc (N-acetylhexosamine) of asparagine present in a N-X-S/T motif). Peptide identification required at least 3 fragment ion matches, the peptide presence in 4 out of 5 replicates, a retention time relative standard deviation of ≤5%, a precursor ion mass tolerance of 10 ppm and peptide maximum length of 30 residues. Relative deuterium uptakes % at the peptide-level were estimated using Dynamix 3.0 as the difference between the uptake (Da) observed for the complex species and the free species divided by the maximum possible uptake of the peptide. Manual check of peptide retention time, charge state and possible peak overlap were also performed. Statistical analysis included a t-Student test and HDX rate differences ≥5% with a p-value ≤0.05 were considered significant. Residues with statistically significant increased or decreased HDX rates were mapped on to a previously described homology model of the P672:CCL8 complex (Eaton, J. R. O. et al., JBC [2018])(generated using the EVA1:CCL3 complex 3FPU (Gault, J. et al., Nature methods [2016]) as template) in which the CCL8 homology structure was replaced by the CCL8 x-ray crystal structure (1ESR)(Kawamura, A. et al., Nat Comms. [2017]). Note that in the case of overlapping peptides, Dynamix 3.0 displays the % Relative Uptake for any given residue as the % Relative Uptake of the shortest peptide. Additionally, in the particular case of overlapping peptides of equal length, the % Relative Uptake refers to that of the peptide in which the residue is closest to the peptide C-terminus. Structural models were created using PyMol2.3.

Biolayer Interferometry

This was carried out as described previously using an OctetRed® system (Singh, K. et al., Sci Rep [2017]). Briefly, affinity determination was evaluated with chemokine concentrations typically ranging from 300 to 0.4 nM, using a non-interacting reference protein to allow for nonspecific binding to the sensor. We used ForteBio Data Analysis 9 software to process the data and calculate association (kₒₙ), dissociation (k_{off}), and affinity (K_{d}) constants. Data with poor curve fits (R²<0.9) were excluded. All biolayer interferometry experiments were performed at least three times.

Fluorescent Peptides

All fluorescent peptides and scrambled (SCR) peptide were purchased from GL Biochem (Shanghai) and were synthesized using standard Fmoc solid phase synthesis to give peptides with a C-terminal amide. The scrambled peptide sequence EFTEVYEFDFKYDAPD is based on BK1.1. All were deemed to be >90% pure by HPLC analysis and verified by LC-MS. Peptides were dissolved in DMSO and the concentration determined using NMR with TSP as an internal standard (Larive, C. K., et al., Applied Spectroscopy [1997]). All peptides were analysed using a Bruker Microflex LRF MALDI-TOF mass spectrometer.

Peptide Synthesis In-House

Amino acids were purchased from CEM. Peptides were purified by HPLC using a Waters SFO system with a Kinetex® 5 mm EVO C18 100 Å (150×21.2 mm) column. All peptides were synthesized with a C-terminal amide on a 50 mmol scale using standard Fmoc protection chemistry on a CEM Liberty Blue automated peptide synthesiser. We used NovaPEG Rink Amide resin (Merck) with N,N′-diisopropylcarbodiimide as a coupling reagent. Following the final Fmoc deprotection step, the resin was washed with N,N-dimethylformamide (5 ml, twice) and dichloromethane (5 ml, twice). The peptides were deprotected and cleaved from the resin by incubation of the beads with a mixture of trifluoroacetic acid/1,3 dimethoxybenzene/triisopropylsilane/water (92.5:2.5:2.5:2.5) at room temperature for 3 h. The cleaved peptides were then precipitated through the addition of 50 mL ice cold diethyl ether, the solution centrifuged for 10 min at 750 g and the resulting pellet resuspended in acetonitrile/water. The peptides were purified by reverse-phase preparative HPLC (5 to 50% B, A: solution of ammonium bicarbonate 0.1 M at pH 8 with 5% acetonitrile in water; B: solution of ammonium bicarbonate 0.1 M at pH 8 with 5% water in acetonitrile). Fractions were analysed using a MALDI-TOF mass spectrometer and peptide containing fractions were combined, dried under vacuum using a Genevac EZ-2 Elite system, and characterised using High Resolution mass spectroscopy. For cyclic peptides, the linear precursor was prepared as described above except following the final Fmoc deprotection step, on-bead chloroacetylation of the N-terminus was achieved through incubating the beads with a 2 mL of a solution of 0.3 M chloroacetic anhydride in N,N-dimethylformamide for 3 h at room temperature. After washing, cleaving and precipitating as described above the N-terminally chloroacetylated linear peptides were dissolved in no more than 2 mL of an adequate mixture of 1 M triethylammonium acetate pH 9.6 buffer and acetonitrile, where the pH was maintained at >9 through the addition of KOH. The cyclic peptide precursors were incubated at 42° C. for 1 h and the cyclisation reaction was monitored using MALDI-TOF mass spectrometry. Once the reaction appeared to have gone to completion the cyclic peptides were purified as above. Peptides were dissolved in DMSO-d6 to form 100 mM stocks. Accurate concentrations were measured by 1H-qNMR in D2O accounting for aromatic protons with TSP as standard and using a Bruker AVII 500 instrument.

Fluorescence Polarization Assays

Fluorescence polarization assays were performed using a Clariostar (BMG Tech) plate reader with the supplied FITC excitation and emission filters using 96 half area well plates (Corning). The buffer used (FP assay buffer) was 50 mM HEPES, 150 mM NaCl, 0.1% BSA, 0.002% TWEEN-20, 0.2% DMSO, pH 7.4 and the final volume in each well was 30 μL. Polarization was converted to anisotropy using the equation A=(2*P)/(3−P) where P is polarization and A is anisotropy. For each peptide tested the gain was set to 35mP and adjusted to a well containing fluorescent peptide only. The polarization of the emitted light in the FITC emission channel was then determined. Experiments were performed as two technical and three biological replicates. Screening of P672 peptide fragments was achieved through incubation of each peptide (50 nM) with 1 μM CCL8 (Peprotech) for 30 minutes in FP assay buffer and the resulting anisotropy of the emitted light determined as above. The chemokine cross binding screen was performed by incubating 1 μM chemokine (Peprotech) with 50 nM BK1.1_{FI}TC for half an hour in FP assay buffer and the resulting anisotropy of the emitted light determined as above. To monitor BK1.1_{FITC} Ala mutants of binding to CCL8, 50 nM labelled peptide was incubated with varying concentrations of recombinant CCL8 (0-25 μM, final) in 30 μL FP assay buffer for 30 minutes and the resulting anisotropy of the emitted light determined as above. The anisotropy was plotted as a function of CCL8 concentration and fitted to the equation: Y=Bmax*X/(K_{D}+X)+NS*X+Background, where Y is the measured anisotropy, X is the concentration of CCL8 added, Bmax is the maximum binding, K_{D} is the equilibrium dissociation constant, NS is the slope of the nonlinear regression and Background is the anisotropy when no CCL8 is present, in Graph Pad Prism. Displacement assays were carried out with CCL7, CCL8, and CCL18 (Peprotech, 1 μM). The chemokines were incubated with BK1.1 (50 μM) or SCR (50 μM) and BK1.1_{FITC} (50 nM) for 30 minutes in FA buffer and the resulting anisotropy of the emitted light determined as above. For all FP assays, the experiments were carried out as two technical and three biological replicates.

Native Mass Spectrometry Analyses

Samples were analysed using a modified Q-Exactive mass spectrometer (Thermo Fisher Scientific) for high-mass range measurements (Gault, J. et al., Nature methods [2016]). CCL8 was buffered exchanged into 200 mM ammonium acetate solution (pH=6.5). BK1.1 obtained in dimethyl sulfoxide (DMSO) was then added to the CCL8 homodimer solution in a 1:1 (CCL8 monomer:BK1.1) ratio. In all cases no more than 0.5% DMSO was present in the final mixture, and a control sample of CCL8 homodimer containing 0.5% DMSO was also analysed. Instrumental parameters were set to: capillary voltage of 1.2 KV, source temperature of 50° C. and 60 V of source induced dissociation (SID). Gas phase dissociation was carried out by applying 35 and 55 V of higher-energy collisional dissociation (HCD) to the most intense charge state after isolation (25 m/z window). Spectra was acquired using a mass resolution of 60,000 for both precursor and dissociated product ions. All measurements were done in triplicates.

AlphaScreen Assay

AlphaScreen® Histidine detection kit was purchased from PerkinElmer (6760619M lot: 2457886) and the assay was set up in white bottom Proxiplate™ 384 Plus microplates (PerkinElmer) following the manufacturer's instructions. The assay buffer used was 50 mM HEPES, 150 mM NaCl, 0.1% BSA, 0.01% Tween20, 1% DMSO, pH 7.5 and the final volume in each well was 20 μL. Briefly, biotinylated chemokine (recombinant, final concentration 1.25 nM (CCL8), 5 nM (CCL2) and 2.5 nM (CCL3)) was pre-incubated at room temperature for 15 min with different concentrations of each peptide. His-tagged P672 (final concentration 2.5 nM (CCL8), 5 nM (CCL2) and 1.25 nM (CCL3)) was then added to each well and the plate was incubated at room temperature for 30 min. Finally, acceptor and donor beads were added as a 1:1 suspension in buffer to each well and the plate was further incubated at RT for 1 h. Data was obtained by reading the plate using a Pherastar FSX plate reader (excitation 680 nm, emission 570 nm) and was analysed using GraphPad Prism.

Fluorescent Chemokine Receptor Blocking Assay

CCL8-647 (2.5 nM, final) or CCL2-657 (1.2 nM, final) was incubated for 30 minutes with varying doses of peptide (0-100 μM, final concentration) in 50 μL assay buffer (RPMI-1640+L-glutamine (4 mM)+10% heat treated fetal bovine serum+0.2% DMSO) at 37° C. This mixture was then added to 50,000 THP-1 cells in a 96-well v-bottomed plated to give a final volume of 100 μL, and everything incubated together for a further 30 minutes at 37° C. Following this time, the plate was centrifuged, the supernatant flicked off, and the cells resuspended in 150 μL ice cold PBSA. This was repeated twice more and the cells were finally resuspended in 150 μL ice cold PBSA. The median fluorescence intensity of 10,000 cells on the RL-1 channel was determined using an ATTUNE flow cytometer and plotted as function of peptide concentration and the data fitted to an inhibitor response curve with 4 parameters using Graph Pad prism 6. Experiments were performed as two technical and three biological replicates.

THP-1 Cell Migration Assays

THP-1 monocyte cell migration assays were carried out as described (Deruaz, M. et al., J Exp Med [2008]). Briefly, effective chemokine concentration (EC) EC₈₀ was determined using a 96-transwell migration plate (5 μM pore size, Corning), with THP-1 cells (300,000) in the top chamber and varying chemokine doses (0-100 nM) in the bottom chamber. The migration buffer used was RPMI 1640+0.5% FCS and 4 mM L-glutamine. This was incubated for four hours and the number of migrating cells in the bottom chamber counted using an ATTUNE flow cytometer. Data were analysed by fitting an agonist response curve with 4 parameters in GraphPad Prism. Neutralization assays were performed using the above system, using an EC₈₀ chemokine dose, and varying evasin or peptide doses in the bottom chamber for 30 min at 37° C. before beginning the assay. In experiments involving peptides, 0.2% DMSO was maintained in the migration buffer to ensure peptide solubility. IC50 was calculated by fitting an inhibitor response curve with 4 parameters in GraphPad Prism. Experiments were performed as 3 technical and 3 biological replicates.

Subcutaneous Dorsal Air Pouch Model

C57BL/6J male mice (25-30 g, 8-10-week-old) were obtained from Charles River (UK). They were group housed in temperature and humidity-controlled rooms, kept on a 12-hr light-dark cycle, and provided with food and water ad libitum. Air pouches were established at the dorsal side of the mice as described (Duarte, D. B. et al., Curr Protoc Pharmacol [2016]). Briefly, mice were anesthetized using isoflurane inhalation and air pouches were produced on day 0 by subcutaneously injecting 4 ml of sterile air into the back of the mice. On day 3, pouches were re inflated with 3 ml of sterile air. On day 6, 0.5 ml of sterile saline solution or 0.5 ml of 25 μg of zymosan (Cat #Z4250, Sigma Aldrich) in saline (w/v) was injected into the air pouch. Five minutes prior to the injection of zymosan, mice were either injected into the air pouch (local) or intra peritoneally (systemic) with 100 μL of 5 mg/kg body weight of either P672 protein or the indicated peptide which was repeated 9 hours later. Mice were sacrificed 24 hours following zymosan injection by isoflurane inhalation followed by cervical dislocation and the air-pouch exudates were collected in 2 ml of saline containing 2 mM EDTA to prevent cell aggregation. Total numbers of leucocytes, neutrophils, monocytes, T-cells and eosinophils were counted using an Attune N×T flow cytometer following staining with cell specific antibodies. Supernatants were kept at −80° C. for chemokine profiling. All animal procedures were approved and carried out in accordance with the UK Home Office Animals (Scientific Procedures) Act 1986, under project licence PPL P973A60F5.

Flow Cytometry Analysis

To characterize the inflammatory subsets in the pouch, multi-colour fluorescence cell staining was conducted using the combination of the following antibodies for various immune cells. Leucocytes (CD45-PE, Cat #130-110-797, Miltenyi Biotec), T-Cells (CD3-FITC, Cat #130-119-798, Miltenyi Biotec), neutrophils (Ly6G-PE.Vio 770, Cat #130-121-438, Miltenyi Biotec), monocytes (Ly-6C-APC, Cat #130-111-779, Miltenyi Biotec) and eosinophils (SigF-APC.Cy7, Cat #565527, BD-Pharmingen). Isotype control antibodies were run parallel (REA-PE, Cat #130-113-450, REA-PE.Vio770, Cat #130-113-452, REA-APC, Cat #130-113-446, REA-FITC, Cat #130-113-449, Miltenyi Biotec, Iso-APC.Cy7, Cat #5527770, BD-Pharmingen). One sample from each group was chosen randomly and labelled for all isotype control antibodies. Briefly, 200 μL cell suspension was pelleted down and before staining with cell surface markers, cells were incubated with an Fc receptor block (2.5 μg/10⁶ cells; BD Pharmingen, cat #553142) to reduce non-specific binding. Then, cells were suspended in 100 μL FACS buffer (0.5% BSA/PBS/2 mM EDTA) containing fluorophore conjugated antibodies (1:50 dilution, Miltenyi Biotec antibodies and 1:100 for BD-Pharmingen antibodies) and stained for 15 minutes on ice in the dark. After washing with FACS buffer, samples were run and analysis was performed on Attune N×T flow cytometer (Life Technologies, USA). Samples were run on an Attune N×T flow cytometer. Data were analysed using FlowJo by an observer blinded to the treatment received. Neutrophils were defined as CD45⁺Ly-6G⁺, monocytes/macrophages were defined as CD45+Ly-6C⁺, T cells were defined as CD45⁺CD3⁺ and eosinophils were defined as CD45⁺Ly-6G⁻ SigF⁺ (FIG. 27). Flow cytometry raw data files are available in the online supplement.

Chemokine Expression in Air Pouch Fluid

Air pouch exudate supernatants were screened for mouse chemokine profile using a mouse chemokine antibody array (RayBiotech C1 array), according to the manufacturer's instructions. The array consists of 25 different mouse chemokine antibodies spotted in duplicate, three positive controls, two negative controls and two blanks. Membranes were incubated overnight with air pouch exudate, washed and incubated with streptavidin-horseradish peroxidase and chemiluminescent reagent mix and then imaged on a BioRad ChemiDoc MP system following the manufacturer's instructions. The intensity of each chemokine spot was determined by Image Lab software (Version 5.0). Background intensity (from blanks) were subtracted from each measurement. Relative intensity to the positive control (set at 100) was calculated (see FIG. 22).

Statistical Analysis

All statistical analyses were performed using GraphPad Prism8. The statistical significance was evaluated by one-way analysis of variance (ANOVA) and P value (probability of a type I error) was adjusted for multiple comparisons with threshold (alpha) for a type I error was P≤0.05. Unless otherwise indicated all data are represented as the mean±s.e.m. of three independent experiments.

TABLE 1

| SEQ | | | Identity with |
|---|---|---|---|
| ID | Peptide | | Evasin1, 4 or 3 |
| NO: | Name | Peptide Sequence | using BLAST |
| 1 | P467_RHIPU | AEKSLDSDSSGEDYELWTQGCPFLVAENRTGFGTTVSCQHNCNGAIE | EVA1(53.09%); |
| | | KVPEGEPCYTIGEDGLGRMKLNLPYNCSLGECSGGVCVPNGRSDVCF | EVA4(35.14%); |
| | | KRTWEENNKAMA | EVA3(35.48%) |
| 2 | P546_AMBCA | ENTQQEEEDYDYGTDTCPFPVLANKTNKAKFVGCHQKCNGGDQKLT | EVA1(41.30%); |
| | | DGTACYVVERKVWDRMTPMLWYSCPLGECKNGVCEDLRKKEECRK | EVA4(36.36%) |
| | | GNGEEK | |
| 3 | P672_RHIPU | VCEVSEQEGVGEDNATEDEDYEDFFKPVTCYFANSTVGPLRPPNCTV | EVA4(36.19%); |
| | | VCTNNTAWWNDTKSDGGHCYSEYRPEKRTHSREIYNCTIGVCGNGT | EVA1(29.21%) |
| | | CIANHTYADCW | |
| 4 | P698_RHISA | NEDDSSDYYDASPMNCSSMSVNSTMGWLSMNCTMSCNGTTFPLSNS | EVA1(30.68%); |
| | | THCFHSYTNLTVQSRMETMTYNCSVGTCSNGTCVENGTTTTCW | EVA4(35.48%) |
| 5 | P943_IXORI | RSKQPTASQSSKNSIKAEFCDTNCTQGTNGAWSGCSEGCFCVHVGNN | EVA3(50.00%) |
| | | TKGRCMKLSSDYDYTTQ | |
| 6 | P974_AMBCA | ENTQQEEQDYDYGTDTCPFPVLANKTNKAKFVGCHQKCNGGDQKLT | EVA1(41.30%); |
| | | DGTACYVVERKVWDRMTPMLWYECPLGECKNGVCEDLRKKEDCRK | EVA4(36.36%) |
| | | GNGEEK | |
| 7 | P983_AMBCA | EDTGTEDDFDYGNTGCPFPVLGNYKSNMTKPVGCKNKCGSGYEVLN | EVA1(39.77%); |
| | | DTTPCYVIDQKVFNNMVPLRQYSKCPLGFCENGECKPNDQAEDCYKG | EVA4(25.00%) |
| | | REEQK | |
| 8 | P985_AMBPA | DEESEELGASTDVDYEELDANCTCPAPALTSTRNNKHYPLGCIYNCSS | EVA1(28.57%); |
| | | YNCTIPDGTPCYVLTLGEVKEHLQIGSTVPNCTCGLCRNGTCVSNGTV | EVA4(29.91%) |
| | | EECFAVEEIEET | |
| 9 | P991_AMBCA | ENGEGTTQPDYDNSTDYYNYEDFKCTCPAPHLNNTNGTVMKPIGCYY | EVA1(30.00%); |
| | | TCNVTRCTAPDTYPCYNLTEHQAKNLTTSPTTLCAVGNCDHGICVPN | EVA4(25.53%) |
| | | GTKELCFKAPNLEE | |
| 10 | P993_AMBPA | HRHYDPNEPCLIAGLKTPRDALPAGCRYDCMIKKNQKLRDGLLCLDV | EVA1(33.85%); |
| | | PEKVVKRMVNYLNYSCPLGTCRKGICKRKHRNVRCQKYPVFYMSPP | EVA4(36.36%) |
| | | K | |
| 11 | P1005_AMBMA | EKDTPKNIPGCGDTGTTAAPPADNPKHFAVYTDKHGCTIKVIGTWMT | EVA4(26.60%); |
| | | KEDHSRLPVSLRRVRASSGRVMLPASCQKICNDTVKNFPEGTPCRLVT | EVA1(27.12%); |
| | | GDPIKGKNHIKDGCIRGYCSSGVCVSDKRNISCYVPPN | EVA3(39.13%) |
| 12 | P1006_AMBCA | DTIGGIPGCGDPATTSAPEDQPKHYAVRTDKNGCKVMVIGTWMTKED | EVA1(38.33%); |
| | | HNLLPPYISKERAPSGKVQLPASCKKNCHGKLKNLPNGTPCREVFGDL | EVA4(20.22%) |
| | | RRRRKHIKDGCKVGKCQNGLCVSEERIISCYLPPNITDPRPTHGPLAE | |
| 13 | P1011_AMBTR | EKDTPKNIPGCEDAGTTAAPPADNPKHYAVYTDKNGCTFKVIGTWMT | EVA4(26.51%); |
| | | KEDHRRLPVSLRRVRASSGRVKLPASCQKICKHSVKKFPEGTPCRLVT | EVA1(23.73%); |
| | | RDPIKGKNHIKNGCIRGYCSSGVCVSDNRSISCYVLPNNTELTSPSGSF | EVA3(34.62%) |
| | | AE | |
| 14 | P1014_AMBCA | DIIGGIPGCGDPTTTSAPEVPPKHFAVRTDKDGCTLMIIGTWMTTEDHN | EVA1(30.23%); |
| | | RLPPDIGEKRAPYGRVQLPASCKKNCHGKVKNLPNGTPCREVFGDPR | EVA4(36.36%); |
| | | RGRKHIKNGCTVGKCQSGLCVTDKRIISCYIPPNITDPIPTHGPWAE | EVA3(33.33%) |
| 15 | P1015_AMBCA | EEAPGPAPGCGEPEPTPPKPRRHGIVTNVNSCNSTILVWNGKEFPALCK | EVA4(32.39%); |
| | | VRCPHKSYRVSDFEPCLKFTNRRFLQERKDETPYKCKLGFCRHGTCIT | EVA1(25.76%) |
| | | SEHSRKVPCKVPADRLDPSE | |
| 16 | P1030_AMBPA | GPPSIPGNESIPGCGDAGTTTAPEDNPKHYGTLTDKKGCTLPIIGTWMT | EVA1(24.59%); |
| | | AVDHQHLQGTRGERRGPTGKVNLPASCRKNCHGRQEILRDGIPCRKV | EVA3(37.93%) |
| | | VGNPKGSKKHLKSGCLRGKCLAGQCVNDGRRISCYVPRNITDTEPTP | |
| | | GLLAE | |
| 17 | P1063_AMBTR | QSEVGKNVPGCGDTETLEAPPQEKPPYYEYKDEEGCTQKVLESWFQA | EVA1(30.61%); |
| | | GERSTGRGQKKRGHGRRPSRVLRTVDCRKNCTVGITALPDGHLCLVP | EVA3(43.48%); |
| | | RGDPFTRGGAIKYGCYLGDCASGHCQHRYETVSCRLPAPDTTAKPYY | EVA4(26.42%) |
| | | VTPEK | |
| 18 | P1146_IXORI | GPDTKGDEESDENELFTVEYCGTNCTQLENGSWTPCSGNNGNCRCFH | None |
| | | ESDKTVGLCLSTEYTDFSEYPDPNSSEIIAAAPLPRERLIQ | detected |
| 19 | P1156_IXORI | ADDDNELFTVQYCGMNCTKDEGGTWTGCTGKKEGCKCYHESGKNY | EVA3(30.23%) |
| | | GLCLSTEYTDFSQYGNPSDSEIEAAKPKRSDTLSH | |
| 20 | P1180_AMBTR | EEPKDGYDYTEGCPFVVLGNGTHAKPAGCSHLCNGAPETLDDNMEC | EVA1(40.74%); |
| | | YNVTEEVAKRMTPDIPYTCWLGWCSKGECKRDNRTEVCYRGSERE | EVA4(28.74%) |
| 21 | P1181_AMBMA | EEREDDNDYGGGCPFVVLGNGTHAKPAGCSHLCNGAPETLDNIECYN | EVA1(45.05%); |
| | | VTEEVAKRMTPDIPYTCWLGWCSKGECKRDNRTEVCYRGSERE | EVA4(24.73%) |
| 22 | P1182_AMBMA | EPKDDNDYGGGCPFVVLGNGTHAKPAGCSHLCNGAPETLDNIECYNV | EVA1(44.19%); |
| | | TEEVAKRMTPGIPYACWLGWCNKGECKRGNRTEVCYRGSEEE | EVA4(24.18%) |
| 23 | P1183_AMBTR | EAPKDDFEYDGGCPFVVLDNGTHVKPAGCSHLCNGAPETLDNIECYN | EVA1(43.02%); |
| | | VTEEVAKRMTPGIPYACWLGWCSKGECKRDNRTEVCYRGSEEE | EVA4(25.00%) |
| 24 | P1209_AMBPA | KTDTKNAAGELPPKVAIPGCEDPATTKAPLPDDPRYYGVTIDKDGCQ | EVA1(35.59%); |
| | | RKVLGSSQRQQRQVQNGRKPGRKGRGRRPVFVDLKLTVDCKRKCNG | EVA4(22.22%) |
| | | TYSQLPDGEPCLVCDGEPYGRHRTIKGGCYQGNCSSGQCHRGERKVN | |
| | | CYIPKNITNNVLNSVNLAE | |
| 25 | P1215_AMBPA | KTDTKNAAGELPPKVVIPGCEDPATTKAPLPDEPPYYGITIDKDGCQR | EVA1(33.90%); |
| | | KVLGSSQRQQRKVQNGRKLDGKKRGRRPVFVDLELTVDCKRKCNGT | EVA4(20.83%) |
| | | YSQLPDGENCLVSDGYPYGRWGTIKGGCYQGNCSSGQCHRGEKKVN | |
| | | CYLPKNITNDVPKSLNLAE | |
| 26 | P1219_IXORI | ASLAKETEDTTLPARALVDSPDSDNCSSPQLPYFDELTYMPLGFLAVN | EVA4(32.00%) |
| | | CTKTCPVGKNGTVVNGNKCIVTWSILDVSTITVLVGSCKNGYCISDGS | |
| | | SECRNITLAGEDSQEEEEEAEEDEEEDDGDEEEDEEEDEEENDDD | |
| 27 | P1220_IXORI | GSTPSAMKTEDILKVLGSTSSLENHTDSSHCRYQELLDITKNIENAGFL | EVA3(30.43%) |
| | | AINCQRSCPNGKQTMVEGYGCIFKIKHATKRGKVKVKEGSCRKGACV | |
| | | RGSTRPPWRLLVLLGESKEEEFL | |
| 28 | P1224_IXORI | SDLCKMEAESSPFKLPQSSLLDAPDEEGCKYQLLFVEAEGPLVVNCTK | EVA1(25.33%); |
| | | DCPNGKIRTVVEGELCIAMVKTSSSGEATGLVGSCKRGSCVKKDDPC | EVA3(33.33%) |
| | | RTFTLSEEGDDDEEDEEEEEDEEEDEEEDEEEEEEDEEEED | |
| 29 | P1243_AMBAM | RNHTEDNSTEYYDYEEARCACPARHLNNTNGTVLKLLGCHYFCNGT | EVA1(32.79%); |
| | | LCTAPDGYPCYNLTAQQVRTLTTYPNTSCAVGVCMKGTCVKNGTME | EVA4(42.86%) |
| | | QCFKTP | |
| 30 | P1252_AMBAM | RGGAASVPANASIPGCGDAQTTPAAPEDQPKHYVVYRDGNGCEVKII | EVA1(30.00%); |
| | | GTWMTTEDYNCLPDTFRKNRAPHGKVKLPASCKKTCGNAVQNLKD | EVA4(24.24%) |
| | | GTPCRKVFGDLGRRRNLIKNGCLVGACQSGLCVSGNRTISCYIPPNST | |
| | | DTRATPGSFAE | |
| 31 | P1283_IXORI | KEPEDTTLPPGALVDSPDSDNCSSPHLPYFDETTNMWMGFLAVNCTK | None |
| | | KCPVGKHVTVVDGNKCIGTWSFLDELTITVLVGSCKDGFCETDGSSEC | detected |
| | | RNITLAEEDSQEEEGAAAEEEDEEDEEDEEEEEAEEEREDDHDDA | |

TABLE 2A

| | | | | Evasin Class I: Novel Evasins binding CC-chemokines: CCL2, CCL13, or CCL20 in addition to other CC chemokines as indicated | | | | | | | |
|---|---|---|---|---|---|---|---|---|---|---|---|
| | | | | Binding assay | | | | | | | |
| | | | | BLI | BLI | BLI | BLI | BLI | BLI | BLI | BLI |
| | | | | SEQ UENCE ID NO. | | | | | | | |
| | Previously described evasins | | | 9 | 29 | 1 | 3 | 20 | 7 | 21 | 8 |
| Chemokine | EVASIN1 | EVASIN4 | EVASIN3 | P991_AMBCA | P1243_AMBAM | P467_RHIPU | P672_RHIPU | P1180_AMBTR | P983_AMBPA | P1181_AMBMA | P985_AMBPA |
| CCL1_HUMAN | | 2E−10 | | | 2.13E−08 | 5.34E−08 | | | | | 8.3E−09 |
| CCL2_HUMAN | | No | | 1.22E−08 | | 1E−12 | 9.1E−09 | 5.3E−08 | 1.4E−08 | 7.4E−08 | 1.7E−08 |
| | | Binding | | | | | | | | | |
| CCL3_HUMAN | 1E−10 | 6E−11 | | 1.08E−09 | 5.3E−09 | 3.63E−10 | 2.6E−09 | 1.1E−09 | 3.5E−09 | 2.2E−09 | 8.9E−09 |
| CCL3L1_HUMAN | 5E−11 | | | 1.40E−09 | 5.05E−09 | 5.24E−10 | 2.7E−09 | 2.2E−09 | 2.1E−09 | 3.3E−09 | 7.5E−09 |
| CCL4_HUMAN | 5E−10 | No | | 5.82E−09 | 1.53E−08 | 1.36E−08 | | 6.6E−08 | 8E−09 | 5.3E−08 | 4.6E−08 |
| | | Binding | | | | | | | | | |
| CCL4L1_HUMAN | | | | 8.27E−10 | 3.54E−08 | 4.72E−09 | | 2E−08 | 2.9E−09 | 3.5E−08 | 2.1E−08 |
| CCL5_HUMAN | | 9E−11 | | 1.47E−09 | 1.74E−09 | 7.67E−09 | | | | | 3.5E−09 |
| CCL7_HUMAN | | 7E−10 | | 5.14E−10 | 8.66E−09 | 2.15E−09 | 1.6E−08 | 9.8E−08 | 3.2E−08 | 1.2E−07 | 6.1E−08 |
| CCL8_HUMAN | | 2.5E−10 | | 8.79E−09 | 2.19E−08 | 2.85E−09 | 3.7E−09 | 3.6E−09 | 7E−09 | 1.4E−08 | 6.5E−09 |
| CCL11_HUMAN | | 3.4E−10 | | 3.43E−09 | 6.03E−09 | 1.4E−09 | | 3.3E−08 | 2.1E−08 | 2E−08 | 8E−09 |
| CCL13_HUMAN | | No | | 4.19E−10 | 7.88E−10 | 2.56E−09 | | 9E−09 | 1.7E−08 | 1.9E−08 | 8.9E−08 |
| | | Binding | | | | | | | | | |
| CCL14_HUMAN | | 1.4E−10 | | 4.30E−10 | 8.16E−10 | 4.13E−09 | 5.5E−09 | 4.4E−09 | 8.1E−08 | 4.7E−08 | |
| CCL15_HUMAN | | 2.31E−09 | | 1.59E−07 | 9.61E−09 | | 3.2E−08 | | | | |
| CCL16_HUMAN | | 2.6E−10 | | 1.28E−09 | 9.26E−10 | 1.41E−08 | 3.3E−09 | | | | 4.5E−08 |
| CCL17_HUMAN | | 6.1E−10 | | 7.66E−09 | 7.34E−10 | | | | | | |
| CCL18_HUMAN | 3E−09 | 3E−11 | | 2.09E−10 | 5.01E−12 | 1.66E−08 | | 5.8E−09 | | 1E−08 | 4.3E−07 |
| CCL19_HUMAN | | 1.4E−10 | | 1.44E−08 | 5.92E−10 | | | | | | |
| CCL20_HUMAN | | No | | | 8.34E−09 | | | | | | |
| | | Binding | | | | | | | | | |
| CCL21_HUMAN | | 1E−11 | | | 6.54E−09 | | | | | | |
| CCL [symbol] 2_HUMAN | | 4.3E−10 | | 3.97E−0 [symbol] | 2.23E−0 [symbol] | | | | | | |
| CCL2 [symbol] _HUMAN | | 4.43E−09 | | 5.58E−0 [symbol] | 6.62E−09 | | [symbol] | | | | |
| CCL24_HUMAN | | 4.3E−10 | | 6.5 [symbol] E−09 | 7. [symbol] E−09 | | | | | | 4.1E−0 [symbol] |
| CCL25_HUMAN | | 6.96E−0 [symbol] | | | | | | | | | |
| CCL26_HUMAN | | 8. [symbol] E−10 | | | | | | | | | |
| CCL27_HUMAN | | No | | 3. [symbol] E−08 | 1.51E−08 | | | | | | |
| | | Binding | | | | | | | | | |
| CCL28_HUMAN | | | | | | | | | | | |
| CX [symbol] CL [symbol] _HUMAN | | | | | | | | | | | |
| CXCL1_HUMAN | | | 8.50E−10 | | | | | | | | |
| CXCL2_HUMAN | | | | | | | | | | | |
| CXCL3_HUMAN | | | | | | | | | | | |
| CXCL4_HUMAN | | | | | | | | | | | |
| CXCL5_HUMAN | | | | | | | | | | | |
| CXCL6_HUMAN | | | | | | | | | | | |
| CXCL7_HUMAN | | | | | | | | | | | |
| CXCL8_HUMAN | | | 4.30E−10 | | | | | | | | |
| CXCL9_HUMAN | | | | | | | | | | | |
| CXCL10_HUMAN | | | | | | | | | | | |
| CXCL1 [symbol] _HUMAN | | | | | | | | | | | |
| CXCL12_HUMAN | | | | | | | | | | | |
| CXCL13_HUMAN | | | | | | | | | | | |
| CXCL14_HUMAN | | | | | | | | | | | |
| CXCL1 [symbol] _HUMAN | | | | | | | | | | | |
| CXCL17_HUMAN | | | | | | | | | | | |
| CXCL [symbol] _HUMAN | | | | | | | | | | | |
| KCL1_HUMAN | | | | | | | | | | | |
| KCL [symbol] _HUMAN | | | | | | | | | | | |

| | Evasin Class I: Novel Evasins binding CC-chemokines: CCL2, CCL13, or CCL20 in addition to other CC chemokines as indicated | | | | | | | |
|---|---|---|---|---|---|---|---|---|
| | Binding assay | | | | | | | |
| | BLI | BLI | BLI | BLI | YSD | YSD | YSD | YSD |
| | SEQ UENCE ID NO. | | | | | | | |
| | 6 | 2 | 23 | 22 | 10 | 15 | 16 | 30 |
| Chemokine | P974_AMBCA | P546_AMBCA | P1183_AMBTR | P1182_AMBMA | P993_AMBPA | P1015_AMBCA | P1030_AMBPA | P1252_AMBAM |
| CCL1_HUMAN | 7.05E−08 | 1.6E−07 | | | | | | |
| CCL2_HUMAN | 9.3E−08 | 8.3E−08 | 1.2E−08 | 7.4E−08 | | | | |
| CCL3_HUMAN | 7.97E−09 | 1.5E−08 | | 5.4E−09 | | | YES | |
| CCL3L1_HUMAN | 1.31E−08 | 1.5E−08 | 1.1E−07 | 9.5E−09 | | | | |
| CCL4_HUMAN | 3.02E−08 | 2.7E−08 | 7.8E−08 | 6.3E−08 | | | | |
| CCL4L1_HUMAN | 2.32E−08 | 8.9E−09 | 4.8E−08 | 9.2E−08 | | | | |
| CCL5_HUMAN | 1.6E−07 | | | | YES | | YES | |
| CCL7_HUMAN | 4.12E−08 | 9E−08 | 5.4E−09 | | | | | |
| CCL8_HUMAN | 2.58E−09 | 1.3E−09 | 1.1E−06 | 2.9E−08 | | | YES | |
| CCL11_HUMAN | 1.7E−08 | 8.7E−09 | 8.5E−09 | 2.8E−08 | | | YES | |
| CCL13_HUMAN | 2.29E−08 | 1.3E−08 | 5.7E−09 | 5.6E−08 | | | | |
| CCL14_HUMAN | 4.91E−08 | 2.8E−08 | | 5E−08 | | | | |
| CCL15_HUMAN | | | | | | | | |
| CCL16_HUMAN | 1.1E−08 | 6.7E−09 | | | | | | |
| CCL17_HUMAN | 7.06E−08 | 7E−08 | | | YES | | YES | YES |
| CCL18_HUMAN | 1.7E−08 | 1.6E−08 | | 3.3E−08 | | | | |
| CCL19_HUMAN | | | | | | | | |
| CCL20_HUMAN | | | | | YES | YES | YES | YES |
| CCL21_HUMAN | | | | | | | | |
| CCL22_HUMAN | | 1.6 [symbol] E−08 | 2.3E− [symbol] 8 | | | | | |
| CCL23_HUMAN | | | | | | | | |
| CCL24_HUMAN | | | | | | | | |
| COL_25_HUMAN | | | | | | | | |
| COL_26_HUMAN | | | | | | | | |
| COL_27_HUMAN | | | | | | | | |
| COL_28_HUMAN | | | | | | | | |
| CX [symbol] CL [symbol] _HUMAN | | | | | | | | |
| CXCL1_HUMAN | | | | | | | | |
| CXCL2_HUMAN | | | | | | | | |
| CXCL3_HUMAN | | | | | | | | |
| CXCL4_HUMAN | | | | | | | | |
| CXCL5_HUMAN | | | | | | | | |
| CXCL6_HUMAN | | | | | | | | |
| CXCL7_HUMAN | | | | | | | | |
| CXCL8_HUMAN | | | | | | YES | | YES |
| CXCL9_HUMAN | | | | | | | | |
| CXCL10_HUMAN | | | | | | | | |
| CXCL11_HUMAN | | | | | | | | |
| CXCL12_HUMAN | | | | | | | YES | |
| CXCL13_HUMAN | | | | | | | | |
| CXCL14_HUMAN | | | | | | | | |
| CXCL1 [symbol] _HUMAN | | | | | | | | |
| CXCL17_HUMAN | | | | | | | | |
| CXCL [symbol] _HUMAN | | | | | | | | |
| KCL1_HUMAN | | | | | | | | |
| KCL [symbol] _HUMAN | | | | | | | | |

[symbol]

indicates data missing or illegible when filed

TABLE 2B

| | | | | Evasin Class II: Novel Evasins binding CXC-chemokines, 3, 10 or 12 in addition to other CXC chemokines as indicated | | | | | | |
|---|---|---|---|---|---|---|---|---|---|---|
| | | | | Binding assay | | | | | | |
| | | | | BLI | BLI | YSD | YSD | YSD | YSD | YSD |
| | | | | SEQ UENCE ID NO. | | | | | | |
| | Previously described evasins | | | 5 | 19 | 24 | 25 | 26 | 28 | 31 |
| Chemokine | EVASIN1 | EVASIN4 | EVASIN3 | P943_IXORI | P1156_IXORI | P1209_AMBPA | P1215_AMBPA | P1219_IXORI | P1224_IXORI | P1283_IXORI |

| | | | | | | | |
|---|---|---|---|---|---|---|---|
| CCL1_HUMAN | | 2E−10 | | | | YES | |
| CCL2_HUMAN | | No Binding | | | | | |
| CCL3_HUMAN | 1E−10 | 6E−11 | | | | | |
| CCL3L1_HUMAN | 5E−11 | | | | | | |
| CCL4_HUMAN | 5E−10 | No Binding | | | | | |
| CCL4L1_HUMAN | | | | | | | |
| CCL5_HUMAN | | 9E−11 | | | | YES | YES |
| CCL7_HUMAN | | 7E−10 | | | | | |
| CCL8_HUMAN | | 2.5E−10 | | | | | |
| CCL11_HUMAN | | 3.4E−10 | | | | YES | YES |
| CCL13_HUMAN | | No Binding | | | | | |
| CCL14_HUMAN | | 1.4E−10 | | | | | |
| CCL15_HUMAN | | 2.31E−09 | | | | | |
| CCL16_HUMAN | | 2.6E−10 | | | | | |
| CCL17_HUMAN | | 6.1E−10 | | | | | |
| CCL18_HUMAN | 3E−09 | 3E−11 | | | | YES | YES |
| CCL22_HUMAN | | 4.3E−10 | | | | | |
| CCL23_HUMAN | | 4.43E−09 | | | | | |
| CCL24_HUMAN | | 4.1E−10 | | | | | |
| CCL25_HUMAN | | 6.96E−08 | | | | | |
| CCL26_HUMAN | | 8.8E−10 | | | | | |
| CCL27_HUMAN | | No Binding | | | | | |
| CCL28_HUMAN | | | | | | | |
| CX3CL1_HUMAN | | | | | | | |
| CXCL1_HUMAN | | | 8.50E−10 | 5.4E−07 | 3.25E−09 | | |
| CXCL2_HUMAN | | | | | | | |
| CXCL3_HUMAN | | | | 2.7E−07 | 8.10E−08 | | |
| CXCL4_HUMAN | | | | | | | |
| CXCL5_HUMAN | | | | | | | |
| CXCL6_HUMAN | | | | | | | |
| CXCL7_HUMAN | | | | | | | |
| CXCL8_HUMAN | | | 4.30E−10 | | 4.60E−09 | | |
| CXCL9_HUMAN | | | | | | | |
| CXCL10_HUMAN | | | | | | | |
| CXCL11_HUMAN | | | | | | | |
| CXCL12_HUMAN | | | | | | YES | YES |
| CXCL13_HUMAN | | | | | | | |
| CXCL14_HUMAN | | | | | | | |
| CXCL16_HUMAN | | | | | | | |
| CXCL17_HUMAN | | | | | | | |
| CXCL4L1_HUMAN | | | | | | | |
| XCL1_HUMAN | | | | | | | |
| XCL2_HUMAN | | | | | | | |

TABLE 2C

| | | | | Evasin Class III: Other novel evasin sequences | | | | | | | |
|---|---|---|---|---|---|---|---|---|---|---|---|
| | | | | Binding assay | | | | | | | |
| | | | | YSD | YSD | YSD | YSD | YSD | YSD | YSD | YSD |
| | | | | SEQ UENCE ID NO. | | | | | | | |
| | Previously described evasins | | | 4 | 11 | 12 | 13 | 14 | 17 | 18 | 27 |
| Chemokine | EVASIN1 | EVASIN4 | EVASIN3 | P698_RHISA | P1005_AMBMA | P1006_AMBCA | P1011_AMBTR | P1014_AMBCA | P1063_AMBTR | P1146_IXORI | P1220_IXORI |

| | | | | | | | | | | |
|---|---|---|---|---|---|---|---|---|---|---|
| CCL1_HUMAN | | 2E−10 | | | YES | | | | | |
| CCL2_HUMAN | | No | | | | | | | | |
| | | Binding | | | | | | | | |
| CCL3_HUMAN | 1E−10 | 6E−11 | | | YES | | | | | |
| CCL3L1_HUMAN | 5E−11 | | | | | | | | | |
| CCL4_HUMAN | 5E−10 | No | | | | | | YES | | |
| | | Binding | | | | | | | | |
| CCL4L1_HUMAN | | | | | | | | | | |
| CCL5_HUMAN | | 9E−11 | | YES | YES | YES | YES | | YES | |
| CCL7_HUMAN | | 7E−10 | | | | | | | | |
| CCL8_HUMAN | | 2.5E−10 | | YES | | | | | | |
| CCL11_HUMAN | | 3.4E−10 | | | YES | | YES | | | |
| CCL13_HUMAN | | No | | | | | | | | |
| | | Binding | | | | | | | | |
| CCL14_HUMAN | | 1.4E−10 | | | | | | | | |
| CCL15_HUMAN | | 2.31E−09 | | | | YES | | | | |
| CCL16_HUMAN | | 2.6E−10 | | | | | | | | |
| CCL17_HUMAN | | 6.1E−10 | | | YES | | YES | | | |
| CCL18_HUMAN | 3E−09 | 3E−11 | | | | | YES | | | |
| CCL19_HUMAN | | 1.4E−10 | | | YES | YES | | | | |
| CCL24_HUMAN | | 4.1E−10 | | | | | | | | |
| CCL25_HUMAN | | 6.96E−08 | | | | YES | YES | | | YES |
| CCL26_HUMAN | | 8.8E−10 | | | | | | | | |
| CCL27_HUMAN | | No | | | | | | | | |
| | | Binding | | | | | | | | |
| CCL28_HUMAN | | | | | | | | | | |
| CX3CL1_HUMAN | | | | | | | | | | |
| CXCL1_HUMAN | | | 8.50E−10 | | | | | | | |
| CXCL2_HUMAN | | | | | | | | | | |
| CXCL3_HUMAN | | | | | | | | | | |
| CXCL4_HUMAN | | | | | | | | | | |
| CXCL5_HUMAN | | | | | | | | | | |
| CXCL6_HUMAN | | | | | | | | | | |
| CXCL13_HUMAN | | | | | | | | | | |
| CXCL14_HUMAN | | | | | | | | | | |
| CXCL16_HUMAN | | | | | | | | | | |
| CXCL17_HUMAN | | | | | | | | | | |
| CXCL4L1_HUMAN | | | | | | | | | | |
| XCL1_HUMAN | | | | | | | | | | |
| XCL2_HUMAN | | | | | | | | | | |

TABLE 3

| | SEQUENCE ID NO. | | | | |
|---|---|---|---|---|---|
| | 9 | 29 | 1 | 3 | 7 |
| Chemokine | P991_AMBCA | P1243_AMBAM | P467_RHIPU | P672_RHIPU | P983_AMBPA |
| CCL2_HUMAN | 4.78E−09 | | 3.26E−09 | | 5.95E−09 |
| CCL3_HUMAN | 2.90E−09 | | | 5.74E−09 | |
| CCL3L1_HUMAN | 1.17E−09 | | | | |
| CCL5_HUMAN | 1.84E−09 | 7.87E−09 | 8.74E−09 | | |
| CCL7_HUMAN | 6.51E−09 | | 1.11E−08 | | |
| CCL8_HUMAN | 2.10E−09 | | 1.92E−09 | 2.37E−09 | |

TABLE 4

| SEQ ID | | | Previously described | |
|---|---|---|---|---|
| NO | Peptide Name, origin | Peptide Sequence | chemokine binding | Notes |
| 32 | EVAI_RHISA (evasin 1, | EDDEDYGDLGGCPFLVAENKTGYPTI | CCL3, CCL3L1, CCL4, | |
| | from Rhipicephalus | VACKQDCNGTTETAPNGTRCFSIGDE | CCL18 | |
| | sanguineus), | GLRRMTANLPYDCPLGQCSNGDCIPK | | |
| | | ETYEVCYRRNWRDKKN | | |
| 33 | EVA3_RHISA (evasin 3, | LVSTIESRTSGDGADNFDVVSCNKNC | CXCL1, CXCL8. | |
| | from Rhipicephalus | TSGQNECPEGCFCGLLGQNKKGHCYK | | |
| | sanguineus) | IIGNLSGEPPVVRR | | |
| 34 | EVA4_RHISA (evasin 4, | EVPQMTSSSAPDLEEEDDYTAYAPLT | CCL1, CCL3, CCL5, | |
| | from Rhipicephalus | CYFTNSTLGLLAPPNCSVLCNSTTTW | CCL7, CCL8, CCL11, | |
| | sanguineus) | FNETSPNNASCLLTVDFLTQDAILQE | CCL14, CCL15, CCL16, | |
| | | NQPYNCSVGHCDNGTCAGPPRHAQCW | CCL17, CCL18, CCL19, | |
| | | | CCL21, CCL22, CCL23, | |
| | | | CCL24, CCL25, CCL26 | |
| 35 | IRI-01, Ixodes | GPAPSAKENEKAPLCLPQESLINNRD | CCL11, CCL24, CCL26, | |
| | ricinus | PNGCNYQLLPYFTEDGMGGGFLAIDC | CCL2, CCL8, CCL7 | |
| | | SKSCPEGTHETVVDGNS | | |
| 36 | IHO-01, Ixodes | AFVSSSTEVEIGSTSEHNSNETDEYG | CCL11, CCL24, CCL26, | |
| | holocyclus | YDYNADGLGCPVVGIGGLDNKTWHPN | CCL2, CCL8 | |
| | | CTNECPNSTKLFLLENGTP | | |
| 37 | ATR-02, Amblyomma | GNEVSDPPLTDEDCEYYDPSEDNITC | CCL11, CCL24, CCL26, | |
| | triste | SIRSLNTTGRPIPVGCLATCENSTRR | CCL2, CCL8, CCL7 | |
| | | LHNGTECLGISDQVANRMQGNVTYTC | | |
| | | PVGLCYRGVCORNGLGIDCWHNTPPP | | |
| | | NSTNVTTNASTTPLPTSSRDL | | |
| 38 | AMA-01, Amblyomma | ECEESDTSESTECSTEDYSNRIRDNE | CCL11, CCL24, CCL26, | |
| | maculatum | TCFIGALNTTGHPVPVGCTLDCGNST | CCL2, CCL8, CCL7 | |
| | | RYLPNGTECIDLTQQASDVMQSDVPY | | |
| | | YCPIGLCANGICKRSGLELNCWHDMP | | |
| | | PPVSTDTAIENPTTSISSSAKL | | |
| 39 | ACA-02, Amblyomma | GIEGSGNLATSHEMDDCLDDNSTCVI | CCL11, CCL24, CCL26 | |
| | cajennense | QTLNTTGEPRPVGCVLKCKNSTQHLA | | |
| | | NGTECLGIPELAGVRMQYNVSYTCAV | | |
| | | GLCNAGVCERTGLWIGCWQNEPPPNS | | |
| | | TDVTTTAPTTTTASTSSV | | |
| 40 | ACA-01, Amblyomma | ENTQQEEQDYDYGTDTCPFPVLANKT | CCL11, CCL24, CCL26, | identical to |
| | cajennense | NKAKFVGCHQKCNGGDQKLTDGTACY | CCL2, CCL8, CCL7 | P974_AMBCA, |
| | | VVERKVWDRMTPMLWYECPLGECKNG | | reported in |
| | | VCEDLRKKEDCRKGNGEEK | | PCT/GB2017/050563 |
| | | | | and Singh et al. |
| 41 | AAM-02, Amblyomma | RNHTEDNSTEYYDYEEARCACPARHL | CCL11, CCL24, CCL26, | identical to |
| | americanum | NNTNGTVLKLLGCHYFCNGTLCTAPD | CCL2, CCL7 | P1243_AMBAM, |
| | | GYPCYNLTAQQVRTLTTYPNTSCAVG | | reported in |
| | | VCMKGTCVKNGTMEQCFKTP | | PCT/GB2017/050563 |
| 42 | AAM-01, Amblyomma | ESEGSVSTETEVISYEDDCQDDNSTC | CCL11, CCL24, CCL26, | |
| | americanum | FIQTLNTTGEPRPVGCILECENSTQR | CCL2, CCL8, CCL7 | |
| | | LPNGTECLGLPGLAAVKMQRNVSYTC | | |
| | | SVGLCNGEGVCDRTGLWIGCWTNTPP | | |
| | | PNSTNVTTKPPTTTTASPGTG | | |
| 43 | RPU-02, Rhipicephalus | CEVQNTTLAEEDYDTGCGYNIVITKN | CCL11, CCL24, CCL26, | |
| | pulchellus | KTLVVNCTMDCQPKMLMNESEPCLFN | CCL2, CCL8, CCL7 | |
| | | SSVPYDHMQPHHNYTCMEGICKNGTC | | |
| | | VSPSNNITCWLPPPPVRYYPNETMVT | | |
| | | STIEPEA | | |
| 44 | RPU-01, Rhipicephalus | AEKSLDSDSSGEDYELWTQGCPFLVA | CCL11, CCL24, CCL26, | identical to |
| | pulchellus | ENRTGFGTTVSCQHNCNGAIEKVPEG | CCL2, CCL8, CCL7 | P467_RHIPU, |
| | | EPCYTIGEDGLGRMKLNLPYNCSLGE | | reported in |
| | | CSGGVCVPNGRSDVCFKRTWEENNKA | | PCT/GB2017/050563 |
| | | MA | | and Singh et al. |

TABLE 5

| SEQ | | |
|---|---|---|
| ID | Peptide | |
| NO: | Name | Peptide Sequence |
| 45 | P458_IXORI | GSKQPGAAGSSSDSVEAVFCPTNCTKGTNGAWS |
| | | GCSDDCICVHVGENTEGSCMKFSGDYDYPTPEA |
| 46 | P675_IXORI | GSNQLSGPQSSANSNDAVFCDTNCTQGTDGAWS |
| | | GCRGDCFCVHVGNSTEGRCIELIGDFDYSTPGA |
| | | ED |
| 47 | P942_IXORI | NQLSGPQSSANSNEAVFCDTNCTQGTDEAWSGC |
| | | RGDCFCVYVGNSTEGRCMMLSGDFDYSTPGAED |
| 48 | P1074_IXORI | GSKESSAHQSSDDSIKAEFCDAKCTMKTDGKWT |
| | | QCHGGCFCVHVGNETEGRCMRLDGDYDYPSTQP |
| | | EE |
| 49 | P1077_IXORI | GSKQLIGPQSSTNSIKAEFCDTNCTAGTNGIWN |
| | | GCSGDCFCTHVGNSTEGRCMKITGFDEYPTSEA |
| | | EE |
| 50 | P1078_IXORI | GSKGSSAQQSSHDSIKAEFCETNCTMKTGGKWT |
| | | QCHGGCFCVHVGNETVGRCIKLDGDYDYPSSKH |
| | | EE |
| 51 | P1080_IXORI | SAGSKGSSAPQSSGDSVVAEFCDTNCTMKKDGK |
| | | WTECNGDCFCVHVGNETVGRCMRLDGDYDYTSS |
| | | KTTRRNKKTRNGLCRLDRNRTTVDYPERNTREP |
| 52 | P1086_IXORI | GSKGQRASQVSETSITAEFCDTSCTQGTDKTWS |
| | | GCSGDCFCVHVGNDTEGRCMRWDGDYPSAEEEE |
| 53 | P1090_IXORI | GSKELSGPESSENSIEAAFCDTNCTEGTDGVWS |
| | | GCSAGCFCVHVGNSTVGRCMTFNGVDGG |
| 54 | P1095_IXORI | HSPVAGSEVQKLTSDPNDDIDVSYCGMNCTVVN |
| | | GKSDECSENCKCLHEGDDPKGICVAITYFGDWG |
| | | DPNDDPKINEATPQTQIFEKKRK |
| 55 | P1096_IXORI | HTTVTGSVEGKPNNPNEDIEVSYCRMNCTVENG |
| | | VSSACSGDCVCVHRDNEPNGICVEITYFGDFGD |
| | | PSQDPSIDEAAPRESVSKRRSNGES |
| 56 | P1100_IXORI | GSKGSSASQSSDNSVVAKFCDTNCTINEGGKWT |
| | | ECKGGCFCVHVGNETVGRCMKLDGDYDYPSPKP |
| | | EE |
| 57 | P1101_IXORI | HTTVAGSDEDIEVSYCGMNCTVESGKSSKCSPD |
| | | CVCVHEGNERDGICISITYLGDLGNPLEDPSID |
| | | LATPLAPVFQSSK |
| 58 | P1104_IXORI | ESKEASASQGPGKSFKVEFCETNCTENNGVWSG |
| | | CTGDCICVSVGDSKEGRCMDLGDKVIDTPVAQG |
| 59 | P1124_IXORI | DSKGTSDSQDSTKSIKVDFCETNCTKTDGGWTG |
| | | CTGDCICVSVGDSIEGRCMDFG |
| 60 | P1126_AMBCA | KPQILQRTDKSTDSEWDPQTCPETCIPSKNITC |
| | | SDGCVCVKLGEEEEGTCFNMTGVDWLGSPSDD |
| 61 | P1127_IXORI | AGKDDEHFSVDYCGMNCTQQEDGSWTACSGRNG |
| | | ECRCYHESGKRSGLCLSTTYIDFSEYGNLSDSD |
| | | IAAASPRLSMKESH |
| 62 | P1128_IXORI | KDDEHFSVDYCGMNCTQQEDGSWTACSGRNEEC |
| | | RCYHESGKKNGLCLSTTYIDFSQYGNPSDSDIA |
| | | AASPRP |
| 63 | P1132_IXORI | LSDEDELFSVEYCGTNCTKQDTGSWTTCSGNCT |
| | | CYHEDGKKVGLCLSTEYTDFTKFPKPTSEEIAN |
| | | ARPLPKREKTLN |
| 64 | P1134_IXORI | NEEVFTVEYCGMNCTQKSDGTWTECSGKNKDCR |
| | | CYHESDAREGLCLSTEYTDFSQFETPSNSDLEA |
| | | ATPRPRKTLYPVRNPHGPKTRGLGYDKRILRDR |
| | | VKFLI |
| 65 | P1142_AMBCA | KPQILQRTDHSTDSDWDPQMCPETCNPSKNISC |
| | | SSECLCVTLGGGDETGTCFNMSGVDWLGHAQAS |
| | | DGHNDG |
| 66 | P1162_IXORI | LNDEELFTVDYCGTNCTQQPNGSWTTCPGNCSC |
| | | YHEDGKTDGFCLSTEYTDFTQFPNLTSEEMDAA |
| | | TPRPE |
| 67 | P1166_IXORI | GPETKEDKKSDVYELFTVEYCGTNCTLLTNGRW |
| | | TACTGKKGTCRCYHESGEKVGLCLSTEYTDFSE |
| | | YPNPKSSEIDAAAPLPRETH |
| 68 | P1168_IXORI | GQDTDGKEKSDEYELFTVEYCGTNCTQLENGSW |
| | | TACTGKNGTCRCFHENDKKVGLCLSTEYTDFSE |
| | | YPDPNSEEIKAASPLP |
| 69 | P1170_IXORI | LGDEDQLFSVEYCGTNCTQQDDGKWTPCSGKNG |
| | | KCKCYHEDGKRYGLCLYTEYTDFSQYPNPEGSE |
| | | IENTRPRP |
| 70 | P1172_IXORI | LHEDEIFTVDYCGTNCTKQSNGSWTTCPGNCSC |
| | | YHEDGKTDGFCLSTEYTDFTQFPNLTSEEMDAA |
| | | TPRPE |
| 71 | P1174_IXORI | LNNENELFSVEYCGANCTQQDNGSWTKCKGNCT |
| | | CYHEDGKRYGLCLSTEYTDFTQFPKPTSEEIAD |
| | | ASPRPKETNSH |
| 72 | P1229_IXORI | DDEFFTVDYCGMNCTLQQDGSWTPCTQKNAECK |
| | | CYHESGSSVGLCLSTAYTDFNQFGDPNNSDLDA |
| | | ATPRHPDASSR |

TABLE 6

| SEQ | | Chemokines bound in yeast surface display | % Identity to EVA3, EVA4 or EVA1 |
|---|---|---|---|
| ID NO | Peptide Name | and % cells over threshold fluorescence | over alignment length in residues |
| 45 | P458_IXORI | CXCL9_HUMAN 37% | EVA3_RHISA: 33%_57 |
| 46 | P675_IXORI | CXCL11_HUMAN 55% | EVA3_RHISA: 34%_53 |
| 47 | P942_IXORI | CXCL11_HUMAN 37% | EVA3_RHISA: 32%_53 |
| 48 | P1074_IXORI | CXCL10_HUMAN 71%; CXCL11_HUMAN 33%; | EVA3_RHISA: 27%_56 |
| | | CXCL9_HUMAN 35% | |
| 49 | P1077_IXORI | CXCL1_HUMAN 50% | EVA3_RHISA: 42%_38 |
| 50 | P1078_IXORI | CXCL11_HUMAN 38% | EVA3_RHISA: 32%_47 |
| 51 | P1080_IXORI | CXCL10_HUMAN 70%; CXCL11_HUMAN 45%; | EVA3_RHISA: 33%_40 |
| | | CXCL12_HUMAN 53%; | |
| | | CXCL9_HUMAN 49% | |
| 52 | P1086_IXORI | CXCL10_HUMAN 63%; CXCL9_HUMAN 54% | EVA3_RHISA: 30%_40 |
| 53 | P1090_IXORI | CXCL11_HUMAN 32% | EVA3_RHISA: 31%_45 |
| 54 | P1095_IXORI | CXCL12_HUMAN 47% | EVA3_RHISA: 37%_49 |
| 55 | P1096_IXORI | CXCL10_HUMAN 50% | |
| 56 | P1100_IXORI | CXCL9_HUMAN 55% | EVA3_RHISA: 36%_45 |
| 57 | P1101_IXORI | CXCL10_HUMAN 75% | EVA3_RHISA: 34%_56 |
| 58 | P1104_IXORI | CXCL1_HUMAN 65% | EVA3_RHISA: 29%_63 |
| 59 | P1124_IXORI | CXCL9_HUMAN 31% | EVA3_RHISA: 30%_47 |
| 60 | P1126_AMBCA | CXCL10_HUMAN 66%; CXCL7_HUMAN 74%; | EVA3_RHISA: 31%_52 |
| | | CXCL9_HUMAN 56% | |
| 61 | P1127_IXORI | CXCL8_HUMAN 43% | EVA3_RHISA: 30%_50 |
| 62 | P1128_IXORI | CXCL8_HUMAN 53% | EVA3_RHISA: 29%_45 |
| 63 | P1132_IXORI | CXCL1_HUMAN 68% | |
| 64 | P1134_IXORI | CXCL10_HUMAN 66% | EVA3_RHISA: 34%_35 |
| 65 | P1142_AMBCA | CXCL10_HUMAN 80%; CXCL7_HUMAN 71% | |
| 66 | P1162_IXORI | CXCL1_HUMAN 72% | EVA4_RHISA: 37%_35; EVA3_RHISA: |
| | | | 34%_38 |
| 67 | P1166_IXORI | CXCL1_HUMAN 63% | |
| 68 | P1168_IXORI | CXCL1_HUMAN 66%; CXCL8_HUMAN 24% | EVA3_RHISA: 27%_44 |
| 69 | P1170_IXORI | CXCL8_HUMAN 81% | EVA3_RHISA: 33%_49 |
| 70 | P1172_IXORI | CXCL1_HUMAN 65%; CXCL8_HUMAN 41% | EVA3_RHISA: 34%_38; EVA4_RHISA: |
| | | | 34%_35 |
| 71 | P1174_IXORI | CXCL8_HUMAN 29% | EVA4_RHISA: 26%_31 |
| 72 | P1229_IXORI | CXCL1_HUMAN 66% | |

TABLE 7

| Sequence | | |
|---|---|---|
| ID | Peptide Name | Diseases |
| 1 | P467_RHIPU | alcoholic liver injury, Alzheimer's disease, atherosclerosis, atopic dermatitis, |
| | | breast cancer, colorectal cancer, idiopathic pulmonary fibrosis, inflammatory bowel |
| | | disease, influenza, kidney fibrosis, liver fibrosis, multiple sclerosis, myocardial |
| | | infarction, myocarditis, nonalcoholic steatohepatitis, paracetamol liver injury, |
| | | primary biliary cirrhosis, psoriasis, rheumatoid arthritis, sarcoidosis, skin |
| | | fibrosis, stroke, vasculitis, acute lung injury |
| 2 | P546_AMBCA | alcoholic liver injury, Alzheimer's disease, atherosclerosis, atopic dermatitis, |
| | | breast cancer, colorectal cancer, idiopathic pulmonary fibrosis, inflammatory |
| | | bowel disease, influenza, kidney fibrosis, liver fibrosis, multiple sclerosis, |
| | | myocardial infarction, myocarditis, nonalcoholic steatohepatitis, paracetamol liver |
| | | injury, primary biliary cirrhosis, primary sclerosing cholangitis, psoriasis, |
| | | rheumatoid arthritis, sarcoidosis, skin fibrosis, stroke, vasculitis, acute lung injury |
| 3 | P672_RHIPU | alcoholic liver injury, Alzheimer's disease, atherosclerosis, atopic dermatitis, breast |
| | | cancer, colorectal cancer, idiopathic pulmonary fibrosis, inflammatory bowel disease, |
| | | influenza, kidney fibrosis, liver fibrosis, multiple sclerosis, myocardial infarction, |
| | | myocarditis, nonalcoholic steatohepatitis, paracetamol liver injury, primary biliary |
| | | cirrhosis, psoriasis, rheumatoid arthritis, sarcoidosis, skin fibrosis, stroke, |
| | | vasculitis, acute lung injury |
| 4 | P698_RHISA | Alzheimer's disease, atherosclerosis, atopic dermatitis, breast cancer, idiopathic |
| | | pulmonary fibrosis, inflammatory bowel disease, influenza, kidney fibrosis, liver |
| | | fibrosis, multiple sclerosis, myocardial infarction, myocarditis, nonalcoholic |
| | | steatohepatitis, paracetamol liver injury, rheumatoid arthritis, sarcoidosis, skin |
| | | fibrosis, vasculitis |
| 5 | P943_IXORI | alcoholic liver injury, atherosclerosis, breast cancer, idiopathic pulmonary fibrosis, |
| | | inflammatory bowel disease, influenza, liver allograft rejection, liver fibrosis, |
| | | paracetamol liver injury, psoriasis, rheumatoid arthritis, skin fibrosis, |
| | | stroke, vasculitis, acute lung injury |
| 6 | P974_AMBCA | alcoholic liver injury, Alzheimer's disease, atherosclerosis, atopic dermatitis, |
| | | breast cancer, colorectal cancer, idiopathic pulmonary fibrosis, inflammatory |
| | | bowel disease, influenza, kidney fibrosis, liver fibrosis, multiple sclerosis, |
| | | myocardial infarction, myocarditis, nonalcoholic steatohepatitis, paracetamol |
| | | liver injury, primary biliary cirrhosis, psoriasis, rheumatoid arthritis, |
| | | sarcoidosis, skin fibrosis, stroke, vasculitis, acute lung injury |
| 7 | P983_AMBCA | alcoholic liver injury, Alzheimer's disease, atherosclerosis, atopic dermatitis, breast |
| | | cancer, colorectal cancer, idiopathic pulmonary fibrosis, inflammatory bowel disease, |
| | | influenza, kidney fibrosis, liver fibrosis, multiple sclerosis, myocardial |
| | | infarction, myocarditis, nonalcoholic steatohepatitis, paracetamol liver injury, |
| | | primary biliary cirrhosis, psoriasis, rheumatoid arthritis, sarcoidosis, skin |
| | | fibrosis, stroke, vasculitis, acute lung injury |
| 8 | P985_AMBPA | alcoholic liver injury, Alzheimer's disease, atherosclerosis, atopic dermatitis, |
| | | autoimmune hepatitis, breast cancer, colorectal cancer, idiopathic pulmonary |
| | | fibrosis, inflammatory bowel disease, influenza, kidney fibrosis, liver allograft |
| | | rejection, liver fibrosis, multiple sclerosis, myocardial infarction, myocarditis, |
| | | nonalcoholic steatohepatitis, paracetamol liver injury, primary biliary |
| | | cirrhosis, psoriasis, rheumatoid arthritis, sarcoidosis, skin fibrosis, stroke, |
| | | vasculitis, acute lung injury |
| 9 | P991_AMBCA | alcoholic liver injury, Alzheimer's disease, atherosclerosis, atopic dermatitis, |
| | | autoimmune hepatitis, breast cancer, colorectal cancer, idiopathic pulmonary |
| | | fibrosis, inflammatory bowel disease, influenza, kidney fibrosis, liver fibrosis, |
| | | multiple sclerosis, myocardial infarction, myocarditis, nonalcoholic steatohepatitis, |
| | | paracetamol liver injury, primary biliary cirrhosis, primary sclerosing |
| | | cholangitis, psoriasis, rheumatoid arthritis, sarcoidosis, skin fibrosis, |
| | | stroke, vasculitis, acute lung injury |
| 10 | P993_AMBPA | alcoholic liver injury, Alzheimer's disease, atherosclerosis, atopic dermatitis, |
| | | breast cancer, colorectal cancer, idiopathic pulmonary fibrosis, inflammatory |
| | | bowel disease, influenza, kidney fibrosis, liver fibrosis, multiple sclerosis, |
| | | myocardial infarction, myocarditis, nonalcoholic steatohepatitis, paracetamol |
| | | liver injury, primary biliary cirrhosis, psoriasis, rheumatoid arthritis, |
| | | sarcoidosis, skin fibrosis, stroke, vasculitis, acute lung injury |
| 11 | P1005_AMBMA | alcoholic liver injury, Alzheimer's disease, atherosclerosis, atopic dermatitis, |
| | | breast cancer, idiopathic pulmonary fibrosis, inflammatory bowel disease, influenza, |
| | | kidney fibrosis, liver fibrosis, multiple sclerosis, myocardial infarction, myocarditis, |
| | | nonalcoholic steatohepatitis, paracetamol liver injury, primary biliary cirrhosis, |
| | | psoriasis, rheumatoid arthritis, sarcoidosis, skin fibrosis, stroke, vasculitis, acute |
| | | lung injury |
| 12 | P1006_AMBCA | Alzheimer's disease, atherosclerosis, atopic dermatitis, breast cancer, idiopathic |
| | | pulmonary fibrosis, inflammatory bowel disease, influenza, kidney fibrosis, |
| | | liver fibrosis, multiple sclerosis, myocardial infarction, myocarditis, nonalcoholic |
| | | steatohepatitis, paracetamol liver injury, primary sclerosing cholangitis, rheumatoid arthritis, |
| | | sarcoidosis, skin fibrosis, vasculitis |
| 13 | P1011_AMBTR | Alzheimer's disease, atherosclerosis, atopic dermatitis, breast cancer, idiopathic |
| | | pulmonary fibrosis, inflammatory bowel disease, influenza, kidney fibrosis, liver |
| | | fibrosis, multiple sclerosis, myocardial infarction, myocarditis, nonalcoholic |
| | | steatohepatitis, paracetamol liver injury, primary sclerosing cholangitis, rheumatoid |
| | | arthritis, sarcoidosis, skin fibrosis, vasculitis |
| 14 | P1014_AMBCA | atherosclerosis, breast cancer, idiopathic pulmonary fibrosis, inflammatory bowel disease, |
| | | influenza, multiple sclerosis, myocardial infarction, psoriasis, vasculitis |
| 15 | P1015_AMBCA | alcoholic liver injury, atherosclerosis, autoimmune hepatitis, breast cancer, colorectal cancer, |
| | | idiopathic pulmonary fibrosis, inflammatory bowel disease, influenza, multiple sclerosis, |
| | | myocarditis, primary biliary cirrhosis, primary sclerosing cholangitis, psoriasis, rheumatoid |
| | | arthritis, sarcoidosis, stroke, vasculitis |
| 16 | P1030_AMBPA | alcoholic liver injury, Alzheimer's disease, atherosclerosis, atopic dermatitis, breast cancer, |
| | | colorectal cancer, idiopathic pulmonary fibrosis, inflammatory bowel disease, influenza, kidney |
| | | fibrosis, liver fibrosis, multiple sclerosis, myocardial infarction, myocarditis, |
| | | nonalcoholic steatohepatitis, paracetamol liver injury, primary biliary cirrhosis, |
| | | psoriasis, rheumatoid arthritis, sarcoidosis, skin fibrosis, stroke, vasculitis, acute lung injury |
| 17 | P1063_AMBTR | Alzheimer's disease, atherosclerosis, atopic dermatitis, breast cancer, idiopathic |
| | | pulmonary fibrosis, inflammatory bowel disease, influenza, kidney fibrosis, |
| | | liver fibrosis, multiple sclerosis, myocardial infarction, myocarditis, nonalcoholic |
| | | steatohepatitis, paracetamol liver injury, rheumatoid arthritis, sarcoidosis, skin |
| | | fibrosis, vasculitis |
| 18 | P1146_IXORI | alcoholic liver injury, Alzheimer's disease, atherosclerosis, breast cancer, idiopathic |
| | | pulmonary fibrosis, inflammatory bowel disease, influenza, myocardial infarction, |
| | | myocarditis, nonalcoholic steatohepatitis, paracetamol liver injury, primary biliary |
| | | cirrhosis, psoriasis, rheumatoid arthritis, sarcoidosis, skin fibrosis, stroke, vasculitis, |
| | | acute lung injury |
| 19 | P1156_IXORI | alcoholic liver injury, Alzheimer's disease, atherosclerosis, breast cancer, idiopathic |
| | | pulmonary fibrosis, inflammatory bowel disease, influenza, liver fibrosis, myocardial |
| | | infarction, myocarditis, nonalcoholic steatohepatitis, paracetamol liver injury, primary |
| | | biliary cirrhosis, psoriasis, rheumatoid arthritis, sarcoidosis, skin fibrosis, stroke, |
| | | vasculitis, acute lung injury |
| 20 | P1180_AMBTR | alcoholic liver injury, Alzheimer's disease, atherosclerosis, atopic dermatitis, |
| | | breast cancer, colorectal cancer, idiopathic pulmonary fibrosis, inflammatory |
| | | bowel disease, influenza, kidney fibrosis, liver fibrosis, multiple sclerosis, |
| | | myocardial infarction, myocarditis, nonalcoholic steatohepatitis, paracetamol liver |
| | | injury, primary biliary cirrhosis, psoriasis, rheumatoid arthritis, sarcoidosis, |
| | | skin fibrosis, stroke, vasculitis, acute lung injury |
| 21 | P1181_AMBMA | alcoholic liver injury, Alzheimer's disease, atherosclerosis, atopic dermatitis, |
| | | breast cancer, colorectal cancer, idiopathic pulmonary fibrosis, inflammatory bowel |
| | | disease, influenza, kidney fibrosis, liver fibrosis, multiple sclerosis, myocardial |
| | | infarction, myocarditis, nonalcoholic steatohepatitis, paracetamol liver injury, |
| | | primary biliary cirrhosis, psoriasis, rheumatoid arthritis, sarcoidosis, skin |
| | | fibrosis, stroke, vasculitis, acute lung injury |
| 22 | P1182_AMBMA | alcoholic liver injury, Alzheimer's disease, atherosclerosis, atopic dermatitis, |
| | | breast cancer, idiopathic pulmonary fibrosis, inflammatory bowel disease, |
| | | influenza, kidney fibrosis, liver fibrosis, multiple sclerosis, myocardial |
| | | infarction, myocarditis, nonalcoholic steatohepatitis, paracetamol liver injury, |
| | | primary biliary cirrhosis, psoriasis, rheumatoid arthritis, sarcoidosis, skin |
| | | fibrosis, stroke, vasculitis, acute lung injury |
| 23 | P1183_AMBTR | alcoholic liver injury, Alzheimer's disease, atherosclerosis, atopic dermatitis, |
| | | breast cancer, colorectal cancer, idiopathic pulmonary fibrosis, inflammatory bowel |
| | | disease, influenza, kidney fibrosis, liver fibrosis, multiple sclerosis, myocardial |
| | | infarction, myocarditis, nonalcoholic steatohepatitis, paracetamol liver injury, |
| | | primary biliary cirrhosis, psoriasis, rheumatoid arthritis, sarcoidosis, |
| | | skin fibrosis, stroke, vasculitis, acute lung injury |
| 24 | P1209_AMBPA | alcoholic liver injury, Alzheimer's disease, atherosclerosis, atopic dermatitis, |
| | | autoimmune hepatitis, breast cancer, colorectal cancer, idiopathic pulmonary |
| | | fibrosis, inflammatory bowel disease, influenza, kidney fibrosis, liver fibrosis, |
| | | multiple sclerosis, myocardial infarction, myocarditis, nonalcoholic |
| | | steatohepatitis, paracetamol liver injury, primary biliary cirrhosis, primary sclerosing |
| | | cholangitis, psoriasis, rheumatoid arthritis, sarcoidosis, skin fibrosis, stroke, |
| | | vasculitis, acute lung injury |
| 25 | P1215_AMBPA | Alzheimer's disease, atherosclerosis, atopic dermatitis, autoimmune hepatitis, |
| | | breast cancer, colorectal cancer, idiopathic pulmonary fibrosis, inflammatory |
| | | bowel disease, influenza, kidney fibrosis, liver fibrosis, multiple sclerosis, |
| | | myocardial infarction, myocarditis, nonalcoholic steatohepatitis, paracetamol |
| | | liver injury, primary biliary cirrhosis, primary sclerosing cholangitis, |
| | | rheumatoid arthritis, sarcoidosis, skin fibrosis, vasculitis |
| 26 | P1219_IXORI | alcoholic liver injury, Alzheimer's disease, atherosclerosis, autoimmune hepatitis, |
| | | idiopathic pulmonary fibrosis, inflammatory bowel disease, influenza, liver |
| | | fibrosis, multiple sclerosis, myocardial infarction, myocarditis, |
| | | nonalcoholic steatohepatitis, primary biliary cirrhosis, psoriasis, |
| | | rheumatoid arthritis, sarcoidosis, vasculitis |
| 27 | P1220_IXORI | atherosclerosis, inflammatory bowel disease, primary sclerosing cholangitis |
| 28 | P1224_IXORI | alcoholic liver injury, Alzheimer's disease, atherosclerosis, autoimmune hepatitis, |
| | | idiopathic pulmonary fibrosis, inflammatory bowel disease, influenza, liver |
| | | fibrosis, multiple sclerosis, myocardial infarction, myocarditis, nonalcoholic |
| | | steatohepatitis, primary biliary cirrhosis, psoriasis, rheumatoid arthritis, |
| | | sarcoidosis, vasculitis |
| 29 | P1243_AMBAM | alcoholic liver injury, Alzheimer's disease, atherosclerosis, atopic dermatitis, |
| | | breast cancer, colorectal cancer, idiopathic pulmonary fibrosis, inflammatory |
| | | bowel disease, influenza, kidney fibrosis, liver fibrosis, multiple sclerosis, |
| | | myocardial infarction, myocarditis, nonalcoholic steatohepatitis, paracetamol |
| | | liver injury, primary biliary cirrhosis, psoriasis, rheumatoid arthritis, |
| | | sarcoidosis, skin fibrosis, stroke, vasculitis, acute lung injury |
| 30 | P1252_AMBAM | alcoholic liver injury, atherosclerosis, atopic dermatitis, breast cancer, colorectal |
| | | cancer, idiopathic pulmonary fibrosis, inflammatory bowel disease, influenza, |
| | | myocarditis, primary biliary cirrhosis, psoriasis, rheumatoid arthritis, sarcoidosis, |
| | | skin fibrosis, stroke, vasculitis |
| 31 | P1283_IXORI | alcoholic liver injury, Alzheimer's disease, atherosclerosis, autoimmune hepatitis, |
| | | idiopathic pulmonary fibrosis, inflammatory bowel disease, influenza, liver fibrosis, |
| | | multiple sclerosis, myocardial infarction, myocarditis, nonalcoholic steatohepatitis, |
| | | primary biliary cirrhosis, psoriasis, rheumatoid arthritis, sarcoidosis, vasculitis |

TABLE 8

| Sequence | | |
|---|---|---|
| ID | Peptide Name | Diseases |
| 45 | P458_IXORI | atherosclerosis, autoimmune hepatitis, idiopathic pulmonary fibrosis, influenza, |
| | | multiple sclerosis, myocarditis, nonalcoholic steatohepatitis, primary biliary |
| | | cirrhosis, rheumatoid arthritis, sarcoidosis, vasculitis |
| 46 | P675_IXORI | atherosclerosis, influenza, liver allograft rejection, vasculitis |
| 47 | P942_IXORI | atherosclerosis, influenza, liver allograft rejection, vasculitis |
| 48 | P1074_IXORI | alcoholic liver injury, Alzheimer's disease, atherosclerosis, autoimmune hepatitis, |
| | | breast cancer, idiopathic pulmonary fibrosis, inflammatory bowel disease, |
| | | influenza, liver allograft rejection, liver fibrosis, multiple sclerosis, myocardial |
| | | infarction, myocarditis, nonalcoholic steatohepatitis, paracetamol liver injury, |
| | | primary biliary cirrhosis, psoriasis, rheumatoid arthritis, sarcoidosis, |
| | | skin fibrosis, stroke, vasculitis, acute lung injury |
| 49 | P1077_IXORI | alcoholic liver injury, breast cancer, idiopathic pulmonary fibrosis, inflammatory |
| | | bowel disease, liver fibrosis, paracetamol liver injury, rheumatoid arthritis, skin |
| | | fibrosis, stroke, vasculitis, acute lung injury |
| 50 | P1078_IXORI | atherosclerosis, influenza, liver allograft rejection, vasculitis |
| 51 | P1080_IXORI | alcoholic liver injury, Alzheimer's disease, atherosclerosis, autoimmune hepatitis, |
| | | breast cancer, colorectal cancer, idiopathic pulmonary fibrosis, inflammatory bowel |
| | | disease, influenza, liver allograft rejection, liver fibrosis, multiple sclerosis, |
| | | myocardial infarction, myocarditis, nonalcoholic steatohepatitis, primary biliary |
| | | cirrhosis, primary sclerosing cholangitis, psoriasis, rheumatoid arthritis, sarcoidosis, |
| | | vasculitis |
| 52 | P1086_IXORI | alcoholic liver injury, Alzheimer's disease, atherosclerosis, autoimmune hepatitis, |
| | | idiopathic pulmonary fibrosis, inflammatory bowel disease, influenza, liver fibrosis, |
| | | multiple sclerosis, myocardial infarction, myocarditis, nonalcoholic steatohepatitis, |
| | | primary biliary cirrhosis, psoriasis, rheumatoid arthritis, sarcoidosis, vasculitis |
| 53 | P1090_IXORI | atherosclerosis, influenza, liver allograft rejection, vasculitis |
| 54 | P1095_IXORI | atherosclerosis, autoimmune hepatitis, colorectal cancer, idiopathic pulmonary fibrosis, |
| | | inflammatory bowel disease, multiple sclerosis, myocarditis, primary biliary cirrhosis, |
| | | primary sclerosing cholangitis, rheumatoid arthritis |
| 55 | P1096_IXORI | alcoholic liver injury, Alzheimer's disease, atherosclerosis, autoimmune hepatitis, |
| | | idiopathic pulmonary fibrosis, inflammatory bowel disease, influenza, liver fibrosis, |
| | | multiple sclerosis, myocardial infarction, myocarditis, nonalcoholic steatohepatitis, |
| | | primary biliary cirrhosis, psoriasis, rheumatoid arthritis, sarcoidosis, vasculitis |
| 56 | P1100_IXORI | atherosclerosis, autoimmune hepatitis, idiopathic pulmonary fibrosis, influenza, multiple |
| | | sclerosis, myocarditis, nonalcoholic steatohepatitis, primary biliary cirrhosis, rheumatoid |
| | | arthritis, sarcoidosis, vasculitis |
| 57 | P1101_IXORI | alcoholic liver injury, Alzheimer's disease, atherosclerosis, autoimmune hepatitis, idiopathic |
| | | pulmonary fibrosis, inflammatory bowel disease, influenza, liver fibrosis, multiple sclerosis, |
| | | myocardial infarction, myocarditis, nonalcoholic steatohepatitis, primary biliary |
| | | cirrhosis, psoriasis, rheumatoid arthritis, sarcoidosis, vasculitis |
| 58 | P1104_IXORI | alcoholic liver injury, breast cancer, idiopathic pulmonary fibrosis, inflammatory bowel |
| | | disease, liver fibrosis, paracetamol liver injury, rheumatoid arthritis, skin fibrosis, |
| | | stroke, vasculitis, acute lung injury |
| 59 | P1124_IXORI | atherosclerosis, autoimmune hepatitis, idiopathic pulmonary fibrosis, influenza, multiple |
| | | sclerosis, myocarditis, nonalcoholic steatohepatitis, primary biliary cirrhosis, rheumatoid |
| | | arthritis, sarcoidosis, vasculitis |
| 60 | P1126_AMBCA | alcoholic liver injury, Alzheimer's disease, atherosclerosis, autoimmune hepatitis, colorectal |
| | | cancer, idiopathic pulmonary fibrosis, inflammatory bowel disease, influenza, liver fibrosis, |
| | | multiple sclerosis, myocardial infarction, myocarditis, nonalcoholic steatohepatitis, primary |
| | | biliary cirrhosis, psoriasis, rheumatoid arthritis, sarcoidosis, vasculitis |
| 61 | P1127_IXORI | alcoholic liver injury, Alzheimer's disease, atherosclerosis, breast cancer, idiopathic |
| | | pulmonary fibrosis, inflammatory bowel disease, influenza, myocardial infarction, |
| | | myocarditis, nonalcoholic steatohepatitis, paracetamol liver injury, primary biliary |
| | | cirrhosis, psoriasis, rheumatoid arthritis, sarcoidosis, skin fibrosis, stroke, vasculitis, |
| | | acute lung injury |
| 62 | P1128_IXORI | alcoholic liver injury, Alzheimer's disease, atherosclerosis, breast cancer, idiopathic |
| | | pulmonary fibrosis, inflammatory bowel disease, influenza, myocardial infarction, |
| | | myocarditis, nonalcoholic steatohepatitis, paracetamol liver injury, primary biliary |
| | | cirrhosis, psoriasis, rheumatoid arthritis, sarcoidosis, skin fibrosis, stroke, vasculitis, |
| | | acute lung injury |
| 63 | P1132_IXORI | alcoholic liver injury, breast cancer, idiopathic pulmonary fibrosis, inflammatory |
| | | bowel disease, liver fibrosis, paracetamol liver injury, rheumatoid arthritis, skin |
| | | fibrosis, stroke, vasculitis, acute lung injury |
| 64 | P1134_IXORI | alcoholic liver injury, Alzheimer's disease, atherosclerosis, autoimmune hepatitis, |
| | | idiopathic pulmonary fibrosis, inflammatory bowel disease, influenza, liver fibrosis, |
| | | multiple sclerosis, myocardial infarction, myocarditis, nonalcoholic steatohepatitis, |
| | | primary biliary cirrhosis, psoriasis, rheumatoid arthritis, sarcoidosis, vasculitis |
| 65 | P1142_AMBCA | alcoholic liver injury, Alzheimer's disease, atherosclerosis, autoimmune hepatitis, |
| | | colorectal cancer, idiopathic pulmonary fibrosis, inflammatory bowel disease, influenza, |
| | | liver fibrosis, multiple sclerosis, myocardial infarction, myocarditis, nonalcoholic |
| | | steatohepatitis, primary biliary cirrhosis, psoriasis, rheumatoid arthritis, sarcoidosis, |
| | | vasculitis |
| 66 | P1162_IXORI | alcoholic liver injury, breast cancer, idiopathic pulmonary fibrosis, inflammatory bowel |
| | | disease, liver fibrosis, paracetamol liver injury, rheumatoid arthritis, skin fibrosis, |
| | | stroke, vasculitis, acute lung injury |
| 67 | P1166_IXORI | alcoholic liver injury, breast cancer, idiopathic pulmonary fibrosis, inflammatory bowel |
| | | disease, liver fibrosis, paracetamol liver injury, rheumatoid arthritis, skin fibrosis, |
| | | stroke, vasculitis, acute lung injury |
| 68 | P1168_IXORI | alcoholic liver injury, Alzheimer's disease, atherosclerosis, breast cancer, idiopathic |
| | | pulmonary fibrosis, inflammatory bowel disease, influenza, liver fibrosis, myocardial |
| | | infarction, myocarditis, nonalcoholic steatohepatitis, paracetamol liver injury, primary |
| | | biliary cirrhosis, psoriasis, rheumatoid arthritis, sarcoidosis, skin fibrosis, stroke, |
| | | vasculitis, acute lung injury |
| 69 | P1170_IXORI | alcoholic liver injury, Alzheimer's disease, atherosclerosis, breast cancer, idiopathic |
| | | pulmonary fibrosis, inflammatory bowel disease, influenza, myocardial infarction, |
| | | myocarditis, nonalcoholic steatohepatitis, paracetamol liver injury, primary biliary |
| | | cirrhosis, psoriasis, rheumatoid arthritis, sarcoidosis, skin fibrosis, stroke, |
| | | vasculitis, acute lung injury |
| 70 | P1172_IXORI | alcoholic liver injury, Alzheimer's disease, atherosclerosis, breast cancer, idiopathic |
| | | pulmonary fibrosis, inflammatory bowel disease, influenza, liver fibrosis, myocardial |
| | | infarction, myocarditis, nonalcoholic steatohepatitis, paracetamol liver injury, primary |
| | | biliary cirrhosis, psoriasis, rheumatoid arthritis, sarcoidosis, skin fibrosis, stroke, |
| | | vasculitis, acute lung injury |
| 71 | P1174_IXORI | alcoholic liver injury, Alzheimer's disease, atherosclerosis, breast cancer, idiopathic |
| | | pulmonary fibrosis, inflammatory bowel disease, influenza, myocardial infarction, |
| | | myocarditis, nonalcoholic steatohepatitis, paracetamol liver injury, primary biliary |
| | | cirrhosis, psoriasis, rheumatoid arthritis, sarcoidosis, skin fibrosis, stroke, |
| | | vasculitis, acute lung injury |
| 72 | P1229_IXORI | alcoholic liver injury, breast cancer, idiopathic pulmonary fibrosis, inflammatory |
| | | bowel disease, liver fibrosis, paracetamol liver injury, rheumatoid arthritis, skin |
| | | fibrosis, stroke, vasculitis, acute lung injury |

TABLE 9

| BK1.1_{FITC} | CCL8, K_{d} × 10⁻⁹ (M) | Fold change |
|---|---|---|
| WT | 156 ± 12 | |
| E17A | 188 ± 30 | 1.2 |
| D18A | 387 ± 45 | 2.5 |
| E19A | 331 ± 35 | 2.1 |
| D20A | 238 ± 85 | 1.5 |
| Y21A | 736 ± 20 | 4.7 |
| E22A | 619 ± 66 | 4.0 |
| D23A | 237 ± 44 | 1.5 |
| F24A | 238 ± 19 | 1.5 |
| F25A | 720 ± 19 | 4.6 |
| K26A | 124 ± 25 | 0.8 |
| P27A | — | — |
| V28A | 601 ± 55 | 3.8 |
| T29A | 317 ± 13 | 2.0 |
| Y31A | 601 ± 87 | 3.8 |
| F32A | 360 ± 20 | 2.3 |

The mean ± s.e.m of three independent experiments is shown.
(—) indicates no binding detected.

### REFERENCES

1. Frangogiannis, N. G. (2014) The inflammatory response in myocardial injury, repair, and remodelling. Nat Rev Cardiol 11, 255-265
2. Leuschner, F., Katus, H. A., and Kaya, Z. (2009) Autoimmune myocarditis: past, present and future. Journal of autoimmunity 33, 282-289
3. Zernecke, A., and Weber, C. (2014) Chemokines in atherosclerosis: proceedings resumed. Arteriosclerosis, thrombosis, and vascular biology 34, 742-750
4. Mirabelli-Badenier, M., Braunersreuther, V., Viviani, G. L., Dallegri, F., Quercioli, A., Veneselli, E., Mach, F., and Montecucco, F. (2011) CC and CXC chemokines are pivotal mediators of cerebral injury in ischaemic stroke. Thrombosis and haemostasis 105, 409-420
5. Garin, A., and Proudfoot, A. E. I. (2011) Chemokines as targets for therapy. Experimental Cell Research 317, 602-612
6. Schall, T. J., and Proudfoot, A. E. (2011) Overcoming hurdles in developing successful drugs targeting chemokine receptors. Nat Rev Immunol 11, 355-363
7. Allen, S. J., Crown, S. E., and Handel, T. M. (2007) Chemokine:Receptor Structure, Interactions, and Antagonism. Annu. Rev. Immunol. 25, 787-820
8. Horuk, R. (2009) Chemokine receptor antagonists: overcoming developmental hurdles. Nature Reviews Drug Discovery 8, 23-33
9. Szekanecz, Z., and Koch, A. E. (2016) Successes and failures of chemokine-pathway targeting in rheumatoid arthritis. Nat Rev Rheumatol 12, 5-13
10. von Hundelshausen, P., Agten, S. M., Eckardt, V., Blanchet, X., Schmitt, M. M., Ippel, H., Neideck, C., Bidzhekov, K., Leberzammer, J., Wichapong, K., Faussner, A., Drechsler, M., Grommes, J., van Geffen, J. P., Li, H., Ortega-Gomez, A., Megens, R. T., Naumann, R., Dijkgraaf, I., Nicolaes, G. A., Doring, Y., Soehnlein, O., Lutgens, E., Heemskerk, J. W., Koenen, R. R., Mayo, K. H., Hackeng, T. M., and Weber, C. (2017) Chemokine interactome mapping enables tailored intervention in acute and chronic inflammation. Sci Transl Med 9
11. Proudfoot, A. E., and Uguccioni, M. (2016) Modulation of Chemokine Responses: Synergy and Cooperativity. Front Immunol 7, 183
12. Brandes, M., Klauschen, F., Kuchen, S., and Germain, R. N. (2013) A systems analysis identifies a feedforward inflammatory circuit leading to lethal influenza infection. Cell 154, 197-212
13. Proudfoot, A. E., Bonvin, P., and Power, C. A. (2015) Targeting chemokines: Pathogens can, why can't we? Cytokine 74, 259-267
14. Gonzalez-Motos, V., Kropp, K. A., and Viejo-Borbolla, A. (2016) Chemokine binding proteins: An immunomodulatory strategy going viral. Cytokine & growth factor reviews 30, 71-80
15. Smith, P., Fallon, R. E., Mangan, N. E., Walsh, C. M., Saraiva, M., Sayers, J. R., McKenzie, A. N. J., Alcami, A., and Fallon, P. G. (2005) Schistosoma mansoni secretes a chemokine binding protein with antiinflammatory activity. J. Exp. Med. 202, 1319-1325
16. Bonvin, P., Power, C. A., and Proudfoot, A. E. (2016) Evasins: Therapeutic Potential of a New Family of Chemokine-Binding Proteins from Ticks. Front Immunol 7, 208
17. Singh, K., Davies, G., Alenazi, Y., Eaton, J. R. O., Kawamura, A., and Bhattacharya, S. (2017) Yeast surface display identifies a family of evasins from ticks with novel polyvalent CC chemokine-binding activities. Sci Rep 7, 4267
18. Hayward, J., Sanchez, J., Perry, A., Huang, C., Rodriguez Valle, M., Canals, M., Payne, R. J., and Stone, M. J. (2017) Ticks from diverse genera encode chemokine-inhibitory evasin proteins. The Journal of biological chemistry 292, 15670-15680
19. Heidarieh, H., Hernaez, B., and Alcami, A. (2015) Immune modulation by virus-encoded secreted chemokine binding proteins. Virus Res 209, 67-75
20. Frauenschuh, A., Power, C. A., Déruaz, M., Ferreira, B. R., Silva, J. S., Teixeira, M. M., Dias, J. M., Martin, T., Wells, T. N. C., and Proudfoot, A. E. I. (2007) Molecular cloning and characterization of a highly selective chemokine-binding protein from the tick Rhipicephalus sanguineus. J. Biol. Chem. 282, 27250-27258
21. Deruaz, M., Frauenschuh, A., Alessandri, A. L., Dias, J. M., Coelho, F. M., Russo, R. C., Ferreira, B. R., Graham, G. J., Shaw, J. P., Wells, T. N., Teixeira, M. M., Power, C. A., and Proudfoot, A. E. (2008) Ticks produce highly selective chemokine binding proteins with antiinflammatory activity. J Exp Med 205, 2019-2031
22. Dias, J. M., Losberger, C., Deruaz, M., Power, C. A., Proudfoot, A. E. I., and Shaw, J. P. (2009) Structural Basis of Chemokine Sequestration by a Tick Chemokine Binding Protein: The Crystal Structure of the Complex between Evasin-1 and CCL3. PLoS ONE 4, e8514
23. Vieira, A. T., Fagundes, C. T., Alessandri, A. L., Castor, M. G., Guabiraba, R., Borges, V. O., Silveira, K. D., Vieira, E. L., Goncalves, J. L., Silva, T. A., Deruaz, M., Proudfoot, A. E., Sousa, L. P., and Teixeira, M. M. (2009) Treatment with a novel chemokine-binding protein or eosinophil lineage-ablation protects mice from experimental colitis. Am J Pathol 175, 2382-2391
24. Castor, M. G. M., Rezende, B., Resende, C. B., Alessandri, A. L., Fagundes, C. T., Sousa, L. P., Arantes, R. M. E., Souza, D. G., Silva, T. A., Proudfoot, A. E. I., Teixeira, M. M., and Pinho, V. (2010) The CCL3/macrophage inflammatory protein-lalpha-binding protein evasin-1 protects from graft-versus-host disease but does not modify graft-versus-leukemia in mice. The Journal of Immunology 184, 2646-2654
25. Montecucco, F., Lenglet, S., Braunersreuther, V., Pelli, G., Pellieux, C., Montessuit, C., Lerch, R., Deruaz, M., Proudfoot, A. E., and Mach, F. (2010) Single Administration of the CXC Chemokine-Binding Protein Evasin-3 During Ischemia Prevents Myocardial Reperfusion Injury in Mice. Arteriosclerosis, thrombosis, and vascular biology 30, 1371-1377
26. Russo, R. C., Alessandri, A. L., Garcia, C. C., Cordeiro, B. F., Pinho, V., Cassali, G. D., Proudfoot, A. E. I., and Teixeira, M. M. (2011) Therapeutic Effects of Evasin-1, a Chemokine Binding Protein, in Bleomycin-Induced Pulmonary Fibrosis. Am J Respir Cell Mol Biol 45, 72-80
27. Proudfoot, A., and Power, C. (2010) CXC-chemokine antagonists isolated from Rhipicephalus sanguineus. Google Patents
28. Deruaz, M., and Proudfoot, A. (WO2007051781A1. Publication number, 067221, 12067221, PCT/2006/67939, PCT/EP/2006/067939, PCT/EP/2006/67939,) CXC-chemokine antagonists isolated from Rhipicephalus sanguineus. US Patent Office
29. Deruaz, M., and Proudfoot, A. (WO2007093432 A1, PCT/EP2004/053638, EP1984392B1 2007) Novel cc-chemokine antagonists. US Patent Office
30. Braunersreuther, V., Montecucco, F., Pelli, G., Galan, K., Proudfoot, A. E., Belin, A., Vuilleumier, N., Burger, F., Lenglet, S., Caffa, I., Soncini, D., Nencioni, A., Vallee, J.-P., and Mach, F. (2013) Treatment with the CC chemokine-binding protein Evasin-4 improves post-infarction myocardial injury and survival in mice. Thromb. Haemost. 110, 807-825
31. Copin, J.-C., da Silva, R. F., Fraga-Silva, R. A., Capettini, L., Quintao, S., Lenglet, S., Pelli, G., Galan, K., Burger, F., Braunersreuther, V., Schaller, K., Deruaz, M., Proudfoot, A. E., Dallegri, F., Stergiopulos, N., Santos, R. A. S., Gasche, Y., Mach, F., and Montecucco, F. (2013) Treatment with Evasin-3 reduces atherosclerotic vulnerability for ischemic stroke, but not brain injury in mice. J Cereb. Blood Flow Metab. 33, 490-498
32. Deruaz, M., Bonvin, P., Severin, I. C., Johnson, Z., Krohn, S., Power, C. A., and Proudfoot, A. E. I. (2013) Evasin-4, a tick-derived chemokine-binding protein with broad selectivity can be modified for use in preclinical disease models. FEBS Journal 280, 4876-4887
33. Bonvin, P., Dunn, S. M., Rousseau, F., Dyer, D. P., Shaw, J., Power, C. A., Handel, T. M., and Proudfoot, A. E. I. (2014) Identification of the Pharmacophore of the CC Chemokine-binding Proteins Evasin-1 and-4 Using Phage Display. Journal of Biological Chemistry 289, 31846-31855
34. Montecucco, F., Mach, F., Lenglet, S., Vonlaufen, A., Gomes Quindere, A. L., Pelli, G., Burger, F., Galan, K., Dallegri, F., Carbone, F., Proudfoot, A. E., Vuilleumier, N., and Frossard, J.-L. (2014) Treatment with Evasin-3 abrogates neutrophil-mediated inflammation in mouse acute pancreatitis. Eur J Clin Invest 44, 940-950
35. Weston-Davies, W. H., Nunn, M. A., Pinto, F. O., Ian J Mackie, S., Richards, J., Machin, S. J., Prudo, R., and Hillmen, P. (2014) Clinical and Immunological Characterisation of Coversin, a Novel Small Protein Inhibitor of Complement C5 with Potential As a Therapeutic Agent in PNH and Other Complement Mediated Disorders. Blood 124, 4280
36. Lei, Y., Ripen, A. M., Ishimaru, N., Ohigashi, I., Nagasawa, T., Jeker, L. T., Bosl, M. R., Hollander, G. A., Hayashi, Y., Malefyt Rde, W., Nitta, T., and Takahama, Y. (2011) Aire-dependent production of XCL1 mediates medullary accumulation of thymic dendritic cells and contributes to regulatory T cell development. J Exp Med 208, 383-394
37. Klein, R. S., Izikson, L., Means, T., Gibson, H. D., Lin, E., Sobel, R. A., Weiner, H. L., and Luster, A. D. (2004) IFN-inducible protein 10/CXC chemokine ligand 10-independent induction of experimental autoimmune encephalomyelitis. Journal of immunology 172, 550-559
38. Turnbull, I. C., Eltoukhy, A. A., Fish, K. M., Nonnenmacher, M., Ishikawa, K., Chen, J., Hajjar, R. J., Anderson, D. G., and Costa, K. D. (2016) Myocardial Delivery of Lipidoid Nanoparticle Carrying modRNA Induces Rapid and Transient Expression. Mol Ther 24, 66-75
39. Sultana, N., Magadum, A., Hadas, Y., Kondrat, J., Singh, N., Youssef, E., Calderon, D., Chepurko, E., Dubois, N., Hajjar, R. J., and Zangi, L. (2017) Optimizing Cardiac Delivery of Modified mRNA. Mol Ther 25, 1306-1315
40. Kondrat, J., Sultana, N., and Zangi, L. (2017) Synthesis of Modified mRNA for Myocardial Delivery. Methods Mol Biol 1521, 127-138
41. Turnbull, I. C., Eltoukhy, A. A., Anderson, D. G., and Costa, K. D. (2017) Lipidoid mRNA Nanoparticles for Myocardial Delivery in Rodents. Methods Mol Biol 1521, 153-166
42. Escher, F., Vetter, R., Kuhl, U., Westermann, D., Schultheiss, H. P., and Tschope, C. (2011) Fractalkine in human inflammatory cardiomyopathy. Heart 97, 733-739
43. Fuse, K., Kodama, M., Hanawa, H., Okura, Y., Ito, M., Shiono, T., Maruyama, S., Hirono, S., Kato, K., Watanabe, K., and Aizawa, Y. (2001) Enhanced expression and production of monocyte chemoattractant protein-1 in myocarditis. Clinical and experimental immunology 124, 346-352
44. Lehmann, M. H., Kuhnert, H., Muller, S., and Sigusch, H. H. (1998) Monocyte chemoattractant protein 1 (MCP-1) gene expression in dilated cardiomyopathy. Cytokine 10, 739-746
45. Nogueira, L. G., Santos, R. H., Ianni, B. M., Fiorelli, A. I., Mairena, E. C., Benvenuti, L.

A., Frade, A., Donadi, E., Dias, F., Saba, B., Wang, H. T., Fragata, A., Sampaio, M., Hirata, M. H., Buck, P., Mady, C., Bocchi, E. A., Stolf, N. A., Kalil, J., and Cunha-Neto, E. (2012) Myocardial chemokine expression and intensity of myocarditis in Chagas cardiomyopathy are controlled by polymorphisms in CXCL9 and CXCL10. PLoS neglected tropical diseases 6, e1867
46. Kittleson, M. M., Minhas, K. M., Irizarry, R. A., Ye, S. Q., Edness, G., Breton, E., Conte, J. V., Tomaselli, G., Garcia, J. G., and Hare, J. M. (2005) Gene expression in giant cell myocarditis: Altered expression of immune response genes. International journal of cardiology 102, 333-340
47. Lassner, D., Kuhl, U., Siegismund, C. S., Rohde, M., Elezkurtaj, S., Escher, F., Tschope, C., Gross, U. M., Poller, W., and Schultheiss, H. P. (2014) Improved diagnosis of idiopathic giant cell myocarditis and cardiac sarcoidosis by myocardial gene expression profiling. Eur Heart J 35, 2186-2195
48. Satoh, M., Tamura, G., Segawa, I., Tashiro, A., Hiramori, K., and Satodate, R. (1996) Expression of cytokine genes and presence of enteroviral genomic RNA in endomyocardial biopsy tissues of myocarditis and dilated cardiomyopathy. Virchows Arch 427, 503-509
49. Zuern, C. S., Walker, B., Sauter, M., Schaub, M., Chatterjee, M., Mueller, K., Rath, D., Vogel, S., Tegtmeyer, R., Seizer, P., Geisler, T., Kandolf, R., Lang, F., Klingel, K., Gawaz, M., and Borst, O. (2015) Endomyocardial expression of SDF-1 predicts mortality in patients with suspected myocarditis. Clin Res Cardiol 104, 1033-1043
50. Borst, O., Schaub, M., Walker, B., Sauter, M., Muenzer, P., Gramlich, M., Mueller, K., Geisler, T., Lang, F., Klingel, K., Kandolf, R., Bigalke, B., Gawaz, M., and Zuern, C. S. (2014) CXCL16 is a novel diagnostic marker and predictor of mortality in inflammatory cardiomyopathy and heart failure. International journal of cardiology 176, 896-903
51. de Lemos, J. A., Morrow, D. A., Blazing, M. A., Jarolim, P., Wiviott, S. D., Sabatine, M. S., Califf, R. M., and Braunwald, E. (2007) Serial measurement of monocyte chemoattractant protein-1 after acute coronary syndromes: results from the A to Z trial. Journal of the American College of Cardiology 50, 2117-2124
52. Orn, S., Breland, U. M., Mollnes, T. E., Manhenke, C., Dickstein, K., Aukrust, P., and Ueland, T. (2009) The chemokine network in relation to infarct size and left ventricular remodeling following acute myocardial infarction. Am J Cardiol 104, 1179-1183
53. Haque, N. S., Zhang, X., French, D. L., Li, J., Poon, M., Fallon, J. T., Gabel, B. R., Taubman, M. B., Koschinsky, M., and Harpel, P. C. (2000) CC chemokine I-309 is the principal monocyte chemoattractant induced by apolipoprotein(a) in human vascular endothelial cells. Circulation 102, 786-792
54. Reape, T. J., and Groot, P. H. (1999) Chemokines and atherosclerosis. Atherosclerosis 147, 213-225
55. Haley, K. J., Lilly, C. M., Yang, J. H., Feng, Y., Kennedy, S. P., Turi, T. G., Thompson, J. F., Sukhova, G. H., Libby, P., and Lee, R. T. (2000) Overexpression of eotaxin and the CCR3 receptor in human atherosclerosis: using genomic technology to identify a potential novel pathway of vascular inflammation. Circulation 102, 2185-2189
56. Yu, R., Kim, C.-S., Kawada, T., Kwon, T.-W., Lim, T.-H., Kim, Y.-W., and Kwon, B.-S. (2004) Involvement of leukotactin-1, a novel CC chemokine, in human atherosclerosis. Atherosclerosis 174, 35-42
57. Weber, C., Meiler, S., Doring, Y., Koch, M., Drechsler, M., Megens, R. T. A., Rowinska, Z., Bidzhekov, K., Fecher, C., Ribechini, E., van Zandvoort, M. A. M. J., Binder, C. J., Jelinek, I., Hristov, M., Boon, L., Jung, S., Kom, T., Lutz, M. B., Förster, I., Zenke, M., Hieronymus, T., Junt, T., and Zernecke, A. (2011) CCL17-expressing dendritic cells drive atherosclerosis by restraining regulatory T cell homeostasis in mice. J. Clin. Invest. 121, 2898-2910
58. Calvayrac, O., Rodriguez-Calvo, R., Alonso, J., Orbe, J., Martin-Ventura, J. L., Guadall, A., Gentile, M., Juan-Babot, O., Egido, J., Beloqui, O., Paramo, J. A., Rodriguez, C., and Martinez-Gonzilez, J. (2011) CCL20 is increased in hypercholesterolemic subjects and is upregulated by LDL in vascular smooth muscle cells: role of NF-κB. Arteriosclerosis, thrombosis, and vascular biology 31, 2733-2741
59. Erbel, C., Sato, K., Meyer, F. B., Kopecky, S. L., Frye, R. L., Goronzy, J. J., and Weyand, C. M. (2007) Functional profile of activated dendritic cells in unstable atherosclerotic plaque. Basic Res Cardiol 102, 123-132
60. Kimura, S., Tanimoto, A., Wang, K.-Y., Shimajiri, S., Guo, X., Tasaki, T., Yamada, S., and Sasaguri, Y. (2012) Expression of macrophage-derived chemokine (CCL22) in atherosclerosis and regulation by histamine via the H2 receptor. Pathol. Int. 62, 675-683
61. Kim, C.-S., Kang, J.-H., Cho, H.-R., Blankenship, T. N., Erickson, K. L., Kawada, T., and Yu, R. (2011) Potential involvement of CCL23 in atherosclerotic lesion formation/progression by the enhancement of chemotaxis, adhesion molecule expression, and MMP-2 release from monocytes. Inflamm. Res. 60, 889-895
62. Abd Alla, J., Langer, A., Elzahwy, S. S., Arman-Kalcek, G., Streichert, T., and Quitterer, U. (2010) Angiotensin-converting enzyme inhibition down-regulates the pro-atherogenic chemokine receptor 9 (CCR9)-chemokine ligand 25 (CCL25) axis. Journal of Biological Chemistry 285, 23496-23505
63. Zhang, X., Feng, X., Cai, W., Liu, T., Liang, Z., Sun, Y., Yan, C., and Han, Y. (2015) Chemokine CX3CL1 and its receptor CX3CR1 are associated with human atherosclerotic lesion volnerability. Thromb. Res. 135, 1147-1153
64. Mach, F., Sauty, A., Iarossi, A. S., Sukhova, G. K., Neote, K., Libby, P., and Luster, A. D. (1999) Differential expression of three T lymphocyte-activating CXC chemokines by human atheroma-associated cells. J Clin. Invest. 104, 1041-1050
65. Abi-Younes, S., Sauty, A., Mach, F., Sukhova, G. K., Libby, P., and Luster, A. D. (2000) The stromal cell-derived factor-1 chemokine is a potent platelet agonist highly expressed in atherosclerotic plaques. Circ. Res. 86, 131-138
66. Smedbakken, L. M., Halvorsen, B., Daissormont, I., Ranheim, T., Michelsen, A. E., Skjelland, M., Sagen, E. L., Folkersen, L., Krohg-Sorensen, K., Russell, D., Holm, S., Ueland, T., Fevang, B., Hedin, U., Yndestad, A., Gullestad, L., Hansson, G. K., Biessen, E. A., and Aukrust, P. (2012) Increased levels of the homeostatic chemokine CXCL13 in human atherosclerosis—Potential role in plaque stabilization. Atherosclerosis 224, 266-273
67. Minami, M., Kume, N., Shimaoka, T., Kataoka, H., Hayashida, K., Akiyama, Y., Nagata, I., Ando, K., Nobuyoshi, M., Hanyuu, M., Komeda, M., Yonehara, S., and Kita, T. (2001) Expression of SR-PSOX, a novel cell-surface scavenger receptor for phosphatidylserine and oxidized LDL in human atherosclerotic lesions. Arteriosclerosis, thrombosis, and vascular biology 21, 1796-1800
68. Eardley, K. S., Smith, S. W., and Cockwell, P. (2009) Chemokines in vasculitis. Front Biosci (Elite Ed) 1, 26-35
69. Brix, S. R., Stege, G., Disteldorf, E., Hoxha, E., Krebs, C., Krohn, S., Otto, B., Klatschke, K., Herden, E., Heymann, F., Lira, S. A., Tacke, F., Wolf, G., Busch, M., Jabs, W. J., Ozcan, F., Keller, F., Beige, J., Wagner, K., Helmchen, U., Noriega, M., Wiech, T., Panzer, U., and Stahl, R. A. (2015) CC Chemokine Ligand 18 in ANCA-Associated Crescentic GN. J Am Soc Nephrol 26, 2105-2117
70. Dallos, T., Heiland, G. R., Strehl, J., Karonitsch, T., Gross, W. L., Moosig, F., Holl-Ulrich, C., Distler, J. H., Manger, B., Schett, G., and Zwerina, J. (2010) CCL17/thymus and activation-related chemokine in Churg-Strauss syndrome. Arthritis and rheumatism 62, 3496-3503
71. Eriksson, P., Andersson, C., Cassel, P., Nystrom, S., and Ernerudh, J. (2015) Increase in Th17-associated CCL20 and decrease in Th2-associated CCL22 plasma chemokines in active ANCA-associated vasculitis. Scandinavian journal of rheumatology 44, 80-83
72. Corbera-Bellalta, M., Planas-Rigol, E., Lozano, E., Terrades-García, N., Alba, M. A., Prieto-Gonzilez, S., Garcia-Martinez, A., Albero, R., Enjuanes, A., Espigol-Frigole, G., Hernindez-Rodriguez, J., Roux-Lombard, P., Ferlin, W. G., Dayer, J.-M., Kosco-Vilbois, M. H., and Cid, M. C. (2016) Blocking interferon γ reduces expression of chemokines CXCL9, CXCL10 and CXCL11 and decreases macrophage infiltration in ex vivo cultured arteries from patients with giant cell arteritis. Annals of the rheumatic diseases 75, 1177-1186
73. Blaschke, S., Brandt, P., Wessels, J. T., and Muller, G. A. (2009) Expression and function of the C-class chemokine lymphotactin (XCL1) in Wegener's granulomatosis. J Rheumatol 36, 2491-2500
74. Krumbholz, M., Theil, D., Cepok, S., Hemmer, B., Kivisakk, P., Ransohoff, R. M., Hofbauer, M., Farina, C., Derfuss, T., Hartle, C., Newcombe, J., Hohlfeld, R., and Meinl, E. (2006) Chemokines in multiple sclerosis: CXCL12 and CXCL13 up-regulation is differentially linked to CNS immune cell recruitment. Brain 129, 200-211
75. Miyagishi, R., Kikuchi, S., Fukazawa, T., and Tashiro, K. (1995) Macrophage inflammatory protein-1 alpha in the cerebrospinal fluid of patients with multiple sclerosis and other inflammatory neurological diseases. J Neurol Sci 129, 223-227
76. Simpson, J. E., Newcombe, J., Cuzner, M. L., and Woodroofe, M. N. (2000) Expression of the interferon-gamma-inducible chemokines IP-10 and Mig and their receptor, CXCR3, in multiple sclerosis lesions. Neuropathol Appl Neurobiol 26, 133-142
77. Liu, C., Cui, G., Zhu, M., Kang, X., and Guo, H. (2014) Neuroinflammation in Alzheimer's disease: chemokines produced by astrocytes and chemokine receptors. Int J Clin Exp Pathol 7, 8342-8355
78. Sasaki, M., Miyakoshi, M., Sato, Y., and Nakanuma, Y. (2014) Chemokine-chemokine receptor CCL2-CCR2 and CX3CL1-CX3CR1 axis may play a role in the aggravated inflammation in primary biliary cirrhosis. Dig Dis Sci 59, 358-364
79. Dyson, J. K., Hirschfield, G. M., Adams, D. H., Beuers, U., Mann, D. A., Lindor, K. D., and Jones, D. E. (2015) Novel therapeutic targets in primary biliary cirrhosis. Nat Rev Gastroenterol Hepatol 12, 147-158
80. Landi, A., Weismuller, T. J., Lankisch, T. O., Santer, D. M., Tyrrell, D. L., Manns, M. P., and Houghton, M. (2014) Differential serum levels of eosinophilic eotaxins in primary sclerosing cholangitis, primary biliary cirrhosis, and autoimmune hepatitis. J Interferon Cytokine Res 34, 204-214
81. Borchers, A. T., Shimoda, S., Bowlus, C., Keen, C. L., and Gershwin, M. E. (2009) Lymphocyte recruitment and homing to the liver in primary biliary cirrhosis and primary sclerosing cholangitis. Semin Immunopathol 31, 309-322
82. Hirschfield, G. M., and Gershwin, M. E. (2013) The immunobiology and pathophysiology of primary biliary cirrhosis. Annu Rev Pathol 8, 303-330
83. Li, Y., Wang, W., Tang, L., He, X., Yan, X., Zhang, X., Zhu, Y., Sun, J., Shi, Y., Ma, X., Mackay, I. R., Gershwin, M. E., Han, Y., and Hou, J. (2015) Chemokine (C-X-C motif) ligand 13 promotes intrahepatic chemokine (C-X-C motif) receptor 5+ lymphocyte homing and aberrant B-cell immune responses in primary biliary cirrhosis. Hepatology 61, 1998-2007
84. Heydtmann, M., Lalor, P. F., Eksteen, J. A., Hubscher, S. G., Briskin, M., and Adams, D. H. (2005) CXC Chemokine Ligand 16 Promotes Integrin-Mediated Adhesion of Liver-Infiltrating Lymphocytes to Cholangiocytes and Hepatocytes within the Inflamed Human Liver. The Journal of Immunology 174, 1055-1062
85. Eksteen, B., Grant, A. J., Miles, A., Curbishley, S. M., Lalor, P. F., Hubscher, S. G., Briskin, M., Salmon, M., and Adams, D. H. (2004) Hepatic endothelial CCL25 mediates the recruitment of CCR9+ gut-homing lymphocytes to the liver in primary sclerosing cholangitis. J Exp Med 200, 1511-1517
86. Sahin, H., and Wasmuth, H. E. (2013) Chemokines in tissue fibrosis. Biochim. Biophys. Acta 1832, 1041-1048
87. Czaja, A. J. (2014) Review article: chemokines as orchestrators of autoimmune hepatitis and potential therapeutic targets. Alimentary pharmacology & therapeutics 40, 261-279
88. Braunersreuther, V., Viviani, G. L., Mach, F., and Montecucco, F. (2012) Role of cytokines and chemokines in non-alcoholic fatty liver disease. World J Gastroenterol 18, 727-735
89. Xu, Z., Zhang, X., Lau, J., and Yu, J. (2016) C-X-C motif chemokine 10 in non-alcoholic steatohepatitis: role as a pro-inflammatory factor and clinical implication. Expert Rev Mol Med 18, e16
90. Krenkel, O., Mossanen, J. C., and Tacke, F. (2014) Immune mechanisms in acetaminophen-induced acute liver failure. Hepatobiliary Surg Nutr 3, 331-343
91. Marra, F., and Tacke, F. (2014) Roles for chemokines in liver disease. Gastroenterology 147, 577-594 e571
92. Hard, D., Griese, M., Nicolai, T., Zissel, G., Prell, C., Reinhardt, D., Schendel, D. J., and Krauss-Etschmann, S. (2005) A role for MCP-1/CCR2 in interstitial lung disease in children. Respir Res 6, 93
93. Schmidt, K., Martinez-Gamboa, L., Meier, S., Witt, C., Meisel, C., Hanitsch, L. G., Becker, M. O., Huscher, D., Burmester, G. R., and Riemekasten, G. (2009) Bronchoalveoloar lavage fluid cytokines and chemokines as markers and predictors for the outcome of interstitial lung disease in systemic sclerosis patients. Arthritis research & therapy 11, R111
94. Capelli, A., Di Stefano, A., Gnemmi, I., and Donner, C. F. (2005) CCR5 expression and CC chemokine levels in idiopathic pulmonary fibrosis. Eur Respir J 25, 701-707
95. Willems, S., Verleden, S. E., Vanaudenaerde, B. M., Wynants, M., Dooms, C., Yserbyt, J., Somers, J., Verbeken, E. K., Verleden, G. M., and Wuyts, W. A. (2013) Multiplex protein profiling of bronchoalveolar lavage in idiopathic pulmonary fibrosis and hypersensitivity pneumonitis. Ann Thorac Med 8, 38-45
96. DePianto, D. J., Chandriani, S., Abbas, A. R., Jia, G., N'Diaye, E. N., Caplazi, P., Kauder, S. E., Biswas, S., Karnik, S. K., Ha, C., Modrusan, Z., Matthay, M. A., Kukreja, J., Collard, H. R., Egen, J. G., Wolters, P. J., and Arron, J. R. (2015) Heterogeneous gene expression signatures correspond to distinct lung pathologies and biomarkers of disease severity in idiopathic pulmonary fibrosis. Thorax 70, 48-56
97. Schupp, J. C., Binder, H., Jager, B., Cillis, G., Zissel, G., Muller-Quernheim, J., and Prasse, A. (2015) Macrophage activation in acute exacerbation of idiopathic pulmonary fibrosis. PLoS One 10, e0116775
98. Petrek, M., Pantelidis, P., Southcott, A. M., Lympany, P., Safranek, P., Black, C. M., Kolek, V., Weigl, E., and du Bois, R. M. (1997) The source and role of RANTES in interstitial lung disease. Eur Respir J 10, 1207-1216
99. Belperio, J. A., Dy, M., Murray, L., Burdick, M. D., Xue, Y. Y., Strieter, R. M., and Keane, M. P. (2004) The Role of the Th2 CC Chemokine Ligand CCL17 in Pulmonary Fibrosis. The Journal of Immunology 173, 4692-4698
100. Antoniou, K. M., Tzouvelekis, A., Alexandrakis, M. G., Sfiridaki, K., Tsiligianni, I., Rachiotis, G., Tzanakis, N., Bouros, D., Milic-Emili, J., and Siafakas, N. M. (2006) Different angiogenic activity in pulmonary sarcoidosis and idiopathic pulmonary fibrosis. Chest 130, 982-988
101. Vasakova, M., Sterclova, M., Kolesar, L., Slavcev, A., Pohunek, P., Sulc, J., Skibova, J., and Striz, I. (2009) Bronchoalveolar lavage fluid cellular characteristics, functional parameters and cytokine and chemokine levels in interstitial lung diseases. Scand J Immunol 69, 268-274
102. Vuga, L. J., Tedrow, J. R., Pandit, K. V., Tan, J., Kass, D. J., Xue, J., Chandra, D., Leader, J. K., Gibson, K. F., Kaminski, N., Sciurba, F. C., and Duncan, S. R. (2014) C-X-C motif chemokine 13 (CXCL13) is a prognostic biomarker of idiopathic pulmonary fibrosis. Am J Respir Crit Care Med 189, 966-974
103. Williams, A. E., Jose, R. J., Mercer, P. F., Brealey, D., Parekh, D., Thickett, D. R., O'Kane, C., McAuley, D. F., and Chambers, R. C. (2017) Evidence for chemokine synergy during neutrophil migration in ARDS. Thorax 72, 66-73
104. Bhatia, M., Zemans, R. L., and Jeyaseelan, S. (2012) Role of chemokines in the pathogenesis of acute lung injury. Am J Respir Cell Mol Biol 46, 566-572
105. Petrek, M., Kolek, V., Szotkowska, J., and du Bois, R. M. (2002) CC and C chemokine expression in pulmonary sarcoidosis. European Respiratory Journal 20, 1206-1212
106. Palchevskiy, V., Hashemi, N., Weigt, S. S., Xue, Y. Y., Derhovanessian, A., Keane, M. P., Strieter, R. M., Fishbein, M. C., Deng, J. C., Lynch, J. P., 3rd, Elashoff, R., and Belperio, J. A. (2011) Immune response CC chemokines CCL2 and CCL5 are associated with pulmonary sarcoidosis. Fibrogenesis & tissue repair 4, 10
107. Arakelyan, A., Kriegova, E., Kubistova, Z., Mrazek, F., Kverka, M., du Bois, R. M., Kolek, V., and Petrek, M. (2009) Protein levels of CC chemokine ligand (CCL)15, CCL16 and macrophage stimulating protein in patients with sarcoidosis. Clinical and experimental immunology 155, 457-465
108. Cai, M., Bonella, F., He, X., Sixt, S. U., Sarria, R., Guzman, J., and Costabel, U. (2013) CCL18 in serum, BAL fluid and alveolar macrophage culture supernatant in interstitial lung diseases. Respir Med 107, 1444-1452
109. Gibejova, A., Mrazek, F., Subrtova, D., Sekerova, V., Szotkowska, J., Kolek, V., du Bois, R. M., and Petrek, M. (2003) Expression of macrophage inflammatory protein-3 beta/CCL19 in pulmonary sarcoidosis. Am J Respir Crit Care Med 167, 1695-1703
110. Facco, M., Baesso, I., Miorin, M., Bortoli, M., Cabrelle, A., Boscaro, E., Gurrieri, C., Trentin, L., Zambello, R., Calabrese, F., Cassatella, M. A., Semenzato, G., and Agostini, C. (2007) Expression and role of CCR6/CCL20 chemokine axis in pulmonary sarcoidosis. J Leukoc Biol 82, 946-955
111. Agostini, C., Cassatella, M., Zambello, R., Trentin, L., Gasperini, S., Perin, A., Piazza, F., Siviero, M., Facco, M., Dziejman, M., Chilosi, M., Qin, S., Luster, A. D., and Semenzato, G. (1998) Involvement of the IP-10 chemokine in sarcoid granulomatous reactions. Journal of immunology 161, 6413-6420
112. de Jong, M. D., Simmons, C. P., Thanh, T. T., Hien, V. M., Smith, G. J., Chau, T. N., Hoang, D. M., Chau, N. V., Khanh, T. H., Dong, V. C., Qui, P. T., Cam, B. V., Ha do, Q., Guan, Y., Peiris, J. S., Chinh, N. T., Hien, T. T., and Farrar, J. (2006) Fatal outcome of human influenza A (H5N1) is associated with high viral load and hypercytokinemia. Nat Med 12, 1203-1207
113. Takano, T., Tajiri, H., Kashiwagi, Y., Kimura, S., and Kawashima, H. (2011) Cytokine and chemokine response in children with the 2009 pandemic influenza A (H1N1) virus infection. Eur J Clin Microbiol Infect Dis 30, 117-120
114. Lee, N., Wong, C. K., Chan, P. K., Chan, M. C., Wong, R. Y., Lun, S. W., Ngai, K. L., Lui, G. C., Wong, B. C., Lee, S. K., Choi, K. W., and Hui, D. S. (2011) Cytokine response patterns in severe pandemic 2009 H1N1 and seasonal influenza among hospitalized adults. PLoS One 6, e26050
115. Wang, Z., Zhang, A., Wan, Y., Liu, X., Qiu, C., Xi, X., Ren, Y., Wang, J., Dong, Y., Bao, M., Li, L., Zhou, M., Yuan, S., Sun, J., Zhu, Z., Chen, L., Li, Q., Zhang, Z., Zhang, X., Lu, S., Doherty, P. C., Kedzierska, K., and Xu, J. (2014) Early hypercytokinemia is associated with interferon-induced transmembrane protein-3 dysfunction and predictive of fatal H7N9 infection. Proceedings of the National Academy of Sciences of the United States of America 111, 769-774
116. Gao, R., Bhatnagar, J., Blau, D. M., Greer, P., Rollin, D. C., Denison, A. M., Deleon-Carnes, M., Shieh, W. J., Sambhara, S., Tumpey, T. M., Patel, M., Liu, L., Paddock, C., Drew, C., Shu, Y., Katz, J. M., and Zaki, S. R. (2013) Cytokine and chemokine profiles in lung tissues from fatal cases of 2009 pandemic influenza A (H1N1): role of the host immune response in pathogenesis. Am J Pathol 183, 1258-1268
117. Zeng, H., Belser, J. A., Goldsmith, C. S., Gustin, K. M., Veguilla, V., Katz, J. M., and Tumpey, T. M. (2015) A(H7N9) virus results in early induction of proinflammatory cytokine responses in both human lung epithelial and endothelial cells and shows increased human adaptation compared with avian H5N1 virus. J Virol 89, 4655-4667
118. Marion, T., Elbahesh, H., Thomas, P. G., DeVincenzo, J. P., Webby, R., and Schughart, K. (2016) Respiratory Mucosal Proteome Quantification in Human Influenza Infections. PLoS One 11, e0153674
119. Lee, N., Wong, C. K., Chan, P. K., Lun, S. W., Lui, G., Wong, B., Hui, D. S., Lam, C. W., Cockram, C. S., Choi, K. W., Yeung, A. C., Tang, J. W., and Sung, J. J. (2007) Hypercytokinemia and hyperactivation of phospho-p38 mitogen-activated protein kinase in severe human influenza A virus infection. Clin Infect Dis 45, 723-731
120. Kim, Y. H., Kim, J. E., and Hyun, M. C. (2011) Cytokine response in pediatric patients with pandemic influenza H1N1 2009 virus infection and pneumonia: comparison with pediatric pneumonia without H1N1 2009 infection. Pediatr Pulmonol 46, 1233-1239
121. Bian, J. R., Nie, W., Zang, Y. S., Fang, Z., Xiu, Q. Y., and Xu, X. X. (2014) Clinical aspects and cytokine response in adults with seasonal influenza infection. Int J Clin Exp Med 7, 5593-5602
122. Hagau, N., Slavcovici, A., Gonganau, D. N., Oltean, S., Dirzu, D. S., Brezoszki, E. S., Maxim, M., Ciuce, C., Mlesnite, M., Gavrus, R. L., Laslo, C., Hagau, R., Petrescu, M., and Studnicska, D. M. (2010) Clinical aspects and cytokine response in severe H1N1 influenza A virus infection. Crit Care 14, R203
123. Mazzucchelli, L., Hauser, C., Zgraggen, K., Wagner, H. E., Hess, M. W., Laissue, J. A., and Mueller, C. (1996) Differential in situ expression of the genes encoding the chemokines MCP-1 and RANTES in human inflammatory bowel disease. The Journal of pathology 178, 201-206
124. Banks, C., Bateman, A., Payne, R., Johnson, P., and Sheron, N. (2003) Chemokine expression in IBD. Mucosal chemokine expression is unselectively increased in both ulcerative colitis and Crohn's disease. The Journal of pathology 199, 28-35
125. Reinecker, H. C., Loh, E. Y., Ringler, D. J., Mehta, A., Rombeau, J. L., and MacDermott, R. P. (1995) Monocyte-chemoattractant protein 1 gene expression in intestinal epithelial cells and inflammatory bowel disease mucosa. Gastroenterology 108, 40-50
126. Uguccioni, M., Gionchetti, P., Robbiani, D. F., Rizzello, F., Peruzzo, S., Campieri, M., and Baggiolini, M. (1999) Increased expression of IP-10, IL-8, MCP-1, and MCP-3 in ulcerative colitis. Am J Pathol 155, 331-336
127. Ahrens, R., Waddell, A., Seidu, L., Blanchard, C., Carey, R., Forbes, E., Lampinen, M., Wilson, T., Cohen, E., Stringer, K., Ballard, E., Munitz, A., Xu, H., Lee, N., Lee, J. J., Rothenberg, M. E., Denson, L., and Hogan, S. P. (2008) Intestinal macrophage/epithelial cell-derived CCL11/eotaxin-1 mediates eosinophil recruitment and function in pediatric ulcerative colitis. Journal of immunology 181, 7390-7399
128. Kotarsky, K., Sitnik, K. M., Stenstad, H., Kotarsky, H., Schmidtchen, A., Koslowski, M., Wehkamp, J., and Agace, W. W. (2010) A novel role for constitutively expressed epithelial-derived chemokines as antibacterial peptides in the intestinal mucosa. Mucosal immunology 3, 40-48
129. Puleston, J., Cooper, M., Murch, S., Bid, K., Makh, S., Ashwood, P., Bingham, A. H., Green, H., Moss, P., Dhillon, A., Morris, R., Strobel, S., Gelinas, R., Pounder, R. E., and Platt, A. (2005) A distinct subset of chemokines dominates the mucosal chemokine response in inflammatory bowel disease. Alimentary pharmacology & therapeutics 21, 109-120
130. Papadakis, K. A., Prehn, J., Moreno, S. T., Cheng, L., Kouroumalis, E. A., Deem, R., Breaverman, T., Ponath, P. D., Andrew, D. P., Green, P. H., Hodge, M. R., Binder, S. W., and Targan, S. R. (2001) CCR9-positive lymphocytes and thymus-expressed chemokine distinguish small bowel from colonic Crohn's disease. Gastroenterology 121, 246-254
131. Sans, M., Danese, S., de la Motte, C., de Souza, H. S., Rivera-Reyes, B. M., West, G. A., Phillips, M., Katz, J. A., and Fiocchi, C. (2007) Enhanced recruitment of CX3CR1+ T cells by mucosal endothelial cell-derived fractalkine in inflammatory bowel disease. Gastroenterology 132, 139-153
132. Izzo, R. S., Witkon, K., Chen, A. I., Hadjiyane, C., Weinstein, M. I., and Pellecchia, C. (1993) Neutrophil-activating peptide (interleukin-8) in colonic mucosa from patients with Crohn's disease. Scandinavian journal of gastroenterology 28, 296-300
133. Mahida, Y. R., Ceska, M., Effenberger, F., Kurlak, L., Lindley, I., and Hawkey, C. J. (1992) Enhanced synthesis of neutrophil-activating peptide-1/interleukin-8 in active ulcerative colitis. Clinical science 82, 273-275
134. Dotan, I., Werner, L., Vigodman, S., Weiss, S., Brazowski, E., Maharshak, N., Chen, O., Tulchinsky, H., Halpern, Z., and Guzner-Gur, H. (2010) CXCL12 is a constitutive and inflammatory chemokine in the intestinal immune system. Inflammatory bowel diseases 16, 583-592
135. Rau, B., Baumgart, K., Kruger, C. M., Schilling, M., and Beger, H. G. (2003) CC-chemokine activation in acute pancreatitis: enhanced release of monocyte chemoattractant protein-1 in patients with local and systemic complications. Intensive Care Med 29, 622-629
136. Saurer, L., Reber, P., Schaffner, T., Buchler, M. W., Buri, C., Kappeler, A., Walz, A., Friess, H., and Mueller, C. (2000) Differential expression of chemokines in normal pancreas and in chronic pancreatitis. Gastroenterology 118, 356-367
137. Nomura, I., Gao, B., Boguniewicz, M., Darst, M. A., Travers, J. B., and Leung, D. Y. (2003) Distinct patterns of gene expression in the skin lesions of atopic dermatitis and psoriasis: a gene microarray analysis. The Journal of allergy and clinical immunology 112, 1195-1202
138. Brunner, P. M., Glitzner, E., Reininger, B., Klein, I., Stary, G., Mildner, M., Uhrin, P., Sibilia, M., and Stingl, G. (2015) CCL7 contributes to the TNF-alpha-dependent inflammation of lesional psoriatic skin. Experimental dermatology 24, 522-528
139. Hedrick, M. N., Lonsdorf, A. S., Hwang, S. T., and Farber, J. M. (2010) CCR6 as a possible therapeutic target in psoriasis. Expert Opin Ther Targets 14, 911-922
140. Congjun, J., Yanmei, Z., Huiling, J., Zhen, Y., and Shuo, L. (2015) Elevated Local and Serum CX3CL1(Fractalkine) Expression and Its Association with Disease Severity in Patients with Psoriasis. Ann Clin Lab Sci 45, 556-561
141. Ferrari, S. M., Ruffilli, I., Colaci, M., Antonelli, A., Ferri, C., and Fallahi, P. (2015) CXCL10 in psoriasis. Adv Med Sci 60, 349-354
142. Owczarek, W., Paplinska, M., Targowski, T., Jahnz-Rozyk, K., Paluchowska, E., Kucharczyk, A., and Kasztalewicz, B. (2010) Analysis of eotaxin 1/CCL11, eotaxin 2/CCL24 and eotaxin 3/CCL26 expression in lesional and non-lesional skin of patients with atopic dermatitis. Cytokine 50, 181-185
143. Hijnen, D., De Bruin-Weller, M., Oosting, B., Lebre, C., De Jong, E., Bruijnzeel-Koomen, C., and Knol, E. (2004) Serum thymus and activation-regulated chemokine (TARC) and cutaneous T cell-attracting chemokine (CTACK) levels in allergic diseases: TARC and CTACK are disease-specific markers for atopic dermatitis. The Journal of allergy and clinical immunology 113, 334-340
144. Pivarcsi, A., Gombert, M., Dieu-Nosjean, M. C., Lauerma, A., Kubitza, R., Meller, S., Rieker, J., Muller, A., Da Cunha, L., Haahtela, A., Sonkoly, E., Fridman, W. H., Alenius, H., Kemeny, L., Ruzicka, T., Zlotnik, A., and Homey, B. (2004) CC chemokine ligand 18, an atopic dermatitis-associated and dendritic cell-derived chemokine, is regulated by staphylococcal products and allergen exposure. Journal of immunology 173, 5810-5817
145. Park, C. O., Lee, H. J., Lee, J. H., Wu, W. H., Chang, N. S., Hua, L., Lee, M. G., and Lee, K. H. (2008) Increased expression of CC chemokine ligand 18 in extrinsic atopic dermatitis patients. Experimental dermatology 17, 24-29
146. Kakinuma, T., Saeki, H., Tsunemi, Y., Fujita, H., Asano, N., Mitsui, H., Tada, Y., Wakugawa, M., Watanabe, T., Torii, H., Komine, M., Asahina, A., Nakamura, K., and Tamaki, K. (2003) Increased serum cutaneous T cell-attracting chemokine (CCL27) levels in patients with atopic dermatitis and psoriasis vulgaris. The Journal of allergy and clinical immunology 111, 592-597
147. Echigo, T., Hasegawa, M., Shimada, Y., Takehara, K., and Sato, S. (2004) Expression of fractalkine and its receptor, CX3CR1, in atopic dermatitis: possible contribution to skin inflammation. The Journal of allergy and clinical immunology 113, 940-948
148. Ueno, T., Toi, M., Saji, H., Muta, M., Bando, H., Kuroi, K., Koike, M., Inadera, H., and Matsushima, K. (2000) Significance of macrophage chemoattractant protein-1 in macrophage recruitment, angiogenesis, and survival in human breast cancer. Clin Cancer Res 6, 3282-3289
149. Soria, G., Yaal-Hahoshen, N., Azenshtein, E., Shina, S., Leider-Trejo, L., Ryvo, L., Cohen-Hillel, E., Shtabsky, A., Ehrlich, M., Meshel, T., Keydar, I., and Ben-Baruch, A. (2008) Concomitant expression of the chemokines RANTES and MCP-1 in human breast cancer: a basis for tumor-promoting interactions. Cytokine 44, 191-200
150. Chavey, C., Bibeau, F., Gourgou-Bourgade, S., Burlinchon, S., Boissiere, F., Laune, D., Roques, S., and Lazennec, G. (2007) Oestrogen receptor negative breast cancers exhibit high cytokine content. Breast Cancer Res 9, R15
151. Luboshits, G., Shina, S., Kaplan, O., Engelberg, S., Nass, D., Lifshitz-Mercer, B., Chaitchik, S., Keydar, I., and Ben-Baruch, A. (1999) Elevated expression of the CC chemokine regulated on activation, normal T cell expressed and secreted (RANTES) in advanced breast carcinoma. Cancer Res 59, 4681-4687
152. Chen, J., Yao, Y., Gong, C., Yu, F., Su, S., Chen, J., Liu, B., Deng, H., Wang, F., Lin, L., Yao, H., Su, F., Anderson, K. S., Liu, Q., Ewen, M. E., Yao, X., and Song, E. (2011) CCL18 from tumor-associated macrophages promotes breast cancer metastasis via PITPNM3. Cancer Cell 19, 541-555
153. Bell, D., Chomarat, P., Broyles, D., Netto, G., Harb, G. M., Lebecque, S., Valladeau, J., Davoust, J., Palucka, K. A., and Banchereau, J. (1999) In breast carcinoma tissue, immature dendritic cells reside within the tumor, whereas mature dendritic cells are located in peritumoral areas. J Exp Med 190, 1417-1426
154. Bieche, I., Chavey, C., Andrieu, C., Busson, M., Vacher, S., Le Corre, L., Guinebretiere, J. M., Burlinchon, S., Lidereau, R., and Lazennec, G. (2007) CXC chemokines located in the 4q21 region are up-regulated in breast cancer. Endocr Relat Cancer 14, 1039-1052
155. Cho, Y. B., Lee, W. Y., Choi, S. J., Kim, J., Hong, H. K., Kim, S. H., Choi, Y. L., Kim, H. C., Yun, S. H., Chun, H. K., and Lee, K. U. (2012) CC chemokine ligand 7 expression in liver metastasis of colorectal cancer. Oncol Rep 28, 689-694
156. Nomiyama, H., Hieshima, K., Nakayama, T., Sakaguchi, T., Fujisawa, R., Tanase, S., Nishiura, H., Matsuno, K., Takamori, H., Tabira, Y., Yamamoto, T., Miura, R., and Yoshie, O. (2001) Human CC chemokine liver-expressed chemokine/CCL16 is a functional ligand for CCR1, CCR2 and CCR5, and constitutively expressed by hepatocytes. International immunology 13, 1021-1029
157. Rubie, C., Oliveira, V., Kempf, K., Wagner, M., Tilton, B., Rau, B., Kruse, B., Konig, J., and Schilling, M. (2006) Involvement of chemokine receptor CCR6 in colorectal cancer metastasis. Tumour Biol 27, 166-174
158. Desurmont, T., Skrypek, N., Duhamel, A., Jonckheere, N., Millet, G., Leteurtre, E., Gosset, P., Duchene, B., Ramdane, N., Hebbar, M., Van Seuningen, I., Pruvot, F. R., Huet, G., and Truant, S. (2015) Overexpression of chemokine receptor CXCR2 and ligand CXCL7 in liver metastases from colon cancer is correlated to shorter disease-free and overall survival. Cancer Sci 106, 262-269
159. Kim, J., Mori, T., Chen, S. L., Amersi, F. F., Martinez, S. R., Kuo, C., Turner, R. R., Ye, X., Bilchik, A. J., Morton, D. L., and Hoon, D. S. (2006) Chemokine receptor CXCR4 expression in patients with melanoma and colorectal cancer liver metastases and the association with disease outcome. Ann Surg 244, 113-120
160. Webb, B., and Sali, A. (2014) Protein structure modeling with MODELLER. Methods Mol Biol 1137, 1-15
161. Janson, G., Zhang, C., Prado, M. G., and Paiardini, A. (2017) PyMod 2.0: improvements in protein sequence-structure analysis and homology modeling within PyMOL. Bioinformatics 33, 444-446
162. Krissinel, E., and Henrick, K. (2007) Inference of macromolecular assemblies from crystalline state. Journal of molecular biology 372, 774-797
163. Alenazi, Y., Singh, K., Davies, G., Eaton, J. R. O., Elders, P., Kawamura, A., and Bhattacharya, S. (2018) Genetically engineered two-warhead evasins provide a method to achieve precision targeting of disease-relevant chemokine subsets. Sci Rep 8, 6333
164. Bachelerie, F., Ben-Baruch, A., Burkhardt, A. M., Combadiere, C., Farber, J. M., Graham, G. J., Horuk, R., Sparre-Ulrich, A. H., Locati, M., Luster, A. D., Mantovani, A., Matsushima, K., Murphy, P. M., Nibbs, R., Nomiyama, H., Power, C. A., Proudfoot, A. E., Rosenkilde, M. M., Rot, A., Sozzani, S., Thelen, M., Yoshie, O., and Zlotnik, A. (2014) International Union of Basic and Clinical Pharmacology. LXXXIX. Update on the extended family of chemokine receptors and introducing a new nomenclature for atypical chemokine receptors. Pharmacol Rev 66, 1-79
165. Concepcion, J., Witte, K., Wartchow, C., Choo, S., Yao, D., Persson, H., Wei, J., Li, P., Heidecker, B., Ma, W., Varma, R., Zhao, L. S., Perillat, D., Carricato, G., Recknor, M., Du, K., Ho, H., Ellis, T., Gamez, J., Howes, M., Phi-Wilson, J., Lockard, S., Zuk, R., and Tan, H. (2009) Label-free detection of biomolecular interactions using BioLayer interferometry for kinetic characterization. Comb Chem High Throughput Screen 12, 791-800
166. Chao, G., Lau, W. L., Hackel, B. J., Sazinsky, S. L., Lippow, S. M., and Wittrup, K. D. (2006) Isolating and engineering human antibodies using yeast surface display. Nat Protoc 1, 755-768
167. Webb, B., and Sali, A. (2014) Comparative Protein Structure Modeling Using MODELLER. Curr Protoc Bioinformatics 47, 561-5632
168. Yang, J., and Zhang, Y. (2015) Protein Structure and Function Prediction Using I-TASSER. Curr Protoc Bioinformatics 52, 581-15
169. Kelley, L. A., Mezulis, S., Yates, C. M., Wass, M. N., and Sternberg, M. J. (2015) The Phyre2 web portal for protein modeling, prediction and analysis. Nat Protoc 10, 845-858
170. Bhatia, M., and Moochhala, S. (2004) Role of inflammatory mediators in the pathophysiology of acute respiratory distress syndrome. The Journal of pathology 202, 145-156
171. Belperio, J. A., Keane, M. P., Burdick, M. D., Lynch, J. P., 3rd, Xue, Y. Y., Berlin, A., Ross, D. J., Kunkel, S. L., Charo, I. F., and Strieter, R. M. (2001) Critical role for the chemokine MCP-1/CCR2 in the pathogenesis of bronchiolitis obliterans syndrome. The Journal of clinical investigation 108, 547-556
172. Belperio, J. A., Burdick, M. D., Keane, M. P., Xue, Y. Y., Lynch, J. P., Daugherty, B. L., Kunkel, S. L., and Strieter, R. M. (2000) The Role of the CC Chemokine, RANTES, in Acute Lung Allograft Rejection. The Journal of Immunology 165, 461-472
173. Belperio, J. A., Keane, M. P., Burdick, M. D., Gomperts, B. N., Xue, Y. Y., Hong, K., Mestas, J., Zisman, D., Ardehali, A., Saggar, R., Lynch, J. P., Ross, D. J., and Strieter, R. M. (2005) CXCR2/CXCR2 Ligand Biology during Lung Transplant Ischemia-Reperfusion Injury. The Journal of Immunology 175, 6931-6939
174. Ben-Abraham, R., Weinbroum, A. A., Dekel, B., and Paret, G. (2003) Chemokines and the inflammatory response following cardiopulmonary bypass—a new target for therapeutic intervention?—A review. Paediatr Anaesth 13, 655-661
175. Tu, R., Peng, Y., Wang, Y., Tang, X., and Wang, S. (2018) The stromal cell-derived factor 1/C-X-C chemokine receptor type 4 axis is important in neutrophil migration caused by cardiopulmonary bypass in children. Interact Cardiovasc Thorac Surg 26, 431-437
176. Morrison, M. I., Pither, T. L., and Fisher, A. J. (2017) Pathophysiology and classification of primary graft dysfunction after lung transplantation. J Thorac Dis 9, 4084-4097
177. Sadaria, M. R., Smith, P. D., Fullerton, D. A., Justison, G. A., Lee, J. H., Puskas, F., Grover, F. L., Cleveland, J. C., Jr., Reece, T. B., and Weyant, M. J. (2011) Cytokine expression profile in human lungs undergoing normothermic ex-vivo lung perfusion. Ann Thorac Surg 92, 478-484
178. Jansen, M. A., Otten, H. G., de Weger, R. A., and Huibers, M. M. (2015) Immunological and Fibrotic Mechanisms in Cardiac Allograft Vasculopathy. Transplantation 99, 2467-2475
179. Shimizu, K., and Mitchell, R. N. (2008) The role of chemokines in transplant graft arterial disease. Arteriosclerosis, thrombosis, and vascular biology 28, 1937-1949
180. Buch, M. H., Bingham, S. J., Bryer, D., and Emery, P. (2007) Long-term infliximab treatment in rheumatoid arthritis: subsequent outcome of initial responders. Rheumatology (Oxford) 46, 1153-1156
181. McInnes, I. B., and Schett, G. (2011) The pathogenesis of rheumatoid arthritis. The New England journal of medicine 365, 2205-2219
182. Feagan, B. G., Rutgeerts, P., Sands, B. E., Hanauer, S., Colombel, J. F., Sandborn, W. J., Van Assche, G., Axler, J., Kim, H. J., Danese, S., Fox, I., Milch, C., Sankoh, S., Wyant, T., Xu, J., Parikh, A., and Group, G. S. (2013) Vedolizumab as induction and maintenance therapy for ulcerative colitis. The New England journal of medicine 369, 699-710
183. Boder, E. T., and Wittrup, K. D. (1997) Yeast surface display for screening combinatorial polypeptide libraries. Nat Biotechnol 15, 553-557
184. Sleep, D., Cameron, J., and Evans, L. R. (2013) Albumin as a versatile platform for drug half-life extension. Biochim Biophys Acta 1830, 5526-5534
185. Skalko-Basnet, N. (2014) Biologics: the role of delivery systems in improved therapy. Biologics 8, 107-114

1-43. (canceled)

44. A composition of matter which comprises:
(i) a hybrid polypeptide comprising an amino acid sequence of a first tick CKBP polypeptide or a variant thereof and an amino acid sequence of a second tick CKBP polypeptide or a variant thereof, wherein said hybrid polypeptide has an altered chemokine binding profile compared to the first or second tick CKBP polypeptide;
(ii) a polypeptide comprising (a) all or part of an amino acid sequence shown in any one of SEQ ID NOs 45-72 or (b) all or part of an amino acid sequence having at least 70% homology or identity to a sequence of (a) over its entire length, wherein said polypeptide binds at least one CXC chemokine;
(iii) a polypeptide comprising (a) all or part of an amino acid sequence selected from SEQ ID NO: 88, 89 and 103 to 109 or (b) all or part of an amino acid sequence having at least 70% homology or identity to a sequence of (a) over its entire length, wherein said polypeptide binds at least one chemokine selected from CCL8, CCL7 and CCL18, preferably wherein said polypeptide binds all said chemokines, preferably wherein said polypeptide comprises all or part of an amino acid sequence shown in SEQ ID NO: 106;
(iv) a combination of two or more polypeptides according to (i), (ii) or (iii);
(v) a polynucleotide which encodes a polypeptide according to (i), (ii) or (iii) or a combination according to (iv);
(vi) a combination of two or more polynucleotides each of which encodes a polypeptide according to (i), (ii) or (iii);
(vii) a vector which comprises a polynucleotide according to (v) or a combination according to (vi);
(viii) a host cell which comprises a polynucleotide according to (v), a combination of two or more polynucleotides according to (vi) or a vector according to (vii);
(ix) a pharmaceutical composition comprising (a) a polypeptide according to (i), (ii) or (iii), a combination according to (iv) or (vi), a polynucleotide according to (v), a vector according to (vii) or a host cell according to (viii) and (b) a pharmaceutically acceptable carrier or diluent; or
(x) an antibody or a fragment thereof which specifically binds a polypeptide according to (i), (ii) or (iii).

45. A composition of matter according to claim 44(i), (iv), (v), (vi), (vii), (viii), (ix) or (x), where said first and second tick CKBP polypeptides comprise a CC chemokine-binding tick CKBP and a CXC chemokine binding tick CKBP.

46. A composition of matter according to claim 44(i), (iv), (v), (vi), (vii), (viii), (ix) or (x), wherein said hybrid polypeptide binds:
(a) at least one CC chemokine and at least one CXC chemokine;
(b) a reduced number of chemokines compared to said first and second tick CKBP polypeptides in combination; or
(c) all of the chemokines bound by the first and second tick CKBP polypeptides in combination.

47. A composition of matter according to claim 44(i), (iv), (v), (vi), (vii), (viii), (ix) or (x), wherein said hybrid polypeptide comprises:
(a) a fusion of said amino acid sequence of a first tick CKBP polypeptide or variant thereof and said amino acid sequence of a second tick CKBP polypeptide or a variant thereof; or
(b) a substitution of a chemokine-binding sequence of said second tick CKBP polypeptide or variant thereof into the amino acid sequence of said first tick CKBP polypeptide or variant thereof.

48. A composition of matter according to claim 44(i), (iv), (v), (vi), (vii), (viii), (ix) or (x), wherein said hybrid polypeptide comprises an amino acid sequence of at least one additional tick CKBP polypeptide or a variant thereof.

49. A composition of matter according to claim 44(i), (iv), (v), (vi), (vii), (viii), (ix) or (x), wherein:
(a) a said variant amino acid sequence comprises a part of the amino acid sequence of said tick CKBP polypeptide or an amino acid sequence having at least 70% homology or identity over its entire length to the whole or part of the amino acid sequence of said tick CKBP polypeptide; and/or
(b) said tick CKBP polypeptides are tick Evasin polypeptides.

50. A composition of matter according to claim 44(i), (iv), (v), (vi), (vii), (viii), (ix) or (x), wherein said hybrid polypeptide comprises:
(a) all or part of a first amino acid sequence selected from any one of SEQ ID NOs: 1 to 72 or all or part of an amino acid sequence having at least 70% homology or identity to said first amino acid sequence over its entire length; and
(b) all or part of a second amino acid sequence shown in any one of SEQ ID NOs: 1 to 72 and not selected in (a), or all or part of an amino acid sequence having at least 70% homology or identity to said second sequence over its entire length.

51. A composition of matter according to claim 50, wherein said polypeptide comprises:
(a) a first amino acid sequence selected from any one of SEQ ID NOs 1-3, 6-9, 20-23, 29, 32 and 34-44, and a second amino acid sequence is selected from any one of SEQ ID NOs 5, 18, 19, 33 and 45-72; or
(b) a first or second amino acid sequence selected from SEQ ID NO: 88, 89 and 103 to 109 or a variant thereof.

52. A composition of matter according to claim 44(i), (iv), (v), (vi), (vii), (viii), (ix) or (x), wherein said hybrid polypeptide comprises an amino acid sequence selected from any one of SEQ ID NOs 73, 74, 76-87, 92-93 and 95 or a variant thereof, optionally having at least 70% homology or identity to said amino acid sequence over its entire length.

53. A composition of matter according to claim 44(ii), (iv), (v), (vi), (vii), (viii), (ix) or (x), wherein a said polypeptide is in accordance with (ii) and said amino acid sequence of (a) is an amino acid sequence shown in any one of SEQ ID NOs 45-60 and 64-65, and wherein said polypeptide binds one or more human chemokines selected from CXCL7, CXCL9, CXCL10, CXCL11 and CXCL12, preferably wherein said sequence of (a) is an amino acid sequence shown in any one of SEQ ID NOs 45-48, 51-53, 56, 59, 60, and 65 and wherein said polypeptide binds one or more human chemokines selected from CXCL7, CXCL9, and CXCL11.

54. A composition of matter according to claim 44(iii), (iv), (v), (vi), (vii), (viii), or (ix), wherein a said polypeptide is in accordance with (iii) and wherein said polypeptide is in a cyclic or stapled form and/or is fused to a carrier, such as albumin.

55. A composition of matter according to claim 44(v), (vi), (vii), (viii), or (ix), wherein a said polynucleotide is in accordance with (vi) and wherein said polynucleotide is a ribonucleic acid modified to reduce immunogenicity and increase stability for instance by substitution of uridine and cytidine with 1-methylpseudouridine and 5-methylcytidine, and/or placing an Anti-Reverse Cap Analog (ARCA) cap at the 5′ end.

56. A method which is:
(a) a method of producing a polypeptide according to claim 44(i), (ii) or (iii) or a combination according to claim 44(iv) comprising culturing a host cell according to claim 44(viii) under conditions which produce the polypeptide or the combination; or
(b) a method of inhibiting the signalling of one or more chemokines in an in vitro culture, the method comprising contacting the culture with a polypeptide according to claim 44(i), (ii) or (iii), a combination according to claim 44(iv) or (vi), a polynucleotide according to claim 44(v), a vector according to claim 44(vii) or a host cell according to claim 44(viii).

57. A method of inhibiting the signalling of one or more chemokines in a subject, the method comprising administering to the subject a polypeptide according to claim 44(i), (ii) or (iii), a combination according to claim 44(iv) or (vi), a polynucleotide according to claim 44(v), a vector according to claim 44(vii) or a host cell according to claim 44(viii).

58. A method according to claim 57, wherein:
(a) the polypeptide is as defined in claim 44(iii), 51(b) or 54, and said method is for inhibiting the signalling of one or more of CCL8, CCL7 and CCL18 in a subject, preferably all said chemokines; or
(b) the method comprises administering a polypeptide comprising the amino acid sequence of SEQ ID NO: 92 or 93 or a variant thereof as defined in claim 52, or a polynucleotide encoding said polypeptide as defined in claim 44(v) or 55, and said method is for inhibiting the signalling of three or more of CCL2, CCL5, CCL8, CXCL8, CXCL10 and CXCL1 in a subject.

59. A method of treating or preventing in a subject one or more diseases associated with one or more chemokines, the method comprising administering to the subject a polypeptide according to claim 44(i), (ii) or (iii), a combination according to claim 44(iv) or (vi), a polynucleotide according to claim 44(v), a vector according to claim 44(vii) or a host cell according to claim 44(viii).

60. A method according to claim 59, wherein:
(a) the polypeptide, the combination, the polynucleotide, the vector or the host cell is administered in combination with another therapy;
(b) the disease comprises expression of CC and CXC chemokines and the polypeptide is a hybrid polypeptide according to claim 45 or 51(a);
(c) the one or more chemokines are selected from CXCL1, CXCL7, CXCL8, CXCL9, CXCL10, CXCL11 and CXCL2, and the polypeptide is a polypeptide according to claims 44(ii) or 53;
(d) the method comprises treating or preventing an inflammatory disease, or any disease selected from any one of myocarditis, myocardial infarction, atherosclerosis, vasculitis, stroke, multiple sclerosis, Alzheimer's disease, autoimmune hepatitis, primary biliary cirrhosis, primary schlerosing cholangitis, liver fibrosis, non alcoholic steatohepatitis, paracetamol liver injury, alcohol liver injury, idiopathic pulmonary fibrosis, acute lung injury, cardiac allograft vasculopathy, sarcoidosis, influenza, inflammatory bowel disease, pancreatitis, rheumatoid arthritis, psoriasis, skin fibrosis, breast cancer and colorectal cancer;
(e) the polypeptide is as defined in claim 44(iii), 51(b) or 54, and said method is for treating or preventing a disease selected from any one of alcoholic liver injury, Alzheimer's disease, atherosclerosis, atopic dermatitis, breast cancer, colorectal cancer, idiopathic pulmonary fibrosis, inflammatory bowel disease, influenza, kidney fibrosis, liver fibrosis, multiple sclerosis, myocardial infarction, myocarditis, non-alcoholic steatohepatitis, paracetamol liver injury, primary biliary cirrhosis, psoriasis, rheumatoid arthritis, sarcoidosis, skin fibrosis, stroke, vasculitis, acute lung injury; or
(f) the method comprises administering a polypeptide comprising the amino acid sequence of SEQ ID NO: 92 or 93 or a variant thereof as defined in claim 52, or a polynucleotide encoding said polypeptide as defined in claim 44(v) or 55, and said method is for treating or preventing a disease selected from any one of myocardial infarction, myocarditis, myocardial ischemia, and acute lung injury.

61. A method of detecting one or more chemokines in a tissue, comprising contacting the tissue with a detectably-labelled polypeptide according to claim 44 (i), (ii) or (iii) or a detectably-labelled combination according to claim 44(iv) and detecting the binding of the polypeptide or the combination to one or more chemokines.

62. A method according to claim 61, wherein:
(a) the one or more chemokines are selected from any chemokines shown in Tables 2-4 and 6; or
(b) the method is for diagnosing or prognosing one or more diseases associated with one or more chemokines.


SEQUENCE LISTING (112 sequences)

SEQ ID NO: 1 - protein, 106 aa, Rhipicephalus pulchellus
AEKSLDSDSSGEDYELWTQGCPFLVAENRTGFGTTVSCQHNCNGAIEKVPEGEPCYTIGEDGLGRMKLNLPYNCSLGECSGGVCVPNGRSDVCFKRTWEENNKAMA

SEQ ID NO: 2 - protein, 97 aa, Amblyomma cajennense
ENTQQEEEDYDYGTDTCPFPVLANKTNKAKFVGCHQKCNGGDQKLTDGTACYVVERKVWDRMTPMLWYSCPLGECKNGVCEDLRKKEECRKGNGEEK

SEQ ID NO: 3 - protein, 104 aa, Rhipicephalus pulchellus
VCEVSEQEGVGEDNATEDEDYEDFFKPVTCYFANSTVGPLRPPNCTVVCTNNTAWWNDTKSDGGHCYSEYRPEKRTHSREIYNCTIGVCGNGTCIANHTYADCW

SEQ ID NO: 4 - protein, 89 aa, Rhipicephalus sanguineus
NEDDSSDYYDASPMNCSSMSVNSTMGWLSMNCTMSCNGTTFPLSNSTHCFHSYTNLTVQSRMETMTYNCSVGTCSNGTCVENGTTTTCW

SEQ ID NO: 5 - protein, 64 aa, Ixodes ricinus
RSKQPTASQSSKNSIKAEFCDTNCTQGTNGAWSGCSEGCFCVHVGNNTKGRCMKLSSDYDYTTQ

SEQ ID NO: 6 - protein, 97 aa, Amblyomma cajennense
ENTQQEEQDYDYGTDTCPFPVLANKTNKAKFVGCHQKCNGGDQKLTDGTACYVVERKVWDRMTPMLWYECPLGECKNGVCEDLRKKEDCRKGNGEEK

SEQ ID NO: 7 - protein, 98 aa, Amblyomma cajennense
EDTGTEDDFDYGNTGCPFPVLGNYKSNMTKPVGCKNKCGSGYEVLNDTTPCYVIDQKVFNNMVPLRQYSKCPLGFCENGECKPNDQAEDCYKGREEQK

SEQ ID NO: 8 - protein, 108 aa, Amblyomma parvum
DEESEELGASTDVDYEELDANCTCPAPALTSTRNNKHYPLGCIYNCSSYNCTIPDGTPCYVLTLGEVKEHLQIGSTVPNCTCGLCRNGTCVSNGTVEECFAVEEIEET

SEQ ID NO: 9 - protein, 108 aa, Amblyomma cajennense
ENGEGTTQPDYDNSTDYYNYEDFKCTCPAPHLNNTNGTVMKPIGCYYTCNVTRCTAPDTYPCYNLTEHQAKNLTTSPTTLCAVGNCDHGICVPNGTKELCFKAPNLEE

SEQ ID NO: 10 - protein, 94 aa, Amblyomma parvum
HRHYDPNEPCLIAGLKTPRDALPAGCRYDCMIKKNQKLRDGLLCLDVPEKVVKRMVNYLNYSCPLGTCRKGICKRKHRNVRCQKYPVFYMSPPK

SEQ ID NO: 11 - protein, 133 aa, Amblyomma maculatum
EKDTPKNIPGCGDTGTTAAPPADNPKHFAVYTDKHGCTIKVIGTWMTKEDHSRLPVSLRRVRASSGRVMLPASCQKICNDTVKNFPEGTPCRLVTGDPIKGKNHIKDGCIRGYCSSGVCVSDKRNISCYVPPN

SEQ ID NO: 12 - protein, 143 aa, Amblyomma cajennense
DTIGGIPGCGDPATTSAPEDQPKHYAVRTDKNGCKVMVIGTWMTKEDHNLLPPYISKERAPSGKVQLPASCKKNCHGKLKNLPNGTPCREVFGDLRRRRKHIKDGCKVGKCQNGLCVSEERIISCYLPPNITDPRPTHGPLAE

SEQ ID NO: 13 - protein, 146 aa, Amblyomma triste
EKDTPKNIPGCEDAGTTAAPPADNPKHYAVYTDKNGCTFKVIGTWMTKEDHRRLPVSLRRVRASSGRVKLPASCQKICKHSVKKFPEGTPCRLVTRDPIKGKNHIKNGCIRGYCSSGVCVSDNRSISCYVLPNNTELTSPSGSFAE

SEQ ID NO: 14 - protein, 143 aa, Amblyomma cajennense
DIIGGIPGCGDPTTTSAPEVPPKHFAVRTDKDGCTLMIIGTWMTTEDHNRLPPDIGEKRAPYGRVQLPASCKKNCHGKVKNLPNGTPCREVFGDPRRGRKHIKNGCTVGKCQSGLCVTDKRIISCYIPPNITDPIPTHGPWAE

SEQ ID NO: 15 - protein, 117 aa, Amblyomma cajennense
EEAPGPAPGCGEPEPTPPKPRRHGIVTNVNSCNSTILVWNGKEFPALCKVRCPHKSYRVSDFEPCLKFTNRRFLQERKDETPYKCKLGFCRHGTCITSEHSRKVPCKVPADRLDPSE

SEQ ID NO: 16 - protein, 148 aa, Amblyomma parvum
GPPSIPGNESIPGCGDAGTTTAPEDNPKHYGTLTDKKGCTLPIIGTWMTAVDHQHLQGTRGERRGPTGKVNLPASCRKNCHGRQEILRDGIPCRKVVGNPKGSKKHLKSGCLRGKCLAGQCVNDGRRISCYVPRNITDTEPTPGLLAE

SEQ ID NO: 17 - protein, 146 aa, Amblyomma triste
QSEVGKNVPGCGDTETLEAPPQEKPPYYEYKDEEGCTQKVLESWFQAGERSTGRGQKKRGHGRRPSRVLRTVDCRKNCTVGITALPDGHLCLVPRGDPFTRGGAIKYGCYLGDCASGHCQHRYETVSCRLPAPDTTAKPYYVTPEK

SEQ ID NO: 18 - protein, 88 aa, Ixodes ricinus
GPDTKGDEESDENELFTVEYCGTNCTQLENGSWTPCSGNNGNCRCFHESDKTVGLCLSTEYTDFSEYPDPNSSEIIAAAPLPRERLIQ

SEQ ID NO: 19 - protein, 80 aa, Ixodes ricinus
ADDDNELFTVQYCGMNCTKDEGGTWTGCTGKKEGCKCYHESGKNYGLCLSTEYTDFSQYGNPSDSEIEAAKPKRSDTLSH

SEQ ID NO: 20 - protein, 91 aa, Amblyomma triste
EEPKDGYDYTEGCPFVVLGNGTHAKPAGCSHLCNGAPETLDDNMECYNVTEEVAKRMTPDIPYTCWLGWCSKGECKRDNRTEVCYRGSERE

SEQ ID NO: 21 - protein, 90 aa, Amblyomma maculatum
EEREDDNDYGGGCPFVVLGNGTHAKPAGCSHLCNGAPETLDNIECYNVTEEVAKRMTPDIPYTCWLGWCSKGECKRDNRTEVCYRGSERE

SEQ ID NO: 22 - protein, 89 aa, Amblyomma maculatum
EPKDDNDYGGGCPFVVLGNGTHAKPAGCSHLCNGAPETLDNIECYNVTEEVAKRMTPGIPYACWLGWCNKGECKRGNRTEVCYRGSEEE

SEQ ID NO: 23 - protein, 90 aa, Amblyomma triste
EAPKDDFEYDGGCPFVVLDNGTHVKPAGCSHLCNGAPETLDNIECYNVTEEVAKRMTPGIPYACWLGWCSKGECKRDNRTEVCYRGSEEE

SEQ ID NO: 24 - protein, 159 aa, Amblyomma parvum
KTDTKNAAGELPPKVAIPGCEDPATTKAPLPDDPRYYGVTIDKDGCQRKVLGSSQRQQRQVQNGRKPGRKGRGRRPVFVDLKLTVDCKRKCNGTYSQLPDGEPCLVCDGEPYGRHRTIKGGCYQGNCSSGQCHRGERKVNCYIPKNITNNVLNSVNLAE

SEQ ID NO: 25 - protein, 159 aa, Amblyomma parvum
KTDTKNAAGELPPKVVIPGCEDPATTKAPLPDEPPYYGITIDKDGCQRKVLGSSQRQQRKVQNGRKLDGKKRGRRPVFVDLELTVDCKRKCNGTYSQLPDGENCLVSDGYPYGRWGTIKGGCYQGNCSSGQCHRGEKKVNCYLPKNITNDVPKSLNLAE

SEQ ID NO: 26 - protein, 141 aa, Ixodes ricinus
ASLAKETEDTTLPARALVDSPDSDNCSSPQLPYFDELTYMPLGFLAVNCTKTCPVGKNGTVVNGNKCIVTWSILDVSTITVLVGSCKNGYCISDGSSECRNITLAGEDSQEEEEEAEEDEEEDDGDEEEDEEEDEEENDDD

SEQ ID NO: 27 - protein, 119 aa, Ixodes ricinus
GSTPSAMKTEDILKVLGSTSSLENHTDSSHCRYQELLDITKNIENAGFLAINCQRSCPNGKQTMVEGYGCIFKIKHATKRGKVKVKEGSCRKGACVRGSTRPPWRLLVLLGESKEEEFL

SEQ ID NO: 28 - protein, 136 aa, Ixodes ricinus
SDLCKMEAESSPFKLPQSSLLDAPDEEGCKYQLLFVEAEGPLVVNCTKDCPNGKIRTVVEGELCIAMVKTSSSGEATGLVGSCKRGSCVKKDDPCRTFTLSEEGDDDEEDEEEEEDEEEDEEEDEEEEEEDEEEED

SEQ ID NO: 29 - protein, 98 aa, Amblyomma americanum
RNHTEDNSTEYYDYEEARCACPARHLNNTNGTVLKLLGCHYFCNGTLCTAPDGYPCYNLTAQQVRTLTTYPNTSCAVGVCMKGTCVKNGTMEQCFKTP

SEQ ID NO: 30 - protein, 151 aa, Amblyomma americanum
RGGAASVPANASIPGCGDAQTTPAAPEDQPKHYVVYRDGNGCEVKIIGTWMTTEDYNCLPDTFRKNRAPHGKVKLPASCKKTCGNAVQNLKDGTPCRKVFGDLGRRRNLIKNGCLVGACQSGLCVSGNRTISCYIPPNSTDTRATPGSFAE

SEQ ID NO: 31 - protein, 140 aa, Ixodes ricinus
KEPEDTTLPPGALVDSPDSDNCSSPHLPYFDETTNMWMGFLAVNCTKKCPVGKHVTVVDGNKCIGTWSFLDELTITVLVGSCKDGFCETDGSSECRNITLAEEDSQEEEGAAAEEEDEEDEEDEEEEEAEEEREDDHDDA

SEQ ID NO: 32 - protein, 94 aa, Rhipicephalus sanguineus
EDDEDYGDLGGCPFLVAENKTGYPTIVACKQDCNGTTETAPNGTRCFSIGDEGLRRMTANLPYDCPLGQCSNGDCIPKETYEVCYRRNWRDKKN

SEQ ID NO: 33 - protein, 66 aa, Rhipicephalus sanguineus
LVSTIESRTSGDGADNFDVVSCNKNCTSGQNECPEGCFCGLLGQNKKGHCYKIIGNLSGEPPVVRR

SEQ ID NO: 34 - protein, 104 aa, Rhipicephalus sanguineus
EVPQMTSSSAPDLEEEDDYTAYAPLTCYFTNSTLGLLAPPNCSVLCNSTTTWFNETSPNNASCLLTVDFLTQDAILQENQPYNCSVGHCDNGTCAGPPRHAQCW

SEQ ID NO: 35 - protein, 69 aa, Ixodes ricinus
GPAPSAKENEKAPLCLPQESLINNRDPNGCNYQLLPYFTEDGMGGGFLAIDCSKSCPEGTHETVVDGNS

SEQ ID NO: 36 - protein, 71 aa, Ixodes holocyclus
AFVSSSTEVEIGSTSEHNSNETDEYGYDYNADGLGCPVVGIGGLDNKTWHPNCTNECPNSTKLFLLENGTP

SEQ ID NO: 37 - protein, 125 aa, Amblyomma triste
GNEVSDPPLTDEDCEYYDPSEDNITCSIRSLNTTGRPIPVGCLATCENSTRRLHNGTECLGISDQVANRMQGNVTYTCPVGLCYRGVCQRNGLGIDCWHNTPPPNSTNVTTNASTTPLPTSSRDL

SEQ ID NO: 38 - protein, 126 aa, Amblyomma maculatum
ECEESDTSESTECSTEDYSNRIRDNETCFIGALNTTGHPVPVGCTLDCGNSTRYLPNGTECIDLTQQASDVMQSDVPYYCPIGLCANGICKRSGLELNCWHDMPPPVSTDTAIENPTTSISSSAKL

SEQ ID NO: 39 - protein, 122 aa, Amblyomma cajennense
GIEGSGNLATSHEMDDCLDDNSTCVIQTLNTTGEPRPVGCVLKCKNSTQHLANGTECLGIPELAGVRMQYNVSYTCAVGLCNAGVCERTGLWIGCWQNEPPPNSTDVTTTAPTTTTASTSSV

SEQ ID NO: 40 - protein, 97 aa, Amblyomma cajennense; identical to SEQ ID NO: 6

SEQ ID NO: 41 - protein, 98 aa, Amblyomma americanum; identical to SEQ ID NO: 29

SEQ ID NO: 42 - protein, 125 aa, Amblyomma americanum
ESEGSVSTETEVISYEDDCQDDNSTCFIQTLNTTGEPRPVGCILECENSTQRLPNGTECLGLPGLAAVKMQRNVSYTCSVGLCNGEGVCDRTGLWIGCWTNTPPPNSTNVTTKPPTTTTASPGTG

SEQ ID NO: 43 - protein, 111 aa, Rhipicephalus pulchellus
CEVQNTTLAEEDYDTGCGYNIVITKNKTLVVNCTMDCQPKMLMNESEPCLFNSSVPYDHMQPHHNYTCMEGICKNGTCVSPSNNITCWLPPPPVRYYPNETMVTSTIEPEA

SEQ ID NO: 44 - protein, 106 aa, Rhipicephalus pulchellus; identical to SEQ ID NO: 1

SEQ ID NO: 45 - protein, 66 aa, Ixodes ricinus
GSKQPGAAGSSSDSVEAVFCPTNCTKGTNGAWSGCSDDCICVHVGENTEGSCMKFSGDYDYPTPEA

SEQ ID NO: 46 - protein, 68 aa, Ixodes ricinus
GSNQLSGPQSSANSNDAVFCDTNCTQGTDGAWSGCRGDCFCVHVGNSTEGRCIELIGDFDYSTPGAED

SEQ ID NO: 47 - protein, 66 aa, Ixodes ricinus
NQLSGPQSSANSNEAVFCDTNCTQGTDEAWSGCRGDCFCVYVGNSTEGRCMMLSGDFDYSTPGAED

SEQ ID NO: 48 - protein, 68 aa, Ixodes ricinus
GSKESSAHQSSDDSIKAEFCDAKCTMKTDGKWTQCHGGCFCVHVGNETEGRCMRLDGDYDYPSTQPEE

SEQ ID NO: 49 - protein, 68 aa, Ixodes ricinus
GSKQLIGPQSSTNSIKAEFCDTNCTAGTNGIWNGCSGDCFCTHVGNSTEGRCMKITGFDEYPTSEAEE

SEQ ID NO: 50 - protein, 68 aa, Ixodes ricinus
GSKGSSAQQSSHDSIKAEFCETNCTMKTGGKWTQCHGGCFCVHVGNETVGRCIKLDGDYDYPSSKHEE

SEQ ID NO: 51 - protein, 99 aa, Ixodes ricinus
SAGSKGSSAPQSSGDSVVAEFCDTNCTMKKDGKWTECNGDCFCVHVGNETVGRCMRLDGDYDYTSSKTTRRNKKTRNGLCRLDRNRTTVDYPERNTREP

SEQ ID NO: 52 - protein, 66 aa, Ixodes ricinus
GSKGQRASQVSETSITAEFCDTSCTQGTDKTWSGCSGDCFCVHVGNDTEGRCMRWDGDYPSAEEEE

SEQ ID NO: 53 - protein, 61 aa, Ixodes ricinus
GSKELSGPESSENSIEAAFCDTNCTEGTDGVWSGCSAGCFCVHVGNSTVGRCMTFNGVDGG

SEQ ID NO: 54 - protein, 89 aa, Ixodes ricinus
HSPVAGSEVQKLTSDPNDDIDVSYCGMNCTVVNGKSDECSENCKCLHEGDDPKGICVAITYFGDWGDPNDDPKINEATPQTQIFEKKRK

SEQ ID NO: 55 - protein, 91 aa, Ixodes ricinus
HTTVTGSVEGKPNNPNEDIEVSYCRMNCTVENGVSSACSGDCVCVHRDNEPNGICVEITYFGDFGDPSQDPSIDEAAPRESVSKRRSNGES

SEQ ID NO: 56 - protein, 68 aa, Ixodes ricinus
GSKGSSASQSSDNSVVAKFCDTNCTINEGGKWTECKGGCFCVHVGNETVGRCMKLDGDYDYPSPKPEE

SEQ ID NO: 57 - protein, 79 aa, Ixodes ricinus
HTTVAGSDEDIEVSYCGMNCTVESGKSSKCSPDCVCVHEGNERDGICISITYLGDLGNPLEDPSIDLATPLAPVFQSSK

SEQ ID NO: 58 - protein, 66 aa, Ixodes ricinus
ESKEASASQGPGKSFKVEFCETNCTENNGVWSGCTGDCICVSVGDSKEGRCMDLGDKVIDTPVAQG

SEQ ID NO: 59 - protein, 55 aa, Ixodes ricinus
DSKGTSDSQDSTKSIKVDFCETNCTKTDGGWTGCTGDCICVSVGDSIEGRCMDFG

SEQ ID NO: 60 - protein, 65 aa, Amblyomma cajennense
KPQILQRTDKSTDSEWDPQTCPETCIPSKNITCSDGCVCVKLGEEEEGTCFNMTGVDWLGSPSDD

SEQ ID NO: 61 - protein, 80 aa, Ixodes ricinus
AGKDDEHFSVDYCGMNCTQQEDGSWTACSGRNGECRCYHESGKRSGLCLSTTYIDFSEYGNLSDSDIAAASPRLSMKESH

SEQ ID NO: 62 - protein, 72 aa, Ixodes ricinus
KDDEHFSVDYCGMNCTQQEDGSWTACSGRNEECRCYHESGKKNGLCLSTTYIDFSQYGNPSDSDIAAASPRP

SEQ ID NO: 63 - protein, 78 aa, Ixodes ricinus
LSDEDELFSVEYCGTNCTKQDTGSWTTCSGNCTCYHEDGKKVGLCLSTEYTDFTKFPKPTSEEIANARPLPKREKTLN

SEQ ID NO: 64 - protein, 104 aa, Ixodes ricinus
NEEVFTVEYCGMNCTQKSDGTWTECSGKNKDCRCYHESDAREGLCLSTEYTDFSQFETPSNSDLEAATPRPRKTLYPVRNPHGPKTRGLGYDKRILRDRVKFLI

SEQ ID NO: 65 - protein, 72 aa, Amblyomma cajennense
KPQILQRTDHSTDSDWDPQMCPETCNPSKNISCSSECLCVTLGGGDETGTCFNMSGVDWLGHAQASDGHNDG

SEQ ID NO: 66 - protein, 71 aa, Ixodes ricinus
LNDEELFTVDYCGTNCTQQPNGSWTTCPGNCSCYHEDGKTDGFCLSTEYTDFTQFPNLTSEEMDAATPRPE

SEQ ID NO: 67 - protein, 86 aa, Ixodes ricinus
GPETKEDKKSDVYELFTVEYCGTNCTLLTNGRWTACTGKKGTCRCYHESGEKVGLCLSTEYTDFSEYPNPKSSEIDAAAPLPRETH

SEQ ID NO: 68 - protein, 82 aa, Ixodes ricinus
GQDTDGKEKSDEYELFTVEYCGTNCTQLENGSWTACTGKNGTCRCFHENDKKVGLCLSTEYTDFSEYPDPNSEEIKAASPLP

SEQ ID NO: 69 - protein, 74 aa, Ixodes ricinus
LGDEDQLFSVEYCGTNCTQQDDGKWTPCSGKNGKCKCYHEDGKRYGLCLYTEYTDFSQYPNPEGSEIENTRPRP

SEQ ID NO: 70 - protein, 71 aa, Ixodes ricinus
LHEDEIFTVDYCGTNCTKQSNGSWTTCPGNCSCYHEDGKTDGFCLSTEYTDFTQFPNLTSEEMDAATPRPE

SEQ ID NO: 71 - protein, 77 aa, Ixodes ricinus
LNNENELFSVEYCGANCTQQDNGSWTKCKGNCTCYHEDGKRYGLCLSTEYTDFTQFPKPTSEEIADASPRPKETNSH

SEQ ID NO: 72 - protein, 77 aa, Ixodes ricinus
DDEFFTVDYCGMNCTLQQDGSWTPCTQKNAECKCYHESGSSVGLCLSTAYTDFNQFGDPNNSDLDAATPRHPDASSR

SEQ ID NO: 73 - protein, 193 aa, synthetic construct
  REGION 1..193: note = hybrid polypeptide
ENGEGTTQPDYDNSTDYYNYEDFKCTCPAPHLNNTNGTVMKPIGCYYTCNVTRCTAPDTYPCYNLTEHQAKNLTTSPTTLCAVGNCDHGICVPNGTKELCFKAPNLEEGGGGSADDDNELFTVQYCGMNCTKDEGGTWTGCTGKKEGCKCYHESGKNYGLCLSTEYTDFSQYGNPSDSEIEAAKPKRSDTLSH

SEQ ID NO: 74 - protein, 183 aa, synthetic construct
  REGION 1..183: note = hybrid polypeptide
RNHTEDNSTEYYDYEEARCACPARHLNNTNGTVLKLLGCHYFCNGTLCTAPDGYPCYNLTAQQVRTLTTYPNTSCAVGVCMKGTCVKNGTMEQCFKTPGGGGSADDDNELFTVQYCGMNCTKDEGGTWTGCTGKKEGCKCYHESGKNYGLCLSTEYTDFSQYGNPSDSEIEAAKPKRSDTLSH

SEQ ID NO: 75 - protein, 5 aa, synthetic construct
  REGION 1..5: note = Linker sequence
GGGGS

SEQ ID NO: 76 - protein, 110 aa, synthetic construct
  REGION 1..110: note = hybrid polypeptide comprising a substitution of an amino acid sequence of a second tick CKBP polypeptide into the amino acid sequence of a first tick CKBP polypeptide
VCEVSEQEGVGEDNATEDEDYEDFFKPVTCYFANSTVGPLRPPNCKQDCNGTTETAPNGTRCFSIGDEGLRRMTANLPYDCPLGQCSNGDCIPKETYEVCYRRNWRDEKN

SEQ ID NO: 77 - protein, 44 aa, synthetic construct
  REGION 1..44: note = chemokine binding sequence
VCEVSEQEGVGEDNATEDEDYEDFFKPVTCYFANSTVGPLRPPN

SEQ ID NO: 78 - protein, 66 aa, synthetic construct
  REGION 1..66: note = residual recipient sequence
CKQDCNGTTETAPNGTRCFSIGDEGLRRMTANLPYDCPLGQCSNGDCIPKETYEVCYRRNWRDEKN

SEQ ID NO: 79 - protein, 28 aa, synthetic construct
  REGION 1..28: note = chemokine-binding sequence
EDDEDYGDLGGCPFLVAENKTGYPTIVA

SEQ ID NO: 80 - protein, 193 aa, synthetic construct
  REGION 1..193: note = hybrid polypeptide
ADDDNELFTVQYCGMNCTKDEGGTWTGCTGKKEGCKCYHESGKNYGLCLSTEYTDFSQYGNPSDSEIEAAKPKRSDTLSHGGGGSENGEGTTQPDYDNSTDYYNYEDFKCTCPAPHLNNTNGTVMKPIGCYYTCNVTRCTAPDTYPCYNLTEHQAKNLTTSPTTLCAVGNCDHGICVPNGTKELCFKAPNLEE

SEQ ID NO: 81 - protein, 183 aa, synthetic construct
  REGION 1..183: note = hybrid polypeptide
ADDDNELFTVQYCGMNCTKDEGGTWTGCTGKKEGCKCYHESGKNYGLCLSTEYTDFSQYGNPSDSEIEAAKPKRSDTLSHGGGGSRNHTEDNSTEYYDYEEARCACPARHLNNTNGTVLKLLGCHYFCNGTLCTAPDGYPCYNLTAQQVRTLTTYPNTSCAVGVCMKGTCVKNGTMEQCFKTP

SEQ ID NO: 82 - protein, 113 aa, synthetic construct
  REGION 1..113: note = tick CKBP sequence
VCEVSEQEGVGEDNATEDEDYEDFFKPVTCYFANSTVGPLRPPNCTVVCTNNTAWWNDTKSDGGHCYSEYRPEKRTHSREIYNCTIGVCGNGDCIPKETYEVCYRRNWRDEKN

SEQ ID NO: 83 - protein, 114 aa, synthetic construct
  REGION 1..114: note = tick CKBP sequence
VCEVSEQEGVGEDNATEDEDYEDFFKPVTCYFANSTVGPLRPPNCTVVCTNNTAWWNDTKSDGGHCFSIGDEGLRRMTANLPYDCPLGQCSNGDCIPKETYEVCYRRNWRDEKN

SEQ ID NO: 84 - protein, 92 aa, synthetic construct
  REGION 1..92: note = introduced and recipient tick CKBP sequence
VCEVSEQEGVGEDNATEDEDYEDFFKPVTCYFANSTVGPLRPPNCTVVCTNNTAWWNDTKSDGGHCYSEYRPEKRTHSREIYNCTIGVCGNG

SEQ ID NO: 85 - protein, 21 aa, synthetic construct
  REGION 1..21: note = introduced and recipient tick CKBP sequence
DCIPKETYEVCYRRNWRDEKN

SEQ ID NO: 86 - protein, 66 aa, synthetic construct
  REGION 1..66: note = introduced and recipient tick CKBP sequence
VCEVSEQEGVGEDNATEDEDYEDFFKPVTCYFANSTVGPLRPPNCTVVCTNNTAWWNDTKSDGGHC

SEQ ID NO: 87 - protein, 48 aa, synthetic construct
  REGION 1..48: note = introduced and recipient tick CKBP sequence
FSIGDEGLRRMTANLPYDCPLGQCSNGDCIPKETYEVCYRRNWRDEKN

SEQ ID NO: 88 - protein, 16 aa, synthetic construct
  REGION 1..16: note = P672_PEP
EDEDYEDFFKPVTAYF

SEQ ID NO: 89 - protein, 16 aa, synthetic construct
  REGION 1..16: note = variant
EDEDYEDFFKPVTCYF

SEQ ID NO: 90 - protein, 16 aa, synthetic construct
  REGION 1..16: note = P672_PEP-FITC
  SITE 1: note = MISC_FEATURE - N-terminally labelled with FITC
EDEDYEDFFKPVTAYF

SEQ ID NO: 91 - protein, 16 aa, synthetic construct
  REGION 1..16: note = P672_PEP_SCRAM
EFTEVYEFDFKYDAPD

SEQ ID NO: 92 - protein, 268 aa, synthetic construct
  REGION 1..268: note = P1820 - Three warhead evasin
KPQILQRTDHSTDSDWDPQMCPETCNPSKNISCSSECLCVTLGGGDETGTCFNMSGVDWLGHAQASDGHNDGGGGGSADDDNELFTVQYCGMNCTKDEGGTWTGCTGKKEGCKCYHESGKNYGLCLSTEYTDFSQYGNPSDSEIEAAKPKRSDTLSHGGGGSAEKSLDSDSSGEDYELWTQGCPFLVAENRTGFGTTVSCQHNCNGAIEKVPEGEPCYTIGEDGLGRMKLNLPYNCSLGECSGGVCVPNGRSDVCFKRTWEENNKAMA

SEQ ID NO: 93 - protein, 270 aa, synthetic construct
  REGION 1..270: note = P1821 - Three warhead evasin
KPQILQRTDHSTDSDWDPQMCPETCNPSKNISCSSECLCVTLGGGDETGTCFNMSGVDWLGHAQASDGHNDGGGGGSADDDNELFTVQYCGMNCTKDEGGTWTGCTGKKEGCKCYHESGKNYGLCLSTEYTDFSQYGNPSDSEIEAAKPKRSDTLSHGGGGSENGEGTTQPDYDNSTDYYNYEDFKCTCPAPHLNNTNGTVMKPIGCYYTCNVTRCTAPDTYPCYNLTEHQAKNLTTSPTTLCAVGNCDHGICVPNGTKELCFKAPNLEE

SEQ ID NO: 94 - protein, 194 aa, synthetic construct
  REGION 1..194: note = hybrid polypeptide
ADDDNELFTVQYCGMNCTKDEGGTWTGCTGKKEGCKCYHESGKNYGLCLSTEYTDFSQYGNPSDSEIEAAKPKRSDTLSHGGGGSENGEGTTQPDYDNSTDYYNYEDFKCTCPAPHLNNTNGTVMKPIGCYYTCNVTRCTAPDTYPCYNLTEHQAKNLTTSPTTLCAVGNCDHGICVPNGTKELCFKAPNLEEG

SEQ ID NO: 95 - protein, 97 aa, synthetic construct
  REGION 1..97: note = EVA1 containing P672 E22-E32
EDDEDYEDFFKPVTCYFLVAENKTGYPTIVACKQDCNGTTETAPNGTRCFSIGDEGLRRMTANLPYDCPLGQCSNGDCIPKETYEVCYRRNWRDKKN

SEQ ID NO: 96 - protein, 10 aa, synthetic construct
  REGION 1..10: note = BK2 peptide
EDEDYEDFFK

SEQ ID NO: 97 - protein, 13 aa, synthetic construct
  REGION 1..13: note = BK3 peptide
EDEDYEDFFKPVT

SEQ ID NO: 98 - protein, 6 aa, synthetic construct
  REGION 1..6: note = BK4 peptide
DYEDFF

SEQ ID NO: 99 - protein, 10 aa, synthetic construct
  REGION 1..10: note = BK5 peptide
DYEDFFKPVT

SEQ ID NO: 100 - protein, 13 aa, synthetic construct
  REGION 1..13: note = BK6 peptide
DYEDFFKPVTAYF

SEQ ID NO: 101 - protein, 8 aa, synthetic construct
  REGION 1..8: note = BK7
  SITE 1: note = MISC_FEATURE - N-terminally labelled with FITC
EDEDYEDF

SEQ ID NO: 102 - protein, 8 aa, synthetic construct
  REGION 1..8: note = BK8
FKPVTAYF

SEQ ID NO: 103 - protein, 12 aa, synthetic construct
  REGION 1..12: note = Y21F32 peptide
YEDFFKPVTAYF

SEQ ID NO: 104 - protein, 12 aa, synthetic construct
  REGION 1..12: note = Y21 F32 C30A peptide
YEDFFKPVTCYF

SEQ ID NO: 105 - protein, 17 aa, synthetic construct
  REGION 1..17: note = BK1.2 peptide
  REGION 1..15: note = MISC_FEATURE - Tyr-1 to Cys-15 thioether cyclization
YEDEDYEDFFKPVTCYF

SEQ ID NO: 106 - protein, 17 aa, synthetic construct
  REGION 1..17: note = BK1.3 peptide
YEDEDYEDFFKPVTCYF

SEQ ID NO: 107 - protein, 16 aa, synthetic construct
  REGION 1..16: note = BK1.4 peptide
  REGION 1..14: note = MISC_FEATURE - Glu-1 to Cys-14 thioether cyclization
EDEDYEDFFKPVTCYF

SEQ ID NO: 108 - protein, 11 aa, synthetic construct
  REGION 1..11: note = E22-F32 peptide from P672_RHIPU
EDFFKPVTCYF

SEQ ID NO: 109 - protein, 17 aa, synthetic construct
  REGION 1..17: note = BK1.3 peptide dimer
  DISULFID 15: note = Cys-15 disulfide with second BK1.3 peptide
YEDEDYEDFFKPVTCYF

SEQ ID NO: 110 - protein, 90 aa, unidentified
  REGION 1..90: note = CCL8 protein sequence
QPDSVSIPITCCVINFNRKIPIQRLESYTRITNIQCPKEAVIFKTKRGKEVCADPKERWVRDSMKHLDQIFQNLKPSSKGGYGLNDIFEA

SEQ ID NO: 111 - protein, 130 aa, synthetic construct
  REGION 1..130: note = Synthetic construct of P672_RHIPU
ETGHHHHHHHHSAWSHPQFEKGGGGSVCEVSEQEGVGEDNATEDEDYEDFFKPVTCYFANSTVGPLRPPNCTVVCTNNTAWWNDTKSDGGHCYSEYRPEKRTHSREIYNCTIGVCGNGTCIANHTYADCW

SEQ ID NO: 112 - protein, 97 aa, unidentified
  REGION 1..97: note = CCL8 protein sequence
QPDSVSIPITCCVINFNRKIPIQRLESYTRITNIQCPKEAVIFKTKRGKEVCADPKERWVRDSMKHLDQIFQNLKPSSKGGYGLNDIFEAQKIEWHE